(19) United States
(12) Patent Application Publication
(10) Pub. No.: US 2003/0073219 A1
(43) Pub. Date: Apr. 17, 2003

(54) Crystal structure

(76) Inventors: Tom L. Blundell, Royston (GB); Christopher Abell, Cambridge (GB); Dijana Matak-Vinkovic, Zagreb (HR)

Correspondence Address: NIXON & VANDERYE P.C., 8th Floor, 1100 North Glebe Road, Arlington, VA, 22201-4714, US

(21) Appl. No.: 09/860,760

(22) Filed: May 21, 2001

(51) Int. Cl. G06F 19/00; G01N 33/48; G01N 33/50; C12N 9/02

(52) U.S. Cl. 435/189; 702/19

(57) ABSTRACT

The invention relates to crystals of the enzyme ketopantoate reductase, and in particular its crystal structure and the use of this structure in drug discovery.

### FIELD OF THE INVENTION

The present invention relates to the enzyme ketopantoate reductase, and in particular its crystal structure and the use of this structure in drug discovery.

### BACKGROUND OF THE INVENTION

Pantothenic acid (vitamin B₅) is found in coenzyme A (CoA) and the acyl carrier protein (ACP), both of which are involved in fatty acid metabolism.

Pantothenic acid can be synthesised by plants and microorganisms but animals are apparently unable to make the vitamin, and require it in their diet. However, all organisms are able to convert pantothenic acid to its metabolically active form, coenzyme A.

The pathway for the synthesis of pantothenic acid is shown in FIG. 1. It provides a potential target for the treatment of infectious disease, since inhibitors of the pathway should be damaging to bacteria and fungi but not to human or animal subjects infected by bacteria.

Of specific interest is the enzyme ketopantoate reductase. Ketopantoate reductase (EC 1.1.1.169, KPR, PanE) is the third enzyme on the biosynthetic pathway to pantothenate, vitamin B5, the precursor of the phosphopantetheine, the acyl carrier in coenzyme A and on acyl carrier proteins. In animals, pantothenate is supplied in the diet; in plants and microorganisms, it is synthesised de novo. In Escherichia coli pantothenate is synthesized from its precursors α-keto-isovalerate and aspartate by a four-step pathway (Jackowski et al., 1996). Three genes out of four involved in this biosynthesis, panB, panC and panD were identified and mapped on the E. coli chromosome at 4 min (Berlyn et al., 1996). The last gene in the pathway to be identified was panE. It comprises a 912 bp coding region which specifies a protein of 303 amino acid with deduced molecular mass of 33.8 kD (Elischewski et al., 1999).

The enzyme substrate, ketopantoic acid can exist either as the cyclic ketopantoyl lactone or as the ring opened hydroxyacid, ketopantoate. Both ketopantoate reductases and ketopantoyl lactone reductases have been reported (King and Wilken, 1972). Ketopantoyl lactone reductase has been purified from yeast (King et al., 1974), Norcardia asteroides (Kataoka et al., 1992) and spinach (Julliard, 1994). The spinach enzyme is reported to be soluble and monomeric with a molecular mass of 33-36 kDa. In contrast the Norcardia enzyme is membrane-bound with a subunit molecular mass of 42 kDa. The molecular weight of the native enzyme is reported to be over 600 kDa. The role of ketopantoyl lactone reductase in pantothenate biosynthesis is unclear as the cyclic product pantoyl lactone is not a direct substrate for pantothenate synthetase from plants (Genschel, 1997).

Ketopantoate reductase, the enzyme which reduces the ring open form of the substrate, was initially purified from yeast (King and Wilken, 1972) and subsequently from Pseudomonas maltophilia 845 (Shimizu et al., 1988). The subunit molecular mass of the Pseudomonas enzyme was approximately 30.5 kDa. Experiments to determine the molecular mass of the native protein were inconclusive and gave values between 87-160 KDa.

It has recently been demonstrated that the panE gene is allelic to apbA in Salmonella typhimurium and maps to minute 10 in the genome (Frodyma and Downs, 1998b ). The alternative pyrimidine biosynthesis pathway provides a second route for the generation of the pyrimidine part of thiamine, other than by de novo purine biosynthesis. It has been proposed that the apbA gene product is ketopantoate reductase (Frodmya and Downs, 1998a). The ApbA protein from S. typhimurium has been purified to homogeneity. It is a monomer with a molecular mass of 31.1 kDa. The enzyme has apparent K_{M}s of 0.77 and 0.74 mM for NADPH and ketopantoate respectively. It has been reported that, under conditions where apbA mutants required thiamine, pantoate was sufficient to restore growth (Frodmya and Downs, 1998a). Nevertheless, the role of pantoate in thiamine biosynthesis remains unclear, and may be indirect. Using gene replacement and transposon mutagenesis (Tn5) an E. coli ilvc panE double mutant was isolated which was devoid of ketopantoate reductase activity Manch, J. N. (1981) Can J Microbiol 27, 1231-1233; Primerano, D., and Burns, R. O. (1993) J. Bacteriol. 153, 259-269). This mutant was used to isolate the panE gene by functional complementation. This mutant was rescued by functional complementation using either apbA or ilvC, providing further circumstantial support that apbA and panE are identical. The panE gene was expressed under the control of a tac promoter. A three fold increase in activity was observed. This in turn led to a 3.5 fold increase in the secretion of pantothenate, which could be enhanced further by the addition of ketopantoate (Elischewski et al., 1999). Inhibitors (whether competitive, non-competitive, uncompetitive or irreversible) of pantothenate synthetase would be of significant technical and commercial interest.

Knowledge of the structure of KPR would significantly assist the rational design of novel therapeutics based on modulators of KPR activity.

### DESCRIPTION OF THE DRAWINGS

FIG. 1 illustrates the pantothenate synthesis pathway.

FIG. 2 shows (A) the binding of NADPH and (B) the binding of ketopantoate to KPR.

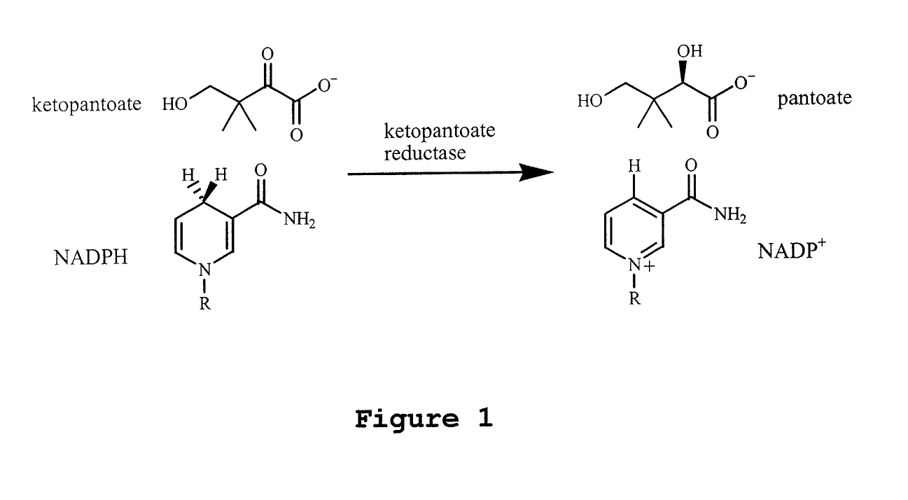

### SUMMARY OF THE INVENTION

The present invention is at based on the novel determination of a high resolution X-ray crystal structure of KPR. The invention is based upon a KPR crystal which has a resolution better than (i.e. numerically lower than) 2 Å.

In one aspect, the invention provides a KPR crystal having a tetragonal space group P4₁2₁2 with unit cell dimensions of a=103.7, b=103.7, c=55.7 Å, or more generally, a=103.7±0.2, b=103.7±0.2, c=55.7±0.2 Å. Such a structure includes the KPR crystal of Table 1. An advantageous feature of the structure is that it has a high resolution, of about 1.7 Å. Another feature of the structure is that it was obtained in a method which utilised selenomethionine substituted enzyme. These features, both singly and in combination all contribute to features of the invention which are advantageous.

The coordinates of Table 1 provide a measure of atomic location in Angstroms, to a third decimal place. The coordinates are a relative set of positions that define a shape in three dimensions, so it is possible that an entirely different set of coordinates having a different origin and/or axes could define a similar or identical shape. Furthermore, varying the relative atomic positions of the atoms of the structure so that the root mean square deviation of the residue backbone atoms (i.e. the nitrogen-carbon-carbon backbone atoms of the protein amino acid residues) is less than 1.5 Å (preferably less than 1.0 Å and more preferably less than 0.5 Å) when superimposed on the coordinates provided in Table 1 for the residue backbone atoms, will generally result in a structure which is substantially the same as the structure of Table 1 in terms of both its structural characteristics and potency for structure-based design of KPR inhibitors. Likewise changing the number and/or positions of the water molecules and/or substrate molecules of Table 1 will not generally affect the potency of the structure for structure-based design of KPR inhibitors. Thus for the purposes described herein as being aspects of the present invention, it is within the scope of the invention if: the Table 1 coordinates are transposed to a different origin and/or axes; the relative atomic positions of the atoms of the structure are varied so that the root mean square deviation of residue backbone atoms is less than 1.5 Å (preferably less than 1.0 Å and more preferably less than 0.5 Å) when superimposed on the coordinates provided in Table 1 for the residue backbone atoms; and/or the number and/or positions of water molecules and/or substrate molecules is varied. Reference herein to the coordinate data of Table 1 thus includes the coordinate data in which one or more individual values of the Table are varied in this way. By “root mean square deviation” we mean the square root of the arithmetic mean of the squares of the deviations from the mean.

The provision of the high resolution structure of Table 1 provides those of skill in the art with a detailed insight into the mechanisms of action of KPR. This insight provides a means to design new antibacterial agents which have the potential to inhibit the process of pantothenate synthesis in bacteria and fungi, or to modulate the activity of the enzyme, for example such that the enzyme works more effectively on prodrugs which are converted by KPR into an antibacterial drug.

In a further aspect, the invention provides a method for crystallizing a selenium atom KPR derivative which comprises producing KPR by recombinant production in a bacterial host (e.g. E. coli) in the presence of selenomethionine, recovering a selenium atom KPR derivative from the host and growing crystals from the recovered selenium atom KPR derivative.

Thus, the selenium atom KPR derivative and KPR produced by crystallising native KPR (see the detailed description below) are provided as crystallised proteins suitable for X-ray diffraction analysis.

The crystals may be grown by any suitable method, e.g. the hanging drop method.

The provision of the crystal structure of KPR allows a novel approach for drug discovery for modulators of this enzyme. Accordingly, the invention provides a computer-based method of rational drug design which comprises:

providing the structure of the KPR as defined by the coordinates of Table 1;

providing the structure of a candidate modulator molecule; and

fitting the structure of candidate to the structure of the KPR of Table 1.

In an alternative aspect, the method of the invention may utilise the coordinates of atoms of interest of the KPR which are in the vicinity of a the putative substrate and/or co-factor binding regions in order to model the pocket in which the substrate or co-factor binds. These coordinates may be used to define a space which is then screened “in silico” against a candidate modulator molecule. Thus the invention provides a computer-based method of rational drug design which comprises:

providing the coordinates of at least two atoms of Table 1 of the KPR (“selected coordinates”);

providing the structure of a candidate modulator molecule; and

fitting the structure of candidate to the selected coordinates of the KPR.

In practice, it will be desirable to model a sufficient number of atoms of the KPR as defined by the coordinates of Table 1 which represent a binding pocket. Binding pockets and other features of the interaction of KPR with co-factor are described in the accompanying example. Thus, in this embodiment of the invention, there will preferably be provided the coordinates of at least 5, preferably at least 10, more preferably at least 50 and even more preferably at least 100 atoms of the KPR structure.

Our structure of KPR has allowed us to identify particular sites of interaction of NADPH and ketopantoate. The selected coordinates preferably include at least one of the coordinates identified in the accompanying examples. These include Leu6, Cys8, Gly9, Leu11, Leu30, Arg31, Leu71, Lys72, Gln75, Asn98, His120, Ala122, Arg124, Lys176, Ser244, and Glu256.

In another aspect, the method of the invention may utilise a sub-domain of interest of the KPR which is in the vicinity of a region which binds substrate or co-factor. Thus, the invention provides a computer-based method of rational drug design which comprises:

providing the coordinates of at least a sub-domain of the KPR;

providing the structure of a candidate modulator molecule; and

fitting the structure of the candidate to the coordinates of the KPR sub-domain provided.

### DETAILED DESCRIPTION OF THE INVENTION

By “fitting”, it is meant determining by automatic, or semi-automatic means, interactions between at least one atom of the candidate and at least one atom of the KPR, and calculating the extent to which such an interaction is stable. Interactions include attraction and repulsion, brought about by charge, steric considerations and the like. Various computer-based methods for fitting are described further herein.

By “sub-domain” is meant at least one (e.g. one, two, three or four) complete element(s) of secondary structure, i.e. an alpha helix or a beta sheet, as described in the detailed description below.

Particular regions of the KPR include those identified as putative substrate or cofactor binding regions based on the data provided in Table 1.

As indicated above, modulators of KPR may be inhibitors of the enzyme or compounds which affect its specificity or activity in relation to ketopantoate in other ways. The invention is particularly suitable for the design, screening and development of KPR inhibitor components. It is thus a preferred aspect of the invention that modulators are inhibitors.

If more than one KPR active site is characterised and a plurality of respective compounds are designed or selected, the potential modulator may be formed by linking the respective compounds into a larger compound which maintains the relative positions and orientations of the respective compounds at the active sites. The larger compound may be formed as a real molecule or by computer modelling.

The step of providing the structure of a candidate modulator molecule may involve selecting the compound by computationally screening a database of compounds for interaction with the active site. For example, a 3-D descriptor for the potential modulator may be derived, the descriptor including geometric and functional constraints derived from the architecture and chemical nature of the active site. The descriptor may then be used to interrogate the compound database, a potential modulator being a compound that has a good match to the features of the descriptor. In effect, the descriptor is a type of virtual pharmacophore.

In any event, the determination of the three-dimensional structure of KPR provides a basis for the design of new and specific modulators for KPR. For example, knowing the three-dimensional structure of KPR, computer modelling programs may be used to design different molecules expected to interact with possible or confirmed active sites, such as binding sites or other structural or functional features of KPR.

More specifically, a potential modulator of KPR activity can be examined through the use of computer modelling using a docking program such as GRAM, DOCK, or AUTODOCK (see Walters et al., Drug Discovery Today, Vol.3, No.4, (1998), 160-178, and Dunbrack et al., Folding and Design, 2, (1997), 27-42) to identify potential inhibitors of KPR. This procedure can include computer fitting of potential modulators to KPR to ascertain how well the shape and the chemical structure of the potential inhibitor will bind to the enzyme.

Also computer-assisted, manual examination of the active site structure of KPR may be performed. The use of programs such as GRID (Goodford, J. Med. Chem., 28, (1985), 849-857)—a program that determines probable interaction sites between molecules with various functional groups and the enzyme surface—may also be used to analyse the active site to predict partial structures of modulating compounds.

Computer programs can be employed to estimate the attraction, repulsion, and steric hindrance of the two binding partners (e.g. the KPR and a candidate inhibitor). Generally the tighter the fit, the fewer the steric hindrances, and the greater the attractive forces, the more potent the potential modulator since these properties are consistent with a tighter binding constant. Furthermore, the more specificity in the design of a candidate modulator, the more likely it is that it will not interact with other proteins as well. This will tend to minimise potential side-effects due to unwanted interactions with other proteins.

In another aspect, in place of in silico methods, high throughput screening of compounds to select compounds with binding activity may be undertaken, and those compounds which show binding activity may be selected as possible candidate modulators, and further crystallized with KPR (e.g. by co-crystallization or by soaking) for X-ray analysis. The resulting X-ray structure may be compared with that of Table 1 for a variety of purposes. For example, where the contacts made by such compounds overlap with those may by ketopantoate, novel molecules comprising residues which contain contacts of both ketopantoate and the other compound may be provided.

Having designed or selected possible binding candidate modulators by determining those which have favourable fitting properties (e.g. strong attraction between candidate and KPR), these can then be screened for activity. Consequently, the method preferably further comprises the further steps of:

obtaining or synthesising the candidate modulator; and

contacting the candidate modulator with KPR to determine the ability of the candidate inhibitor to interact with KPR.

More preferably, in latter step the candidate modulator is contacted with KPR under conditions to determine its function.

Instead of, or in addition to, performing such an assay, the method may comprise the further steps of:

obtaining or synthesising said candidate modulator;

forming a complex of KPR and said potential modulator; and

analysing said complex by X-ray crystallography to determine the ability of said candidate modulator to interact with KPR. Detailed structural information can then be obtained about the binding of the candidate modulator to KPR, and in the light of this information adjustments can be made to the structure or functionality of the potential modulator, e.g. to improve binding to the active site. The above steps may be repeated and re-repeated as necessary.

More preferably, in contacting step above the potential modulator is contacted with KPR in the presence of a substrate, and typically a buffer, to determine the ability of said potential modulator to alter the function of KPR. The substrate may be e.g. ketopantoate (or a salt thereof) and/or NADPH. So, for example, an assay mixture for KPR may be produced which comprises the potential modulator, substrate (e.g. NADPH) and buffer.

In another aspect, the invention relates to a method of determining three dimensional structures of KPR homologues of unknown structure by utilising the structural coordinates of Table 1.

For example, if X-ray crystallographic or NMR spectroscopic data is provided for a KPR homologue of unknown structure, the structure of KPR as defined by Table 1 may be used to interpret that data to provide a likely structure for the KPR homologue by techniques which are well known in the art, e.g. phase modelling in the case of X-ray crystallography.

One embodiment of the method comprises the steps of:

(a) aligning a representation of an amino acid sequence of a KPR homologue of unknown structure with the amino acid sequence of KPR to match homologous regions of the amino acid sequences;

(b) modelling the structure of the matched homologous regions of the KPR of unknown structure on the structure as defined by Table 1 of the corresponding regions of KPR; and

(c) determining a conformation (e.g. so that favourable interactions are formed within the KPR of unknown structure and/or so that a low energy conformation is formed) for the KPR of unknown structure which substantially preserves the structure of said matched homologous regions.

The term “homologous regions” describes amino acid residues in two sequences that are identical or have similar (e.g. aliphatic, aromatic, polar, negatively charged, or positively charged) side-chain chemical groups. Identical and similar residues in homologous regions are sometimes described as being respectively “invariant” and “conserved” by those skilled in the art.

Preferably one or all of steps (a) to (c) are performed by computer modelling. Homology modelling is a technique that is well known to those skilled in the art (see e.g. Greer, Science, Vol. 228, (1985), 1055, and Blundell et al., Eur. J. Biochem, Vol. 172, (1988), 513).

In general, comparison of amino acid sequences is accomplished by aligning the amino acid sequence of a polypeptide of a known structure with the amino acid sequence of the polypeptide of unknown structure. Amino acids in the sequences are then compared and groups of amino acids that are homologous are grouped together. This method detects conserved regions of the polypeptides and accounts for amino acid insertions or deletions.

Homology between amino acid sequences can be determined using commercially available algorithms. The programs BLAST, gapped BLAST, BLASTN and PSI-BLAST (provided by the National Center for Biotechnology Information) are widely used in the art for this purpose, and can align homologous regions of two amino acid sequences.

Once the amino acid sequences of the polypeptides with known and unknown structures are aligned, the structures of the conserved amino acids in a computer representation of the polypeptide with known structure are transferred to the corresponding amino acids of the polypeptide whose structure is unknown. For example, a tyrosine in the amino acid sequence of known structure may be replaced by a phenylalanine, the corresponding homologous amino acid in the amino acid sequence of unknown structure.

The structures of amino acids located in non-conserved regions may be assigned manually by using standard peptide geometries or by molecular simulation techniques, such as molecular dynamics. The final step in the process is accomplished by refining the entire structure using molecular dynamics and/or energy minimization.

The aspects of the invention described herein which utilise the KPR structure in silico may be equally applied to homologue models of KPR obtained by the above aspect of the invention, and this application forms a further aspect of the present invention. Thus having determined a conformation of a KPR by the method described above, such a conformation may be used in a computer-based method of rational drug design as described herein.

In another aspect, the invention includes a compound which is identified as a modulator of a KPR by the methods of the invention described above.

In a further aspect, the invention provides a method for determining the structure of a modulator of KPR bound to KPR, said method comprising:

providing a crystal of KPR according to the invention;

soaking the crystal with said modulator; and

determining the structure of said KPR-modulator complex.

Alternatively, the KPR and modulator may be co-crystallized. In either case, ketopantoate and/or NADP or an analogue thereof may optionally be present.

Having obtained and characterized a modulator compound according to the invention, the invention further provides a method for modulating the activity of KPR which method comprises:

providing KPR under conditions where, in the absence of modulator, the KPR is able to synthesize pantoate from ketopantoate;

providing a modulator compound; and

determining the extent to which the activity of KPR is altered by the presence of said compound.

In another aspect, the present invention provides systems, particularly a computer systems, intended to generate structures and/or perform rational drug design for a KPR or a complex of KPR and potential modulator, the systems containing either (a) atomic coordinate data according to Table 1 or derived therefrom by homology modelling, said data defining the three-dimensional structure of a KPR or at least one sub-domain thereof, or (c) structure factor data for KPR, said structure factor data being derivable from the atomic coordinate data of Table 1.

In a further aspect, the present invention provides computer readable media with either (a) atomic coordinate data according to Table 1 or derived from Table 1 by homology modelling recorded thereon, said data defining the three-dimensional structure of KPR, at least one atom or at least one sub-domain thereof, or (b) structure factor data for KPR recorded thereon, the structure factor data being derivable from the atomic coordinate data of Table 1.

As used herein, “computer readable media” refers to any media which can be read and accessed directly by a computer. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

By providing such computer readable media, the atomic coordinate data can be routinely accessed to model KPR or a sub-domain thereof. For example, RASMOL (Sayle et al., TIBS, Vol. 20, (1995), 374) is a publicly available computer software package which allows access and analysis of atomic coordinate data for structure determination and/or rational drug design.

On the other hand, structure factor data, which are derivable from atomic coordinate data (see e.g. Blundell et al., in Protein Crystallography, Academic Press, New York, London and San Francisco, (1976)), are particularly useful for calculating e.g. difference Fourier electron density maps.

As used herein, “a computer system” refers to the hardware means, software means and data storage means used to analyse the atomic coordinate data of the present invention. The minimum hardware means of the computer-based systems of the present invention comprises a central processing unit (CPU), input means, output means and data storage means. Desirably a monitor is provided to visualise structure data. The data storage means may be RAM or means for accessing computer readable media of the invention. Examples of such systems are microcomputer workstations available from Silicon Graphics Incorporated and Sun Microsystems running Unix based, Windows NT or IBM OS/2 operating systems.

In another aspect, the invention provides a method of analysing a complex of KPR and a potential modulator comprising the step of employing (i) X-ray crystallographic diffraction data from the complex and (ii) a three-dimensional structure of KPR, or at least one sub-domain thereof, to generate a difference Fourier electron density map of the complex, the three-dimensional structure being defined by atomic coordinate data according to Table 1.

Therefore, such complexes can be crystallised and analysed using X-ray diffraction methods, e.g. according to the approach described by Greer et al., J. of Medicinal Chemistry, Vol. 37, (1994), 1035-1054, and difference Fourier electron density maps can be calculated based on X-ray diffraction patterns of soaked or co-crystallised KPR and the solved structure of uncomplexed KPR. These maps can then be used to determine whether and where a particular potential modulator binds to KPR and/or changes the conformation of KPR.

Electron density maps can be calculated using programs such as those from the CCP4 computing package (Collaborative Computational Project 4. The CCP4 Suite: Programs for Protein Crystallography, Acta Crystallographica, D50, (1994), 760-763.). For map visualisation and model building programs such as “O” (Jones et al., Acta Crystallograhy, A47, (1991), 110-119) can be used.

The structure is defined in Table 1 which gives atomic coordinate data for KPR and associated water molecules. Table 1 lists each atom by a unique number; the chemical element and (in the case of KPR) its position in each amino acid residue; the amino acid residue in which the element is located; the number of the residue; X, Y and Z coordinates which define, with respect to the crystallographic axes, the atomic position (in D) of the respective atom; the occupancy of the atom in the respective position; “B”, a temperature factor (in D²) which accounts for movement of the atom around its atomic centre; and atomic number.

Structure-Based Drug Design

Determination of the 3D structure of KPR provides important information about the likely active sites of KPR, particularly when comparisons are made with similar enzymes. This information may then be used for rational design of KPR inhibitors, e.g. by computational techniques which identify possible binding ligands for the active sites, by enabling linked-fragment approaches to drug design, and by enabling the identification and location of bound ligands using X-ray crystallographic analysis. These techniques are discussed in more detail below.

Greer et al. mentioned above describes an iterative approach to ligand design based on repeated sequences of computer modelling, protein-ligand complex formation and X-ray analysis. Thus novel thymidylate synthase inhibitor series were designed de novo by Greer et al., and KPR inhibitors may also be designed in the this way. More specifically, using e.g. GRID on the solved 3D structure of KPR, a potential modulator for KPR may be designed that complements the functionalities of the KPR active site(s). The potential modulator compound can then be synthesised, formed into a complex with KPR, and the complex then analysed by X-ray crystallography to identify the actual position of the bound compound.

Determination of the position of the potential modulator in the complex allows determination of the interactions of it with KPR. This will allow those of skill in the art to analyse the affinity and specificity of the compound for KPR, and to propose modifications to the compound to increase or decrease either or both of these properties. Thus the structure and/or functional groups of the compound can then be adjusted, if necessary, in view of the results of the X-ray analysis, and the synthesis and analysis sequence repeated until an optimised compound is obtained. Related approaches to structure-based drug design are also discussed in Bohacek et al., Medicinal Research Reviews, Vol.16, (1996), 3-50.

As a result of the determination of the KPR 3D structure, more purely computational techniques for rational drug design may also be used to design KPR modulators (for an overview of these techniques see e.g. Walters et al. mentioned above). For example, automated ligand-receptor docking programs (discussed e.g. by Jones et al. in Current Opinion in Biotechnology, Vol.6, (1995), 652-656) which require accurate information on the atomic coordinates of target receptors may be used to design potential KPR modulators.

Linked-fragment approaches to drug design also require accurate information on the atomic coordinates of target receptors. Small compounds which have the potential to bind to regions of KPR which in themselves may not be modulator compounds may be assembled by chemical linkage to provide potential modulators. Thus the basic idea behind these approaches is to determine the binding locations of plural ligands to a target molecule, and then construct a molecular scaffold to connect the ligands together in such a way that their relative binding positions are preserved. The ligands may be provided computationally and modelled in a computer system, or provided in an experimental setting, wherein crystals according to the invention are provided and a plurality of ligands soaked separately or in mixed pools into the crystal prior to X-ray analysis and determination of their location.

The binding site of two of more ligands are determined and may be connected to thus form a potential lead compound that can be further refined using e.g. the iterative technique of Greer et al. For a virtual linked-fragment approach see Verlinde et al., J. of Computer-Aided Molecular Design, 6, (1992), 131-147, and for NMR and X-ray approaches see Shuker et al., Science, 274, (1996), 1531-1534 and Stout et al., Structure, 6, (1998), 839-848. The use of these approaches to design KPR modulators is made possible by the determination of the KPR structure.

Many of the techniques and approaches to structure-based drug design described above require X-ray analysis to identify the binding position of a potential modulator in a complex with a protein. A common way of doing this is to perform X-ray crystallography on the complex, produce a difference Fourier electron density map, and associate a particular pattern of electron density with the potential modulator. However, in order to produce the map (as explained e.g. by Blundell et al. mentioned above) it is necessary to know beforehand the protein 3D structure (or at least the protein structure factors).

Therefore, determination of the KPR structure also allows difference Fourier electron density maps of complexes of KPR with a potential modulator to be produced, which can greatly assist the process of rational drug design.

The approaches to structure-based drug design described above all require initial identification of possible compounds for interaction with target bio-molecule (in this case KPR). Sometimes these compounds are known e.g. from the research literature. However, when they are not, or when novel compounds are wanted, a first stage of the drug design program may involve computer-based in silico screening of compound databases (such as the Cambridge Structural Database) with the aim of identifying compounds which interact with the active site or sites of the target bio-molecule. Screening selection criteria may be based on pharmacokinetic properties such as metabolic stability and toxicity. However, determination of the KPR structure allows the architecture and chemical nature of each KPR active site to be identified, which in turn allows the geometric and functional constraints of a descriptor for the potential inhibitor to be derived. The descriptor is, therefore, a type of virtual 3-D pharmacophore, which can also be used as selection criteria or filter for database screening.

Compounds which have a chemical structure selected using the methods of the invention described herein, wherein said compounds are KPR modulators, form a further aspect of the invention. Such compounds may be used in methods of medical treatments, such as in the treatment of bacterial infections in the human or animal body. The compounds may be used alone or in conjunction with other anti-bacterial compounds to enhance their effect.

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention.

The following example illustrates the invention.

Materials and Methods

Cloning of E. coli panE Gene.

The E. coli panE (apbA) gene sequence had been submitted to the EMBL database (Acc N° U34923) and this sequence is part of the plasmid pVJS78, which contains a 6 kb fragment of E. coli genomic DNA (obtained from Dr T Begley, Dept of Chemistry, Cornell University, New York). A PCR product, representing the full-length panE gene, was generated using the following primers EPANENdeF

(5′ ATACATATGAAAATTACCGTATTGGGATG 3′) and EPANEBamHIR

(5′ TATGGATCCTCACCAGGGGCGAGGCAAACC 3′), which had NdeI and

BamHI restriction sites respectively (underlined). This 915 bp PCR product was digested with NdeI/BamHI and ligated into NdeI/BamHI restricted pET24b vector (Novagen). The panE gene was sequenced and found to be identical to that in the database. Crude soluble protein extracts of the bacterial cells harboring pET24b-panE had measurable KPR activity

Protein Expression and Purification

E. coli BL21 Cell culture containing pET24-b-apbA was grown in LB media containing ampicillin (50 mg/mL) at 37° C. to an OD₆₀₀ of 0.6-0.7, and expression of KPR was induced by addition of isopropylthio-β-D-galactoside (IPTG) to a final concentration of 0.5 mM. The cells were harvested after 4 h and resuspended in extraction buffer containing 100 mM KH₂PO₄ pH 7.5, 1 mM EDTA and 1 mM PMSF. Cells were lysed by sonication and centrifuged. Nucleic acids were precipitated by the addition of 5 mL protamine sulfate (2% w/v) and centrifugation. The supernatant was fractionated with solid ammonium sulfate and the precipitate occurring between 5%-40% saturation was collected by centrifugation. The protein was redissolved in 25 mM KH₂PO₄ pH 7.5 containing 1 mM EDTA, dialyzed against 2 L of the same buffer and purified by fast phase liquid chromatography (FPLC). The FPLC purification procedure consisted of passing the protein through two anion exchange and one gel filtration column. Protein, in less than 10 mL of buffer containing 25 mM potassium chloride, was applied to a Hiprep 16/10 Q XL anion exchange column and eluted with a linear 25 mM-500 mM potassium chloride gradient. Fractions containing KPR were concentrated and desalted. The protein solution, in less than 10 mL of buffer containing 25 mM potassium chloride, was then applied to a Source 15Q XV 16/10 anion exchange column and eluted with a linear 25 mM-500 mM potassium chloride gradient. Protein, in less than 5 ml of buffer, was then applied to a Hiload 16/60 Superdex 200 pg gel filtration column and fractions containing KPR were concentrated to greater than 5 mg/mL. Fractions were analyzed by SDS-PAGE, according to the method of Laemmli (Laemmli, 1970). Protein concentration was determined using the Bio-Rad protein assay kit, using bovine serum albumin as a standard and confirmed spectrophotometrically, using A₂₈₀ (ε calculated) (Gill and von Hippel, 1989). Electrospray mass spectra were recorded on a VG BioQ quadrupole mass spectrometer. The mass of the protein calculated from the primary amino acid sequence (33, 870 Da) of the wild type compares well with the ESMS (33, 876 Da) of the recombinant protein. N-terminal sequencing for the first 9 residues as well as amino acid analysis was the same as the wild type.

Selenomethionine-substituted enzyme (SeMet KPR) was prepared by expression of the freshly transformed pET24-β-apbA. The E. coli BL21 cell culture was grown at 37° C. to an OD₆₀₀ of 0.8 in M9 minimal media containing 4% glucose. The medium was then supplemented by 6 amino acids (Lys, Phe, Thr, Ile, Leu, Val) and selenomethionine at 50 mg/L, with 0.5% thiamine (Doublie, S., (1997) Methods Enzymol. 278, 523-530). Cells were induced using IPTG (25 mg/L) for two and a half hours and harvested. Purification of Se-Met KPR was performed as described above with addition of 5 mM DTT to prevent oxidation of the seleno-methionines.

Determination of KPR Activity.

KPR activity was determined at 37° C. in the forward direction by monitoring the decrease in NADPH over time (as absorbance at 340 nm) as described (7). The assay comprised 100 mM potassium phosphate buffer, pH 7.4, 150 μM NADPH, 30 nM KPR and was initiated with 500 μM potassium ketopantoate, in a final volume of 1 mL.

Crystallization

Regular prismatic KPR crystals were prepared by the hanging drop vapor-diffusion technique, using 25-27% PEG 4000, 0.1 M Tris pH 9.4 and 0.20-0.25 M NaOAc as the precipitant solution. Protein concentration was 11 mg/mL in 10 mM Hepes pH 8.0. The hanging drops, consisting of 1 μL of protein solution and 1 μL well solution. were equilibrated at 4° C. for several days. Crystals with dimensions 0.4×0.2×0.2 mm³ grew from clear drops. These belonged to the tetragonal space group P4₂2₁2 with cell parameters a=b=104.2 Å and c=55.8 Å, accommodating one enzyme molecule per asymmetric unit, with a solvent content of 45%.

SeMet KPR crystals, prepared in a similar way to native KPR crystals, were obtained using two different protein concentrations, 8 mg/ml and 16 mg/ml. The Se-Met KPR stock solution contained 15 mM Tris, pH 8 and 2 mM DTT to protect the Se atoms from oxidation. Optimised crystallization conditions are 28-38% PEG 4000, 0.1 M Tris pH 9.4 Ånd 0.25 M NaOAc.

Data Collection and Processing

The structure of ketopantoate reductase (KPR) was solved by MAD method using a selenomethionine derivative. Data to 2.3 Å resolution were collected at 100 K, at 3 wavelengths on the BW7A beamline at the EMBL outstation in DESY, Hamburg. Flash-freezing of the crystal was proceeded by soaking for 30 s in a cryoprotectant solution consisting of 15% glycerol, 30% PEG 4000, 0.1 M Tris pH=9.4 and 0.25 M sodium-acetate. An X-ray fluorescence spectrum was recorded and used to select wavelengths for subsequent MAD data collection. Data were collected at the Se absorption edge λe=0.979272 Å (12 661.00 eV), the absorption peak λp=0.9790501 Å (12 663.77 eV) and at remote reference wavelength λr=0.885612 Å (14 000 eV). The diffraction data were indexed and integrated using the DENZO package (Otwinowski and Minor, 1997). The three data-sets were scaled to the remote data-set using SCALA (CCP4, 1994; Evans, 1997) and structure-factor amplitudes were calculated using TRUNCATE (CCP4, 1994). Statistics of the processed data are listed in Table 2. The native data set was collected to 1.7 Å resolution at the Daresbury SRS beamline 9.6. A cryo-protectant solution contained 30% PEG1500, 100 mM Tris buffer (pH 9.4), 300 mM sodium-acetate and 20% of glycerol.

TABLE 2
X-ray data collection statistics

| | Se-Met derivative | | Native | |
|---|---|---|---|---|
| Dataset | Edge | Peak | Remote | |
| Wavelength (Å) | 0.979272 | 0.9790501 | 0.885612 | 0.87 |
| Resolution (Å) | 2.3 | 2.3 | 2.3 | 1.7 |
| Unique reflections | 13640 | 11259 | 13720 | 32249 |
| Redundancy | 3.77 | 4.5 | 3.95 | 5.4 |
| Completeness (%) | 95.2 | 96.3 | 97.5 | 94.9 |
| Rₐₙₒ (%) | 5.8 | 6.8 | 4.6 | — |
| R_{merge} (%) | 9.9 | 10.8 | 9.4 | 4.3 |
| f′, f″ (experimental) | −11.1873 | −7.5644 | −1.5 | — |
| | 4.1205 | 5.9692 | — | |
| f′, f″ (theoretical) | −11.0597 | −7.46687 | −1.3 | — |
| | 4.38418 | 6.29644 | 3.2 | |
| Phasing power | 2.288 | 2.889 | 2.108 | — |
| R | | | | 0.230 |
| R (free) | | | | 0.289 |

| | | |
|---|---|---|
| No. reflexions: | working set | 30610 |
| | test set | 1639 |
| Ramachandran plot: | % residues favorable | 91.9 |
| | % residues | None |
| | unfavorable | |
| R.m.s. deviation: | Bond lengths | 0.016 |
| | Bond angles | 1.9 |
| | Planarity | 0.024 |

Structure Determination and Refinement

Six selenium sites were found with the program SHELXS96 (Sheldrick, 1996) using direct methods and anomalous difference data of λp Se-Met. Data were phased with SHARP (La Fortelle et al., 1997) using all three wavelength data sets, which also revealed two additional Se sites in the residual maps. Data collected at the remote wavelength were treated as the reference data set and resolution limits of 40 to 2.3 Å were imposed. Theoretical values of f′ and f″ were used and refined during analysis. The resulting values are very similar to the theoretical values and are given in Table 1. Experimental phases were improved by solvent flattening using SOLOMON(CCP4, 1994), via the SUSHI graphical user interface (La Fortelle et al., 1997) with a solvent content of 43%. The final electron-density map was easily interpretable and whole polypeptide chain was assigned based on the initial electron density map.

The polypeptide chain was fitted in MAD electron density map using program O (Jones et al., 1991). Rounds of maximum likelihood refinement with REFMAC (Murshudov et al., 1997) were alternated with visual inspection of electron density and manual rebuilding of side chains. Several rounds of simulated annealing with CNS (Brunger et al., 1998) were included to properly refine the position of the main-chain. The current model contains 291 residues and 272 water molecules.

Results and Discussion

KPR from E. coli was expressed and purified in order to determine its 3D structure using X-ray crystallography. The crystal structure was determined using selenomethionine-substituted KPR and MAD method.

Selenomethionine-substituted KPR was expressed in minimum medium containing a cocktail of six amino acids inhibiting methionine biosynthesis.

Structure Solution and Refinement

Initial phases were calculated by MAD technique using three data sets collected at wavelengths on and away from the selenium Kα absorption edge. After solvent flattening an easily interpretable electron-density map was obtained at 2.3 A resolution. Secondary structural elements were clearly defined in the electron density bones calculated with MAPMAN (Kleywegt & Jones, 1996).

The asymmetric unit consists of one monomeric KPR molecule. The main chain of the molecule was traced using secondary structure template building functionality in O. and the sequence was easily determined from the positions of selenium atoms from SHARP.

The model was improved by restrained isotropic maximum likelihood refinement with REFMAC. Crystallographic refinement was alternated with manual model rebuilding in O. Torsional simulated annealing (starting temperature 3000K) in CNS was used to improve difficult parts of the model. Ordered water molecules were modelled by automated cycles of water addition and removal by ARP (Perrakis et al., 1999) and refinement by REFMAC. A final model containing 291 residues and 272 water molecules was refined to an R factor of 0.230 and R_{free} of 0.289. The last 12 residues at the c-terminus are not visible in electron density maps.

The quality of the model and its geometry were assessed by PROCHECK (Laskowski et al., 1993). There are no Ramachandran outliers, and 91.9% of residues lie in the most favoured regions of the plot.

Overall Structure

The secondary structure assignment of KPR, as analyzed using the DSSP programme (Kabsch and Sander, 1983), shows the molecule to be constructed from twelve α-helices, three 3₁₀-helices and eleven β-strands. The enzyme is monomeric and has two domains separated by a deep cleft. The N-terminal domain has an alpha-beta fold of Rossman type and comprises residues 1-169. It is formed of two β-sheets joined together by β-bridge and three α-helices. The central α-helix is surrounded by the seven-stranded β-sheet of mixed parallel and antiparallel character and also by a smaller four stranded β-sheet that contains two pairs of antiparallel strands. This central α-helix (α1) separates the first two β-strands and comprises a dinucleotide binding unit. The sequence between the end of β1-strand and the α1-helix contains a glycine-rich region. The GCGALG sequence represent the motif GXGXXG that is very often an indicator of the nucleotide binding site in dehydrogenases (Branden, C. Tooze, J. (1991) Introduction to protein structure, Garland Publishing, Inc. New York). The C-terminal domain is all alpha-helical composed of the eight α-helices including remaining residues to the C-terminus (170-291).

Nucleotide-Binding Site

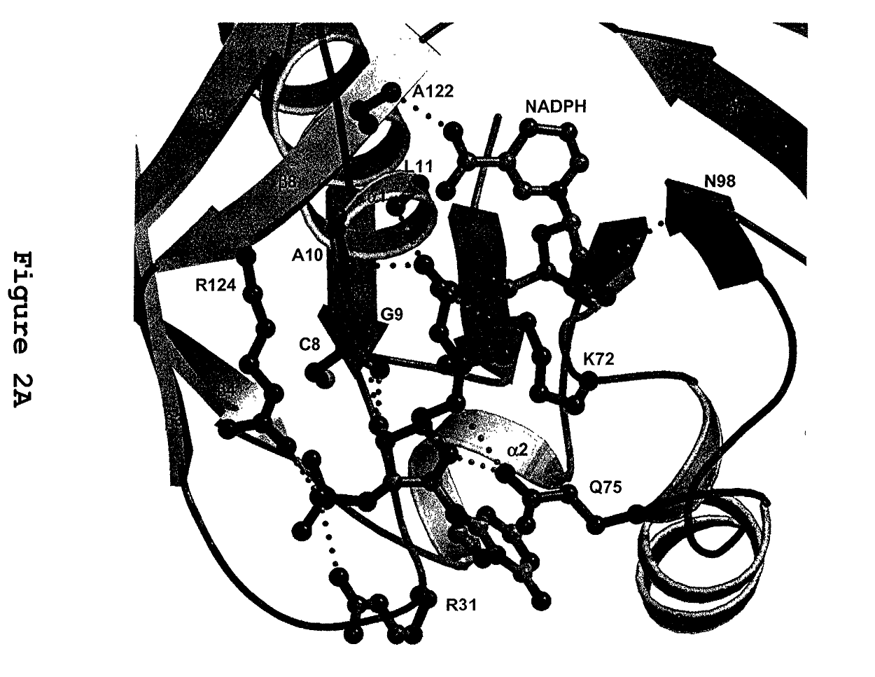

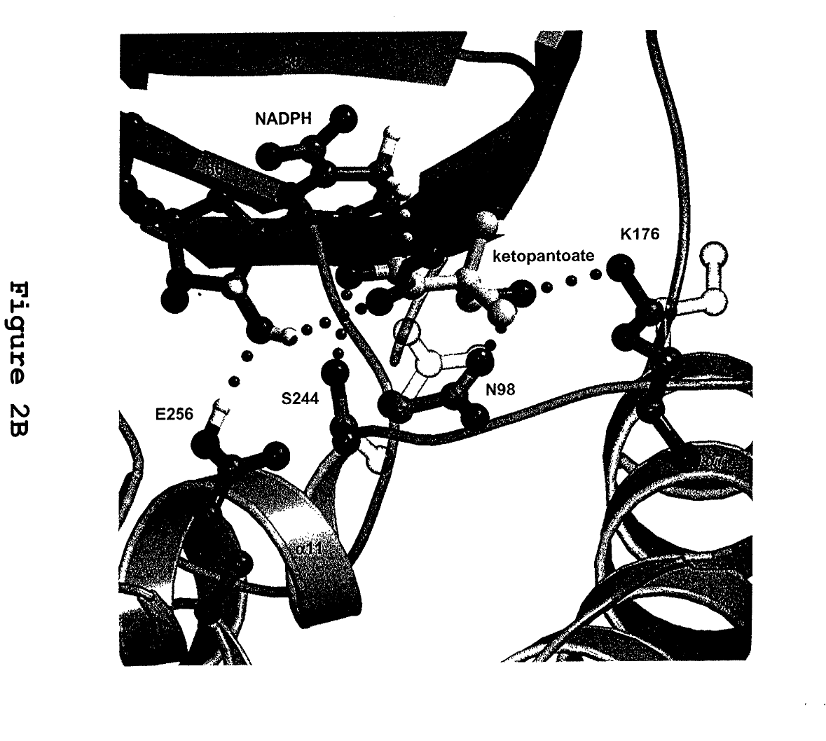

The structural similarity between the N-terminal domains of KPR and human SCHAD enabled us to carry out molecular modelling to identify the cofactor binding site, since the structure of the binary complex of SCHAD with NAD⁺ has been determined (PDB code 2hdh). The SCHAD structure was fitted to the KPR structure and NAD⁺ cofactor extracted from SCHAD to KPR. Water molecules clashing with cofactor were removed and hydrogen atoms added to the model with SYBYL Biopolymer module (Tripos, 1998) for force field optimization purposes. The NAD⁺ molecule was edited to NADPH and optimized as a substructure using the Tripos force field. In the process the pyrophosphate group of NADPH changed conformation to fit into the inter-domain cleft (FIG. 2A). Only side-chains involved in interactions with cofactor moved during geometry optimization. In the resulting model NADPH is largely buried between two domains, with only the adenine moiety of NADPH partially exposed, as seen in many enzymes with the nucleotide binding fold. There is no evidence of direct hydrogen bonding between the adenine ring and the protein. The adenine pocket contains side chains of residues Leu6, Leu7l, Gln75, Leu30 and Arg31. The glycine-rich turn that lies between β-1 of the N-terminal domain and α1 is directly involved in the recognition of the adenine ribose through two main chain hydrogen bonds of hydroxyl groups with main chain nitrogen atoms of residues Cys8 and Gly9. The 2′-phosphate (which distinguishes NADPH from NADH) is involved in two salt bridges with the guanidine moieties of residues Arg31 and Arg124 that are located on the protein surface. The conserved residue Lys72 interacts with the pyrophosphate moiety through three hydrogen bonds, implying that this residue plays a major role in cofactor recognition. This observation is interesting, since it has previously been reported that a Lys72Ala mutant had wild-type enzyme activity in a crude cell free preparation, comparable to their Asp250Ala mutation, which is remote from the active site. A second residue, Leu11, recognises pyrophosphate through its main chain nitrogen, which interacts with nicotinamide ribose phosphate. The nicotinamide portion of the cofactor is anchored in place by hydrogen bonds to its ribose hydroxyl by the conserved residues Glu256 Oε2 atom and Asn98 main chain nitrogen atom (FIG. 2B). Additionally, the nicotinamide ring interacts with main chain nitrogen atom of His120 and Ala122.

Substrate Binding Site and Reaction Mechanism

Although the apoenzyme is in a more open conformation than would be expected for catalysis, it is still possible to identify key catalytic residues and those involved in binding ketopantoate. In particular Lys176 and Glu256, which have previously been identified as being important in catalysis by site directed mutagenesis, are located near the putative NADPH site, some distance apart, and could accommodate ketopantoate between them. From knowledge of the stereochemical course of the reaction, that the proS hydrogen of NADPH is delivered to the si face of ketopantoate, and considering the expected geometry for hydride transfer, it is possible to construct two models for the ternary complex. In one, the carbonyl group of ketopantoate points away from the nitrogen of the dihydronicotinamide ring. This places the carboxyl group towards Lys176 and the hydroxymethyl group towards Glu256. However it introduces unfavorable steric interactions between the gem dimethyl group on ketopantoate and the C-3 primary amide on the dehydronicotinamide ring. The alternative structure is generated from the first one, by rotating ketopantoate in the plane of the carbonyl group by 180 degrees, so that the latter points towards the nitrogen of the dehydronicotinamide ring. This places the carboxyl group of ketopantoate towards Glu256 and the hydroxymethyl group towards Lys176. FIG. 2B shows this structure and highlights other key conserved residues, Asn98 and Ser244, (after some slight adjustment of the side chains of these residues and a larger movement of Lys176). In this structure, there is good hydrogen bonding of Lys176 and Asn98 to the hydroxyl group of ketopantoate, and of Ser244 to the carboxyl. Furthermore Glu256 could act as the acid to protonate the developing negative charge on ketopantoate as hydride is delivered from NADPH, albeit relayed through the C-3 hydroxyl of the ribose.

Structural and Functional Comparison of KPR with Other Enzymes

Comparison of the fold against all structures in the Protein Data Bank using the DALI server (Holm et al., 1995; Holm & Sander, 1998) revealed that the KPR molecule is topographically similar to the N-(1-D-carboxylethyl)-L norvaline dehydrogenase (CENDH) (Britton et al., 1998) in both domains while N-terminal domain shows similarity to some dehydrogenases including human heart short chain L-3-hydroxyacyl-CoA dehydrogenase (SCHAD) (Barycki et al., 1999), 6-phosphogluconate dehydrogenase (6PGDH) (Phillips et al., 1998) and plant acetohydroxy acid isomeroreductase (Biou et al., 1997) as well. Despite the high degree of structural similarity of KPR with CENDH, CENDH is a homodimer (subunit molecular weight of ˜36000), with the α-helical C-terminal domain providing the dimerisation interface while KPR is monomer.

In conclusion, KPR plays a key step on the biosynthesis of pantothenate, and as such is a potential herbicide and antimicrobial target. From the structure of the apoenzyme provided herein it has been possible to construct models of the holoenzyme and the ternary complex, and provide a proposed catalytic mechanism. The model confirms the importance of Lys176 and Glu256 in binding ketopantoate and in the catalytic mechanism. Thus the structure of the present invention will be useful in the design of novel modulators of KPR activity.

### REFERENCES

Barycki, J. J., O'Brien, L. K., Bratt, J. M., Zhang, R., Sanishvili, R., Strauss, A. W., Banaszak, L. J. (1999) Biochemistry 38, 5786-5798.

Berlyn, M. K. B., Low, K. B., Rudd, K. E., 1996. In: Neidhardt, F. C., et al (Eds.), Escherichia coli and Salmonella, 2^{nd} ed., ASM Press, Washington, D.C., pp. 1715-1902.

Biou, V., Dumas, R., Cohen-Addad. C., Douce, R., Job, D., Pebay-Peyroula, E. (1997) EMBO J. 16 (12), 3405-3415.

Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T. & Warren, C. L. (1998). Acta Crystallographica D54, 905-921.

Britton, K. L., Asano, Y. & Rice, D. W. (1998) Nature Struct. Biol. 5 (7), 593-601.

Collaborative Computational Project, Number 4 (1994) Acta Cryst. D50, 760-763.

Dumas, R., Butikofer, M. -C., Job, D. and Douce, R. (1995) Biochemistry 34, 6026-6036.

Elischewski, F, Puller A., Kalinowski J. (1999) J. Biotech. 75, 135-146.

Evans, P. R. (1997). Scaling of MAD data. In Recent Advances in Phasing (ed. K. S. Wilson, G. Davies, A. W. Ashton and S. Bailey), pp. 97-102. Council for the Central Laboratory of the Research Councils Daresbury Laboratory, Daresbury, UK.

Frodyma, M. E., and Downs, D. (1998b) J. Bacteriol. 180, 4757-4759.

Frodyma, M. E., and Downs, D. (1998Å) J. Biol. Chem. 273, 5572-5576.

Genschel, U. (1997). Molecular and biochemical analyses of pantothenic acid biosynthetic enzymes. Ph.D. Thesis, University of Cambridge.

Gill S. C., von Hippel P. H. (1989) Anal. Biochem. 182, 319-326.

Holm, L., & Sander, C. (1998) Nucleic Acids Research 26, 316-319.

Holm, L., de Daruvar, A., Sander, C. & Dodge, C. (1995). The Dali Server at http://www2.ebi.ac.uk/dali/.

Jackowski, S., 1996. In: Neidhardt, F. C., et al (Eds.), Escherichia coli and Salmonella, 2^{nd} ed., ASM Press, Washington, D.C., pp. 687-694.

Jones, T. A., Zou, J. Y., Cowan, S. W. & Kjeldgaard, M. (1991). Acta Crystallographica A47, 110-119.

Julliard, J. H. (1994) Bot. Acta 107, 191-200.

Kataoka, M., Shimizu, S., Yamada, H. (1992) Eur J. Biochem. 204, 799-806.

King, H. L. and Wilken, D. R. (1972) J. Biol. Chem. 247, 4096-4098.

King H. L., Dyar, R. E., Wilken, D. R. (1974) J. Biol. Chem. 249, 4689-4695.

Kleywegt, G. J. & Jones, T. A. (1996) Acta Crystallographica D52, 826-828.

Kabsch, W., Sander, C. (1983) Biopolymers Dec; 22(12), 2577-637.

Laemmli, U. K. (1970) Nature 227, 680-685.

La Fortelle, E. de & Bricogne, G. (1997) Methods in Enzymology 276, 472-494.

La Fortelle, E. d., Irwin, J. J. & Bricogne, G. (1997). Advances in MIR and MAD phasing: Maximum-likelihood refinement in a graphical environment, with SHARP. In CCP4 study week-end: Recent Advances in Phasing. (ed. K. S. Wilson, G. Davies, A. W. Ashton and S. Bailey), Daresbury Laboratory, UK

Laskowski, R. A., Macarthur, M. W., Moss, D. S., Thornton J. M. (1993) J. Appl. Cryst. 26, 283-291.

Murshudov, G. N., Vagin, A. A. & Dodson, E. J. (1997). Acta Crystallographica D53, 240-255.

Otwinowski, Z. and Minor, W. (1997) Methods Enzymol. 276, 307-326.

Perrakis, A., Morris, R. & Lamzin, V. S. (1999). Nature Structural Biology 6, 458-463.

Phillips, C., Dohnalek, J., Gover, S., Barrett, M. P., Adams., M. J. (1998) J. Mol. Biol. 282, 667-681.

Primerano, D., and Burns, R. O. (1993) J. Bacteriol. 153, 259-269.

Sheldrick, M., SHELXS96. Program for the Solution of Crystal Structures. University of Göttingen, Germany, 1996.

Shimizu, S., Kataoka, M., Chung, M. C., and Yamada, H. (1988) J. Biol. Chem. 263, 12077-12084.

Tripos Associates, Sybyl 6.5. The Integrated Molecular Modeling System, St. Louis, Mo., USA, 1998.

TABLE 1

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |

| | | | | | | |
|---|---|---|---|---|---|---|
| CRYST1 | 103.732 | 103.732 | 55.737 | 90.000 | 90.000 | 90.000 |

| | | | | |
|---|---|---|---|---|
| SCALE1 | 0.00964 | 0.00000 | 0.00000 | 0.00000 |
| SCALE2 | 0.00000 | 0.00964 | 0.00000 | 0.00000 |
| SCALE3 | 0.00000 | 0.00000 | 0.01794 | 0.00000 |

| | | | | | | | | | | | |
|---|---|---|---|---|---|---|---|---|---|---|---|
| ATOM | 1 | CB | MET A | 1 | 53.656 | 8.328 | −7.807 | 1.00 | 25.16 | 6 | C |
| ATOM | 2 | CG | MET A | 1 | 53.462 | 8.700 | −6.359 | 1.00 | 27.38 | 6 | C |
| ATOM | 3 | SD | MET A | 1 | 52.363 | 10.015 | −5.974 | 1.00 | 31.67 | 16 | S |
| ATOM | 4 | CE | MET A | 1 | 53.026 | 10.727 | −4.486 | 1.00 | 32.03 | 6 | C |
| ATOM | 5 | C | MET A | 1 | 55.984 | 7.342 | −7.629 | 1.00 | 20.87 | 6 | C |
| ATOM | 6 | O | MET A | 1 | 56.594 | 6.589 | −6.865 | 1.00 | 23.34 | 8 | O |
| ATOM | 7 | N | MET A | 1 | 53.946 | 5.919 | −7.256 | 1.00 | 24.61 | 7 | N |
| ATOM | 8 | CA | MET A | 1 | 54.518 | 7.082 | −8.004 | 1.00 | 23.08 | 6 | C |
| ATOM | 9 | N | LYS A | 2 | 56.562 | 8.341 | −8.294 | 1.00 | 19.63 | 7 | N |
| ATOM | 10 | CA | LYS A | 2 | 57.984 | 8.698 | −8.121 | 1.00 | 22.21 | 6 | C |
| ATOM | 11 | CB | LYS A | 2 | 58.564 | 8.717 | −9.549 | 1.00 | 25.49 | 6 | C |
| ATOM | 12 | CG | LYS A | 2 | 60.078 | 8.679 | −9.528 | 1.00 | 29.33 | 6 | C |
| ATOM | 13 | CD | LYS A | 2 | 60.516 | 8.306 | −10.953 | 1.00 | 29.37 | 6 | C |
| ATOM | 14 | CE | LYS A | 2 | 61.928 | 7.843 | −11.073 | 1.00 | 31.30 | 6 | C |
| ATOM | 15 | NZ | LYS A | 2 | 62.252 | 7.690 | −12.536 | 1.00 | 29.64 | 7 | N |
| ATOM | 16 | C | LYS A | 2 | 58.051 | 10.032 | −7.426 | 1.00 | 17.19 | 6 | C |
| ATOM | 17 | O | LYS A | 2 | 57.404 | 11.028 | −7.852 | 1.00 | 17.21 | 8 | O |
| ATOM | 18 | N | ILE A | 3 | 58.764 | 10.044 | −6.241 | 1.00 | 14.92 | 7 | N |
| ATOM | 19 | CA | ILE A | 3 | 58.705 | 11.287 | −5.475 | 1.00 | 15.39 | 6 | C |
| ATOM | 20 | CB | ILE A | 3 | 57.999 | 11.115 | −4.097 | 1.00 | 17.61 | 6 | C |
| ATOM | 21 | CG2 | ILE A | 3 | 57.944 | 12.447 | −3.357 | 1.00 | 17.47 | 6 | C |
| ATOM | 22 | CG1 | ILE A | 3 | 56.536 | 10.592 | −4.199 | 1.00 | 20.47 | 6 | C |
| ATOM | 23 | CD1 | ILE A | 3 | 56.475 | 9.067 | −4.169 | 1.00 | 21.64 | 6 | C |
| ATOM | 24 | C | ILE A | 3 | 60.147 | 11.685 | −5.129 | 1.00 | 13.79 | 6 | C |
| ATOM | 25 | O | ILE A | 3 | 60.955 | 10.803 | −4.833 | 1.00 | 16.60 | 8 | O |
| ATOM | 26 | N | THR A | 4 | 60.456 | 12.954 | −5.335 | 1.00 | 13.50 | 7 | N |
| ATOM | 27 | CA | THR A | 4 | 61.760 | 13.455 | −4.818 | 1.00 | 12.56 | 6 | C |
| ATOM | 28 | CB | THR A | 4 | 62.444 | 14.378 | −5.800 | 1.00 | 14.61 | 6 | C |
| ATOM | 29 | OG1 | THR A | 4 | 62.917 | 13.532 | −6.904 | 1.00 | 14.84 | 8 | O |
| ATOM | 30 | CG2 | THR A | 4 | 63.682 | 15.024 | −5.232 | 1.00 | 15.01 | 6 | C |
| ATOM | 31 | C | THR A | 4 | 61.481 | 14.247 | −3.552 | 1.00 | 13.83 | 6 | C |
| ATOM | 32 | O | THR A | 4 | 60.592 | 15.091 | −3.541 | 1.00 | 15.98 | 8 | O |
| ATOM | 33 | N | VAL A | 5 | 62.138 | 13.871 | −2.449 | 1.00 | 15.82 | 7 | N |
| ATOM | 34 | CA | VAL A | 5 | 61.931 | 14.584 | −1.162 | 1.00 | 16.34 | 6 | C |
| ATOM | 35 | CB | VAL A | 5 | 61.855 | 13.590 | −0.006 | 1.00 | 16.01 | 6 | C |
| ATOM | 36 | CG1 | VAL A | 5 | 61.594 | 14.392 | 1.273 | 1.00 | 15.34 | 6 | C |
| ATOM | 37 | CG2 | VAL A | 5 | 60.588 | 12.738 | −0.276 | 1.00 | 14.70 | 6 | C |
| ATOM | 38 | C | VAL A | 5 | 63.098 | 15.565 | −0.956 | 1.00 | 13.32 | 6 | C |
| ATOM | 39 | O | VAL A | 5 | 64.229 | 15.061 | −0.885 | 1.00 | 16.84 | 8 | O |
| ATOM | 40 | N | LEU A | 6 | 62.814 | 16.824 | −0.845 | 1.00 | 15.55 | 7 | N |
| ATOM | 41 | CA | LEU A | 6 | 63.888 | 17.841 | −0.764 | 1.00 | 14.62 | 6 | C |
| ATOM | 42 | CB | LEU A | 6 | 63.388 | 19.096 | −1.459 | 1.00 | 17.58 | 6 | C |
| ATOM | 43 | CG | LEU A | 6 | 64.380 | 20.273 | −1.534 | 1.00 | 22.04 | 6 | C |
| ATOM | 44 | CD1 | LEU A | 6 | 65.793 | 19.938 | −1.876 | 1.00 | 18.88 | 6 | C |
| ATOM | 45 | CD2 | LEU A | 6 | 63.812 | 21.299 | −2.524 | 1.00 | 19.30 | 6 | C |
| ATOM | 46 | C | LEU A | 6 | 64.190 | 18.068 | 0.708 | 1.00 | 18.31 | 6 | C |
| ATOM | 47 | O | LEU A | 6 | 63.351 | 18.593 | 1.448 | 1.00 | 20.51 | 8 | O |
| ATOM | 48 | N | GLY A | 7 | 65.360 | 17.604 | 1.139 | 1.00 | 23.20 | 7 | N |
| ATOM | 49 | CA | GLY A | 7 | 65.757 | 17.747 | 2.553 | 1.00 | 24.61 | 6 | C |
| ATOM | 50 | C | GLY A | 7 | 65.807 | 16.365 | 3.176 | 1.00 | 24.52 | 6 | C |
| ATOM | 51 | O | GLY A | 7 | 64.791 | 15.647 | 3.078 | 1.00 | 27.29 | 8 | O |
| ATOM | 52 | N | CYS A | 8 | 66.918 | 15.864 | 3.690 | 1.00 | 25.89 | 7 | N |
| ATOM | 53 | CA | CYS A | 8 | 67.001 | 14.560 | 4.315 | 1.00 | 28.80 | 6 | C |
| ATOM | 54 | C | CYS A | 8 | 67.175 | 14.739 | 5.833 | 1.00 | 25.20 | 6 | C |
| ATOM | 55 | O | CYS A | 8 | 67.938 | 13.961 | 6.413 | 1.00 | 25.70 | 8 | O |
| ATOM | 56 | CB | CYS A | 8 | 68.070 | 13.525 | 3.902 | 1.00 | 34.71 | 6 | C |
| ATOM | 57 | SG | CYS A | 8 | 67.189 | 12.009 | 3.419 | 1.00 | 46.36 | 16 | S |
| ATOM | 58 | N | GLY A | 9 | 66.475 | 15.712 | 6.411 | 1.00 | 24.26 | 7 | N |
| ATOM | 59 | CA | GLY A | 9 | 66.532 | 15.945 | 7.854 | 1.00 | 22.02 | 6 | C |
| ATOM | 60 | C | GLY A | 9 | 65.406 | 15.219 | 8.574 | 1.00 | 20.60 | 6 | C |
| ATOM | 61 | O | GLY A | 9 | 64.955 | 14.239 | 7.986 | 1.00 | 18.40 | 8 | O |
| ATOM | 62 | N | ALA A | 10 | 64.963 | 15.735 | 9.748 | 1.00 | 19.36 | 7 | N |
| ATOM | 63 | CA | ALA A | 10 | 63.921 | 14.868 | 10.426 | 1.00 | 21.05 | 6 | C |
| ATOM | 64 | CB | ALA A | 10 | 63.551 | 15.551 | 11.745 | 1.00 | 21.49 | 6 | C |
| ATOM | 65 | C | ALA A | 10 | 62.636 | 14.662 | 9.666 | 1.00 | 19.44 | 6 | C |
| ATOM | 66 | O | ALA A | 10 | 62.087 | 13.549 | 9.525 | 1.00 | 19.81 | 8 | O |
| ATOM | 67 | N | LEU A | 11 | 62.002 | 15.784 | 9.235 | 1.00 | 20.27 | 7 | N |
| ATOM | 68 | CA | LEU A | 11 | 60.728 | 15.686 | 8.536 | 1.00 | 18.28 | 6 | C |
| ATOM | 69 | CB | LEU A | 11 | 60.139 | 17.119 | 8.405 | 1.00 | 21.71 | 6 | C |
| ATOM | 70 | CG | LEU A | 11 | 58.889 | 17.163 | 7.555 | 1.00 | 22.72 | 6 | C |
| ATOM | 71 | CD1 | LEU A | 11 | 57.771 | 16.356 | 8.192 | 1.00 | 24.38 | 6 | C |
| ATOM | 72 | CD2 | LEU A | 11 | 58.516 | 18.637 | 7.299 | 1.00 | 23.72 | 6 | C |
| ATOM | 73 | C | LEU A | 11 | 60.889 | 15.004 | 7.181 | 1.00 | 17.57 | 6 | C |
| ATOM | 74 | O | LEU A | 11 | 60.011 | 14.158 | 6.766 | 1.00 | 17.25 | 8 | O |
| ATOM | 75 | N | GLY A | 12 | 62.012 | 15.263 | 6.511 | 1.00 | 15.67 | 7 | N |
| ATOM | 76 | CA | GLY A | 12 | 62.254 | 14.590 | 5.248 | 1.00 | 18.30 | 6 | C |
| ATOM | 77 | C | GLY A | 12 | 62.451 | 13.094 | 5.370 | 1.00 | 18.15 | 6 | C |
| ATOM | 78 | O | GLY A | 12 | 61.919 | 12.267 | 4.570 | 1.00 | 17.92 | 8 | O |
| ATOM | 79 | N | GLN A | 13 | 63.199 | 12.653 | 6.397 | 1.00 | 19.55 | 7 | N |
| ATOM | 80 | CA | GLN A | 13 | 63.335 | 11.210 | 6.624 | 1.00 | 18.33 | 6 | C |
| ATOM | 81 | CB | GLN A | 13 | 64.442 | 10.939 | 7.700 | 1.00 | 18.91 | 6 | C |
| ATOM | 82 | CG | GLN A | 13 | 65.818 | 11.293 | 7.063 | 1.00 | 20.08 | 6 | C |
| ATOM | 83 | CD | GLN A | 13 | 66.940 | 10.761 | 7.997 | 1.00 | 22.93 | 6 | C |
| ATOM | 84 | OE1 | GLN A | 13 | 66.808 | 9.659 | 8.507 | 1.00 | 23.62 | 8 | O |
| ATOM | 85 | NE2 | GLN A | 13 | 67.941 | 11.601 | 8.170 | 1.00 | 25.45 | 7 | N |
| ATOM | 86 | C | GLN A | 13 | 62.000 | 10.622 | 7.061 | 1.00 | 15.51 | 6 | C |
| ATOM | 87 | O | GLN A | 13 | 61.745 | 9.472 | 6.751 | 1.00 | 16.12 | 8 | O |
| ATOM | 88 | N | LEU A | 14 | 61.181 | 11.341 | 7.835 | 1.00 | 14.35 | 7 | N |
| ATOM | 89 | CA | LEU A | 14 | 59.860 | 10.711 | 8.172 | 1.00 | 17.19 | 6 | C |
| ATOM | 90 | CB | LEU A | 14 | 59.139 | 11.750 | 9.061 | 1.00 | 16.49 | 6 | C |
| ATOM | 91 | CG | LEU A | 14 | 57.686 | 11.325 | 9.410 | 1.00 | 18.59 | 6 | C |
| ATOM | 92 | CD1 | LEU A | 14 | 57.638 | 10.077 | 10.285 | 1.00 | 21.00 | 6 | C |
| ATOM | 93 | CD2 | LEU A | 14 | 56.942 | 12.463 | 10.096 | 1.00 | 19.96 | 6 | C |
| ATOM | 94 | C | LEU A | 14 | 59.048 | 10.470 | 6.906 | 1.00 | 17.91 | 6 | C |
| ATOM | 95 | O | LEU A | 14 | 58.436 | 9.391 | 6.811 | 1.00 | 15.26 | 8 | O |
| ATOM | 96 | N | TRP A | 15 | 59.039 | 11.449 | 5.978 | 1.00 | 16.91 | 7 | N |
| ATOM | 97 | CA | TRP A | 15 | 58.322 | 11.180 | 4.692 | 1.00 | 16.16 | 6 | C |
| ATOM | 98 | CB | TRP A | 15 | 58.076 | 12.517 | 3.938 | 1.00 | 17.68 | 6 | C |
| ATOM | 99 | CG | TRP A | 15 | 56.910 | 13.278 | 4.501 | 1.00 | 18.00 | 6 | C |
| ATOM | 100 | CD2 | TRP A | 15 | 55.522 | 13.014 | 4.245 | 1.00 | 19.10 | 6 | C |
| ATOM | 101 | CE2 | TRP A | 15 | 54.770 | 13.956 | 4.964 | 1.00 | 18.74 | 6 | C |
| ATOM | 102 | CE3 | TRP A | 15 | 54.873 | 12.034 | 3.467 | 1.00 | 19.08 | 6 | C |
| ATOM | 103 | CD1 | TRP A | 15 | 56.937 | 14.340 | 5.341 | 1.00 | 18.78 | 6 | C |
| ATOM | 104 | NE1 | TRP A | 15 | 55.637 | 14.772 | 5.633 | 1.00 | 19.15 | 7 | N |
| ATOM | 105 | CZ2 | TRP A | 15 | 53.375 | 13.984 | 4.927 | 1.00 | 20.45 | 6 | C |
| ATOM | 106 | CZ3 | TRP A | 15 | 53.480 | 12.066 | 3.411 | 1.00 | 19.42 | 6 | C |
| ATOM | 107 | CH2 | TRP A | 15 | 52.773 | 13.023 | 4.161 | 1.00 | 17.84 | 6 | C |
| ATOM | 108 | C | TRP A | 15 | 58.917 | 10.151 | 3.787 | 1.00 | 16.85 | 6 | C |
| ATOM | 109 | O | TRP A | 15 | 58.248 | 9.258 | 3.153 | 1.00 | 17.10 | 8 | O |
| ATOM | 110 | N | LEU A | 16 | 60.263 | 10.149 | 3.684 | 1.00 | 15.77 | 7 | N |
| ATOM | 111 | CA | LEU A | 16 | 60.975 | 9.137 | 2.892 | 1.00 | 20.28 | 6 | C |
| ATOM | 112 | CB | LEU A | 16 | 62.478 | 9.343 | 2.927 | 1.00 | 20.13 | 6 | C |
| ATOM | 113 | CG | LEU A | 16 | 62.958 | 10.487 | 2.008 | 1.00 | 20.00 | 6 | C |
| ATOM | 114 | CD1 | LEU A | 16 | 64.361 | 10.910 | 2.379 | 1.00 | 21.07 | 6 | C |
| ATOM | 115 | CD2 | LEU A | 16 | 62.876 | 10.141 | 0.529 | 1.00 | 21.60 | 6 | C |
| ATOM | 116 | C | LEU A | 16 | 60.615 | 7.728 | 3.436 | 1.00 | 20.33 | 6 | C |
| ATOM | 117 | O | LEU A | 16 | 60.383 | 6.818 | 2.634 | 1.00 | 19.53 | 8 | O |
| ATOM | 118 | N | THR A | 17 | 60.741 | 7.552 | 4.758 | 1.00 | 19.26 | 7 | N |
| ATOM | 119 | CA | THR A | 17 | 60.371 | 6.280 | 5.398 | 1.00 | 18.67 | 6 | C |
| ATOM | 120 | CB | THR A | 17 | 60.546 | 6.360 | 6.929 | 1.00 | 19.72 | 6 | C |
| ATOM | 121 | OG1 | THR A | 17 | 61.930 | 6.610 | 7.205 | 1.00 | 18.94 | 8 | O |
| ATOM | 122 | CG2 | THR A | 17 | 60.050 | 5.086 | 7.559 | 1.00 | 20.09 | 6 | C |
| ATOM | 123 | C | THR A | 17 | 58.938 | 5.874 | 5.062 | 1.00 | 18.71 | 6 | C |
| ATOM | 124 | O | THR A | 17 | 58.697 | 4.747 | 4.636 | 1.00 | 20.38 | 8 | O |
| ATOM | 125 | N | ALA A | 18 | 57.966 | 6.814 | 5.202 | 1.00 | 17.14 | 7 | N |
| ATOM | 126 | CA | ALA A | 18 | 56.591 | 6.411 | 4.941 | 1.00 | 16.53 | 6 | C |
| ATOM | 127 | CB | ALA A | 18 | 55.614 | 7.586 | 5.288 | 1.00 | 16.23 | 6 | C |
| ATOM | 128 | C | ALA A | 18 | 56.315 | 6.082 | 3.489 | 1.00 | 18.72 | 6 | C |
| ATOM | 129 | O | ALA A | 18 | 55.576 | 5.120 | 3.148 | 1.00 | 17.41 | 8 | O |
| ATOM | 130 | N | LEU A | 19 | 56.908 | 6.858 | 2.596 | 1.00 | 18.01 | 7 | N |
| ATOM | 131 | CA | LEU A | 19 | 56.757 | 6.614 | 1.159 | 1.00 | 18.56 | 6 | C |
| ATOM | 132 | CB | LEU A | 19 | 57.315 | 7.733 | 0.289 | 1.00 | 17.67 | 6 | C |
| ATOM | 133 | CG | LEU A | 19 | 56.585 | 9.039 | 0.338 | 1.00 | 18.41 | 6 | C |
| ATOM | 134 | CD1 | LEU A | 19 | 57.427 | 10.221 | −0.135 | 1.00 | 17.13 | 6 | C |
| ATOM | 135 | CD2 | LEU A | 19 | 55.270 | 9.124 | −0.474 | 1.00 | 19.35 | 6 | C |
| ATOM | 136 | C | LEU A | 19 | 57.410 | 5.294 | 0.745 | 1.00 | 19.60 | 6 | C |
| ATOM | 137 | O | LEU A | 19 | 56.838 | 4.593 | −0.077 | 1.00 | 21.93 | 8 | O |
| ATOM | 138 | N | CYS A | 20 | 58.543 | 5.068 | 1.379 | 1.00 | 21.17 | 7 | N |
| ATOM | 139 | CA | CYS A | 20 | 59.314 | 3.889 | 1.205 | 1.00 | 26.06 | 6 | C |
| ATOM | 140 | CB | CYS A | 20 | 60.662 | 3.921 | 1.943 | 1.00 | 30.09 | 6 | C |
| ATOM | 141 | SG | CYS A | 20 | 61.845 | 4.632 | 0.747 | 1.00 | 38.10 | 16 | S |
| ATOM | 142 | C | CYS A | 20 | 58.579 | 2.672 | 1.706 | 1.00 | 28.22 | 6 | C |
| ATOM | 143 | O | CYS A | 20 | 59.071 | 1.924 | 0.908 | 1.00 | 36.01 | 8 | O |
| ATOM | 144 | N | LYS A | 21 | 57.768 | 2.656 | 2.704 | 1.00 | 24.14 | 7 | N |
| ATOM | 145 | CA | LYS A | 21 | 57.047 | 1.519 | 3.193 | 1.00 | 25.65 | 6 | C |
| ATOM | 146 | CB | LYS A | 21 | 56.646 | 1.747 | 4.660 | 1.00 | 27.10 | 6 | C |
| ATOM | 147 | CG | LYS A | 21 | 57.737 | 1.801 | 5.711 | 1.00 | 27.34 | 6 | C |
| ATOM | 148 | CD | LYS A | 21 | 57.106 | 1.829 | 7.074 | 1.00 | 30.27 | 6 | C |
| ATOM | 149 | CE | LYS A | 21 | 56.401 | 3.129 | 7.419 | 1.00 | 31.99 | 6 | C |
| ATOM | 150 | NZ | LYS A | 21 | 56.267 | 3.203 | 8.950 | 1.00 | 34.09 | 7 | N |
| ATOM | 151 | C | LYS A | 21 | 55.748 | 1.120 | 2.484 | 1.00 | 26.37 | 6 | C |
| ATOM | 152 | O | LYS A | 21 | 55.197 | 0.082 | 2.622 | 1.00 | 28.40 | 8 | O |
| ATOM | 153 | N | GLN A | 22 | 55.102 | 1.902 | 1.657 | 1.00 | 26.00 | 7 | N |
| ATOM | 154 | CA | GLN A | 22 | 54.106 | 1.895 | 0.699 | 1.00 | 25.65 | 6 | C |
| ATOM | 155 | CB | GLN A | 22 | 53.261 | 3.184 | 0.606 | 1.00 | 25.09 | 6 | C |
| ATOM | 156 | CG | GLN A | 22 | 52.420 | 3.247 | 1.925 | 1.00 | 28.14 | 6 | C |
| ATOM | 157 | CD | GLN A | 22 | 51.388 | 2.138 | 1.882 | 1.00 | 30.98 | 6 | C |
| ATOM | 158 | OE1 | GLN A | 22 | 51.164 | 1.365 | 2.805 | 1.00 | 33.33 | 8 | O |
| ATOM | 159 | NE2 | GLN A | 22 | 50.703 | 1.992 | 0.756 | 1.00 | 28.07 | 7 | N |
| ATOM | 160 | C | GLN A | 22 | 54.677 | 1.532 | −0.670 | 1.00 | 25.24 | 6 | C |
| ATOM | 161 | O | GLN A | 22 | 53.814 | 1.430 | −1.551 | 1.00 | 27.33 | 8 | O |
| ATOM | 162 | N | GLY A | 23 | 55.985 | 1.321 | −0.859 | 1.00 | 22.64 | 7 | N |
| ATOM | 163 | CA | GLY A | 23 | 56.396 | 0.840 | −2.172 | 1.00 | 25.64 | 6 | C |
| ATOM | 164 | C | GLY A | 23 | 56.560 | 1.832 | −3.299 | 1.00 | 26.93 | 6 | C |
| ATOM | 165 | O | GLY A | 23 | 56.815 | 1.475 | −4.461 | 1.00 | 26.62 | 8 | O |
| ATOM | 166 | N | HIS A | 24 | 56.570 | 3.131 | −2.954 | 1.00 | 23.72 | 7 | N |
| ATOM | 167 | CA | HIS A | 24 | 56.787 | 4.167 | −3.937 | 1.00 | 19.67 | 6 | C |
| ATOM | 168 | CB | HIS A | 24 | 56.287 | 5.539 | −3.430 | 1.00 | 18.69 | 6 | C |
| ATOM | 169 | CG | HIS A | 24 | 54.813 | 5.561 | −3.226 | 1.00 | 24.69 | 6 | C |
| ATOM | 170 | CD2 | HIS A | 24 | 54.059 | 5.544 | −2.097 | 1.00 | 21.39 | 6 | C |
| ATOM | 171 | ND1 | HIS A | 24 | 53.926 | 5.636 | −4.274 | 1.00 | 25.95 | 7 | N |
| ATOM | 172 | CE1 | HIS A | 24 | 52.679 | 5.687 | −3.786 | 1.00 | 25.47 | 6 | C |
| ATOM | 173 | NE2 | HIS A | 24 | 52.744 | 5.620 | −2.456 | 1.00 | 22.89 | 7 | N |
| ATOM | 174 | C | HIS A | 24 | 58.252 | 4.279 | −4.270 | 1.00 | 19.83 | 6 | C |
| ATOM | 175 | O | HIS A | 24 | 59.089 | 3.816 | −3.529 | 1.00 | 23.43 | 8 | O |
| ATOM | 176 | N | GLU A | 25 | 58.572 | 4.780 | −5.452 | 1.00 | 17.34 | 7 | N |
| ATOM | 177 | CA | GLU A | 25 | 59.954 | 4.966 | −5.910 | 1.00 | 20.52 | 6 | C |
| ATOM | 178 | CB | GLU A | 25 | 60.058 | 5.072 | −7.419 | 1.00 | 20.84 | 6 | C |
| ATOM | 179 | CG | GLU A | 25 | 59.874 | 3.705 | −8.085 | 1.00 | 29.19 | 6 | C |
| ATOM | 180 | CD | GLU A | 25 | 59.997 | 3.925 | −9.594 | 1.00 | 32.13 | 6 | C |
| ATOM | 181 | OE1 | GLU A | 25 | 61.038 | 4.393 | −10.102 | 1.00 | 38.37 | 8 | O |
| ATOM | 182 | OE2 | GLU A | 25 | 58.986 | 3.638 | −10.237 | 1.00 | 37.08 | 8 | O |
| ATOM | 183 | C | GLU A | 25 | 60.311 | 6.318 | −5.303 | 1.00 | 17.03 | 6 | C |
| ATOM | 184 | O | GLU A | 25 | 59.549 | 7.266 | −5.523 | 1.00 | 18.66 | 8 | O |
| ATOM | 185 | N | VAL A | 26 | 61.394 | 6.418 | −4.549 | 1.00 | 16.73 | 7 | N |
| ATOM | 186 | CA | VAL A | 26 | 61.711 | 7.667 | −3.880 | 1.00 | 18.82 | 6 | C |
| ATOM | 187 | CB | VAL A | 26 | 61.228 | 7.674 | −2.413 | 1.00 | 21.87 | 6 | C |
| ATOM | 188 | CG1 | VAL A | 26 | 59.799 | 7.325 | −2.163 | 1.00 | 23.24 | 6 | C |
| ATOM | 189 | CG2 | VAL A | 26 | 62.084 | 6.731 | −1.578 | 1.00 | 25.16 | 6 | C |
| ATOM | 190 | C | VAL A | 26 | 63.214 | 7.980 | −3.894 | 1.00 | 19.86 | 6 | C |
| ATOM | 191 | O | VAL A | 26 | 64.117 | 7.097 | −4.092 | 1.00 | 20.32 | 8 | O |
| ATOM | 192 | N | GLN A | 27 | 63.502 | 9.258 | −3.856 | 1.00 | 16.71 | 7 | N |
| ATOM | 193 | CA | GLN A | 27 | 64.884 | 9.723 | −3.700 | 1.00 | 18.79 | 6 | C |
| ATOM | 194 | CB | GLN A | 27 | 65.643 | 10.142 | −4.955 | 1.00 | 19.11 | 6 | C |
| ATOM | 195 | CG | GLN A | 27 | 65.385 | 11.526 | −5.489 | 1.00 | 18.83 | 6 | C |
| ATOM | 196 | CD | GLN A | 27 | 66.341 | 11.935 | −6.630 | 1.00 | 19.19 | 6 | C |
| ATOM | 197 | OE1 | GLN A | 27 | 67.464 | 11.424 | −6.644 | 1.00 | 22.00 | 8 | O |
| ATOM | 198 | NE2 | GLN A | 27 | 65.775 | 12.832 | −7.417 | 1.00 | 18.74 | 7 | N |
| ATOM | 199 | C | GLN A | 27 | 64.803 | 10.912 | −2.723 | 1.00 | 18.35 | 6 | C |
| ATOM | 200 | O | GLN A | 27 | 63.811 | 11.629 | −2.617 | 1.00 | 17.81 | 8 | O |
| ATOM | 201 | N | GLY A | 28 | 65.981 | 11.156 | −2.062 | 1.00 | 20.09 | 7 | N |
| ATOM | 202 | CA | GLY A | 28 | 66.059 | 12.440 | −1.303 | 1.00 | 19.20 | 6 | C |
| ATOM | 203 | C | GLY A | 28 | 66.936 | 13.387 | −2.146 | 1.00 | 19.03 | 6 | C |
| ATOM | 204 | O | GLY A | 28 | 67.406 | 13.096 | −3.253 | 1.00 | 20.33 | 8 | O |
| ATOM | 205 | N | TRP A | 29 | 67.018 | 14.619 | −1.675 | 1.00 | 20.95 | 7 | N |
| ATOM | 206 | CA | TRP A | 29 | 67.830 | 15.646 | −2.337 | 1.00 | 19.59 | 6 | C |
| ATOM | 207 | CB | TRP A | 29 | 66.947 | 16.516 | −3.232 | 1.00 | 20.98 | 6 | C |
| ATOM | 208 | CG | TRP A | 29 | 67.815 | 17.265 | −4.195 | 1.00 | 23.63 | 6 | C |
| ATOM | 209 | CD2 | TRP A | 29 | 68.091 | 16.950 | −5.559 | 1.00 | 25.60 | 6 | C |
| ATOM | 210 | CE2 | TRP A | 29 | 68.993 | 17.914 | −6.029 | 1.00 | 24.26 | 6 | C |
| ATOM | 211 | CE3 | TRP A | 29 | 67.648 | 15.919 | −6.411 | 1.00 | 24.54 | 6 | C |
| ATOM | 212 | CD1 | TRP A | 29 | 68.546 | 18.401 | −3.902 | 1.00 | 22.61 | 6 | C |
| ATOM | 213 | NE1 | TRP A | 29 | 69.263 | 18.803 | −4.980 | 1.00 | 23.69 | 7 | N |
| ATOM | 214 | CZ2 | TRP A | 29 | 69.454 | 17.872 | −7.328 | 1.00 | 24.60 | 6 | C |
| ATOM | 215 | CZ3 | TRP A | 29 | 68.087 | 15.903 | −7.726 | 1.00 | 25.88 | 6 | C |
| ATOM | 216 | CH2 | TRP A | 29 | 69.006 | 16.888 | −8.138 | 1.00 | 27.86 | 6 | C |
| ATOM | 217 | C | TRP A | 29 | 68.516 | 16.467 | −1.230 | 1.00 | 22.92 | 6 | C |
| ATOM | 218 | O | TRP A | 29 | 67.784 | 17.107 | −0.463 | 1.00 | 22.38 | 8 | O |
| ATOM | 219 | N | LEU A | 30 | 69.846 | 16.551 | −1.269 | 1.00 | 24.34 | 7 | N |
| ATOM | 220 | CA | LEU A | 30 | 70.650 | 17.199 | −0.239 | 1.00 | 27.04 | 6 | C |
| ATOM | 221 | CB | LEU A | 30 | 71.818 | 16.374 | 0.282 | 1.00 | 28.94 | 6 | C |
| ATOM | 222 | CG | LEU A | 30 | 71.574 | 15.166 | 1.178 | 1.00 | 33.56 | 6 | C |
| ATOM | 223 | CD1 | LEU A | 30 | 72.854 | 14.355 | 1.393 | 1.00 | 37.67 | 6 | C |
| ATOM | 224 | CD2 | LEU A | 30 | 70.974 | 15.623 | 2.497 | 1.00 | 34.22 | 6 | C |
| ATOM | 225 | C | LEU A | 30 | 71.270 | 18.505 | −0.754 | 1.00 | 29.02 | 6 | C |
| ATOM | 226 | O | LEU A | 30 | 71.553 | 18.620 | −1.932 | 1.00 | 27.41 | 8 | O |
| ATOM | 227 | N | ALA A | 31 | 71.439 | 19.492 | 0.126 | 1.00 | 32.32 | 7 | N |
| ATOM | 228 | CA | ALA A | 31 | 72.085 | 20.745 | −0.268 | 1.00 | 34.74 | 6 | C |
| ATOM | 229 | CB | ALA A | 31 | 71.906 | 21.785 | 0.827 | 1.00 | 35.48 | 6 | C |
| ATOM | 230 | C | ALA A | 31 | 73.544 | 20.476 | −0.636 | 1.00 | 34.80 | 6 | C |
| ATOM | 231 | O | ALA A | 31 | 74.089 | 21.067 | −1.574 | 1.00 | 36.63 | 8 | O |
| ATOM | 232 | N | VAL A | 32 | 74.193 | 19.536 | 0.020 | 1.00 | 37.59 | 7 | N |
| ATOM | 233 | CA | VAL A | 32 | 75.532 | 19.049 | −0.235 | 1.00 | 39.02 | 6 | C |
| ATOM | 234 | CB | VAL A | 32 | 76.408 | 18.846 | 1.014 | 1.00 | 41.34 | 6 | C |
| ATOM | 235 | CG1 | VAL A | 32 | 77.795 | 18.385 | 0.595 | 1.00 | 42.42 | 6 | C |
| ATOM | 236 | CG2 | VAL A | 32 | 76.521 | 20.102 | 1.864 | 1.00 | 42.34 | 6 | C |
| ATOM | 237 | C | VAL A | 32 | 75.384 | 17.670 | −0.909 | 1.00 | 37.58 | 6 | C |
| ATOM | 238 | O | VAL A | 32 | 75.199 | 16.639 | −0.263 | 1.00 | 38.05 | 8 | O |
| ATOM | 239 | N | PRO A | 33 | 75.462 | 17.665 | −2.226 | 1.00 | 37.22 | 7 | N |
| ATOM | 240 | CD | PRO A | 33 | 75.719 | 18.835 | −3.101 | 1.00 | 37.43 | 6 | C |
| ATOM | 241 | CA | PRO A | 33 | 75.243 | 16.462 | −3.013 | 1.00 | 35.52 | 6 | C |
| ATOM | 242 | CB | PRO A | 33 | 75.530 | 16.861 | −4.452 | 1.00 | 36.13 | 6 | C |
| ATOM | 243 | CG | PRO A | 33 | 75.440 | 18.344 | −4.487 | 1.00 | 37.75 | 6 | C |
| ATOM | 244 | C | PRO A | 33 | 76.011 | 15.249 | −2.566 | 1.00 | 36.25 | 6 | C |
| ATOM | 245 | O | PRO A | 33 | 77.193 | 15.245 | −2.257 | 1.00 | 36.26 | 8 | O |
| ATOM | 246 | N | GLN A | 34 | 75.284 | 14.135 | −2.429 | 1.00 | 34.19 | 7 | N |
| ATOM | 247 | CA | GLN A | 34 | 75.785 | 12.828 | −2.112 | 1.00 | 35.75 | 6 | C |
| ATOM | 248 | CB | GLN A | 34 | 75.750 | 12.408 | −0.649 | 1.00 | 39.04 | 6 | C |
| ATOM | 249 | CG | GLN A | 34 | 76.549 | 13.260 | 0.311 | 1.00 | 43.93 | 6 | C |
| ATOM | 250 | CD | GLN A | 34 | 76.547 | 12.770 | 1.742 | 1.00 | 47.29 | 6 | C |
| ATOM | 251 | OE1 | GLN A | 34 | 75.712 | 13.219 | 2.535 | 1.00 | 50.44 | 8 | O |
| ATOM | 252 | NE2 | GLN A | 34 | 77.461 | 11.877 | 2.088 | 1.00 | 49.07 | 7 | N |
| ATOM | 253 | C | GLN A | 34 | 74.858 | 11.902 | −2.917 | 1.00 | 33.61 | 6 | C |
| ATOM | 254 | O | GLN A | 34 | 73.722 | 12.272 | −3.172 | 1.00 | 32.97 | 8 | O |
| ATOM | 255 | N | PRO A | 35 | 75.370 | 10.758 | −3.305 | 1.00 | 30.29 | 7 | N |
| ATOM | 256 | CD | PRO A | 35 | 76.783 | 10.321 | −3.124 | 1.00 | 31.14 | 6 | C |
| ATOM | 257 | CA | PRO A | 35 | 74.605 | 9.826 | −4.109 | 1.00 | 28.27 | 6 | C |
| ATOM | 258 | CB | PRO A | 35 | 75.683 | 8.843 | −4.606 | 1.00 | 28.55 | 6 | C |
| ATOM | 259 | CG | PRO A | 35 | 76.677 | 8.850 | −3.483 | 1.00 | 29.06 | 6 | C |
| ATOM | 260 | C | PRO A | 35 | 73.521 | 9.134 | −3.324 | 1.00 | 27.10 | 6 | C |
| ATOM | 261 | O | PRO A | 35 | 72.672 | 8.499 | −3.961 | 1.00 | 24.38 | 8 | O |
| ATOM | 262 | N | TYR A | 36 | 73.518 | 9.108 | −1.984 | 1.00 | 26.11 | 7 | N |
| ATOM | 263 | CA | TYR A | 36 | 72.515 | 8.467 | −1.178 | 1.00 | 28.72 | 6 | C |
| ATOM | 264 | CB | TYR A | 36 | 72.976 | 7.107 | −0.635 | 1.00 | 30.45 | 6 | C |
| ATOM | 265 | CG | TYR A | 36 | 73.447 | 6.127 | −1.687 | 1.00 | 30.95 | 6 | C |
| ATOM | 266 | CD1 | TYR A | 36 | 74.785 | 6.123 | −2.064 | 1.00 | 31.40 | 6 | C |
| ATOM | 267 | CE1 | TYR A | 36 | 75.231 | 5.271 | −3.067 | 1.00 | 32.73 | 6 | C |
| ATOM | 268 | CD2 | TYR A | 36 | 72.549 | 5.282 | −2.324 | 1.00 | 32.13 | 6 | C |
| ATOM | 269 | CE2 | TYR A | 36 | 72.997 | 4.412 | −3.321 | 1.00 | 34.36 | 6 | C |
| ATOM | 270 | CZ | TYR A | 36 | 74.330 | 4.407 | −3.657 | 1.00 | 35.08 | 6 | C |
| ATOM | 271 | OH | TYR A | 36 | 74.755 | 3.531 | −4.629 | 1.00 | 36.75 | 8 | O |
| ATOM | 272 | C | TYR A | 36 | 72.161 | 9.277 | 0.082 | 1.00 | 30.06 | 6 | C |
| ATOM | 273 | O | TYR A | 36 | 72.957 | 10.091 | 0.532 | 1.00 | 27.93 | 8 | O |
| ATOM | 274 | N | CYS A | 37 | 70.972 | 9.078 | 0.631 | 1.00 | 31.17 | 7 | N |
| ATOM | 275 | CA | CYS A | 37 | 70.506 | 9.588 | 1.887 | 1.00 | 33.35 | 6 | C |
| ATOM | 276 | C | CYS A | 37 | 70.413 | 8.407 | 2.855 | 1.00 | 32.03 | 6 | C |
| ATOM | 277 | O | CYS A | 37 | 69.647 | 7.469 | 2.556 | 1.00 | 28.66 | 8 | O |
| ATOM | 278 | CB | CYS A | 37 | 69.055 | 10.099 | 1.758 | 1.00 | 38.20 | 6 | C |
| ATOM | 279 | SG | CYS A | 37 | 68.363 | 10.414 | 3.373 | 1.00 | 45.18 | 16 | S |
| ATOM | 280 | N | SER A | 38 | 71.164 | 8.366 | 3.945 | 1.00 | 29.83 | 7 | N |
| ATOM | 281 | CA | SER A | 38 | 71.086 | 7.207 | 4.838 | 1.00 | 30.30 | 6 | C |
| ATOM | 282 | CB | SER A | 38 | 72.375 | 7.035 | 5.676 | 1.00 | 32.01 | 6 | C |
| ATOM | 283 | OG | SER A | 38 | 73.444 | 6.795 | 4.738 | 1.00 | 37.81 | 8 | O |
| ATOM | 284 | C | SER A | 38 | 69.922 | 7.445 | 5.794 | 1.00 | 28.69 | 6 | C |
| ATOM | 285 | O | SER A | 38 | 70.078 | 8.267 | 6.684 | 1.00 | 31.39 | 8 | O |
| ATOM | 286 | N | VAL A | 39 | 68.804 | 6.737 | 5.655 | 1.00 | 26.81 | 7 | N |
| ATOM | 287 | CA | VAL A | 39 | 67.699 | 6.918 | 6.612 | 1.00 | 27.33 | 6 | C |
| ATOM | 288 | CB | VAL A | 39 | 66.395 | 6.658 | 5.811 | 1.00 | 29.60 | 6 | C |
| ATOM | 289 | CG1 | VAL A | 39 | 65.183 | 6.775 | 6.746 | 1.00 | 27.91 | 6 | C |
| ATOM | 290 | CG2 | VAL A | 39 | 66.294 | 7.642 | 4.650 | 1.00 | 28.67 | 6 | C |
| ATOM | 291 | C | VAL A | 39 | 67.767 | 5.996 | 7.806 | 1.00 | 30.13 | 6 | C |
| ATOM | 292 | O | VAL A | 39 | 68.033 | 4.782 | 7.787 | 1.00 | 27.57 | 8 | O |
| ATOM | 293 | N | ASN A | 40 | 67.553 | 6.571 | 8.993 | 1.00 | 28.95 | 7 | N |
| ATOM | 294 | CA | ASN A | 40 | 67.581 | 5.767 | 10.223 | 1.00 | 30.58 | 6 | C |
| ATOM | 295 | CB | ASN A | 40 | 68.953 | 5.725 | 10.906 | 1.00 | 35.11 | 6 | C |
| ATOM | 296 | CG | ASN A | 40 | 68.826 | 4.881 | 12.163 | 1.00 | 36.53 | 6 | C |
| ATOM | 297 | OD1 | ASN A | 40 | 68.026 | 5.242 | 13.066 | 1.00 | 41.25 | 8 | O |
| ATOM | 298 | ND2 | ASN A | 40 | 69.404 | 3.708 | 12.250 | 1.00 | 35.72 | 7 | N |
| ATOM | 299 | C | ASN A | 40 | 66.562 | 6.419 | 11.139 | 1.00 | 30.39 | 6 | C |
| ATOM | 300 | O | ASN A | 40 | 66.914 | 7.361 | 11.855 | 1.00 | 31.27 | 8 | O |
| ATOM | 301 | N | LEU A | 41 | 65.343 | 5.912 | 11.097 | 1.00 | 29.97 | 7 | N |
| ATOM | 302 | CA | LEU A | 41 | 64.313 | 6.501 | 11.907 | 1.00 | 28.42 | 6 | C |
| ATOM | 303 | CB | LEU A | 41 | 63.208 | 6.936 | 10.903 | 1.00 | 30.47 | 6 | C |
| ATOM | 304 | CG | LEU A | 41 | 62.022 | 7.631 | 11.568 | 1.00 | 32.81 | 6 | C |
| ATOM | 305 | CD1 | LEU A | 41 | 61.543 | 8.748 | 10.656 | 1.00 | 35.09 | 6 | C |
| ATOM | 306 | CD2 | LEU A | 41 | 60.942 | 6.582 | 11.755 | 1.00 | 32.60 | 6 | C |
| ATOM | 307 | C | LEU A | 41 | 63.697 | 5.528 | 12.906 | 1.00 | 28.47 | 6 | C |
| ATOM | 308 | O | LEU A | 41 | 63.562 | 4.342 | 12.616 | 1.00 | 25.42 | 8 | O |
| ATOM | 309 | N | VAL A | 42 | 63.438 | 6.021 | 14.104 | 1.00 | 22.39 | 7 | N |
| ATOM | 310 | CA | VAL A | 42 | 62.762 | 5.248 | 15.145 | 1.00 | 22.71 | 6 | C |
| ATOM | 311 | CB | VAL A | 42 | 63.320 | 5.650 | 16.500 | 1.00 | 22.47 | 6 | C |
| ATOM | 312 | CG1 | VAL A | 42 | 62.670 | 4.901 | 17.690 | 1.00 | 20.48 | 6 | C |
| ATOM | 313 | CG2 | VAL A | 42 | 64.847 | 5.383 | 16.540 | 1.00 | 20.32 | 6 | C |
| ATOM | 314 | C | VAL A | 42 | 61.261 | 5.511 | 15.092 | 1.00 | 24.25 | 6 | C |
| ATOM | 315 | O | VAL A | 42 | 60.843 | 6.670 | 15.314 | 1.00 | 22.31 | 8 | O |
| ATOM | 316 | N | GLU A | 43 | 60.445 | 4.479 | 14.893 | 1.00 | 25.16 | 7 | N |
| ATOM | 317 | CA | GLU A | 43 | 58.989 | 4.574 | 14.877 | 1.00 | 26.39 | 6 | C |
| ATOM | 318 | CB | GLU A | 43 | 58.439 | 3.214 | 14.353 | 1.00 | 28.10 | 6 | C |
| ATOM | 319 | CG | GLU A | 43 | 59.005 | 2.782 | 12.984 | 1.00 | 28.95 | 6 | C |
| ATOM | 320 | CD | GLU A | 43 | 58.148 | 3.305 | 11.839 | 1.00 | 33.52 | 6 | C |
| ATOM | 321 | OE1 | GLU A | 43 | 57.372 | 4.287 | 12.009 | 1.00 | 32.49 | 8 | O |
| ATOM | 322 | OE2 | GLU A | 43 | 58.157 | 2.730 | 10.718 | 1.00 | 31.85 | 8 | O |
| ATOM | 323 | C | GLU A | 43 | 58.377 | 4.882 | 16.221 | 1.00 | 27.61 | 6 | C |
| ATOM | 324 | O | GLU A | 43 | 59.039 | 4.736 | 17.258 | 1.00 | 28.26 | 8 | O |
| ATOM | 325 | N | THR A | 44 | 57.092 | 5.292 | 16.355 | 1.00 | 27.54 | 7 | N |
| ATOM | 326 | CA | THR A | 44 | 56.510 | 5.670 | 17.618 | 1.00 | 27.58 | 6 | C |
| ATOM | 327 | CB | THR A | 44 | 55.078 | 6.251 | 17.547 | 1.00 | 29.47 | 6 | C |
| ATOM | 328 | OG1 | THR A | 44 | 54.224 | 5.342 | 16.837 | 1.00 | 29.12 | 8 | O |
| ATOM | 329 | CG2 | THR A | 44 | 55.085 | 7.594 | 16.818 | 1.00 | 29.28 | 6 | C |
| ATOM | 330 | C | THR A | 44 | 56.487 | 4.496 | 18.620 | 1.00 | 30.49 | 6 | C |
| ATOM | 331 | O | THR A | 44 | 56.453 | 4.767 | 19.825 | 1.00 | 32.82 | 8 | O |
| ATOM | 332 | N | ASP A | 45 | 56.541 | 3.262 | 18.104 | 1.00 | 31.19 | 7 | N |
| ATOM | 333 | CA | ASP A | 45 | 56.562 | 2.152 | 19.104 | 1.00 | 33.22 | 6 | C |
| ATOM | 334 | CB | ASP A | 45 | 55.872 | 0.898 | 18.580 | 1.00 | 34.18 | 6 | C |
| ATOM | 335 | CG | ASP A | 45 | 56.565 | 0.236 | 17.422 | 1.00 | 34.94 | 6 | C |
| ATOM | 336 | OD1 | ASP A | 45 | 57.414 | 0.823 | 16.723 | 1.00 | 32.61 | 8 | O |
| ATOM | 337 | OD2 | ASP A | 45 | 56.284 | −0.937 | 17.087 | 1.00 | 40.48 | 8 | O |
| ATOM | 338 | C | ASP A | 45 | 57.998 | 1.799 | 19.465 | 1.00 | 32.01 | 6 | C |
| ATOM | 339 | O | ASP A | 45 | 58.171 | 0.784 | 20.150 | 1.00 | 32.97 | 8 | O |
| ATOM | 340 | N | GLY A | 46 | 58.999 | 2.520 | 18.950 | 1.00 | 27.61 | 7 | N |
| ATOM | 341 | CA | GLY A | 46 | 60.382 | 2.153 | 19.286 | 1.00 | 26.30 | 6 | C |
| ATOM | 342 | C | GLY A | 46 | 61.032 | 1.283 | 18.231 | 1.00 | 25.92 | 6 | C |
| ATOM | 343 | O | GLY A | 46 | 62.231 | 0.945 | 18.310 | 1.00 | 28.47 | 8 | O |
| ATOM | 344 | N | SER A | 47 | 60.296 | 0.831 | 17.232 | 1.00 | 25.17 | 7 | N |
| ATOM | 345 | CA | SER A | 47 | 60.845 | −0.009 | 16.181 | 1.00 | 26.65 | 6 | C |
| ATOM | 346 | CB | SER A | 47 | 59.795 | −0.746 | 15.369 | 1.00 | 28.94 | 6 | C |
| ATOM | 347 | OG | SER A | 47 | 58.668 | 0.008 | 14.970 | 1.00 | 31.45 | 8 | O |
| ATOM | 348 | C | SER A | 47 | 61.771 | 0.827 | 15.302 | 1.00 | 27.69 | 6 | C |
| ATOM | 349 | O | SER A | 47 | 61.673 | 2.052 | 15.261 | 1.00 | 27.44 | 8 | O |
| ATOM | 350 | N | ILE A | 48 | 62.734 | 0.138 | 14.702 | 1.00 | 28.27 | 7 | N |
| ATOM | 351 | CA | ILE A | 48 | 63.641 | 0.937 | 13.818 | 1.00 | 26.68 | 6 | C |
| ATOM | 352 | CB | ILE A | 48 | 65.108 | 0.554 | 14.107 | 1.00 | 28.07 | 6 | C |
| ATOM | 353 | CG2 | ILE A | 48 | 66.073 | 1.364 | 13.261 | 1.00 | 27.07 | 6 | C |
| ATOM | 354 | CG1 | ILE A | 48 | 65.363 | 0.837 | 15.590 | 1.00 | 27.86 | 6 | C |
| ATOM | 355 | CD1 | ILE A | 48 | 66.784 | 0.588 | 16.060 | 1.00 | 30.99 | 6 | C |
| ATOM | 356 | C | ILE A | 48 | 63.277 | 0.663 | 12.375 | 1.00 | 30.36 | 6 | C |
| ATOM | 357 | O | ILE A | 48 | 63.152 | −0.541 | 12.448 | 1.00 | 31.57 | 8 | O |
| ATOM | 358 | N | PHE A | 49 | 63.555 | 1.550 | 11.474 | 1.00 | 26.28 | 7 | N |
| ATOM | 359 | CA | PHE A | 49 | 63.541 | 1.550 | 10.030 | 1.00 | 26.18 | 6 | C |
| ATOM | 360 | CB | PHE A | 49 | 62.460 | 2.397 | 9.352 | 1.00 | 26.59 | 6 | C |
| ATOM | 361 | CG | PHE A | 49 | 62.477 | 2.458 | 7.891 | 1.00 | 26.59 | 6 | C |
| ATOM | 362 | CD1 | PHE A | 49 | 61.729 | 1.568 | 7.117 | 1.00 | 28.67 | 6 | C |
| ATOM | 363 | CD2 | PHE A | 49 | 63.224 | 3.382 | 7.159 | 1.00 | 26.37 | 6 | C |
| ATOM | 364 | CE1 | PHE A | 49 | 61.717 | 1.584 | 5.745 | 1.00 | 25.41 | 6 | C |
| ATOM | 365 | CE2 | PHE A | 49 | 63.237 | 3.412 | 5.776 | 1.00 | 28.69 | 6 | C |
| ATOM | 366 | CZ | PHE A | 49 | 62.474 | 2.511 | 5.050 | 1.00 | 28.49 | 6 | C |
| ATOM | 367 | C | PHE A | 49 | 64.905 | 2.139 | 9.573 | 1.00 | 25.57 | 6 | C |
| ATOM | 368 | O | PHE A | 49 | 65.230 | 3.256 | 9.955 | 1.00 | 27.53 | 8 | O |
| ATOM | 369 | N | ASN A | 50 | 65.630 | 1.405 | 8.704 | 1.00 | 24.76 | 7 | N |
| ATOM | 370 | CA | ASN A | 50 | 66.967 | 1.843 | 8.287 | 1.00 | 27.69 | 6 | C |
| ATOM | 371 | CB | ASN A | 50 | 68.001 | 1.351 | 9.343 | 1.00 | 30.08 | 6 | C |
| ATOM | 372 | CG | ASN A | 50 | 69.412 | 1.805 | 9.002 | 1.00 | 31.06 | 6 | C |
| ATOM | 373 | OD1 | ASN A | 50 | 69.926 | 2.596 | 9.818 | 1.00 | 34.00 | 8 | O |
| ATOM | 374 | ND2 | ASN A | 50 | 70.064 | 1.351 | 7.961 | 1.00 | 30.73 | 7 | N |
| ATOM | 375 | C | ASN A | 50 | 67.303 | 1.324 | 6.944 | 1.00 | 30.93 | 6 | C |
| ATOM | 376 | O | ASN A | 50 | 67.404 | 0.144 | 6.639 | 1.00 | 29.95 | 8 | O |
| ATOM | 377 | N | GLU A | 51 | 67.326 | 2.255 | 5.984 | 1.00 | 29.10 | 7 | N |
| ATOM | 378 | CA | GLU A | 51 | 67.600 | 1.975 | 4.579 | 1.00 | 30.55 | 6 | C |
| ATOM | 379 | CB | GLU A | 51 | 66.297 | 1.865 | 3.773 | 1.00 | 31.87 | 6 | C |
| ATOM | 380 | CC | GLU A | 51 | 65.505 | 0.595 | 4.093 | 1.00 | 38.93 | 6 | C |
| ATOM | 381 | CD | GLU A | 51 | 66.396 | −0.620 | 3.826 | 1.00 | 40.93 | 6 | C |
| ATOM | 382 | OE1 | GLU A | 51 | 67.105 | −0.596 | 2.785 | 1.00 | 43.62 | 8 | O |
| ATOM | 383 | OE2 | GLU A | 51 | 66.411 | −1.517 | 4.669 | 1.00 | 44.47 | 8 | O |
| ATOM | 384 | C | GLU A | 51 | 68.444 | 3.095 | 4.012 | 1.00 | 29.33 | 6 | C |
| ATOM | 385 | O | GLU A | 51 | 68.362 | 4.252 | 4.354 | 1.00 | 29.83 | 8 | O |
| ATOM | 386 | N | SER A | 52 | 69.120 | 2.776 | 2.902 | 1.00 | 28.60 | 7 | N |
| ATOM | 387 | CA | SER A | 52 | 69.899 | 3.708 | 2.102 | 1.00 | 27.45 | 6 | C |
| ATOM | 388 | CB | SER A | 52 | 71.237 | 3.115 | 1.651 | 1.00 | 28.30 | 6 | C |
| ATOM | 389 | OG | SER A | 52 | 72.241 | 3.248 | 2.626 | 1.00 | 30.94 | 8 | O |
| ATOM | 390 | C | SER A | 52 | 69.176 | 4.097 | 0.814 | 1.00 | 26.94 | 6 | C |
| ATOM | 391 | O | SER A | 52 | 69.065 | 3.297 | −0.114 | 1.00 | 28.23 | 8 | O |
| ATOM | 392 | N | LEU A | 53 | 68.739 | 5.339 | 0.654 | 1.00 | 24.84 | 7 | N |
| ATOM | 393 | CA | LEU A | 53 | 68.007 | 5.713 | −0.549 | 1.00 | 24.79 | 6 | C |
| ATOM | 394 | CB | LEU A | 53 | 66.697 | 6.455 | −0.173 | 1.00 | 26.66 | 6 | C |
| ATOM | 395 | CG | LEU A | 53 | 65.820 | 5.727 | 0.848 | 1.00 | 28.60 | 6 | C |
| ATOM | 396 | CD1 | LEU A | 53 | 64.633 | 6.633 | 1.213 | 1.00 | 30.62 | 6 | C |
| ATOM | 397 | CD2 | LEU A | 53 | 65.338 | 4.406 | 0.269 | 1.00 | 30.17 | 6 | C |
| ATOM | 398 | C | LEU A | 53 | 68.805 | 6.548 | −1.536 | 1.00 | 25.47 | 6 | C |
| ATOM | 399 | O | LEU A | 53 | 69.776 | 7.240 | −1.202 | 1.00 | 26.52 | 8 | O |
| ATOM | 400 | N | THR A | 54 | 68.400 | 6.491 | −2.811 | 1.00 | 23.31 | 7 | N |
| ATOM | 401 | CA | THR A | 54 | 68.991 | 7.285 | −3.872 | 1.00 | 23.91 | 6 | C |
| ATOM | 402 | CB | THR A | 54 | 68.194 | 7.155 | −5.216 | 1.00 | 25.87 | 6 | C |
| ATOM | 403 | OG1 | THR A | 54 | 68.498 | 5.838 | −5.702 | 1.00 | 27.15 | 8 | O |
| ATOM | 404 | CG2 | THR A | 54 | 68.672 | 8.153 | −6.252 | 1.00 | 25.02 | 6 | C |
| ATOM | 405 | C | THR A | 54 | 68.874 | 8.773 | −3.519 | 1.00 | 23.03 | 6 | C |
| ATOM | 406 | O | THR A | 54 | 67.920 | 9.109 | −2.851 | 1.00 | 22.88 | 8 | O |
| ATOM | 407 | N | ALA A | 55 | 69.864 | 9.600 | −3.774 | 1.00 | 23.21 | 7 | N |
| ATOM | 408 | CA | ALA A | 55 | 69.750 | 11.042 | −3.548 | 1.00 | 24.90 | 6 | C |
| ATOM | 409 | CB | ALA A | 55 | 70.407 | 11.503 | −2.223 | 1.00 | 22.63 | 6 | C |
| ATOM | 410 | C | ALA A | 55 | 70.402 | 11.794 | −4.693 | 1.00 | 23.12 | 6 | C |
| ATOM | 411 | O | ALA A | 55 | 71.332 | 11.290 | −5.379 | 1.00 | 24.57 | 8 | O |
| ATOM | 412 | N | ASN A | 56 | 69.889 | 12.965 | −5.025 | 1.00 | 21.77 | 7 | N |
| ATOM | 413 | CA | ASN A | 56 | 70.494 | 13.903 | −5.950 | 1.00 | 22.43 | 6 | C |
| ATOM | 414 | CB | ASN A | 56 | 71.792 | 14.458 | −5.353 | 1.00 | 25.10 | 6 | C |
| ATOM | 415 | CG | ASN A | 56 | 71.596 | 15.370 | −4.145 | 1.00 | 25.06 | 6 | C |
| ATOM | 416 | OD1 | ASN A | 56 | 71.184 | 14.930 | −3.095 | 1.00 | 24.70 | 8 | O |
| ATOM | 417 | ND2 | ASN A | 56 | 71.921 | 16.651 | −4.357 | 1.00 | 26.52 | 7 | N |
| ATOM | 418 | C | ASN A | 56 | 70.729 | 13.405 | −7.360 | 1.00 | 22.86 | 6 | C |
| ATOM | 419 | O | ASN A | 56 | 71.663 | 13.778 | −8.064 | 1.00 | 24.56 | 8 | O |
| ATOM | 420 | N | ASP A | 57 | 69.859 | 12.549 | −7.854 | 1.00 | 21.29 | 7 | N |
| ATOM | 421 | CA | ASP A | 57 | 69.841 | 12.006 | −9.202 | 1.00 | 23.50 | 6 | C |
| ATOM | 422 | CB | ASP A | 57 | 69.245 | 10.598 | −9.082 | 1.00 | 24.41 | 6 | C |
| ATOM | 423 | CG | ASP A | 57 | 69.320 | 9.885 | −10.446 | 1.00 | 27.36 | 6 | C |
| ATOM | 424 | OD1 | ASP A | 57 | 69.210 | 10.544 | −11.491 | 1.00 | 27.51 | 8 | O |
| ATOM | 425 | OD2 | ASP A | 57 | 69.421 | 8.641 | −10.351 | 1.00 | 27.67 | 8 | O |
| ATOM | 426 | C | ASP A | 57 | 68.943 | 12.911 | −10.044 | 1.00 | 25.21 | 6 | C |
| ATOM | 427 | O | ASP A | 57 | 67.725 | 12.979 | −9.865 | 1.00 | 21.29 | 8 | O |
| ATOM | 428 | N | PRO A | 58 | 69.537 | 13.711 | −10.964 | 1.00 | 24.99 | 7 | N |
| ATOM | 429 | CD | PRO A | 58 | 70.990 | 13.600 | −11.279 | 1.00 | 26.79 | 6 | C |
| ATOM | 430 | CA | PRO A | 58 | 68.850 | 14.606 | −11.841 | 1.00 | 24.66 | 6 | C |
| ATOM | 431 | CB | PRO A | 58 | 70.008 | 15.325 | −12.578 | 1.00 | 28.21 | 6 | C |
| ATOM | 432 | CG | PRO A | 58 | 71.118 | 14.313 | −12.596 | 1.00 | 28.45 | 6 | C |
| ATOM | 433 | C | PRO A | 58 | 67.872 | 13.933 | −12.814 | 1.00 | 23.24 | 6 | C |
| ATOM | 434 | O | PRO A | 58 | 66.932 | 14.576 | −13.183 | 1.00 | 19.94 | 8 | O |
| ATOM | 435 | N | ASP A | 59 | 68.161 | 12.713 | −13.212 | 1.00 | 24.23 | 7 | N |
| ATOM | 436 | CA | ASP A | 59 | 67.245 | 11.994 | −14.117 | 1.00 | 26.19 | 6 | C |
| ATOM | 437 | CB | ASP A | 59 | 67.961 | 10.851 | −14.821 | 1.00 | 30.36 | 6 | C |
| ATOM | 438 | CG | ASP A | 59 | 69.077 | 11.362 | −15.715 | 1.00 | 32.27 | 6 | C |
| ATOM | 439 | OD1 | ASP A | 59 | 68.814 | 12.259 | −16.532 | 1.00 | 32.01 | 8 | O |
| ATOM | 440 | OD2 | ASP A | 59 | 70.207 | 10.832 | −15.619 | 1.00 | 32.97 | 8 | O |
| ATOM | 441 | C | ASP A | 59 | 66.007 | 11.534 | −13.362 | 1.00 | 22.45 | 6 | C |
| ATOM | 442 | O | ASP A | 59 | 64.934 | 11.545 | −13.936 | 1.00 | 23.55 | 8 | O |
| ATOM | 443 | N | PHE A | 60 | 66.145 | 11.138 | −12.109 | 1.00 | 24.18 | 7 | N |
| ATOM | 444 | CA | PHE A | 60 | 65.022 | 10.719 | −11.266 | 1.00 | 20.30 | 6 | C |
| ATOM | 445 | CB | PHE A | 60 | 65.605 | 10.154 | −9.945 | 1.00 | 20.19 | 6 | C |
| ATOM | 446 | CG | PHE A | 60 | 64.583 | 9.499 | −9.052 | 1.00 | 19.97 | 6 | C |
| ATOM | 447 | CD1 | PHE A | 60 | 64.581 | 8.136 | −8.894 | 1.00 | 26.19 | 6 | C |
| ATOM | 448 | CD2 | PHE A | 60 | 63.613 | 10.260 | −8.375 | 1.00 | 20.25 | 6 | C |
| ATOM | 449 | CE1 | PHE A | 60 | 63.622 | 7.510 | −8.077 | 1.00 | 26.55 | 6 | C |
| ATOM | 450 | CE2 | PHE A | 60 | 62.667 | 9.674 | −7.556 | 1.00 | 21.54 | 6 | C |
| ATOM | 451 | CZ | PHE A | 60 | 62.692 | 8.304 | −7.431 | 1.00 | 24.51 | 6 | C |
| ATOM | 452 | C | PHE A | 60 | 64.175 | 11.974 | −10.932 | 1.00 | 19.89 | 6 | C |
| ATOM | 453 | O | PHE A | 60 | 62.948 | 12.041 | −11.052 | 1.00 | 19.19 | 8 | O |
| ATOM | 454 | N | LEU A | 61 | 64.871 | 13.131 | −10.698 | 1.00 | 18.98 | 7 | N |
| ATOM | 455 | CA | LEU A | 61 | 64.122 | 14.371 | −10.522 | 1.00 | 18.49 | 6 | C |
| ATOM | 456 | CB | LEU A | 61 | 65.094 | 15.529 | −10.179 | 1.00 | 19.12 | 6 | C |
| ATOM | 457 | CG | LEU A | 61 | 64.440 | 16.918 | −10.060 | 1.00 | 19.99 | 6 | C |
| ATOM | 458 | CD1 | LEU A | 61 | 63.482 | 16.970 | −8.880 | 1.00 | 19.96 | 6 | C |
| ATOM | 459 | CD2 | LEU A | 61 | 65.627 | 17.916 | −9.948 | 1.00 | 20.79 | 6 | C |
| ATOM | 460 | C | LEU A | 61 | 63.315 | 14.765 | −11.740 | 1.00 | 19.01 | 6 | C |
| ATOM | 461 | O | LEU A | 61 | 62.162 | 15.182 | −11.660 | 1.00 | 16.82 | 8 | O |
| ATOM | 462 | N | ALA A | 62 | 63.920 | 14.597 | −12.962 | 1.00 | 21.18 | 7 | N |
| ATOM | 463 | CA | ALA A | 62 | 63.249 | 14.967 | −14.193 | 1.00 | 22.64 | 6 | C |
| ATOM | 464 | CB | ALA A | 62 | 64.258 | 14.800 | −15.334 | 1.00 | 23.13 | 6 | C |
| ATOM | 465 | C | ALA A | 62 | 62.068 | 14.038 | −14.516 | 1.00 | 23.74 | 6 | C |
| ATOM | 466 | O | ALA A | 62 | 61.287 | 14.417 | −15.402 | 1.00 | 21.55 | 8 | O |
| ATOM | 467 | N | THR A | 63 | 61.889 | 12.913 | −13.854 | 1.00 | 20.73 | 7 | N |
| ATOM | 468 | CA | THR A | 63 | 60.741 | 12.038 | −14.064 | 1.00 | 21.42 | 6 | C |
| ATOM | 469 | CB | THR A | 63 | 61.187 | 10.641 | −14.571 | 1.00 | 24.63 | 6 | C |
| ATOM | 470 | OG1 | THR A | 63 | 62.298 | 10.243 | −13.764 | 1.00 | 25.73 | 8 | O |
| ATOM | 471 | CG2 | THR A | 63 | 61.583 | 10.705 | −16.033 | 1.00 | 28.77 | 6 | C |
| ATOM | 472 | C | THR A | 63 | 59.877 | 11.818 | −12.833 | 1.00 | 21.48 | 6 | C |
| ATOM | 473 | O | THR A | 63 | 59.056 | 10.886 | −12.654 | 1.00 | 20.77 | 8 | O |
| ATOM | 474 | N | SER A | 64 | 60.012 | 12.694 | −11.829 | 1.00 | 18.51 | 7 | N |
| ATOM | 475 | CA | SER A | 64 | 59.237 | 12.633 | −10.614 | 1.00 | 18.29 | 6 | C |
| ATOM | 476 | CB | SER A | 64 | 59.970 | 13.489 | −9.522 | 1.00 | 15.65 | 6 | C |
| ATOM | 477 | OG | SER A | 64 | 61.100 | 12.794 | −9.045 | 1.00 | 16.92 | 8 | O |
| ATOM | 478 | C | SER A | 64 | 57.832 | 13.205 | −10.766 | 1.00 | 17.95 | 6 | C |
| ATOM | 479 | O | SER A | 64 | 57.545 | 14.117 | −11.508 | 1.00 | 20.47 | 8 | O |
| ATOM | 480 | N | ASP A | 65 | 56.884 | 12.593 | −10.057 | 1.00 | 17.41 | 7 | N |
| ATOM | 481 | CA | ASP A | 65 | 55.487 | 12.904 | −10.022 | 1.00 | 17.41 | 6 | C |
| ATOM | 482 | CB | ASP A | 65 | 54.648 | 11.577 | −9.941 | 1.00 | 19.70 | 6 | C |
| ATOM | 483 | CG | ASP A | 65 | 54.993 | 10.654 | −11.118 | 1.00 | 22.29 | 6 | C |
| ATOM | 484 | OD1 | ASP A | 65 | 54.820 | 11.083 | −12.274 | 1.00 | 23.75 | 8 | O |
| ATOM | 485 | OD2 | ASP A | 65 | 55.491 | 9.566 | −10.830 | 1.00 | 22.12 | 8 | O |
| ATOM | 486 | C | ASP A | 65 | 55.052 | 13.754 | −8.849 | 1.00 | 16.95 | 6 | C |
| ATOM | 487 | O | ASP A | 65 | 53.963 | 14.325 | −8.840 | 1.00 | 16.63 | 8 | O |
| ATOM | 488 | N | LEU A | 66 | 56.033 | 14.011 | −7.963 | 1.00 | 17.15 | 7 | N |
| ATOM | 489 | CA | LEU A | 66 | 55.779 | 14.936 | −6.826 | 1.00 | 16.87 | 6 | C |
| ATOM | 490 | CB | LEU A | 66 | 54.960 | 14.271 | −5.709 | 1.00 | 17.02 | 6 | C |
| ATOM | 491 | CG | LEU A | 66 | 54.874 | 15.039 | −4.364 | 1.00 | 14.65 | 6 | C |
| ATOM | 492 | CD1 | LEU A | 66 | 53.871 | 16.247 | −4.524 | 1.00 | 14.57 | 6 | C |
| ATOM | 493 | CD2 | LEU A | 66 | 54.291 | 14.204 | −3.233 | 1.00 | 14.66 | 6 | C |
| ATOM | 494 | C | LEU A | 66 | 57.135 | 15.380 | −6.256 | 1.00 | 15.24 | 6 | C |
| ATOM | 495 | O | LEU A | 66 | 58.078 | 14.575 | −6.303 | 1.00 | 16.38 | 8 | O |
| ATOM | 496 | N | LEU A | 67 | 57.268 | 16.653 | −5.981 | 1.00 | 17.25 | 7 | N |
| ATOM | 497 | CA | LEU A | 67 | 58.461 | 17.226 | −5.313 | 1.00 | 15.53 | 6 | C |
| ATOM | 498 | CB | LEU A | 67 | 59.094 | 18.430 | −5.976 | 1.00 | 13.95 | 6 | C |
| ATOM | 499 | CG | LEU A | 67 | 60.232 | 19.155 | −5.200 | 1.00 | 16.50 | 6 | C |
| ATOM | 500 | CD1 | LEU A | 67 | 61.370 | 18.132 | −5.018 | 1.00 | 16.64 | 6 | C |
| ATOM | 501 | CD2 | LEU A | 67 | 60.750 | 20.346 | −6.039 | 1.00 | 17.22 | 6 | C |
| ATOM | 502 | C | LEU A | 67 | 57.905 | 17.582 | −3.916 | 1.00 | 14.98 | 6 | C |
| ATOM | 503 | O | LEU A | 67 | 57.023 | 18.458 | −3.810 | 1.00 | 14.63 | 8 | O |
| ATOM | 504 | N | LEU A | 68 | 58.367 | 16.857 | −2.868 | 1.00 | 14.52 | 7 | N |
| ATOM | 505 | CA | LEU A | 68 | 57.845 | 17.122 | −1.522 | 1.00 | 15.85 | 6 | C |
| ATOM | 506 | CB | LEU A | 68 | 57.540 | 15.810 | −0.796 | 1.00 | 17.84 | 6 | C |
| ATOM | 507 | CG | LEU A | 68 | 56.662 | 15.861 | 0.470 | 1.00 | 23.41 | 6 | C |
| ATOM | 508 | CD1 | LEU A | 68 | 56.378 | 14.362 | 0.765 | 1.00 | 25.33 | 6 | C |
| ATOM | 509 | CD2 | LEU A | 68 | 57.556 | 16.260 | 1.636 | 1.00 | 22.62 | 6 | C |
| ATOM | 510 | C | LEU A | 68 | 58.928 | 17.915 | −0.729 | 1.00 | 14.52 | 6 | C |
| ATOM | 511 | O | LEU A | 68 | 59.964 | 17.291 | −0.513 | 1.00 | 15.28 | 8 | O |
| ATOM | 512 | N | VAL A | 69 | 58.693 | 19.125 | −0.291 | 1.00 | 13.09 | 7 | N |
| ATOM | 513 | CA | VAL A | 69 | 59.789 | 19.898 | 0.346 | 1.00 | 13.59 | 6 | C |
| ATOM | 514 | CB | VAL A | 69 | 59.705 | 21.331 | −0.270 | 1.00 | 15.34 | 6 | C |
| ATOM | 515 | CG1 | VAL A | 69 | 60.811 | 22.215 | 0.292 | 1.00 | 16.51 | 6 | C |
| ATOM | 516 | CG2 | VAL A | 69 | 59.759 | 21.246 | −1.798 | 1.00 | 16.33 | 6 | C |
| ATOM | 517 | C | VAL A | 69 | 59.620 | 20.034 | 1.853 | 1.00 | 16.08 | 6 | C |
| ATOM | 518 | O | VAL A | 69 | 58.531 | 20.359 | 2.346 | 1.00 | 17.15 | 8 | O |
| ATOM | 519 | N | THR A | 70 | 60.788 | 19.703 | 2.485 | 1.00 | 13.35 | 7 | N |
| ATOM | 520 | CA | THR A | 70 | 60.722 | 19.582 | 3.961 | 1.00 | 14.54 | 6 | C |
| ATOM | 521 | CB | THR A | 70 | 60.842 | 18.132 | 4.482 | 1.00 | 15.96 | 6 | C |
| ATOM | 522 | OG1 | THR A | 70 | 62.065 | 17.512 | 4.066 | 1.00 | 19.36 | 8 | O |
| ATOM | 523 | CG2 | THR A | 70 | 59.740 | 17.233 | 3.879 | 1.00 | 15.54 | 6 | C |
| ATOM | 524 | C | THR A | 70 | 61.868 | 20.353 | 4.554 | 1.00 | 17.39 | 6 | C |
| ATOM | 525 | O | THR A | 70 | 62.185 | 20.112 | 5.757 | 1.00 | 23.55 | 8 | O |
| ATOM | 526 | N | LEU A | 71 | 62.265 | 21.411 | 3.884 | 1.00 | 17.46 | 7 | N |
| ATOM | 527 | CA | LEU A | 71 | 63.316 | 22.278 | 4.473 | 1.00 | 19.45 | 6 | C |
| ATOM | 528 | CB | LEU A | 71 | 63.950 | 22.962 | 3.250 | 1.00 | 21.65 | 6 | C |
| ATOM | 529 | CG | LEU A | 71 | 64.694 | 22.077 | 2.273 | 1.00 | 20.39 | 6 | C |
| ATOM | 530 | CD1 | LEU A | 71 | 65.196 | 22.884 | 1.088 | 1.00 | 25.73 | 6 | C |
| ATOM | 531 | CD2 | LEU A | 71 | 65.860 | 21.340 | 2.955 | 1.00 | 26.91 | 6 | C |
| ATOM | 532 | C | LEU A | 71 | 62.787 | 23.307 | 5.416 | 1.00 | 20.85 | 6 | C |
| ATOM | 533 | O | LEU A | 71 | 61.591 | 23.568 | 5.479 | 1.00 | 18.79 | 8 | O |
| ATOM | 534 | N | LYS A | 72 | 63.679 | 23.993 | 6.213 | 1.00 | 20.21 | 7 | N |
| ATOM | 535 | CA | LYS A | 72 | 63.212 | 25.089 | 7.027 | 1.00 | 22.60 | 6 | C |
| ATOM | 536 | CB | LYS A | 72 | 64.357 | 25.614 | 7.909 | 1.00 | 25.53 | 6 | C |
| ATOM | 537 | CG | LYS A | 72 | 65.059 | 24.491 | 8.677 | 1.00 | 29.68 | 6 | C |
| ATOM | 538 | CD | LYS A | 72 | 64.106 | 24.080 | 9.787 | 1.00 | 33.74 | 6 | C |
| ATOM | 539 | CE | LYS A | 72 | 64.024 | 22.555 | 9.795 | 1.00 | 38.42 | 6 | C |
| ATOM | 540 | NZ | LYS A | 72 | 63.270 | 22.103 | 11.027 | 1.00 | 41.23 | 7 | N |
| ATOM | 541 | C | LYS A | 72 | 62.826 | 26.247 | 6.083 | 1.00 | 22.00 | 6 | C |
| ATOM | 542 | O | LYS A | 72 | 63.355 | 26.350 | 4.980 | 1.00 | 21.07 | 8 | O |
| ATOM | 543 | N | ALA A | 73 | 61.957 | 27.098 | 6.580 | 1.00 | 21.11 | 7 | N |
| ATOM | 544 | CA | ALA A | 73 | 61.360 | 28.118 | 5.727 | 1.00 | 19.00 | 6 | C |
| ATOM | 545 | CB | ALA A | 73 | 60.406 | 28.962 | 6.538 | 1.00 | 20.88 | 6 | C |
| ATOM | 546 | C | ALA A | 73 | 62.354 | 29.016 | 4.992 | 1.00 | 19.18 | 6 | C |
| ATOM | 547 | O | ALA A | 73 | 62.112 | 29.286 | 3.787 | 1.00 | 17.71 | 8 | O |
| ATOM | 548 | N | TRP A | 74 | 63.406 | 29.441 | 5.658 | 1.00 | 20.62 | 7 | N |
| ATOM | 549 | CA | TRP A | 74 | 64.359 | 30.366 | 4.992 | 1.00 | 20.81 | 6 | C |
| ATOM | 550 | CB | TRP A | 74 | 65.279 | 30.968 | 6.104 | 1.00 | 23.28 | 6 | C |
| ATOM | 551 | CG | TRP A | 74 | 66.030 | 29.984 | 6.928 | 1.00 | 22.49 | 6 | C |
| ATOM | 552 | CD2 | TRP A | 74 | 67.316 | 29.409 | 6.699 | 1.00 | 26.07 | 6 | C |
| ATOM | 553 | CE2 | TRP A | 74 | 67.596 | 28.552 | 7.779 | 1.00 | 27.46 | 6 | C |
| ATOM | 554 | CE3 | TPP A | 74 | 68.288 | 29.608 | 5.705 | 1.00 | 27.76 | 6 | C |
| ATOM | 555 | CD1 | TRP A | 74 | 65.611 | 29.443 | 8.113 | 1.00 | 24.70 | 6 | C |
| ATOM | 556 | NE1 | TRP A | 74 | 66.506 | 28.548 | 8.609 | 1.00 | 27.52 | 7 | N |
| ATOM | 557 | CZ2 | TRP A | 74 | 68.780 | 27.832 | 7.882 | 1.00 | 31.03 | 6 | C |
| ATOM | 558 | CZ3 | TRP A | 74 | 69.476 | 28.888 | 5.816 | 1.00 | 31.29 | 6 | C |
| ATOM | 559 | CH2 | TRP A | 74 | 69.699 | 28.016 | 6.889 | 1.00 | 30.15 | 6 | C |
| ATOM | 560 | C | TRP A | 74 | 65.182 | 29.745 | 3.899 | 1.00 | 22.12 | 6 | C |
| ATOM | 561 | O | TRP A | 74 | 65.915 | 30.384 | 3.090 | 1.00 | 21.36 | 8 | O |
| ATOM | 562 | N | GLN A | 75 | 65.153 | 28.417 | 3.745 | 1.00 | 20.22 | 7 | N |
| ATOM | 563 | CA | GLN A | 75 | 65.858 | 27.646 | 2.771 | 1.00 | 22.57 | 6 | C |
| ATOM | 564 | CB | GLN A | 75 | 66.286 | 26.282 | 3.342 | 1.00 | 25.07 | 6 | C |
| ATOM | 565 | CG | GLN A | 75 | 67.311 | 26.385 | 4.485 | 1.00 | 28.42 | 6 | C |
| ATOM | 566 | CD | GLN A | 75 | 67.484 | 25.102 | 5.258 | 1.00 | 33.15 | 6 | C |
| ATOM | 567 | OE1 | GLN A | 75 | 66.670 | 24.161 | 5.307 | 1.00 | 34.09 | 8 | O |
| ATOM | 568 | NE2 | GLN A | 75 | 68.588 | 24.993 | 6.019 | 1.00 | 34.82 | 7 | N |
| ATOM | 569 | C | GLN A | 75 | 64.949 | 27.366 | 1.556 | 1.00 | 22.13 | 6 | C |
| ATOM | 570 | O | GLN A | 75 | 65.583 | 26.773 | 0.631 | 1.00 | 23.16 | 8 | O |
| ATOM | 571 | N | VAL A | 76 | 63.673 | 27.283 | 1.746 | 1.00 | 20.65 | 7 | N |
| ATOM | 572 | CA | VAL A | 76 | 62.779 | 26.823 | 0.707 | 1.00 | 18.83 | 6 | C |
| ATOM | 573 | CB | VAL A | 76 | 61.339 | 26.862 | 1.262 | 1.00 | 19.12 | 6 | C |
| ATOM | 574 | CG1 | VAL A | 76 | 60.278 | 26.693 | 0.178 | 1.00 | 19.10 | 6 | C |
| ATOM | 575 | CG2 | VAL A | 76 | 61.172 | 25.912 | 2.456 | 1.00 | 20.33 | 6 | C |
| ATOM | 576 | C | VAL A | 76 | 62.867 | 27.486 | −0.653 | 1.00 | 19.86 | 6 | C |
| ATOM | 577 | O | VAL A | 76 | 62.994 | 26.682 | −1.622 | 1.00 | 22.31 | 8 | O |
| ATOM | 578 | N | SER A | 77 | 62.631 | 28.760 | −0.748 | 1.00 | 20.94 | 7 | N |
| ATOM | 579 | CA | SER A | 77 | 62.424 | 29.328 | −2.104 | 1.00 | 22.51 | 6 | C |
| ATOM | 580 | CB | SER A | 77 | 61.834 | 30.735 | −1.945 | 1.00 | 22.72 | 6 | C |
| ATOM | 581 | OG | SER A | 77 | 62.875 | 31.614 | −1.526 | 1.00 | 28.58 | 8 | O |
| ATOM | 582 | C | SER A | 77 | 63.626 | 29.194 | −2.999 | 1.00 | 24.42 | 6 | C |
| ATOM | 583 | O | SER A | 77 | 63.503 | 28.771 | −4.168 | 1.00 | 25.80 | 8 | O |
| ATOM | 584 | N | ASP A | 78 | 64.864 | 29.387 | −2.487 | 1.00 | 26.64 | 7 | N |
| ATOM | 585 | CA | ASP A | 78 | 66.049 | 29.200 | −3.323 | 1.00 | 27.37 | 6 | C |
| ATOM | 586 | CB | ASP A | 78 | 67.327 | 29.651 | −2.624 | 1.00 | 30.35 | 6 | C |
| ATOM | 587 | CG | ASP A | 78 | 67.474 | 31.131 | −2.350 | 1.00 | 34.66 | 6 | C |
| ATOM | 588 | OD1 | ASP A | 78 | 67.050 | 32.001 | −3.125 | 1.00 | 36.48 | 8 | O |
| ATOM | 589 | OD2 | ASP A | 78 | 68.014 | 31.488 | −1.273 | 1.00 | 35.74 | 8 | O |
| ATOM | 590 | C | ASP A | 78 | 66.228 | 27.756 | −3.792 | 1.00 | 26.83 | 6 | C |
| ATOM | 591 | O | ASP A | 78 | 66.577 | 27.507 | −4.979 | 1.00 | 26.62 | 8 | O |
| ATOM | 592 | N | ALA A | 79 | 66.031 | 26.778 | −2.890 | 1.00 | 24.51 | 7 | N |
| ATOM | 593 | CA | ALA A | 79 | 66.223 | 25.391 | −3.245 | 1.00 | 23.01 | 6 | C |
| ATOM | 594 | CB | ALA A | 79 | 66.218 | 24.440 | −2.065 | 1.00 | 23.43 | 6 | C |
| ATOM | 595 | C | ALA A | 79 | 65.141 | 24.952 | −4.279 | 1.00 | 21.89 | 6 | C |
| ATOM | 596 | O | ALA A | 79 | 65.539 | 24.282 | −5.234 | 1.00 | 20.72 | 8 | O |
| ATOM | 597 | N | VAL A | 80 | 63.899 | 25.340 | −4.100 | 1.00 | 18.80 | 7 | N |
| ATOM | 598 | CA | VAL A | 80 | 62.847 | 25.004 | −5.069 | 1.00 | 20.18 | 6 | C |
| ATOM | 599 | CB | VAL A | 80 | 61.454 | 25.459 | −4.601 | 1.00 | 20.16 | 6 | C |
| ATOM | 600 | CG1 | VAL A | 80 | 60.329 | 25.289 | −5.644 | 1.00 | 19.66 | 6 | C |
| ATOM | 601 | CG2 | VAL A | 80 | 61.066 | 24.569 | −3.390 | 1.00 | 20.18 | 6 | C |
| ATOM | 602 | C | VAL A | 80 | 63.186 | 25.640 | −6.418 | 1.00 | 21.67 | 6 | C |
| ATOM | 603 | O | VAL A | 80 | 63.113 | 24.934 | −7.456 | 1.00 | 22.95 | 8 | O |
| ATOM | 604 | N | LYS A | 81 | 63.593 | 26.908 | −6.439 | 1.00 | 21.12 | 7 | N |
| ATOM | 605 | CA | LYS A | 81 | 63.887 | 27.547 | −7.780 | 1.00 | 20.40 | 6 | C |
| ATOM | 606 | CB | LYS A | 81 | 64.131 | 29.027 | −7.546 | 1.00 | 21.88 | 6 | C |
| ATOM | 607 | CG | LYS A | 81 | 62.954 | 29.895 | −7.201 | 1.00 | 24.11 | 6 | C |
| ATOM | 608 | CD | LYS A | 81 | 63.388 | 31.305 | −6.859 | 1.00 | 28.17 | 6 | C |
| ATOM | 609 | CE | LYS A | 81 | 62.253 | 32.257 | −6.649 | 1.00 | 32.11 | 6 | C |
| ATOM | 610 | NZ | LYS A | 81 | 62.755 | 33.492 | −5.946 | 1.00 | 32.28 | 7 | N |
| ATOM | 611 | C | LYS A | 81 | 65.013 | 26.801 | −8.461 | 1.00 | 23.68 | 6 | C |
| ATOM | 612 | O | LYS A | 81 | 64.933 | 26.629 | −9.714 | 1.00 | 22.41 | 8 | O |
| ATOM | 613 | N | SER A | 82 | 66.031 | 26.288 | −7.756 | 1.00 | 24.42 | 7 | N |
| ATOM | 614 | CA | SER A | 82 | 67.129 | 25.555 | −8.299 | 1.00 | 27.30 | 6 | C |
| ATOM | 615 | CB | SER A | 82 | 68.192 | 25.145 | −7.282 | 1.00 | 31.44 | 6 | C |
| ATOM | 616 | OG | SER A | 82 | 67.931 | 24.117 | −6.337 | 1.00 | 35.47 | 8 | O |
| ATOM | 617 | C | SER A | 82 | 66.677 | 24.270 | −9.031 | 1.00 | 27.43 | 6 | C |
| ATOM | 618 | O | SER A | 82 | 67.431 | 23.789 | −9.876 | 1.00 | 29.76 | 8 | O |
| ATOM | 619 | N | LEU A | 83 | 65.666 | 23.600 | −8.509 | 1.00 | 25.35 | 7 | N |
| ATOM | 620 | CA | LEU A | 83 | 65.225 | 22.342 | −9.105 | 1.00 | 25.04 | 6 | C |
| ATOM | 621 | CB | LEU A | 83 | 64.726 | 21.428 | −7.958 | 1.00 | 23.04 | 6 | C |
| ATOM | 622 | CG | LEU A | 83 | 65.718 | 21.028 | −6.887 | 1.00 | 25.94 | 6 | C |
| ATOM | 623 | CD1 | LEU A | 83 | 65.141 | 19.787 | −6.137 | 1.00 | 25.74 | 6 | C |
| ATOM | 624 | CD2 | LEU A | 83 | 67.088 | 20.630 | −7.381 | 1.00 | 24.66 | 6 | C |
| ATOM | 625 | C | LEU A | 83 | 64.083 | 22.483 | −10.078 | 1.00 | 25.04 | 6 | C |
| ATOM | 626 | O | LEU A | 83 | 63.723 | 21.487 | −10.703 | 1.00 | 24.30 | 8 | O |
| ATOM | 627 | N | ALA A | 84 | 63.383 | 23.603 | −10.149 | 1.00 | 25.31 | 7 | N |
| ATOM | 628 | CA | ALA A | 84 | 62.197 | 23.734 | −10.936 | 1.00 | 27.21 | 6 | C |
| ATOM | 629 | CB | ALA A | 84 | 61.364 | 24.938 | −10.515 | 1.00 | 29.05 | 6 | C |
| ATOM | 630 | C | ALA A | 84 | 62.419 | 23.669 | −12.450 | 1.00 | 27.24 | 6 | C |
| ATOM | 631 | O | ALA A | 84 | 61.469 | 23.277 | −13.149 | 1.00 | 26.80 | 8 | O |
| ATOM | 632 | N | SER A | 85 | 63.617 | 24.022 | −12.933 | 1.00 | 26.34 | 7 | N |
| ATOM | 633 | CA | SER A | 85 | 63.800 | 23.860 | −14.389 | 1.00 | 27.31 | 6 | C |
| ATOM | 634 | CB | SER A | 85 | 64.988 | 24.741 | −14.845 | 1.00 | 28.02 | 6 | C |
| ATOM | 635 | OG | SER A | 85 | 66.134 | 23.998 | −14.398 | 1.00 | 34.04 | 8 | O |
| ATOM | 636 | C | SER A | 85 | 64.014 | 22.445 | −14.823 | 1.00 | 27.16 | 6 | C |
| ATOM | 637 | O | SER A | 85 | 63.832 | 22.013 | −15.974 | 1.00 | 27.41 | 8 | O |
| ATOM | 638 | N | THR A | 86 | 64.350 | 21.521 | −13.904 | 1.00 | 25.79 | 7 | N |
| ATOM | 639 | CA | THR A | 86 | 64.597 | 20.123 | −14.147 | 1.00 | 23.33 | 6 | C |
| ATOM | 640 | CB | THR A | 86 | 65.800 | 19.610 | −13.339 | 1.00 | 25.22 | 6 | C |
| ATOM | 641 | OG1 | THR A | 86 | 66.991 | 20.316 | −13.756 | 1.00 | 26.39 | 8 | O |
| ATOM | 642 | CG2 | THR A | 86 | 66.019 | 18.127 | −13.577 | 1.00 | 24.19 | 6 | C |
| ATOM | 643 | C | THR A | 86 | 63.358 | 19.268 | −13.854 | 1.00 | 22.48 | 6 | C |
| ATOM | 644 | O | THR A | 86 | 63.074 | 18.350 | −14.630 | 1.00 | 22.47 | 8 | O |
| ATOM | 645 | N | LEU A | 87 | 62.663 | 19.608 | −12.775 | 1.00 | 19.32 | 7 | N |
| ATOM | 646 | CA | LEU A | 87 | 61.422 | 18.821 | −12.459 | 1.00 | 20.09 | 6 | C |
| ATOM | 647 | CB | LEU A | 87 | 60.910 | 19.490 | −11.140 | 1.00 | 20.09 | 6 | C |
| ATOM | 648 | CG | LEU A | 87 | 59.658 | 18.896 | −10.518 | 1.00 | 21.12 | 6 | C |
| ATOM | 649 | CD1 | LEU A | 87 | 60.074 | 17.573 | −9.827 | 1.00 | 18.86 | 6 | C |
| ATOM | 650 | CD2 | LEU A | 87 | 58.949 | 19.786 | −9.498 | 1.00 | 17.84 | 6 | C |
| ATOM | 651 | C | LEU A | 87 | 60.433 | 19.058 | −13.589 | 1.00 | 19.47 | 6 | C |
| ATOM | 652 | O | LEU A | 87 | 60.404 | 20.261 | −14.069 | 1.00 | 22.51 | 8 | O |
| ATOM | 653 | N | PRO A | 88 | 59.577 | 18.118 | −13.961 | 1.00 | 18.84 | 7 | N |
| ATOM | 654 | CD | PRO A | 88 | 59.523 | 16.734 | −13.439 | 1.00 | 17.45 | 6 | C |
| ATOM | 655 | CA | PRO A | 88 | 58.601 | 18.425 | −14.993 | 1.00 | 20.15 | 6 | C |
| ATOM | 656 | CB | PRO A | 88 | 57.882 | 17.064 | −15.092 | 1.00 | 19.07 | 6 | C |
| ATOM | 657 | CG | PRO A | 88 | 58.739 | 15.970 | −14.502 | 1.00 | 17.86 | 6 | C |
| ATOM | 658 | C | PRO A | 88 | 57.703 | 19.545 | −14.640 | 1.00 | 21.15 | 6 | C |
| ATOM | 659 | O | PRO A | 88 | 57.253 | 19.837 | −13.538 | 1.00 | 19.76 | 8 | O |
| ATOM | 660 | N | VAL A | 89 | 57.392 | 20.445 | −15.608 | 1.00 | 23.63 | 7 | N |
| ATOM | 661 | CA | VAL A | 89 | 56.536 | 21.599 | −15.441 | 1.00 | 26.38 | 6 | C |
| ATOM | 662 | CB | VAL A | 89 | 56.595 | 22.487 | −16.723 | 1.00 | 27.05 | 6 | C |
| ATOM | 663 | CG1 | VAL A | 89 | 55.751 | 21.820 | −17.789 | 1.00 | 29.86 | 6 | C |
| ATOM | 664 | CG2 | VAL A | 89 | 56.133 | 23.874 | −16.385 | 1.00 | 29.49 | 6 | C |
| ATOM | 665 | C | VAL A | 89 | 55.064 | 21.305 | −15.186 | 1.00 | 29.77 | 6 | C |
| ATOM | 666 | O | VAL A | 89 | 54.159 | 22.137 | −15.235 | 1.00 | 38.50 | 8 | O |
| ATOM | 667 | N | THR A | 90 | 54.756 | 20.030 | −15.001 | 1.00 | 26.08 | 7 | N |
| ATOM | 668 | CA | THR A | 90 | 53.440 | 19.488 | −14.787 | 1.00 | 24.78 | 6 | C |
| ATOM | 669 | CB | THR A | 90 | 53.325 | 18.396 | −15.882 | 1.00 | 25.50 | 6 | C |
| ATOM | 670 | OG1 | THR A | 90 | 54.555 | 17.681 | −16.060 | 1.00 | 33.86 | 8 | O |
| ATOM | 671 | CG2 | THR A | 90 | 52.838 | 19.015 | −17.176 | 1.00 | 28.70 | 6 | C |
| ATOM | 672 | C | THR A | 90 | 53.396 | 18.876 | −13.392 | 1.00 | 23.61 | 6 | C |
| ATOM | 673 | O | THR A | 90 | 52.360 | 18.483 | −12.935 | 1.00 | 21.44 | 8 | O |
| ATOM | 674 | N | THR A | 91 | 54.573 | 18.689 | −12.814 | 1.00 | 21.25 | 7 | N |
| ATOM | 675 | CA | THR A | 91 | 54.634 | 18.000 | −11.536 | 1.00 | 21.12 | 6 | C |
| ATOM | 676 | CB | THR A | 91 | 56.015 | 17.352 | −11.352 | 1.00 | 21.89 | 6 | C |
| ATOM | 677 | OG1 | THR A | 91 | 56.191 | 16.244 | −12.273 | 1.00 | 20.52 | 8 | O |
| ATOM | 678 | CG2 | THR A | 91 | 56.276 | 16.827 | −9.939 | 1.00 | 20.92 | 6 | C |
| ATOM | 679 | C | THR A | 91 | 54.375 | 18.922 | −10.344 | 1.00 | 19.28 | 6 | C |
| ATOM | 680 | O | THR A | 91 | 55.088 | 19.931 | −10.180 | 1.00 | 20.13 | 8 | O |
| ATOM | 681 | N | PRO A | 92 | 53.470 | 18.556 | −9.435 | 1.00 | 17.51 | 7 | N |
| ATOM | 682 | CD | PRO A | 92 | 52.608 | 17.371 | −9.580 | 1.00 | 18.10 | 6 | C |
| ATOM | 683 | CA | PRO A | 92 | 53.164 | 19.354 | −8.252 | 1.00 | 16.18 | 6 | C |
| ATOM | 684 | CB | PRO A | 92 | 51.957 | 18.676 | −7.577 | 1.00 | 18.76 | 6 | C |
| ATOM | 685 | CG | PRO A | 92 | 51.720 | 17.422 | −8.384 | 1.00 | 22.31 | 6 | C |
| ATOM | 686 | C | PRO A | 92 | 54.281 | 19.400 | −7.202 | 1.00 | 17.00 | 6 | C |
| ATOM | 687 | O | PRO A | 92 | 55.109 | 18.501 | −7.079 | 1.00 | 16.44 | 8 | O |
| ATOM | 688 | N | ILE A | 93 | 54.450 | 20.593 | −6.632 | 1.00 | 16.56 | 7 | N |
| ATOM | 689 | CA | ILE A | 93 | 55.419 | 20.845 | −5.546 | 1.00 | 16.18 | 6 | C |
| ATOM | 690 | CB | ILE A | 93 | 56.278 | 22.079 | −5.936 | 1.00 | 17.62 | 6 | C |
| ATOM | 691 | CG2 | ILE A | 93 | 57.202 | 22.434 | −4.759 | 1.00 | 19.90 | 6 | C |
| ATOM | 692 | CG1 | ILE A | 93 | 57.120 | 21.833 | −7.176 | 1.00 | 17.46 | 6 | C |
| ATOM | 693 | CD1 | ILE A | 93 | 57.934 | 23.073 | −7.671 | 1.00 | 16.56 | 6 | C |
| ATOM | 694 | C | ILE A | 93 | 54.653 | 21.106 | −4.265 | 1.00 | 17.19 | 6 | C |
| ATOM | 695 | O | ILE A | 93 | 53.826 | 22.004 | −4.204 | 1.00 | 17.52 | 8 | O |
| ATOM | 696 | N | LEU A | 94 | 54.900 | 20.285 | −3.217 | 1.00 | 14.75 | 7 | N |
| ATOM | 697 | CA | LEU A | 94 | 54.238 | 20.522 | −1.940 | 1.00 | 15.00 | 6 | C |
| ATOM | 698 | CB | LEU A | 94 | 53.592 | 19.181 | −1.498 | 1.00 | 16.18 | 6 | C |
| ATOM | 699 | CG | LEU A | 94 | 53.105 | 19.166 | −0.071 | 1.00 | 17.15 | 6 | C |
| ATOM | 700 | CD1 | LEU A | 94 | 51.981 | 20.200 | 0.116 | 1.00 | 17.51 | 6 | C |
| ATOM | 701 | CD2 | LEU A | 94 | 52.576 | 17.802 | 0.321 | 1.00 | 17.70 | 6 | C |
| ATOM | 702 | C | LEU A | 94 | 55.229 | 21.042 | −0.909 | 1.00 | 16.00 | 6 | C |
| ATOM | 703 | O | LEU A | 94 | 56.191 | 20.303 | −0.673 | 1.00 | 15.06 | 8 | O |
| ATOM | 704 | N | LEU A | 95 | 54.887 | 22.184 | −0.300 | 1.00 | 15.63 | 7 | N |
| ATOM | 705 | CA | LEU A | 95 | 55.826 | 22.695 | 0.702 | 1.00 | 15.01 | 6 | C |
| ATOM | 706 | CB | LEU A | 95 | 55.822 | 24.239 | 0.534 | 1.00 | 16.98 | 6 | C |
| ATOM | 707 | CG | LEU A | 95 | 56.179 | 24.782 | −0.855 | 1.00 | 17.83 | 6 | C |
| ATOM | 708 | CD1 | LEU A | 95 | 56.083 | 26.305 | −0.768 | 1.00 | 18.15 | 6 | C |
| ATOM | 709 | CD2 | LEU A | 95 | 57.481 | 24.292 | −1.475 | 1.00 | 14.31 | 6 | C |
| ATOM | 710 | C | LEU A | 95 | 55.317 | 22.370 | 2.102 | 1.00 | 16.42 | 6 | C |
| ATOM | 711 | O | LEU A | 95 | 54.135 | 22.675 | 2.349 | 1.00 | 17.96 | 8 | O |
| ATOM | 712 | N | ILE A | 96 | 56.116 | 21.685 | 2.946 | 1.00 | 13.74 | 7 | N |
| ATOM | 713 | CA | ILE A | 96 | 55.661 | 21.494 | 4.320 | 1.00 | 16.86 | 6 | C |
| ATOM | 714 | CB | ILE A | 96 | 55.474 | 20.011 | 4.704 | 1.00 | 16.96 | 6 | C |
| ATOM | 715 | CG2 | ILE A | 96 | 54.927 | 19.855 | 6.122 | 1.00 | 17.31 | 6 | C |
| ATOM | 716 | CG1 | ILE A | 96 | 54.476 | 19.270 | 3.747 | 1.00 | 18.40 | 6 | C |
| ATOM | 717 | CD1 | ILE A | 96 | 54.504 | 17.736 | 4.031 | 1.00 | 17.83 | 6 | C |
| ATOM | 718 | C | ILE A | 96 | 56.654 | 22.200 | 5.287 | 1.00 | 16.68 | 6 | C |
| ATOM | 719 | O | ILE A | 96 | 57.801 | 21.779 | 5.404 | 1.00 | 16.25 | 8 | O |
| ATOM | 720 | N | HIS A | 97 | 56.173 | 23.233 | 5.955 | 1.00 | 16.14 | 7 | N |
| ATOM | 721 | CA | HIS A | 97 | 57.060 | 23.933 | 6.923 | 1.00 | 18.56 | 6 | C |
| ATOM | 722 | CB | HIS A | 97 | 58.150 | 24.723 | 6.156 | 1.00 | 16.61 | 6 | C |
| ATOM | 723 | CG | HIS A | 97 | 57.621 | 25.860 | 5.339 | 1.00 | 19.07 | 6 | C |
| ATOM | 724 | CD2 | HIS A | 97 | 57.079 | 27.019 | 5.735 | 1.00 | 15.06 | 6 | C |
| ATOM | 725 | ND1 | HIS A | 97 | 57.504 | 25.861 | 3.954 | 1.00 | 20.25 | 7 | N |
| ATOM | 726 | CE1 | HIS A | 97 | 56.924 | 27.007 | 3.555 | 1.00 | 17.08 | 6 | C |
| ATOM | 727 | NE2 | HIS A | 97 | 56.672 | 27.750 | 4.649 | 1.00 | 21.50 | 7 | N |
| ATOM | 728 | C | HIS A | 97 | 56.283 | 24.826 | 7.852 | 1.00 | 20.66 | 6 | C |
| ATOM | 729 | O | HIS A | 97 | 55.100 | 25.083 | 7.630 | 1.00 | 23.11 | 8 | O |
| ATOM | 730 | N | ASN A | 98 | 56.964 | 25.302 | 8.928 | 1.00 | 19.34 | 7 | N |
| ATOM | 731 | CA | ASN A | 98 | 56.241 | 26.216 | 9.818 | 1.00 | 23.32 | 6 | C |
| ATOM | 732 | CB | ASN A | 98 | 56.799 | 26.008 | 11.230 | 1.00 | 28.80 | 6 | C |
| ATOM | 733 | CG | ASN A | 98 | 56.175 | 24.787 | 11.864 | 1.00 | 32.56 | 6 | C |
| ATOM | 734 | OD1 | ASN A | 98 | 54.924 | 24.624 | 11.706 | 1.00 | 37.71 | 8 | O |
| ATOM | 735 | ND2 | ASN A | 98 | 56.870 | 23.936 | 12.580 | 1.00 | 34.54 | 7 | N |
| ATOM | 736 | C | ASN A | 98 | 56.484 | 27.639 | 9.313 | 1.00 | 22.28 | 6 | C |
| ATOM | 737 | O | ASN A | 98 | 57.395 | 27.813 | 8.503 | 1.00 | 22.49 | 8 | O |
| ATOM | 738 | N | GLY A | 99 | 55.668 | 28.603 | 9.687 | 1.00 | 23.56 | 7 | N |
| ATOM | 739 | CA | GLY A | 99 | 55.868 | 29.936 | 9.111 | 1.00 | 21.71 | 6 | C |
| ATOM | 740 | C | GLY A | 99 | 55.246 | 30.101 | 7.709 | 1.00 | 22.72 | 6 | C |
| ATOM | 741 | O | GLY A | 99 | 54.657 | 29.189 | 7.085 | 1.00 | 18.49 | 8 | O |
| ATOM | 742 | N | MET A | 100 | 55.218 | 31.387 | 7.351 | 1.00 | 20.67 | 7 | N |
| ATOM | 743 | CA | MET A | 100 | 54.584 | 31.824 | 6.088 | 1.00 | 20.09 | 6 | C |
| ATOM | 744 | CB | MET A | 100 | 53.231 | 32.418 | 6.360 | 1.00 | 18.47 | 6 | C |
| ATOM | 745 | CG | MET A | 100 | 52.191 | 31.467 | 7.008 | 1.00 | 21.61 | 6 | C |
| ATOM | 746 | SD | MET A | 100 | 50.691 | 32.333 | 7.598 | 1.00 | 22.79 | 16 | S |
| ATOM | 747 | CE | MET A | 100 | 49.925 | 32.298 | 5.967 | 1.00 | 22.69 | 6 | C |
| ATOM | 748 | C | MET A | 100 | 55.485 | 32.869 | 5.390 | 1.00 | 20.73 | 6 | C |
| ATOM | 749 | O | MET A | 100 | 56.407 | 33.482 | 5.946 | 1.00 | 22.44 | 8 | O |
| ATOM | 750 | N | GLY A | 101 | 55.268 | 32.954 | 4.060 | 1.00 | 20.68 | 7 | N |
| ATOM | 751 | CA | GLY A | 101 | 56.025 | 33.894 | 3.251 | 1.00 | 22.08 | 6 | C |
| ATOM | 752 | C | GLY A | 101 | 56.858 | 33.269 | 2.141 | 1.00 | 22.61 | 6 | C |
| ATOM | 753 | O | GLY A | 101 | 57.514 | 34.026 | 1.418 | 1.00 | 21.68 | 8 | O |
| ATOM | 754 | N | THR A | 102 | 56.771 | 31.965 | 1.947 | 1.00 | 18.22 | 7 | N |
| ATOM | 755 | CA | THR A | 102 | 57.582 | 31.276 | 0.915 | 1.00 | 19.19 | 6 | C |
| ATOM | 756 | CB | THR A | 102 | 58.022 | 29.841 | 1.245 | 1.00 | 19.68 | 6 | C |
| ATOM | 757 | OG1 | THR A | 102 | 56.848 | 29.067 | 1.529 | 1.00 | 17.55 | 8 | O |
| ATOM | 758 | CG2 | THR A | 102 | 59.033 | 29.812 | 2.396 | 1.00 | 21.14 | 6 | C |
| ATOM | 759 | C | THR A | 102 | 56.777 | 31.383 | −0.389 | 1.00 | 18.24 | 6 | C |
| ATOM | 760 | O | THR A | 102 | 57.402 | 31.332 | −1.451 | 1.00 | 18.19 | 8 | O |
| ATOM | 761 | N | ILE A | 103 | 55.439 | 31.399 | −0.308 | 1.00 | 18.81 | 7 | N |
| ATOM | 762 | CA | ILE A | 103 | 54.629 | 31.460 | −1.543 | 1.00 | 19.46 | 6 | C |
| ATOM | 763 | CB | ILE A | 103 | 53.137 | 31.296 | −1.228 | 1.00 | 21.01 | 6 | C |
| ATOM | 764 | CG2 | ILE A | 103 | 52.341 | 31.547 | −2.544 | 1.00 | 23.49 | 6 | C |
| ATOM | 765 | CG1 | ILE A | 103 | 52.891 | 29.856 | −0.773 | 1.00 | 22.16 | 6 | C |
| ATOM | 766 | CD1 | ILE A | 103 | 53.624 | 28.775 | −1.645 | 1.00 | 24.52 | 6 | C |
| ATOM | 767 | C | ILE A | 103 | 54.872 | 32.768 | −2.299 | 1.00 | 23.85 | 6 | C |
| ATOM | 768 | O | ILE A | 103 | 54.998 | 32.791 | −3.532 | 1.00 | 21.71 | 8 | O |
| ATOM | 769 | N | GLU A | 104 | 55.021 | 33.861 | −1.552 | 1.00 | 23.16 | 7 | N |
| ATOM | 770 | CA | GLU A | 104 | 55.341 | 35.146 | −2.184 | 1.00 | 25.98 | 6 | C |
| ATOM | 771 | CB | GLU A | 104 | 55.309 | 36.202 | −1.055 | 1.00 | 29.89 | 6 | C |
| ATOM | 772 | CG | GLU A | 104 | 53.885 | 36.435 | −0.592 | 1.00 | 34.28 | 6 | C |
| ATOM | 773 | CD | GLU A | 104 | 53.257 | 35.373 | 0.250 | 1.00 | 36.85 | 6 | C |
| ATOM | 774 | OE1 | GLU A | 104 | 52.096 | 35.576 | 0.690 | 1.00 | 36.90 | 8 | O |
| ATOM | 775 | OE2 | GLU A | 104 | 53.877 | 34.306 | 0.450 | 1.00 | 37.42 | 8 | O |
| ATOM | 776 | C | GLU A | 104 | 56.663 | 35.114 | −2.895 | 1.00 | 26.03 | 6 | C |
| ATOM | 777 | O | GLU A | 104 | 56.839 | 35.826 | −3.919 | 1.00 | 27.72 | 8 | O |
| ATOM | 778 | N | GLU A | 105 | 57.642 | 34.347 | −2.444 | 1.00 | 21.54 | 7 | N |
| ATOM | 779 | CA | GLU A | 105 | 58.934 | 34.165 | −3.036 | 1.00 | 21.43 | 6 | C |
| ATOM | 780 | CB | GLU A | 105 | 60.000 | 33.614 | −2.064 | 1.00 | 22.40 | 6 | C |
| ATOM | 781 | CG | GLU A | 105 | 60.179 | 34.582 | −0.855 | 1.00 | 23.22 | 6 | C |
| ATOM | 782 | CD | GLU A | 105 | 61.000 | 33.973 | 0.287 | 1.00 | 25.80 | 6 | C |
| ATOM | 783 | OE1 | GLU A | 105 | 60.968 | 32.769 | 0.476 | 1.00 | 26.56 | 8 | O |
| ATOM | 784 | OE2 | GLU A | 105 | 61.543 | 34.840 | 1.005 | 1.00 | 27.46 | 8 | O |
| ATOM | 785 | C | GLU A | 105 | 58.888 | 33.215 | −4.251 | 1.00 | 25.62 | 6 | C |
| ATOM | 786 | O | GLU A | 105 | 59.902 | 33.122 | −4.943 | 1.00 | 26.81 | 8 | O |
| ATOM | 787 | N | LEU A | 106 | 57.806 | 32.479 | −4.401 | 1.00 | 23.68 | 7 | N |
| ATOM | 788 | CA | LEU A | 106 | 57.663 | 31.484 | −5.466 | 1.00 | 24.45 | 6 | C |
| ATOM | 789 | CB | LEU A | 106 | 57.511 | 30.088 | −4.816 | 1.00 | 23.41 | 6 | C |
| ATOM | 790 | CG | LEU A | 106 | 58.730 | 29.520 | −4.101 | 1.00 | 20.11 | 6 | C |
| ATOM | 791 | CD1 | LEU A | 106 | 58.328 | 28.272 | −3.300 | 1.00 | 22.13 | 6 | C |
| ATOM | 792 | CD2 | LEU A | 106 | 59.906 | 29.150 | −5.017 | 1.00 | 19.79 | 6 | C |
| ATOM | 793 | C | LEU A | 106 | 56.508 | 31.773 | −6.413 | 1.00 | 25.80 | 6 | C |
| ATOM | 794 | O | LEU A | 106 | 55.960 | 30.883 | −7.108 | 1.00 | 22.72 | 8 | O |
| ATOM | 795 | N | GLN A | 107 | 56.069 | 33.044 | −6.559 | 1.00 | 27.10 | 7 | N |
| ATOM | 796 | CA | GLN A | 107 | 54.918 | 33.325 | −7.372 | 1.00 | 32.34 | 6 | C |
| ATOM | 797 | CB | GLN A | 107 | 54.249 | 34.672 | −7.123 | 1.00 | 35.01 | 6 | C |
| ATOM | 798 | CG | GLN A | 107 | 55.086 | 35.899 | −7.292 | 1.00 | 41.84 | 6 | C |
| ATOM | 799 | CD | GLN A | 107 | 54.240 | 37.155 | −7.067 | 1.00 | 45.49 | 6 | C |
| ATOM | 800 | OE1 | GLN A | 107 | 53.762 | 37.446 | −5.969 | 1.00 | 47.72 | 8 | O |
| ATOM | 801 | NE2 | GLN A | 107 | 54.040 | 37.935 | −8.129 | 1.00 | 47.24 | 7 | N |
| ATOM | 802 | C | GLN A | 107 | 55.077 | 33.105 | −8.875 | 1.00 | 32.83 | 6 | C |
| ATOM | 803 | O | GLN A | 107 | 54.015 | 33.015 | −9.522 | 1.00 | 36.23 | 8 | O |
| ATOM | 804 | N | ASN A | 108 | 56.296 | 33.011 | −9.379 | 1.00 | 29.89 | 7 | N |
| ATOM | 805 | CA | ASN A | 108 | 56.411 | 32.771 | −10.831 | 1.00 | 28.94 | 6 | C |
| ATOM | 806 | CB | ASN A | 108 | 57.584 | 33.617 | −11.350 | 1.00 | 30.62 | 6 | C |
| ATOM | 807 | CG | ASN A | 108 | 57.118 | 35.076 | −11.339 | 1.00 | 35.75 | 6 | C |
| ATOM | 808 | OD1 | ASN A | 108 | 55.887 | 35.266 | −11.319 | 1.00 | 37.19 | 8 | O |
| ATOM | 809 | ND2 | ASN A | 108 | 57.940 | 36.092 | −11.334 | 1.00 | 36.26 | 7 | N |
| ATOM | 810 | C | ASN A | 108 | 56.643 | 31.305 | −11.157 | 1.00 | 25.33 | 6 | C |
| ATOM | 811 | O | ASN A | 108 | 56.854 | 30.981 | −12.333 | 1.00 | 23.65 | 8 | O |
| ATOM | 812 | N | ILE A | 109 | 56.620 | 30.422 | −10.154 | 1.00 | 22.74 | 7 | N |
| ATOM | 813 | CA | ILE A | 109 | 56.775 | 28.993 | −10.390 | 1.00 | 22.01 | 6 | C |
| ATOM | 814 | CB | ILE A | 109 | 56.690 | 28.267 | −9.013 | 1.00 | 21.76 | 6 | C |
| ATOM | 815 | CG2 | ILE A | 109 | 56.267 | 26.811 | −9.130 | 1.00 | 24.11 | 6 | C |
| ATOM | 816 | CG1 | ILE A | 109 | 58.019 | 28.363 | −8.275 | 1.00 | 22.06 | 6 | C |
| ATOM | 817 | CD1 | ILE A | 109 | 59.195 | 27.564 | −8.815 | 1.00 | 21.82 | 6 | C |
| ATOM | 818 | C | ILE A | 109 | 55.668 | 28.513 | −11.325 | 1.00 | 20.34 | 6 | C |
| ATOM | 819 | O | ILE A | 109 | 54.502 | 28.793 | −11.087 | 1.00 | 22.89 | 8 | O |
| ATOM | 820 | N | GLN A | 110 | 56.056 | 27.791 | −12.371 | 1.00 | 23.85 | 7 | N |
| ATOM | 821 | CA | GLN A | 110 | 55.067 | 27.279 | −13.315 | 1.00 | 22.34 | 6 | C |
| ATOM | 822 | CB | GLN A | 110 | 55.779 | 26.865 | −14.609 | 1.00 | 27.91 | 6 | C |
| ATOM | 823 | CG | GLN A | 110 | 56.460 | 28.025 | −15.364 | 1.00 | 34.45 | 6 | C |
| ATOM | 824 | CD | GLN A | 110 | 56.847 | 27.447 | −16.723 | 1.00 | 39.75 | 6 | C |
| ATOM | 825 | OE1 | GLN A | 110 | 56.055 | 27.530 | −17.659 | 1.00 | 42.27 | 8 | O |
| ATOM | 826 | NE2 | GLN A | 110 | 57.992 | 26.774 | −16.821 | 1.00 | 41.49 | 7 | N |
| ATOM | 827 | C | GLN A | 110 | 54.319 | 26.052 | −12.828 | 1.00 | 20.76 | 6 | C |
| ATOM | 828 | O | GLN A | 110 | 53.146 | 25.807 | −13.158 | 1.00 | 22.00 | 8 | O |
| ATOM | 829 | N | GLN A | 111 | 55.038 | 25.201 | −12.069 | 1.00 | 18.88 | 7 | N |
| ATOM | 830 | CA | GLN A | 111 | 54.355 | 23.990 | −11.564 | 1.00 | 19.71 | 6 | C |
| ATOM | 831 | CB | GLN A | 111 | 55.377 | 23.170 | −10.733 | 1.00 | 17.41 | 6 | C |
| ATOM | 832 | CG | GLN A | 111 | 56.511 | 22.429 | −11.406 | 1.00 | 17.72 | 6 | C |
| ATOM | 833 | CD | GLN A | 111 | 57.708 | 23.254 | −11.844 | 1.00 | 19.26 | 6 | C |
| ATOM | 834 | OE1 | GLN A | 111 | 57.807 | 24.462 | −11.501 | 1.00 | 21.21 | 8 | O |
| ATOM | 835 | NE2 | GLN A | 111 | 58.668 | 22.569 | −12.461 | 1.00 | 19.89 | 7 | N |
| ATOM | 836 | C | GLN A | 111 | 53.237 | 24.317 | −10.603 | 1.00 | 18.64 | 6 | C |
| ATOM | 837 | O | GLN A | 111 | 53.096 | 25.360 | −9.924 | 1.00 | 19.84 | 8 | O |
| ATOM | 838 | N | PRO A | 112 | 52.316 | 23.355 | −10.350 | 1.00 | 19.32 | 7 | N |
| ATOM | 839 | CD | PRO A | 112 | 52.273 | 22.049 | −11.085 | 1.00 | 19.54 | 6 | C |
| ATOM | 840 | CA | PRO A | 112 | 51.287 | 23.413 | −9.350 | 1.00 | 17.67 | 6 | C |
| ATOM | 841 | CB | PRO A | 112 | 50.594 | 22.057 | −9.388 | 1.00 | 19.86 | 6 | C |
| ATOM | 842 | CG | PRO A | 112 | 50.850 | 21.610 | −10.813 | 1.00 | 18.51 | 6 | C |
| ATOM | 843 | C | PRO A | 112 | 52.025 | 23.497 | −7.977 | 1.00 | 19.87 | 6 | C |
| ATOM | 844 | O | PRO A | 112 | 53.018 | 22.785 | −7.860 | 1.00 | 19.45 | 8 | O |
| ATOM | 845 | N | LEU A | 113 | 51.580 | 24.401 | −7.169 | 1.00 | 17.14 | 7 | N |
| ATOM | 846 | CA | LEU A | 113 | 52.202 | 24.624 | −5.850 | 1.00 | 19.51 | 6 | C |
| ATOM | 847 | CB | LEU A | 113 | 52.870 | 26.002 | −5.999 | 1.00 | 21.10 | 6 | C |
| ATOM | 848 | CG | LEU A | 113 | 53.662 | 26.552 | −4.853 | 1.00 | 24.03 | 6 | C |
| ATOM | 849 | CD1 | LEU A | 113 | 54.900 | 25.632 | −4.677 | 1.00 | 19.16 | 6 | C |
| ATOM | 850 | CD2 | LEU A | 113 | 54.083 | 27.998 | −5.120 | 1.00 | 24.04 | 6 | C |
| ATOM | 851 | C | LEU A | 113 | 51.158 | 24.508 | −4.801 | 1.00 | 17.03 | 6 | C |
| ATOM | 852 | O | LEU A | 113 | 50.116 | 25.162 | −4.683 | 1.00 | 16.75 | 8 | O |
| ATOM | 853 | N | LEU A | 114 | 51.522 | 23.700 | −3.740 | 1.00 | 18.49 | 7 | N |
| ATOM | 854 | CA | LEU A | 114 | 50.664 | 23.451 | −2.634 | 1.00 | 17.95 | 6 | C |
| ATOM | 855 | CB | LEU A | 114 | 50.143 | 22.034 | −2.424 | 1.00 | 19.85 | 6 | C |
| ATOM | 856 | CG | LEU A | 114 | 49.460 | 21.423 | −3.695 | 1.00 | 23.60 | 6 | C |
| ATOM | 857 | CD1 | LEU A | 114 | 48.843 | 20.154 | −3.102 | 1.00 | 26.64 | 6 | C |
| ATOM | 858 | CD2 | LEU A | 114 | 48.382 | 22.345 | −4.172 | 1.00 | 21.17 | 6 | C |
| ATOM | 859 | C | LEU A | 114 | 51.419 | 23.777 | −1.296 | 1.00 | 16.88 | 6 | C |
| ATOM | 860 | O | LEU A | 114 | 52.638 | 23.612 | −1.303 | 1.00 | 19.10 | 8 | O |
| ATOM | 861 | N | MET A | 115 | 50.651 | 24.260 | −0.366 | 1.00 | 16.64 | 7 | N |
| ATOM | 862 | CA | MET A | 115 | 51.342 | 24.700 | 0.865 | 1.00 | 22.56 | 6 | C |
| ATOM | 863 | CB | MET A | 115 | 51.003 | 26.206 | 0.696 | 1.00 | 28.53 | 6 | C |
| ATOM | 864 | CG | MET A | 115 | 49.907 | 26.765 | 1.596 | 1.00 | 34.11 | 6 | C |
| ATOM | 865 | SD | MET A | 115 | 49.652 | 28.550 | 1.466 | 1.00 | 37.01 | 16 | S |
| ATOM | 866 | CE | MET A | 115 | 51.135 | 29.351 | 1.982 | 1.00 | 39.07 | 6 | C |
| ATOM | 867 | C | MET A | 115 | 50.775 | 23.830 | 1.998 | 1.00 | 20.36 | 6 | C |
| ATOM | 868 | O | MET A | 115 | 49.633 | 23.417 | 1.899 | 1.00 | 21.26 | 8 | O |
| ATOM | 869 | N | GLY A | 116 | 51.565 | 23.625 | 3.073 | 1.00 | 19.46 | 7 | N |
| ATOM | 870 | CA | GLY A | 116 | 51.102 | 22.711 | 4.116 | 1.00 | 18.10 | 6 | C |
| ATOM | 871 | C | GLY A | 116 | 51.961 | 22.796 | 5.403 | 1.00 | 18.47 | 6 | C |
| ATOM | 872 | O | GLY A | 116 | 52.978 | 23.469 | 5.415 | 1.00 | 16.76 | 8 | O |
| ATOM | 873 | N | THR A | 117 | 51.360 | 22.195 | 6.420 | 1.00 | 17.51 | 7 | N |
| ATOM | 874 | CA | THR A | 117 | 52.076 | 22.228 | 7.730 | 1.00 | 19.25 | 6 | C |
| ATOM | 875 | CB | THR A | 117 | 51.737 | 23.548 | 8.444 | 1.00 | 21.89 | 6 | C |
| ATOM | 876 | OG1 | THR A | 117 | 52.507 | 23.641 | 9.658 | 1.00 | 26.41 | 8 | O |
| ATOM | 877 | CG2 | THR A | 117 | 50.272 | 23.539 | 8.776 | 1.00 | 25.02 | 6 | C |
| ATOM | 878 | C | THR A | 117 | 51.620 | 20.989 | 8.482 | 1.00 | 18.36 | 6 | C |
| ATOM | 879 | O | THR A | 117 | 50.579 | 20.398 | 8.140 | 1.00 | 21.62 | 8 | O |
| ATOM | 880 | N | THR A | 118 | 52.471 | 20.531 | 9.427 | 1.00 | 18.75 | 7 | N |
| ATOM | 881 | CA | THR A | 118 | 52.126 | 19.278 | 10.091 | 1.00 | 16.38 | 6 | C |
| ATOM | 882 | CB | THR A | 118 | 53.093 | 18.158 | 9.577 | 1.00 | 16.51 | 6 | C |
| ATOM | 883 | OG1 | THR A | 118 | 52.519 | 16.881 | 9.952 | 1.00 | 18.48 | 8 | O |
| ATOM | 884 | CG2 | THR A | 118 | 54.527 | 18.234 | 10.036 | 1.00 | 16.97 | 6 | C |
| ATOM | 885 | C | THR A | 118 | 52.213 | 19.422 | 11.608 | 1.00 | 19.31 | 6 | C |
| ATOM | 886 | O | THR A | 118 | 52.986 | 20.236 | 12.109 | 1.00 | 19.95 | 8 | O |
| ATOM | 887 | N | THR A | 119 | 51.516 | 18.530 | 12.289 | 1.00 | 17.85 | 7 | N |
| ATOM | 888 | CA | THR A | 119 | 51.603 | 18.471 | 13.738 | 1.00 | 20.62 | 6 | C |
| ATOM | 889 | CB | THR A | 119 | 50.306 | 18.508 | 14.510 | 1.00 | 21.64 | 6 | C |
| ATOM | 890 | OG1 | THR A | 119 | 49.405 | 17.452 | 14.107 | 1.00 | 21.73 | 8 | O |
| ATOM | 891 | CG2 | THR A | 119 | 49.552 | 19.832 | 14.282 | 1.00 | 24.91 | 6 | C |
| ATOM | 892 | C | THR A | 119 | 52.293 | 17.139 | 14.097 | 1.00 | 22.31 | 6 | C |
| ATOM | 893 | O | THR A | 119 | 52.425 | 16.856 | 15.310 | 1.00 | 24.98 | 8 | O |
| ATOM | 894 | N | HIS A | 120 | 52.768 | 16.441 | 13.073 | 1.00 | 21.78 | 7 | N |
| ATOM | 895 | CA | HIS A | 120 | 53.632 | 15.286 | 13.365 | 1.00 | 19.23 | 6 | C |
| ATOM | 896 | CB | HIS A | 120 | 54.077 | 14.504 | 12.138 | 1.00 | 18.17 | 6 | C |
| ATOM | 897 | CG | HIS A | 120 | 53.142 | 13.400 | 11.723 | 1.00 | 17.11 | 6 | C |
| ATOM | 898 | CD2 | HIS A | 120 | 53.227 | 12.102 | 12.102 | 1.00 | 16.15 | 6 | C |
| ATOM | 899 | ND1 | HIS A | 120 | 52.066 | 13.447 | 10.881 | 1.00 | 20.80 | 7 | N |
| ATOM | 900 | CE1 | HIS A | 120 | 51.504 | 12.252 | 10.723 | 1.00 | 16.47 | 6 | C |
| ATOM | 901 | NE2 | HIS A | 120 | 52.231 | 11.403 | 11.501 | 1.00 | 20.33 | 7 | N |
| ATOM | 902 | C | HIS A | 120 | 54.851 | 15.888 | 14.043 | 1.00 | 19.92 | 6 | C |
| ATOM | 903 | O | HIS A | 120 | 55.330 | 16.956 | 13.615 | 1.00 | 21.65 | 8 | O |
| ATOM | 904 | N | ALA A | 121 | 55.542 | 15.129 | 14.895 | 1.00 | 19.64 | 7 | N |
| ATOM | 905 | CA | ALA A | 121 | 56.722 | 15.603 | 15.596 | 1.00 | 20.97 | 6 | C |
| ATOM | 906 | CB | ALA A | 121 | 56.447 | 15.713 | 17.091 | 1.00 | 20.83 | 6 | C |
| ATOM | 907 | C | ALA A | 121 | 57.877 | 14.606 | 15.379 | 1.00 | 20.32 | 6 | C |
| ATOM | 908 | O | ALA A | 121 | 57.661 | 13.416 | 15.530 | 1.00 | 20.48 | 8 | O |
| ATOM | 909 | N | ALA A | 122 | 58.948 | 15.085 | 14.841 | 1.00 | 20.43 | 7 | N |
| ATOM | 910 | CA | ALA A | 122 | 60.133 | 14.319 | 14.482 | 1.00 | 18.93 | 6 | C |
| ATOM | 911 | CB | ALA A | 122 | 60.296 | 14.084 | 12.985 | 1.00 | 23.71 | 6 | C |
| ATOM | 912 | C | ALA A | 122 | 61.384 | 15.071 | 14.922 | 1.00 | 21.86 | 6 | C |
| ATOM | 913 | O | ALA A | 122 | 61.629 | 16.224 | 14.543 | 1.00 | 23.59 | 8 | O |
| ATOM | 914 | N | ARG A | 123 | 62.209 | 14.422 | 15.742 | 1.00 | 22.93 | 7 | N |
| ATOM | 915 | CA | ARG A | 123 | 63.429 | 15.000 | 16.302 | 1.00 | 23.09 | 6 | C |
| ATOM | 916 | CB | ARG A | 123 | 63.416 | 14.740 | 17.846 | 1.00 | 23.23 | 6 | C |
| ATOM | 917 | CG | ARG A | 123 | 64.439 | 15.643 | 18.523 | 1.00 | 29.39 | 6 | C |
| ATOM | 918 | CD | ARG A | 123 | 64.440 | 15.454 | 20.029 | 1.00 | 29.72 | 6 | C |
| ATOM | 919 | NE | ARG A | 123 | 64.915 | 14.095 | 20.372 | 1.00 | 31.81 | 7 | N |
| ATOM | 920 | CZ | ARG A | 123 | 64.191 | 13.171 | 20.969 | 1.00 | 31.03 | 6 | C |
| ATOM | 921 | NH1 | ARG A | 123 | 62.957 | 13.363 | 21.353 | 1.00 | 32.54 | 7 | N |
| ATOM | 922 | NH2 | ARG A | 123 | 64.757 | 11.982 | 21.176 | 1.00 | 31.02 | 7 | N |
| ATOM | 923 | C | ARG A | 123 | 64.696 | 14.378 | 15.744 | 1.00 | 21.31 | 6 | C |
| ATOM | 924 | O | ARG A | 123 | 64.755 | 13.186 | 15.473 | 1.00 | 21.06 | 8 | O |
| ATOM | 925 | N | ARG A | 124 | 65.738 | 15.202 | 15.492 | 1.00 | 21.81 | 7 | N |
| ATOM | 926 | CA | ARG A | 124 | 66.982 | 14.627 | 14.981 | 1.00 | 22.14 | 6 | C |
| ATOM | 927 | CB | ARG A | 124 | 67.537 | 15.375 | 13.766 | 1.00 | 26.55 | 6 | C |
| ATOM | 928 | CG | ARG A | 124 | 68.974 | 15.092 | 13.354 | 1.00 | 29.85 | 6 | C |
| ATOM | 929 | CD | ARG A | 124 | 69.297 | 15.837 | 12.065 | 1.00 | 36.14 | 6 | C |
| ATOM | 930 | NE | ARG A | 124 | 70.279 | 15.186 | 11.256 | 1.00 | 38.31 | 7 | N |
| ATOM | 931 | CZ | ARG A | 124 | 70.445 | 15.060 | 9.959 | 1.00 | 42.15 | 6 | C |
| ATOM | 932 | NH1 | ARG A | 124 | 71.533 | 14.378 | 9.575 | 1.00 | 43.04 | 7 | N |
| ATOM | 933 | NH2 | ARG A | 124 | 69.643 | 15.525 | 9.016 | 1.00 | 40.89 | 7 | N |
| ATOM | 934 | C | ARG A | 124 | 68.019 | 14.637 | 16.108 | 1.00 | 23.57 | 6 | C |
| ATOM | 935 | O | ARG A | 124 | 68.272 | 15.673 | 16.740 | 1.00 | 24.93 | 8 | O |
| ATOM | 936 | N | ASP A | 125 | 68.523 | 13.439 | 16.435 | 1.00 | 21.32 | 7 | N |
| ATOM | 937 | CA | ASP A | 125 | 69.604 | 13.276 | 17.399 | 1.00 | 22.50 | 6 | C |
| ATOM | 938 | CB | ASP A | 125 | 69.177 | 12.237 | 18.461 | 1.00 | 26.84 | 6 | C |
| ATOM | 939 | CG | ASP A | 125 | 69.872 | 12.410 | 19.794 | 1.00 | 33.28 | 6 | C |
| ATOM | 940 | OD1 | ASP A | 125 | 71.081 | 12.757 | 19.821 | 1.00 | 36.62 | 8 | O |
| ATOM | 941 | OD2 | ASP A | 125 | 69.269 | 12.241 | 20.883 | 1.00 | 36.16 | 8 | O |
| ATOM | 942 | C | ASP A | 125 | 70.833 | 12.756 | 16.681 | 1.00 | 22.87 | 6 | C |
| ATOM | 943 | O | ASP A | 125 | 70.986 | 11.610 | 16.378 | 1.00 | 21.22 | 8 | O |
| ATOM | 944 | N | GLY A | 126 | 71.701 | 13.672 | 16.168 | 1.00 | 22.62 | 7 | N |
| ATOM | 945 | CA | GLY A | 126 | 72.859 | 13.236 | 15.376 | 1.00 | 25.05 | 6 | C |
| ATOM | 946 | C | GLY A | 126 | 72.361 | 12.694 | 14.033 | 1.00 | 26.03 | 6 | C |
| ATOM | 947 | O | GLY A | 126 | 71.685 | 13.393 | 13.286 | 1.00 | 27.91 | 8 | O |
| ATOM | 948 | N | ASN A | 127 | 72.634 | 11.422 | 13.752 | 1.00 | 25.62 | 7 | N |
| ATOM | 949 | CA | ASN A | 127 | 72.215 | 10.753 | 12.538 | 1.00 | 28.77 | 6 | C |
| ATOM | 950 | CB | ASN A | 127 | 73.334 | 9.949 | 11.887 | 1.00 | 34.37 | 6 | C |
| ATOM | 951 | CG | ASN A | 127 | 74.520 | 10.856 | 11.544 | 1.00 | 38.21 | 6 | C |
| ATOM | 952 | OD1 | ASN A | 127 | 74.254 | 11.920 | 10.968 | 1.00 | 41.87 | 8 | O |
| ATOM | 953 | ND2 | ASN A | 127 | 75.728 | 10.471 | 11.907 | 1.00 | 41.91 | 7 | N |
| ATOM | 954 | C | ASN A | 127 | 70.991 | 9.865 | 12.808 | 1.00 | 26.63 | 6 | C |
| ATOM | 955 | O | ASN A | 127 | 70.666 | 9.018 | 11.983 | 1.00 | 27.36 | 8 | O |
| ATOM | 956 | N | VAL A | 128 | 70.452 | 10.046 | 14.010 | 1.00 | 22.18 | 7 | N |
| ATOM | 957 | CA | VAL A | 128 | 69.205 | 9.291 | 14.332 | 1.00 | 18.19 | 6 | C |
| ATOM | 958 | CB | VAL A | 128 | 69.205 | 8.608 | 15.726 | 1.00 | 18.38 | 6 | C |
| ATOM | 959 | CG1 | VAL A | 128 | 67.897 | 7.843 | 15.987 | 1.00 | 19.12 | 6 | C |
| ATOM | 960 | CG2 | VAL A | 128 | 70.310 | 7.548 | 15.766 | 1.00 | 20.01 | 6 | C |
| ATOM | 961 | C | VAL A | 128 | 67.986 | 10.191 | 14.274 | 1.00 | 18.73 | 6 | C |
| ATOM | 962 | O | VAL A | 128 | 68.025 | 11.216 | 14.929 | 1.00 | 19.21 | 8 | O |
| ATOM | 963 | N | ILE A | 129 | 66.885 | 9.791 | 13.620 | 1.00 | 19.98 | 7 | N |
| ATOM | 964 | CA | ILE A | 129 | 65.668 | 10.583 | 13.613 | 1.00 | 17.06 | 6 | C |
| ATOM | 965 | CB | ILE A | 129 | 65.085 | 10.783 | 12.210 | 1.00 | 16.56 | 6 | C |
| ATOM | 966 | CG2 | ILE A | 129 | 63.764 | 11.544 | 12.262 | 1.00 | 17.69 | 6 | C |
| ATOM | 967 | CG1 | ILE A | 129 | 66.083 | 11.492 | 11.324 | 1.00 | 17.86 | 6 | C |
| ATOM | 968 | CD1 | ILE A | 129 | 66.738 | 12.760 | 11.797 | 1.00 | 18.89 | 6 | C |
| ATOM | 969 | C | ILE A | 129 | 64.648 | 9.847 | 14.465 | 1.00 | 18.59 | 6 | C |
| ATOM | 970 | O | ILE A | 129 | 64.437 | 8.643 | 14.258 | 1.00 | 21.80 | 8 | O |
| ATOM | 971 | N | ILE A | 130 | 63.969 | 10.532 | 15.338 | 1.00 | 17.75 | 7 | N |
| ATOM | 972 | CA | ILE A | 130 | 62.945 | 9.921 | 16.198 | 1.00 | 18.22 | 6 | C |
| ATOM | 973 | CB | ILE A | 130 | 63.234 | 10.203 | 17.675 | 1.00 | 18.34 | 6 | C |
| ATOM | 974 | CG2 | ILE A | 130 | 62.171 | 9.527 | 18.566 | 1.00 | 23.73 | 6 | C |
| ATOM | 975 | CG1 | ILE A | 130 | 64.638 | 9.672 | 18.046 | 1.00 | 19.70 | 6 | C |
| ATOM | 976 | CD1 | ILE A | 130 | 65.800 | 10.582 | 17.806 | 1.00 | 23.85 | 6 | C |
| ATOM | 977 | C | ILE A | 130 | 61.572 | 10.470 | 15.833 | 1.00 | 18.69 | 6 | C |
| ATOM | 978 | O | ILE A | 130 | 61.332 | 11.697 | 15.844 | 1.00 | 21.39 | 8 | O |
| ATOM | 979 | N | HIS A | 131 | 60.682 | 9.538 | 15.458 | 1.00 | 17.63 | 7 | N |
| ATOM | 980 | CA | HIS A | 131 | 59.289 | 9.953 | 15.209 | 1.00 | 19.41 | 6 | C |
| ATOM | 981 | CB | HIS A | 131 | 58.556 | 8.966 | 14.288 | 1.00 | 19.44 | 6 | C |
| ATOM | 982 | CG | HIS A | 131 | 57.242 | 9.438 | 13.782 | 1.00 | 16.44 | 6 | C |
| ATOM | 983 | CD2 | HIS A | 131 | 56.590 | 10.631 | 13.899 | 1.00 | 18.30 | 6 | C |
| ATOM | 984 | ND1 | HIS A | 131 | 56.287 | 8.643 | 13.136 | 1.00 | 19.15 | 7 | N |
| ATOM | 985 | CE1 | HIS A | 131 | 55.167 | 9.295 | 12.831 | 1.00 | 19.09 | 6 | C |
| ATOM | 986 | NE2 | HIS A | 131 | 55.364 | 10.520 | 13.285 | 1.00 | 17.79 | 7 | N |
| ATOM | 987 | C | HIS A | 131 | 58.652 | 10.025 | 16.597 | 1.00 | 22.70 | 6 | C |
| ATOM | 988 | O | HIS A | 131 | 58.289 | 9.010 | 17.211 | 1.00 | 23.54 | 8 | O |
| ATOM | 989 | N | VAL A | 132 | 58.575 | 11.222 | 17.167 | 1.00 | 23.13 | 7 | N |
| ATOM | 990 | CA | VAL A | 132 | 58.088 | 11.444 | 18.514 | 1.00 | 24.97 | 6 | C |
| ATOM | 991 | CB | VAL A | 132 | 58.486 | 12.851 | 19.001 | 1.00 | 25.58 | 6 | C |
| ATOM | 992 | CG1 | VAL A | 132 | 57.829 | 13.230 | 20.328 | 1.00 | 27.80 | 6 | C |
| ATOM | 993 | CG2 | VAL A | 132 | 60.009 | 12.970 | 19.046 | 1.00 | 24.87 | 6 | C |
| ATOM | 994 | C | VAL A | 132 | 56.569 | 11.307 | 18.636 | 1.00 | 26.11 | 6 | C |
| ATOM | 995 | O | VAL A | 132 | 56.053 | 10.833 | 19.650 | 1.00 | 26.81 | 8 | O |
| ATOM | 996 | N | ALA A | 133 | 55.795 | 11.897 | 17.717 | 1.00 | 23.08 | 7 | N |
| ATOM | 997 | CA | ALA A | 133 | 54.369 | 11.749 | 17.758 | 1.00 | 21.40 | 6 | C |
| ATOM | 998 | CB | ALA A | 133 | 53.722 | 12.855 | 18.588 | 1.00 | 24.59 | 6 | C |
| ATOM | 999 | C | ALA A | 133 | 53.709 | 11.847 | 16.360 | 1.00 | 20.17 | 6 | C |
| ATOM | 1000 | O | ALA A | 133 | 54.233 | 12.463 | 15.494 | 1.00 | 20.54 | 8 | O |
| ATOM | 1001 | N | ASN A | 134 | 52.590 | 11.105 | 16.312 | 1.00 | 20.07 | 7 | N |
| ATOM | 1002 | CA | ASN A | 134 | 51.788 | 11.077 | 15.094 | 1.00 | 21.32 | 6 | C |
| ATOM | 1003 | CB | ASN A | 134 | 50.815 | 9.868 | 15.149 | 1.00 | 22.44 | 6 | C |
| ATOM | 1004 | CG | ASN A | 134 | 51.568 | 8.568 | 14.909 | 1.00 | 27.18 | 6 | C |
| ATOM | 1005 | OD1 | ASN A | 134 | 52.184 | 8.360 | 13.889 | 1.00 | 31.36 | 8 | O |
| ATOM | 1006 | ND2 | ASN A | 134 | 51.422 | 7.659 | 15.866 | 1.00 | 32.50 | 7 | N |
| ATOM | 1007 | C | ASN A | 134 | 50.959 | 12.338 | 15.079 | 1.00 | 20.24 | 6 | C |
| ATOM | 1008 | O | ASN A | 134 | 50.953 | 13.027 | 16.096 | 1.00 | 23.52 | 8 | O |
| ATOM | 1009 | N | GLY A | 135 | 50.354 | 12.710 | 13.965 | 1.00 | 20.08 | 7 | N |
| ATOM | 1010 | CA | GLY A | 135 | 49.564 | 13.959 | 13.948 | 1.00 | 20.50 | 6 | C |
| ATOM | 1011 | C | GLY A | 135 | 48.911 | 14.083 | 12.577 | 1.00 | 20.77 | 6 | C |
| ATOM | 1012 | O | GLY A | 135 | 48.600 | 13.088 | 11.917 | 1.00 | 19.40 | 8 | O |
| ATOM | 1013 | N | ILE A | 136 | 48.589 | 15.318 | 12.207 | 1.00 | 19.11 | 7 | N |
| ATOM | 1014 | CA | ILE A | 136 | 47.860 | 15.595 | 10.973 | 1.00 | 21.63 | 6 | C |
| ATOM | 1015 | CB | ILE A | 136 | 46.424 | 16.113 | 11.252 | 1.00 | 23.68 | 6 | C |
| ATOM | 1016 | CG2 | ILE A | 136 | 46.492 | 17.392 | 12.068 | 1.00 | 26.67 | 6 | C |
| ATOM | 1017 | CG1 | ILE A | 136 | 45.604 | 16.306 | 9.958 | 1.00 | 25.90 | 6 | C |
| ATOM | 1018 | CD1 | ILE A | 136 | 44.088 | 16.306 | 10.319 | 1.00 | 31.29 | 6 | C |
| ATOM | 1019 | C | ILE A | 136 | 48.636 | 16.587 | 10.104 | 1.00 | 19.20 | 6 | C |
| ATOM | 1020 | O | ILE A | 136 | 49.258 | 17.522 | 10.601 | 1.00 | 20.74 | 8 | O |
| ATOM | 1021 | N | THR A | 137 | 48.583 | 16.374 | 8.813 | 1.00 | 19.40 | 7 | N |
| ATOM | 1022 | CA | THR A | 137 | 49.274 | 17.279 | 7.885 | 1.00 | 15.74 | 6 | C |
| ATOM | 1023 | CB | THR A | 137 | 50.191 | 16.495 | 6.929 | 1.00 | 18.54 | 6 | C |
| ATOM | 1024 | OG1 | THR A | 137 | 51.216 | 15.809 | 7.698 | 1.00 | 16.49 | 8 | O |
| ATOM | 1025 | CG2 | THR A | 137 | 50.861 | 17.443 | 5.925 | 1.00 | 16.61 | 6 | C |
| ATOM | 1026 | C | THR A | 137 | 48.150 | 17.983 | 7.065 | 1.00 | 16.50 | 6 | C |
| ATOM | 1027 | O | THR A | 137 | 47.336 | 17.245 | 6.496 | 1.00 | 16.25 | 8 | O |
| ATOM | 1028 | N | HIS A | 138 | 48.128 | 19.296 | 7.206 | 1.00 | 17.18 | 7 | N |
| ATOM | 1029 | CA | HIS A | 138 | 47.058 | 20.022 | 6.485 | 1.00 | 18.37 | 6 | C |
| ATOM | 1030 | CB | HIS A | 138 | 46.523 | 21.200 | 7.336 | 1.00 | 23.83 | 6 | C |
| ATOM | 1031 | CG | HIS A | 138 | 45.821 | 20.780 | 8.598 | 1.00 | 25.62 | 6 | C |
| ATOM | 1032 | CD2 | HIS A | 138 | 44.634 | 20.213 | 8.773 | 1.00 | 25.94 | 6 | C |
| ATOM | 1033 | ND1 | HIS A | 138 | 46.355 | 20.881 | 9.873 | 1.00 | 29.72 | 7 | N |
| ATOM | 1034 | CE1 | HIS A | 138 | 45.479 | 20.408 | 10.747 | 1.00 | 28.06 | 6 | C |
| ATOM | 1035 | NE2 | HIS A | 138 | 44.399 | 19.993 | 10.114 | 1.00 | 28.38 | 7 | N |
| ATOM | 1036 | C | HIS A | 138 | 47.649 | 20.597 | 5.212 | 1.00 | 19.00 | 6 | C |
| ATOM | 1037 | O | HIS A | 138 | 48.706 | 21.216 | 5.300 | 1.00 | 18.61 | 8 | O |
| ATOM | 1038 | N | ILE A | 139 | 47.060 | 20.376 | 4.036 | 1.00 | 18.69 | 7 | N |
| ATOM | 1039 | CA | ILE A | 139 | 47.665 | 20.954 | 2.823 | 1.00 | 16.45 | 6 | C |
| ATOM | 1040 | CB | ILE A | 139 | 48.345 | 19.873 | 1.975 | 1.00 | 19.05 | 6 | C |
| ATOM | 1041 | CG2 | ILE A | 139 | 49.419 | 19.219 | 2.843 | 1.00 | 17.33 | 6 | C |
| ATOM | 1042 | CG1 | ILE A | 139 | 47.343 | 18.786 | 1.489 | 1.00 | 19.82 | 6 | C |
| ATOM | 1043 | CD1 | ILE A | 139 | 47.976 | 17.968 | 0.362 | 1.00 | 24.74 | 6 | C |
| ATOM | 1044 | C | ILE A | 139 | 46.582 | 21.657 | 2.016 | 1.00 | 16.59 | 6 | C |
| ATOM | 1045 | O | ILE A | 139 | 45.392 | 21.438 | 2.272 | 1.00 | 17.78 | 8 | O |
| ATOM | 1046 | N | GLY A | 140 | 46.942 | 22.455 | 1.030 | 1.00 | 17.82 | 7 | N |
| ATOM | 1047 | CA | GLY A | 140 | 45.962 | 23.146 | 0.204 | 1.00 | 17.52 | 6 | C |
| ATOM | 1048 | C | GLY A | 140 | 46.691 | 23.966 | −0.851 | 1.00 | 17.87 | 6 | C |
| ATOM | 1049 | O | GLY A | 140 | 47.873 | 24.039 | −0.949 | 1.00 | 16.45 | 8 | O |
| ATOM | 1050 | N | PRO A | 141 | 45.890 | 24.672 | −1.697 | 1.00 | 16.51 | 7 | N |
| ATOM | 1051 | CD | PRO A | 141 | 44.406 | 24.623 | −1.629 | 1.00 | 19.58 | 6 | C |
| ATOM | 1052 | CA | PRO A | 141 | 46.463 | 25.356 | −2.815 | 1.00 | 18.64 | 6 | C |
| ATOM | 1053 | CB | PRO A | 141 | 45.212 | 25.506 | −3.699 | 1.00 | 19.10 | 6 | C |
| ATOM | 1054 | CG | PRO A | 141 | 44.068 | 25.614 | −2.764 | 1.00 | 20.80 | 6 | C |
| ATOM | 1055 | C | PRO A | 141 | 47.140 | 26.668 | −2.466 | 1.00 | 17.22 | 6 | C |
| ATOM | 1056 | O | PRO A | 141 | 46.535 | 27.477 | −1.743 | 1.00 | 23.01 | 8 | O |
| ATOM | 1057 | N | ALA A | 142 | 48.326 | 26.891 | −2.991 | 1.00 | 17.82 | 7 | N |
| ATOM | 1058 | CA | ALA A | 142 | 49.064 | 28.137 | −2.732 | 1.00 | 23.14 | 6 | C |
| ATOM | 1059 | CB | ALA A | 142 | 50.519 | 27.971 | −3.124 | 1.00 | 25.44 | 6 | C |
| ATOM | 1060 | C | ALA A | 142 | 48.425 | 29.321 | −3.426 | 1.00 | 27.19 | 6 | C |
| ATOM | 1061 | O | ALA A | 142 | 48.214 | 30.393 | −2.836 | 1.00 | 30.17 | 8 | O |
| ATOM | 1062 | N | ARG A | 143 | 48.031 | 29.114 | −4.688 | 1.00 | 27.39 | 7 | N |
| ATOM | 1063 | CA | ARG A | 143 | 47.435 | 30.206 | −5.474 | 1.00 | 31.04 | 6 | C |
| ATOM | 1064 | CB | ARG A | 143 | 48.432 | 30.602 | −6.585 | 1.00 | 30.87 | 6 | C |
| ATOM | 1065 | CG | ARG A | 143 | 49.836 | 30.972 | −6.164 | 1.00 | 31.39 | 6 | C |
| ATOM | 1066 | CD | ARG A | 143 | 50.833 | 30.738 | −7.285 | 1.00 | 31.39 | 6 | C |
| ATOM | 1067 | NE | ARG A | 143 | 50.873 | 29.353 | −7.750 | 1.00 | 27.23 | 7 | N |
| ATOM | 1068 | CZ | ARG A | 143 | 51.779 | 28.869 | −8.610 | 1.00 | 25.80 | 6 | C |
| ATOM | 1069 | NH1 | ARG A | 143 | 52.760 | 29.628 | −9.081 | 1.00 | 25.09 | 7 | N |
| ATOM | 1070 | NH2 | ARG A | 143 | 51.745 | 27.595 | −8.978 | 1.00 | 24.32 | 7 | N |
| ATOM | 1071 | C | ARG A | 143 | 46.107 | 29.759 | −6.026 | 1.00 | 30.38 | 6 | C |
| ATOM | 1072 | O | ARG A | 143 | 45.716 | 28.600 | −6.054 | 1.00 | 29.56 | 8 | O |
| ATOM | 1073 | N | GLN A | 144 | 45.388 | 30.683 | −6.744 | 1.00 | 34.97 | 7 | N |
| ATOM | 1074 | CA | GLN A | 144 | 44.096 | 30.284 | −7.307 | 1.00 | 37.01 | 6 | C |
| ATOM | 1075 | CB | GLN A | 144 | 43.444 | 31.577 | −7.862 | 1.00 | 42.13 | 6 | C |
| ATOM | 1076 | CG | GLN A | 144 | 43.274 | 32.527 | −6.683 | 1.00 | 46.82 | 6 | C |
| ATOM | 1077 | CD | GLN A | 144 | 42.412 | 33.744 | −6.935 | 1.00 | 50.54 | 6 | C |
| ATOM | 1078 | OE1 | GLN A | 144 | 42.502 | 34.392 | −7.992 | 1.00 | 52.59 | 8 | O |
| ATOM | 1079 | NE2 | GLN A | 144 | 41.572 | 34.087 | −5.953 | 1.00 | 50.31 | 7 | N |
| ATOM | 1080 | C | GLN A | 144 | 44.186 | 29.186 | −8.330 | 1.00 | 35.09 | 6 | C |
| ATOM | 1081 | O | GLN A | 144 | 43.439 | 28.231 | −8.458 | 1.00 | 35.19 | 8 | O |
| ATOM | 1082 | N | GLN A | 145 | 45.277 | 29.212 | −9.098 | 1.00 | 35.05 | 7 | N |
| ATOM | 1083 | CA | GLN A | 145 | 45.584 | 28.232 | −10.114 | 1.00 | 31.72 | 6 | C |
| ATOM | 1084 | CB | GLN A | 145 | 46.605 | 28.813 | −11.078 | 1.00 | 34.36 | 6 | C |
| ATOM | 1085 | CG | GLN A | 145 | 48.042 | 28.996 | −10.680 | 1.00 | 33.82 | 6 | C |
| ATOM | 1086 | CD | GLN A | 145 | 48.463 | 30.393 | −10.265 | 1.00 | 36.58 | 6 | C |
| ATOM | 1087 | OE1 | GLN A | 145 | 47.779 | 31.296 | −9.770 | 1.00 | 36.44 | 8 | O |
| ATOM | 1088 | NE2 | GLN A | 145 | 49.755 | 30.685 | −10.421 | 1.00 | 36.77 | 7 | N |
| ATOM | 1089 | C | GLN A | 145 | 46.039 | 26.855 | −9.616 | 1.00 | 28.92 | 6 | C |
| ATOM | 1090 | O | GLN A | 145 | 46.190 | 25.988 | −10.468 | 1.00 | 26.42 | 8 | O |
| ATOM | 1091 | N | ASP A | 146 | 46.177 | 26.628 | −8.297 | 1.00 | 24.03 | 7 | N |
| ATOM | 1092 | CA | ASP A | 146 | 46.550 | 25.278 | −7.822 | 1.00 | 22.12 | 6 | C |
| ATOM | 1093 | CB | ASP A | 146 | 47.570 | 25.571 | −6.678 | 1.00 | 22.29 | 6 | C |
| ATOM | 1094 | CG | ASP A | 146 | 48.725 | 26.364 | −7.251 | 1.00 | 19.59 | 6 | C |
| ATOM | 1095 | OD1 | ASP A | 146 | 49.373 | 26.005 | −8.273 | 1.00 | 23.09 | 8 | O |
| ATOM | 1096 | OD2 | ASP A | 146 | 48.984 | 27.431 | −6.658 | 1.00 | 21.75 | 8 | O |
| ATOM | 1097 | C | ASP A | 146 | 45.439 | 24.484 | −7.205 | 1.00 | 22.09 | 6 | C |
| ATOM | 1098 | O | ASP A | 146 | 45.552 | 23.429 | −6.524 | 1.00 | 21.95 | 8 | O |
| ATOM | 1099 | N | GLY A | 147 | 44.188 | 24.933 | −7.307 | 1.00 | 20.41 | 7 | N |
| ATOM | 1100 | CA | GLY A | 147 | 43.078 | 24.336 | −6.616 | 1.00 | 22.44 | 6 | C |
| ATOM | 1101 | C | GLY A | 147 | 42.648 | 22.966 | −7.094 | 1.00 | 20.12 | 6 | C |
| ATOM | 1102 | O | GLY A | 147 | 41.821 | 22.407 | −6.393 | 1.00 | 20.35 | 8 | O |
| ATOM | 1103 | N | ASP A | 148 | 43.150 | 22.417 | −8.220 | 1.00 | 18.21 | 7 | N |
| ATOM | 1104 | CA | ASP A | 148 | 42.597 | 21.129 | −8.666 | 1.00 | 19.50 | 6 | C |
| ATOM | 1105 | CB | ASP A | 148 | 42.619 | 21.018 | −10.198 | 1.00 | 19.46 | 6 | C |
| ATOM | 1106 | CG | ASP A | 148 | 41.656 | 22.017 | −10.872 | 1.00 | 19.59 | 6 | C |
| ATOM | 1107 | OD1 | ASP A | 148 | 40.773 | 22.496 | −10.126 | 1.00 | 23.55 | 8 | O |
| ATOM | 1108 | OD2 | ASP A | 148 | 41.857 | 22.327 | −12.054 | 1.00 | 22.94 | 8 | O |
| ATOM | 1109 | C | ASP A | 148 | 43.462 | 19.934 | −8.202 | 1.00 | 20.82 | 6 | C |
| ATOM | 1110 | O | ASP A | 148 | 43.276 | 18.830 | −8.704 | 1.00 | 19.57 | 8 | O |
| ATOM | 1111 | N | TYR A | 149 | 44.287 | 20.152 | −7.171 | 1.00 | 17.71 | 7 | N |
| ATOM | 1112 | CA | TYR A | 149 | 45.253 | 19.083 | −6.803 | 1.00 | 19.31 | 6 | C |
| ATOM | 1113 | CB | TYR A | 149 | 46.673 | 19.764 | −6.813 | 1.00 | 19.25 | 6 | C |
| ATOM | 1114 | CG | TYR A | 149 | 47.105 | 19.891 | −8.276 | 1.00 | 18.61 | 6 | C |
| ATOM | 1115 | CD1 | TYR A | 149 | 46.719 | 20.962 | −9.052 | 1.00 | 18.99 | 6 | C |
| ATOM | 1116 | CE1 | TYR A | 149 | 47.061 | 21.054 | −10.388 | 1.00 | 20.38 | 6 | C |
| ATOM | 1117 | CD2 | TYR A | 149 | 47.918 | 18.921 | −8.882 | 1.00 | 18.56 | 6 | C |
| ATOM | 1118 | CE2 | TYR A | 149 | 48.266 | 18.974 | −10.202 | 1.00 | 21.43 | 6 | C |
| ATOM | 1119 | CZ | TYR A | 149 | 47.839 | 20.061 | −10.955 | 1.00 | 21.46 | 6 | C |
| ATOM | 1120 | OH | TYR A | 149 | 48.131 | 20.116 | −12.304 | 1.00 | 24.87 | 8 | O |
| ATOM | 1121 | C | TYR A | 149 | 44.966 | 18.399 | −5.496 | 1.00 | 19.78 | 6 | C |
| ATOM | 1122 | O | TYR A | 149 | 45.795 | 17.754 | −4.838 | 1.00 | 19.59 | 8 | O |
| ATOM | 1123 | N | SER A | 150 | 43.685 | 18.336 | −5.118 | 1.00 | 19.04 | 7 | N |
| ATOM | 1124 | CA | SER A | 150 | 43.201 | 17.703 | −3.893 | 1.00 | 18.29 | 6 | C |
| ATOM | 1125 | CB | SER A | 150 | 41.778 | 18.114 | −3.619 | 1.00 | 21.78 | 6 | C |
| ATOM | 1126 | OG | SER A | 150 | 40.839 | 17.596 | −4.580 | 1.00 | 24.40 | 8 | O |
| ATOM | 1127 | C | SER A | 150 | 43.418 | 16.201 | −3.879 | 1.00 | 19.10 | 6 | C |
| ATOM | 1128 | O | SER A | 150 | 43.538 | 15.581 | −2.797 | 1.00 | 16.86 | 8 | O |
| ATOM | 1129 | N | TYR A | 151 | 43.630 | 15.584 | −5.098 | 1.00 | 17.41 | 7 | N |
| ATOM | 1130 | CA | TYR A | 151 | 43.992 | 14.192 | −5.117 | 1.00 | 18.39 | 6 | C |
| ATOM | 1131 | CB | TYR A | 151 | 44.143 | 13.607 | −6.535 | 1.00 | 19.38 | 6 | C |
| ATOM | 1132 | CG | TYR A | 151 | 45.412 | 14.035 | −7.229 | 1.00 | 19.51 | 6 | C |
| ATOM | 1133 | CD1 | TYR A | 151 | 46.527 | 13.165 | −7.130 | 1.00 | 18.99 | 6 | C |
| ATOM | 1134 | CE1 | TYR A | 151 | 47.723 | 13.514 | −7.732 | 1.00 | 20.45 | 6 | C |
| ATOM | 1135 | CD2 | TYR A | 151 | 45.561 | 15.226 | −7.900 | 1.00 | 19.91 | 6 | C |
| ATOM | 1136 | CE2 | TYR A | 151 | 46.763 | 15.573 | −8.520 | 1.00 | 17.70 | 6 | C |
| ATOM | 1137 | CZ | TYR A | 151 | 47.839 | 14.704 | −8.408 | 1.00 | 20.22 | 6 | C |
| ATOM | 1138 | OH | TYR A | 151 | 49.008 | 15.082 | −9.008 | 1.00 | 21.10 | 8 | O |
| ATOM | 1139 | C | TYR A | 151 | 45.260 | 13.871 | −4.315 | 1.00 | 19.82 | 6 | C |
| ATOM | 1140 | O | TYR A | 151 | 45.371 | 12.734 | −3.846 | 1.00 | 18.80 | 8 | O |
| ATOM | 1141 | N | LEU A | 152 | 46.251 | 14.778 | −4.265 | 1.00 | 18.06 | 7 | N |
| ATOM | 1142 | CA | LEU A | 152 | 47.412 | 14.487 | −3.435 | 1.00 | 17.83 | 6 | C |
| ATOM | 1143 | CB | LEU A | 152 | 48.389 | 15.681 | −3.520 | 1.00 | 18.77 | 6 | C |
| ATOM | 1144 | CG | LEU A | 152 | 48.991 | 15.943 | −4.885 | 1.00 | 20.30 | 6 | C |
| ATOM | 1145 | CD1 | LEU A | 152 | 49.729 | 17.268 | −4.940 | 1.00 | 22.80 | 6 | C |
| ATOM | 1146 | CD2 | LEU A | 152 | 49.969 | 14.799 | −5.168 | 1.00 | 22.59 | 6 | C |
| ATOM | 1147 | C | LEU A | 152 | 47.073 | 14.230 | −1.984 | 1.00 | 17.28 | 6 | C |
| ATOM | 1148 | O | LEU A | 152 | 47.821 | 13.450 | −1.336 | 1.00 | 17.59 | 8 | O |
| ATOM | 1149 | N | ALA A | 153 | 46.021 | 14.831 | −1.393 | 1.00 | 15.70 | 7 | N |
| ATOM | 1150 | CA | ALA A | 153 | 45.725 | 14.550 | 0.011 | 1.00 | 16.83 | 6 | C |
| ATOM | 1151 | CB | ALA A | 153 | 44.783 | 15.594 | 0.556 | 1.00 | 16.96 | 6 | C |
| ATOM | 1152 | C | ALA A | 153 | 45.173 | 13.116 | 0.193 | 1.00 | 17.84 | 6 | C |
| ATOM | 1153 | O | ALA A | 153 | 45.460 | 12.457 | 1.201 | 1.00 | 19.86 | 8 | O |
| ATOM | 1154 | N | ASP A | 154 | 44.451 | 12.613 | −0.837 | 1.00 | 17.86 | 7 | N |
| ATOM | 1155 | CA | ASP A | 154 | 44.077 | 11.213 | −0.746 | 1.00 | 18.40 | 6 | C |
| ATOM | 1156 | CB | ASP A | 154 | 43.075 | 10.857 | −1.890 | 1.00 | 23.21 | 6 | C |
| ATOM | 1157 | CG | ASP A | 154 | 41.826 | 11.731 | −1.783 | 1.00 | 27.81 | 6 | C |
| ATOM | 1158 | OD1 | ASP A | 154 | 41.377 | 11.886 | −0.635 | 1.00 | 29.48 | 8 | O |
| ATOM | 1159 | OD2 | ASP A | 154 | 41.322 | 12.207 | −2.825 | 1.00 | 29.29 | 8 | O |
| ATOM | 1160 | C | ASP A | 154 | 45.281 | 10.286 | −0.872 | 1.00 | 17.75 | 6 | C |
| ATOM | 1161 | O | ASP A | 154 | 45.276 | 9.260 | −0.169 | 1.00 | 19.35 | 8 | O |
| ATOM | 1162 | N | ILE A | 155 | 46.233 | 10.545 | −1.791 | 1.00 | 18.07 | 7 | N |
| ATOM | 1163 | CA | ILE A | 155 | 47.363 | 9.600 | −1.891 | 1.00 | 18.81 | 6 | C |
| ATOM | 1164 | CB | ILE A | 155 | 48.268 | 10.046 | −3.051 | 1.00 | 18.97 | 6 | C |
| ATOM | 1165 | CG2 | ILE A | 155 | 49.584 | 9.278 | −3.003 | 1.00 | 21.14 | 6 | C |
| ATOM | 1166 | CG1 | ILE A | 155 | 47.573 | 9.892 | −4.403 | 1.00 | 19.05 | 6 | C |
| ATOM | 1167 | CD1 | ILE A | 155 | 48.320 | 10.463 | −5.609 | 1.00 | 21.72 | 6 | C |
| ATOM | 1168 | C | ILE A | 155 | 48.155 | 9.631 | −0.574 | 1.00 | 19.63 | 6 | C |
| ATOM | 1169 | O | ILE A | 155 | 48.599 | 8.626 | −0.012 | 1.00 | 18.45 | 8 | O |
| ATOM | 1170 | N | LEU A | 156 | 48.514 | 10.877 | −0.163 | 1.00 | 18.51 | 7 | N |
| ATOM | 1171 | CA | LEU A | 156 | 49.330 | 11.051 | 1.062 | 1.00 | 18.42 | 6 | C |
| ATOM | 1172 | CB | LEU A | 156 | 49.886 | 12.498 | 1.167 | 1.00 | 17.08 | 6 | C |
| ATOM | 1173 | CG | LEU A | 156 | 50.696 | 12.921 | −0.066 | 1.00 | 18.25 | 6 | C |
| ATOM | 1174 | CD1 | LEU A | 156 | 51.137 | 14.361 | −0.133 | 1.00 | 18.42 | 6 | C |
| ATOM | 1175 | CD2 | LEU A | 156 | 51.981 | 12.051 | −0.126 | 1.00 | 17.93 | 6 | C |
| ATOM | 1176 | C | LEU A | 156 | 48.661 | 10.469 | 2.311 | 1.00 | 20.17 | 6 | C |
| ATOM | 1177 | O | LEU A | 156 | 49.388 | 10.083 | 3.271 | 1.00 | 17.32 | 8 | O |
| ATOM | 1178 | N | GLN A | 157 | 47.341 | 10.474 | 2.415 | 1.00 | 17.74 | 7 | N |
| ATOM | 1179 | CA | GLN A | 157 | 46.660 | 9.866 | 3.556 | 1.00 | 20.92 | 6 | C |
| ATOM | 1180 | CB | GLN A | 157 | 45.165 | 9.975 | 3.328 | 1.00 | 20.02 | 6 | C |
| ATOM | 1181 | CG | GLN A | 157 | 44.351 | 9.149 | 4.326 | 1.00 | 23.11 | 6 | C |
| ATOM | 1182 | CD | GLN A | 157 | 44.402 | 9.703 | 5.739 | 1.00 | 26.23 | 6 | C |
| ATOM | 1183 | OE1 | GLN A | 157 | 44.563 | 10.899 | 6.002 | 1.00 | 26.75 | 8 | O |
| ATOM | 1184 | NE2 | GLN A | 157 | 44.167 | 8.825 | 6.716 | 1.00 | 25.25 | 7 | N |
| ATOM | 1185 | C | GLN A | 157 | 47.115 | 8.399 | 3.682 | 1.00 | 20.20 | 6 | C |
| ATOM | 1186 | O | GLN A | 157 | 47.256 | 7.940 | 4.825 | 1.00 | 24.89 | 8 | O |
| ATOM | 1187 | N | THR A | 158 | 47.184 | 7.690 | 2.560 | 1.00 | 21.62 | 7 | N |
| ATOM | 1188 | CA | THR A | 158 | 47.620 | 6.298 | 2.561 | 1.00 | 23.71 | 6 | C |
| ATOM | 1189 | CB | THR A | 158 | 47.393 | 5.565 | 1.207 | 1.00 | 24.12 | 6 | C |
| ATOM | 1190 | OG1 | THR A | 158 | 48.340 | 6.029 | 0.221 | 1.00 | 26.48 | 8 | O |
| ATOM | 1191 | CG2 | THR A | 158 | 45.978 | 5.718 | 0.677 | 1.00 | 24.75 | 6 | C |
| ATOM | 1192 | C | THR A | 158 | 49.045 | 6.078 | 2.999 | 1.00 | 22.40 | 6 | C |
| ATOM | 1193 | O | THR A | 158 | 49.491 | 4.921 | 3.279 | 1.00 | 22.94 | 8 | O |
| ATOM | 1194 | N | VAL A | 159 | 49.915 | 7.105 | 2.970 | 1.00 | 18.84 | 7 | N |
| ATOM | 1195 | CA | VAL A | 159 | 51.313 | 7.010 | 3.279 | 1.00 | 17.40 | 6 | C |
| ATOM | 1196 | CB | VAL A | 159 | 52.025 | 8.038 | 2.343 | 1.00 | 20.38 | 6 | C |
| ATOM | 1197 | CG1 | VAL A | 159 | 53.483 | 8.258 | 2.615 | 1.00 | 18.85 | 6 | C |
| ATOM | 1198 | CG2 | VAL A | 159 | 51.795 | 7.503 | 0.946 | 1.00 | 20.55 | 6 | C |
| ATOM | 1199 | C | VAL A | 159 | 51.642 | 7.413 | 4.693 | 1.00 | 18.65 | 6 | C |
| ATOM | 1200 | O | VAL A | 159 | 52.420 | 6.732 | 5.370 | 1.00 | 17.88 | 8 | O |
| ATOM | 1201 | N | LEU A | 160 | 51.139 | 8.516 | 5.238 | 1.00 | 18.36 | 7 | N |
| ATOM | 1202 | CA | LEU A | 160 | 51.415 | 9.006 | 6.556 | 1.00 | 14.96 | 6 | C |
| ATOM | 1203 | CB | LEU A | 160 | 52.592 | 10.035 | 6.502 | 1.00 | 14.95 | 6 | C |
| ATOM | 1204 | CG | LEU A | 160 | 52.952 | 10.554 | 7.920 | 1.00 | 15.76 | 6 | C |
| ATOM | 1205 | CD1 | LEU A | 160 | 53.677 | 9.452 | 8.679 | 1.00 | 18.53 | 6 | C |
| ATOM | 1206 | CD2 | LEU A | 160 | 53.818 | 11.827 | 7.863 | 1.00 | 17.23 | 6 | C |
| ATOM | 1207 | C | LEU A | 160 | 50.162 | 9.655 | 7.122 | 1.00 | 19.50 | 6 | C |
| ATOM | 1208 | O | LEU A | 160 | 49.937 | 10.845 | 7.007 | 1.00 | 17.83 | 8 | O |
| ATOM | 1209 | N | PRO A | 161 | 49.242 | 8.791 | 7.623 | 1.00 | 20.05 | 7 | N |
| ATOM | 1210 | CD | PRO A | 161 | 49.447 | 7.346 | 7.746 | 1.00 | 22.88 | 6 | C |
| ATOM | 1211 | CA | PRO A | 161 | 47.987 | 9.317 | 8.188 | 1.00 | 22.07 | 6 | C |
| ATOM | 1212 | CB | PRO A | 161 | 47.427 | 8.034 | 8.827 | 1.00 | 23.37 | 6 | C |
| ATOM | 1213 | CG | PRO A | 161 | 48.104 | 6.871 | 8.206 | 1.00 | 25.78 | 6 | C |
| ATOM | 1214 | C | PRO A | 161 | 48.269 | 10.387 | 9.197 | 1.00 | 17.48 | 6 | C |
| ATOM | 1215 | O | PRO A | 161 | 49.206 | 10.280 | 10.005 | 1.00 | 18.09 | 8 | O |
| ATOM | 1216 | N | ASP A | 162 | 47.441 | 11.408 | 9.434 | 1.00 | 17.11 | 7 | N |
| ATOM | 1217 | CA | ASP A | 162 | 46.275 | 11.733 | 8.634 | 1.00 | 20.91 | 6 | C |
| ATOM | 1218 | CB | ASP A | 162 | 45.110 | 12.191 | 9.533 | 1.00 | 21.15 | 6 | C |
| ATOM | 1219 | CG | ASP A | 162 | 44.606 | 11.011 | 10.384 | 1.00 | 23.93 | 6 | C |
| ATOM | 1220 | OD1 | ASP A | 162 | 44.427 | 11.224 | 11.597 | 1.00 | 26.09 | 8 | O |
| ATOM | 1221 | OD2 | ASP A | 162 | 44.359 | 9.974 | 9.747 | 1.00 | 25.38 | 8 | O |
| ATOM | 1222 | C | ASP A | 162 | 46.635 | 12.948 | 7.754 | 1.00 | 20.14 | 6 | C |
| ATOM | 1223 | O | ASP A | 162 | 47.409 | 13.818 | 8.216 | 1.00 | 17.58 | 8 | O |
| ATOM | 1224 | N | VAL A | 163 | 45.983 | 13.117 | 6.591 | 1.00 | 18.77 | 7 | N |
| ATOM | 1225 | CA | VAL A | 163 | 46.256 | 14.288 | 5.761 | 1.00 | 20.26 | 6 | C |
| ATOM | 1226 | CB | VAL A | 163 | 46.955 | 13.882 | 4.449 | 1.00 | 19.23 | 6 | C |
| ATOM | 1227 | CG1 | VAL A | 163 | 47.205 | 15.131 | 3.548 | 1.00 | 18.64 | 6 | C |
| ATOM | 1228 | CG2 | VAL A | 163 | 48.279 | 13.185 | 4.773 | 1.00 | 17.67 | 6 | C |
| ATOM | 1229 | C | VAL A | 163 | 44.912 | 14.950 | 5.418 | 1.00 | 25.61 | 6 | C |
| ATOM | 1230 | O | VAL A | 163 | 43.982 | 14.148 | 5.193 | 1.00 | 26.99 | 8 | O |
| ATOM | 1231 | N | ALA A | 164 | 44.802 | 16.252 | 5.477 | 1.00 | 23.09 | 7 | N |
| ATOM | 1232 | CA | ALA A | 164 | 43.520 | 16.903 | 5.130 | 1.00 | 25.35 | 6 | C |
| ATOM | 1233 | CB | ALA A | 164 | 42.885 | 17.556 | 6.366 | 1.00 | 26.07 | 6 | C |
| ATOM | 1234 | C | ALA A | 164 | 43.742 | 18.004 | 4.101 | 1.00 | 25.43 | 6 | C |
| ATOM | 1235 | O | ALA A | 164 | 44.669 | 18.783 | 4.256 | 1.00 | 21.31 | 8 | O |
| ATOM | 1236 | N | TRP A | 165 | 42.884 | 18.080 | 3.094 | 1.00 | 25.90 | 7 | N |
| ATOM | 1237 | CA | TRP A | 165 | 42.910 | 19.161 | 2.119 | 1.00 | 24.31 | 6 | C |
| ATOM | 1238 | CB | TRP A | 165 | 42.277 | 18.601 | 0.828 | 1.00 | 24.64 | 6 | C |
| ATOM | 1239 | CG | TRP A | 165 | 42.165 | 19.645 | −0.249 | 1.00 | 23.94 | 6 | C |
| ATOM | 1240 | CD2 | TRP A | 165 | 43.210 | 20.066 | −1.134 | 1.00 | 23.27 | 6 | C |
| ATOM | 1241 | CE2 | TRP A | 165 | 42.664 | 21.063 | −1.987 | 1.00 | 24.22 | 6 | C |
| ATOM | 1242 | CE3 | TRP A | 165 | 44.562 | 19.693 | −1.303 | 1.00 | 21.75 | 6 | C |
| ATOM | 1243 | CD1 | TRP A | 165 | 41.047 | 20.380 | −0.564 | 1.00 | 24.22 | 6 | C |
| ATOM | 1244 | NE1 | TRP A | 165 | 41.358 | 21.237 | −1.562 | 1.00 | 24.36 | 7 | N |
| ATOM | 1245 | CZ2 | TRP A | 165 | 43.434 | 21.668 | −2.951 | 1.00 | 24.21 | 6 | C |
| ATOM | 1246 | CZ3 | TRP A | 165 | 45.293 | 20.315 | −2.316 | 1.00 | 23.39 | 6 | C |
| ATOM | 1247 | CH2 | TRP A | 165 | 44.731 | 21.317 | −3.143 | 1.00 | 22.79 | 6 | C |
| ATOM | 1248 | C | TRP A | 165 | 42.023 | 20.330 | 2.597 | 1.00 | 23.95 | 6 | C |
| ATOM | 1249 | O | TRP A | 165 | 40.956 | 20.025 | 3.102 | 1.00 | 24.83 | 8 | O |
| ATOM | 1250 | N | HIS A | 166 | 42.533 | 21.523 | 2.498 | 1.00 | 21.92 | 7 | N |
| ATOM | 1251 | CA | HIS A | 166 | 41.763 | 22.731 | 2.813 | 1.00 | 26.16 | 6 | C |
| ATOM | 1252 | CB | HIS A | 166 | 42.503 | 23.520 | 3.923 | 1.00 | 28.94 | 6 | C |
| ATOM | 1253 | CG | HIS A | 166 | 42.416 | 23.009 | 5.311 | 1.00 | 31.73 | 6 | C |
| ATOM | 1254 | CD2 | HIS A | 166 | 41.819 | 23.535 | 6.397 | 1.00 | 32.29 | 6 | C |
| ATOM | 1255 | ND1 | HIS A | 166 | 43.012 | 21.846 | 5.744 | 1.00 | 35.64 | 7 | N |
| ATOM | 1256 | CE1 | HIS A | 166 | 42.752 | 21.671 | 7.021 | 1.00 | 33.06 | 6 | C |
| ATOM | 1257 | NE2 | HIS A | 166 | 42.009 | 22.662 | 7.460 | 1.00 | 35.48 | 7 | N |
| ATOM | 1258 | C | HIS A | 166 | 41.769 | 23.651 | 1.600 | 1.00 | 27.45 | 6 | C |
| ATOM | 1259 | O | HIS A | 166 | 42.867 | 24.027 | 1.162 | 1.00 | 24.48 | 8 | O |
| ATOM | 1260 | N | ASN A | 167 | 40.577 | 24.065 | 1.151 | 1.00 | 27.45 | 7 | N |
| ATOM | 1261 | CA | ASN A | 167 | 40.602 | 25.027 | 0.031 | 1.00 | 28.70 | 6 | C |
| ATOM | 1262 | CB | ASN A | 167 | 39.215 | 25.180 | −0.565 | 1.00 | 32.95 | 6 | C |
| ATOM | 1263 | CG | ASN A | 167 | 38.685 | 23.910 | −1.201 | 1.00 | 37.78 | 6 | C |
| ATOM | 1264 | OD1 | ASN A | 167 | 37.750 | 23.235 | −0.724 | 1.00 | 43.16 | 8 | O |
| ATOM | 1265 | ND2 | ASN A | 167 | 39.249 | 23.490 | −2.322 | 1.00 | 38.01 | 7 | N |
| ATOM | 1266 | C | ASN A | 167 | 41.224 | 26.367 | 0.396 | 1.00 | 29.07 | 6 | C |
| ATOM | 1267 | O | ASN A | 167 | 41.787 | 27.080 | −0.446 | 1.00 | 29.87 | 8 | O |
| ATOM | 1268 | N | ASN A | 168 | 41.151 | 26.758 | 1.656 | 1.00 | 28.01 | 7 | N |
| ATOM | 1269 | CA | ASN A | 168 | 41.735 | 27.986 | 2.191 | 1.00 | 27.50 | 6 | C |
| ATOM | 1270 | CB | ASN A | 168 | 40.586 | 28.843 | 2.716 | 1.00 | 30.31 | 6 | C |
| ATOM | 1271 | CG | ASN A | 168 | 41.110 | 30.176 | 3.249 | 1.00 | 34.40 | 6 | C |
| ATOM | 1272 | OD1 | ASN A | 168 | 40.418 | 30.801 | 4.029 | 1.00 | 35.33 | 8 | O |
| ATOM | 1273 | ND2 | ASN A | 168 | 42.277 | 30.557 | 2.754 | 1.00 | 34.66 | 7 | N |
| ATOM | 1274 | C | ASN A | 168 | 42.675 | 27.612 | 3.337 | 1.00 | 25.27 | 6 | C |
| ATOM | 1275 | O | ASN A | 168 | 42.411 | 27.775 | 4.535 | 1.00 | 25.28 | 8 | O |
| ATOM | 1276 | N | ILE A | 169 | 43.817 | 27.070 | 2.946 | 1.00 | 23.01 | 7 | N |
| ATOM | 1277 | CA | ILE A | 169 | 44.824 | 26.571 | 3.869 | 1.00 | 22.62 | 6 | C |
| ATOM | 1278 | CB | ILE A | 169 | 45.870 | 25.743 | 3.111 | 1.00 | 22.81 | 6 | C |
| ATOM | 1279 | CG2 | ILE A | 169 | 46.662 | 26.592 | 2.150 | 1.00 | 24.55 | 6 | C |
| ATOM | 1280 | CG1 | ILE A | 169 | 46.825 | 25.062 | 4.140 | 1.00 | 22.70 | 6 | C |
| ATOM | 1281 | CD1 | ILE A | 169 | 46.171 | 24.064 | 5.042 | 1.00 | 24.31 | 6 | C |
| ATOM | 1282 | C | ILE A | 169 | 45.447 | 27.697 | 4.705 | 1.00 | 21.77 | 6 | C |
| ATOM | 1283 | O | ILE A | 169 | 45.858 | 27.466 | 5.841 | 1.00 | 17.89 | 8 | O |
| ATOM | 1284 | N | ARG A | 170 | 45.464 | 28.937 | 4.194 | 1.00 | 24.71 | 7 | N |
| ATOM | 1285 | CA | ARG A | 170 | 46.002 | 30.060 | 4.980 | 1.00 | 26.16 | 6 | C |
| ATOM | 1286 | CB | ARG A | 170 | 45.977 | 31.408 | 4.246 | 1.00 | 25.22 | 6 | C |
| ATOM | 1287 | CG | ARG A | 170 | 47.035 | 31.472 | 3.136 | 1.00 | 26.01 | 6 | C |
| ATOM | 1288 | CD | ARG A | 170 | 46.739 | 32.618 | 2.183 | 1.00 | 28.66 | 6 | C |
| ATOM | 1289 | NE | ARG A | 170 | 47.777 | 32.796 | 1.170 | 1.00 | 28.74 | 7 | N |
| ATOM | 1290 | CZ | ARG A | 170 | 47.921 | 32.047 | 0.078 | 1.00 | 28.10 | 6 | C |
| ATOM | 1291 | NH1 | ARG A | 170 | 47.112 | 31.054 | −0.198 | 1.00 | 27.50 | 7 | N |
| ATOM | 1292 | NH2 | ARG A | 170 | 48.916 | 32.299 | −0.773 | 1.00 | 26.55 | 7 | N |
| ATOM | 1293 | C | ARG A | 170 | 45.278 | 30.230 | 6.316 | 1.00 | 23.86 | 6 | C |
| ATOM | 1294 | O | ARG A | 170 | 45.888 | 30.743 | 7.289 | 1.00 | 22.23 | 8 | O |
| ATOM | 1295 | N | ALA A | 171 | 44.008 | 29.907 | 6.462 | 1.00 | 25.00 | 7 | N |
| ATOM | 1296 | CA | ALA A | 171 | 43.328 | 29.992 | 7.754 | 1.00 | 25.32 | 6 | C |
| ATOM | 1297 | CB | ALA A | 171 | 41.885 | 29.555 | 7.584 | 1.00 | 26.49 | 6 | C |
| ATOM | 1298 | C | ALA A | 171 | 44.029 | 29.135 | 8.811 | 1.00 | 26.63 | 6 | C |
| ATOM | 1299 | O | ALA A | 171 | 44.307 | 29.602 | 9.939 | 1.00 | 25.59 | 8 | O |
| ATOM | 1300 | N | GLU A | 172 | 44.318 | 27.865 | 8.467 | 1.00 | 25.88 | 7 | N |
| ATOM | 1301 | CA | GLU A | 172 | 45.043 | 27.018 | 9.427 | 1.00 | 27.61 | 6 | C |
| ATOM | 1302 | CB | GLU A | 172 | 44.953 | 25.537 | 8.990 | 1.00 | 32.05 | 6 | C |
| ATOM | 1303 | CG | GLU A | 172 | 44.147 | 24.675 | 10.005 | 1.00 | 39.62 | 6 | C |
| ATOM | 1304 | CD | GLU A | 172 | 45.075 | 24.212 | 11.102 | 1.00 | 43.09 | 6 | C |
| ATOM | 1305 | OE1 | GLU A | 172 | 46.310 | 24.301 | 10.824 | 1.00 | 46.76 | 8 | O |
| ATOM | 1306 | OE2 | GLU A | 172 | 44.926 | 23.763 | 12.243 | 1.00 | 46.39 | 8 | O |
| ATOM | 1307 | C | GLU A | 172 | 46.496 | 27.484 | 9.632 | 1.00 | 23.72 | 6 | C |
| ATOM | 1308 | O | GLU A | 172 | 46.985 | 27.429 | 10.785 | 1.00 | 23.95 | 8 | O |
| ATOM | 1309 | N | LEU A | 173 | 47.171 | 27.918 | 8.556 | 1.00 | 21.07 | 7 | N |
| ATOM | 1310 | CA | LEU A | 173 | 48.528 | 28.435 | 8.732 | 1.00 | 22.25 | 6 | C |
| ATOM | 1311 | CB | LEU A | 173 | 49.153 | 28.820 | 7.406 | 1.00 | 21.01 | 6 | C |
| ATOM | 1312 | CG | LEU A | 173 | 49.162 | 27.731 | 6.323 | 1.00 | 20.24 | 6 | C |
| ATOM | 1313 | CD1 | LEU A | 173 | 49.773 | 28.278 | 5.023 | 1.00 | 20.08 | 6 | C |
| ATOM | 1314 | CD2 | LEU A | 173 | 49.855 | 26.479 | 6.849 | 1.00 | 22.60 | 6 | C |
| ATOM | 1315 | C | LEU A | 173 | 48.544 | 29.611 | 9.706 | 1.00 | 22.82 | 6 | C |
| ATOM | 1316 | O | LEU A | 173 | 49.421 | 29.704 | 10.555 | 1.00 | 21.34 | 8 | O |
| ATOM | 1317 | N | TRP A | 174 | 47.609 | 30.566 | 9.539 | 1.00 | 22.83 | 7 | N |
| ATOM | 1318 | CA | TRP A | 174 | 47.650 | 31.707 | 10.516 | 1.00 | 23.51 | 6 | C |
| ATOM | 1319 | CB | TRP A | 174 | 46.559 | 32.725 | 10.134 | 1.00 | 23.11 | 6 | C |
| ATOM | 1320 | CG | TRP A | 174 | 47.069 | 33.716 | 9.124 | 1.00 | 26.78 | 6 | C |
| ATOM | 1321 | CD2 | TRP A | 174 | 47.989 | 34.778 | 9.399 | 1.00 | 28.58 | 6 | C |
| ATOM | 1322 | CE2 | TRP A | 174 | 48.192 | 35.459 | 8.176 | 1.00 | 30.00 | 6 | C |
| ATOM | 1323 | CE3 | TRP A | 174 | 48.652 | 35.210 | 10.553 | 1.00 | 28.70 | 6 | C |
| ATOM | 1324 | CD1 | TRP A | 174 | 46.743 | 33.809 | 7.813 | 1.00 | 27.80 | 6 | C |
| ATOM | 1325 | NE1 | TRP A | 174 | 47.419 | 34.852 | 7.221 | 1.00 | 29.52 | 7 | N |
| ATOM | 1326 | CZ2 | TRP A | 174 | 49.034 | 36.571 | 8.081 | 1.00 | 31.38 | 6 | C |
| ATOM | 1327 | CZ3 | TRP A | 174 | 49.507 | 36.332 | 10.443 | 1.00 | 29.43 | 6 | C |
| ATOM | 1328 | CH2 | TRP A | 174 | 49.681 | 36.968 | 9.209 | 1.00 | 30.53 | 6 | C |
| ATOM | 1329 | C | TRP A | 174 | 47.375 | 31.312 | 11.950 | 1.00 | 25.29 | 6 | C |
| ATOM | 1330 | O | TRP A | 174 | 47.823 | 32.005 | 12.866 | 1.00 | 23.65 | 8 | O |
| ATOM | 1331 | N | ARG A | 175 | 46.584 | 30.275 | 12.205 | 1.00 | 24.50 | 7 | N |
| ATOM | 1332 | CA | ARG A | 175 | 46.257 | 29.876 | 13.568 | 1.00 | 26.52 | 6 | C |
| ATOM | 1333 | CB | ARG A | 175 | 45.290 | 28.700 | 13.664 | 1.00 | 28.84 | 6 | C |
| ATOM | 1334 | CG | ARG A | 175 | 44.985 | 28.364 | 15.144 | 1.00 | 33.06 | 6 | C |
| ATOM | 1335 | CD | ARG A | 175 | 44.082 | 27.143 | 15.260 | 1.00 | 37.50 | 6 | C |
| ATOM | 1336 | NE | ARG A | 175 | 44.737 | 25.928 | 14.797 | 1.00 | 41.71 | 7 | N |
| ATOM | 1337 | CZ | ARG A | 175 | 45.692 | 25.235 | 15.382 | 1.00 | 43.60 | 6 | C |
| ATOM | 1338 | NH1 | ARG A | 175 | 46.220 | 25.593 | 16.553 | 1.00 | 44.31 | 7 | N |
| ATOM | 1339 | NH2 | ARG A | 175 | 46.196 | 24.150 | 14.815 | 1.00 | 44.59 | 7 | N |
| ATOM | 1340 | C | ARG A | 175 | 47.567 | 29.468 | 14.251 | 1.00 | 25.51 | 6 | C |
| ATOM | 1341 | O | ARG A | 175 | 47.898 | 29.911 | 15.334 | 1.00 | 25.39 | 8 | O |
| ATOM | 1342 | N | LYS A | 176 | 48.355 | 28.625 | 13.559 | 1.00 | 23.60 | 7 | N |
| ATOM | 1343 | CA | LYS A | 176 | 49.640 | 28.258 | 14.145 | 1.00 | 23.20 | 6 | C |
| ATOM | 1344 | CB | LYS A | 176 | 50.234 | 27.029 | 13.428 | 1.00 | 26.07 | 6 | C |
| ATOM | 1345 | CG | LYS A | 176 | 49.285 | 25.821 | 13.676 | 1.00 | 34.60 | 6 | C |
| ATOM | 1346 | CD | LYS A | 176 | 50.194 | 24.602 | 13.582 | 1.00 | 40.07 | 6 | C |
| ATOM | 1347 | CE | LYS A | 176 | 50.524 | 24.233 | 12.142 | 1.00 | 43.53 | 6 | C |
| ATOM | 1348 | NZ | LYS A | 176 | 51.993 | 23.880 | 12.082 | 1.00 | 47.01 | 7 | N |
| ATOM | 1349 | C | LYS A | 176 | 50.646 | 29.381 | 14.188 | 1.00 | 21.35 | 6 | C |
| ATOM | 1350 | O | LYS A | 176 | 51.231 | 29.615 | 15.238 | 1.00 | 22.23 | 8 | O |
| ATOM | 1351 | N | LEU A | 177 | 50.764 | 30.193 | 13.122 | 1.00 | 18.60 | 7 | N |
| ATOM | 1352 | CA | LEU A | 177 | 51.716 | 31.332 | 13.150 | 1.00 | 18.23 | 6 | C |
| ATOM | 1353 | CB | LEU A | 177 | 51.715 | 32.043 | 11.793 | 1.00 | 22.54 | 6 | C |
| ATOM | 1354 | CG | LEU A | 177 | 52.784 | 33.167 | 11.675 | 1.00 | 24.69 | 6 | C |
| ATOM | 1355 | CD1 | LEU A | 177 | 53.234 | 33.294 | 10.245 | 1.00 | 27.74 | 6 | C |
| ATOM | 1356 | CD2 | LEU A | 177 | 52.041 | 34.461 | 12.047 | 1.00 | 30.03 | 6 | C |
| ATOM | 1357 | C | LEU A | 177 | 51.310 | 32.360 | 14.203 | 1.00 | 18.36 | 6 | C |
| ATOM | 1358 | O | LEU A | 177 | 52.190 | 32.931 | 14.870 | 1.00 | 20.29 | 8 | O |
| ATOM | 1359 | N | ALA A | 178 | 50.041 | 32.560 | 14.495 | 1.00 | 21.31 | 7 | N |
| ATOM | 1360 | CA | ALA A | 178 | 49.680 | 33.541 | 15.560 | 1.00 | 23.94 | 6 | C |
| ATOM | 1361 | CB | ALA A | 178 | 48.157 | 33.635 | 15.703 | 1.00 | 24.01 | 6 | C |
| ATOM | 1362 | C | ALA A | 178 | 50.308 | 33.185 | 16.907 | 1.00 | 23.64 | 6 | C |
| ATOM | 1363 | O | ALA A | 178 | 50.852 | 34.064 | 17.630 | 1.00 | 22.84 | 8 | O |
| ATOM | 1364 | N | VAL A | 179 | 50.168 | 31.929 | 17.282 | 1.00 | 21.39 | 7 | N |
| ATOM | 1365 | CA | VAL A | 179 | 50.732 | 31.438 | 18.549 | 1.00 | 22.43 | 6 | C |
| ATOM | 1366 | CB | VAL A | 179 | 50.343 | 29.981 | 18.861 | 1.00 | 22.10 | 6 | C |
| ATOM | 1367 | CG1 | VAL A | 179 | 50.951 | 29.530 | 20.187 | 1.00 | 23.11 | 6 | C |
| ATOM | 1368 | CG2 | VAL A | 179 | 48.820 | 29.834 | 18.937 | 1.00 | 23.47 | 6 | C |
| ATOM | 1369 | C | VAL A | 179 | 52.245 | 31.537 | 18.469 | 1.00 | 20.48 | 6 | C |
| ATOM | 1370 | O | VAL A | 179 | 52.908 | 31.945 | 19.413 | 1.00 | 19.73 | 8 | O |
| ATOM | 1371 | N | ASN A | 180 | 52.891 | 31.153 | 17.338 | 1.00 | 19.20 | 7 | N |
| ATOM | 1372 | CA | ASN A | 180 | 54.335 | 31.162 | 17.284 | 1.00 | 19.90 | 6 | C |
| ATOM | 1373 | CB | ASN A | 180 | 54.741 | 30.494 | 15.925 | 1.00 | 20.96 | 6 | C |
| ATOM | 1374 | CG | ASN A | 180 | 54.641 | 28.976 | 16.044 | 1.00 | 27.73 | 6 | C |
| ATOM | 1375 | OD1 | ASN A | 180 | 54.775 | 28.408 | 17.135 | 1.00 | 30.37 | 8 | O |
| ATOM | 1376 | ND2 | ASN A | 180 | 54.399 | 28.360 | 14.905 | 1.00 | 29.03 | 7 | N |
| ATOM | 1377 | C | ASN A | 180 | 54.935 | 32.565 | 17.346 | 1.00 | 18.72 | 6 | C |
| ATOM | 1378 | O | ASN A | 180 | 56.027 | 32.746 | 17.883 | 1.00 | 19.07 | 8 | O |
| ATOM | 1379 | N | CYS A | 181 | 54.171 | 33.577 | 16.899 | 1.00 | 18.21 | 7 | N |
| ATOM | 1380 | CA | CYS A | 181 | 54.684 | 34.954 | 17.001 | 1.00 | 19.81 | 6 | C |
| ATOM | 1381 | CB | CYS A | 181 | 53.695 | 35.935 | 16.354 | 1.00 | 19.04 | 6 | C |
| ATOM | 1382 | SG | CYS A | 181 | 53.758 | 35.997 | 14.522 | 1.00 | 20.27 | 16 | S |
| ATOM | 1383 | C | CYS A | 181 | 54.804 | 35.383 | 18.475 | 1.00 | 20.92 | 6 | C |
| ATOM | 1384 | O | CYS A | 181 | 55.605 | 36.297 | 18.689 | 1.00 | 20.72 | 8 | O |
| ATOM | 1385 | N | VAL A | 182 | 54.112 | 34.755 | 19.402 | 1.00 | 21.32 | 7 | N |
| ATOM | 1386 | CA | VAL A | 182 | 54.237 | 35.149 | 20.819 | 1.00 | 22.16 | 6 | C |
| ATOM | 1387 | CB | VAL A | 182 | 52.909 | 34.884 | 21.539 | 1.00 | 24.56 | 6 | C |
| ATOM | 1388 | CG1 | VAL A | 182 | 53.106 | 35.037 | 23.070 | 1.00 | 24.16 | 6 | C |
| ATOM | 1389 | CG2 | VAL A | 182 | 51.784 | 35.745 | 21.041 | 1.00 | 24.61 | 6 | C |
| ATOM | 1390 | C | VAL A | 182 | 55.290 | 34.282 | 21.489 | 1.00 | 22.03 | 6 | C |
| ATOM | 1391 | O | VAL A | 182 | 56.317 | 34.682 | 22.044 | 1.00 | 23.31 | 8 | O |
| ATOM | 1392 | N | ILE A | 183 | 55.035 | 32.961 | 21.459 | 1.00 | 21.31 | 7 | N |
| ATOM | 1393 | CA | ILE A | 183 | 55.914 | 32.016 | 22.141 | 1.00 | 23.41 | 6 | C |
| ATOM | 1394 | CB | ILE A | 183 | 55.275 | 30.592 | 22.170 | 1.00 | 25.25 | 6 | C |
| ATOM | 1395 | CG2 | ILE A | 183 | 56.195 | 29.575 | 22.843 | 1.00 | 21.69 | 6 | C |
| ATOM | 1396 | CG1 | ILE A | 183 | 53.937 | 30.690 | 22.897 | 1.00 | 22.10 | 6 | C |
| ATOM | 1397 | CD1 | ILE A | 183 | 53.169 | 29.346 | 22.962 | 1.00 | 26.09 | 6 | C |
| ATOM | 1398 | C | ILE A | 183 | 57.348 | 31.988 | 21.670 | 1.00 | 24.52 | 6 | C |
| ATOM | 1399 | O | ILE A | 183 | 58.305 | 31.941 | 22.438 | 1.00 | 24.88 | 8 | O |
| ATOM | 1400 | N | ASN A | 184 | 57.668 | 31.937 | 20.363 | 1.00 | 19.27 | 7 | N |
| ATOM | 1401 | CA | ASN A | 184 | 58.998 | 31.742 | 19.876 | 1.00 | 23.27 | 6 | C |
| ATOM | 1402 | CB | ASN A | 184 | 59.013 | 31.461 | 18.363 | 1.00 | 25.05 | 6 | C |
| ATOM | 1403 | CG | ASN A | 184 | 58.715 | 30.015 | 18.026 | 1.00 | 29.29 | 6 | C |
| ATOM | 1404 | OD1 | ASN A | 184 | 59.110 | 29.138 | 18.781 | 1.00 | 31.28 | 8 | O |
| ATOM | 1405 | ND2 | ASN A | 184 | 58.066 | 29.666 | 16.912 | 1.00 | 31.45 | 7 | N |
| ATOM | 1406 | C | ASN A | 184 | 59.967 | 32.890 | 20.219 | 1.00 | 22.82 | 6 | C |
| ATOM | 1407 | O | ASN A | 184 | 60.979 | 32.560 | 20.809 | 1.00 | 23.61 | 8 | O |
| ATOM | 1408 | N | PRO A | 185 | 59.659 | 34.106 | 19.855 | 1.00 | 24.35 | 7 | N |
| ATOM | 1409 | CD | PRO A | 185 | 58.493 | 34.554 | 19.103 | 1.00 | 23.26 | 6 | C |
| ATOM | 1410 | CA | PRO A | 185 | 60.586 | 35.233 | 20.135 | 1.00 | 24.56 | 6 | C |
| ATOM | 1411 | CB | PRO A | 185 | 60.046 | 36.410 | 19.381 | 1.00 | 25.90 | 6 | C |
| ATOM | 1412 | CG | PRO A | 185 | 58.639 | 36.065 | 19.048 | 1.00 | 25.05 | 6 | C |
| ATOM | 1413 | C | PRO A | 185 | 60.671 | 35.542 | 21.642 | 1.00 | 25.36 | 6 | C |
| ATOM | 1414 | O | PRO A | 185 | 61.821 | 35.786 | 22.059 | 1.00 | 23.15 | 8 | O |
| ATOM | 1415 | N | LEU A | 186 | 59.590 | 35.511 | 22.388 | 1.00 | 23.66 | 7 | N |
| ATOM | 1416 | CA | LEU A | 186 | 59.723 | 35.775 | 23.848 | 1.00 | 25.26 | 6 | C |
| ATOM | 1417 | CB | LEU A | 186 | 58.381 | 35.851 | 24.519 | 1.00 | 26.65 | 6 | C |
| ATOM | 1418 | CG | LEU A | 186 | 57.497 | 37.047 | 24.176 | 1.00 | 26.42 | 6 | C |
| ATOM | 1419 | CD1 | LEU A | 186 | 56.179 | 36.947 | 24.905 | 1.00 | 27.09 | 6 | C |
| ATOM | 1420 | CD2 | LEU A | 186 | 58.210 | 38.384 | 24.491 | 1.00 | 27.26 | 6 | C |
| ATOM | 1421 | C | LEU A | 186 | 60.626 | 34.776 | 24.510 | 1.00 | 26.39 | 6 | C |
| ATOM | 1422 | O | LEU A | 186 | 61.553 | 35.200 | 25.272 | 1.00 | 26.22 | 8 | O |
| ATOM | 1423 | N | THR A | 187 | 60.424 | 33.463 | 24.363 | 1.00 | 24.66 | 7 | N |
| ATOM | 1424 | CA | THR A | 187 | 61.294 | 32.450 | 24.874 | 1.00 | 26.73 | 6 | C |
| ATOM | 1425 | CB | THR A | 187 | 60.902 | 30.977 | 24.565 | 1.00 | 25.80 | 6 | C |
| ATOM | 1426 | OG1 | THR A | 187 | 60.767 | 30.740 | 23.150 | 1.00 | 26.21 | 8 | O |
| ATOM | 1427 | CG2 | THR A | 187 | 59.607 | 30.774 | 25.365 | 1.00 | 25.53 | 6 | C |
| ATOM | 1428 | C | THR A | 187 | 62.747 | 32.613 | 24.398 | 1.00 | 28.97 | 6 | C |
| ATOM | 1429 | O | THR A | 187 | 63.725 | 32.170 | 25.010 | 1.00 | 28.90 | 8 | O |
| ATOM | 1430 | N | ALA A | 188 | 62.893 | 33.071 | 23.141 | 1.00 | 27.15 | 7 | N |
| ATOM | 1431 | CA | ALA A | 188 | 64.179 | 33.247 | 22.547 | 1.00 | 28.24 | 6 | C |
| ATOM | 1432 | CB | ALA A | 188 | 64.013 | 33.638 | 21.079 | 1.00 | 26.69 | 6 | C |
| ATOM | 1433 | C | ALA A | 188 | 64.929 | 34.392 | 23.276 | 1.00 | 26.56 | 6 | C |
| ATOM | 1434 | O | ALA A | 188 | 66.085 | 34.131 | 23.538 | 1.00 | 31.03 | 8 | O |
| ATOM | 1435 | N | ILE A | 189 | 64.247 | 35.480 | 23.520 | 1.00 | 28.59 | 7 | N |
| ATOM | 1436 | CA | ILE A | 189 | 64.847 | 36.681 | 24.089 | 1.00 | 31.95 | 6 | C |
| ATOM | 1437 | CB | ILE A | 189 | 63.944 | 37.886 | 23.862 | 1.00 | 34.61 | 6 | C |
| ATOM | 1438 | CG2 | ILE A | 189 | 64.349 | 39.093 | 24.682 | 1.00 | 39.21 | 6 | C |
| ATOM | 1439 | CG1 | ILE A | 189 | 63.972 | 38.315 | 22.366 | 1.00 | 35.95 | 6 | C |
| ATOM | 1440 | CD1 | ILE A | 189 | 62.719 | 39.074 | 22.016 | 1.00 | 38.04 | 6 | C |
| ATOM | 1441 | C | ILE A | 189 | 65.027 | 36.539 | 25.602 | 1.00 | 33.11 | 6 | C |
| ATOM | 1442 | O | ILE A | 189 | 65.931 | 37.189 | 26.149 | 1.00 | 33.25 | 8 | O |
| ATOM | 1443 | N | TRP A | 190 | 64.171 | 35.748 | 26.225 | 1.00 | 29.51 | 7 | N |
| ATOM | 1444 | CA | TRP A | 190 | 64.323 | 35.624 | 27.681 | 1.00 | 31.44 | 6 | C |
| ATOM | 1445 | CB | TRP A | 190 | 63.025 | 35.600 | 28.489 | 1.00 | 28.21 | 6 | C |
| ATOM | 1446 | CG | TRP A | 190 | 62.310 | 36.907 | 28.440 | 1.00 | 27.33 | 6 | C |
| ATOM | 1447 | CD2 | TRP A | 190 | 60.897 | 37.076 | 28.507 | 1.00 | 27.98 | 6 | C |
| ATOM | 1448 | CE2 | TRP A | 190 | 60.647 | 38.462 | 28.418 | 1.00 | 27.50 | 6 | C |
| ATOM | 1449 | CE3 | TRP A | 190 | 59.830 | 36.184 | 28.630 | 1.00 | 27.50 | 6 | C |
| ATOM | 1450 | CD1 | TRP A | 190 | 62.867 | 38.144 | 28.327 | 1.00 | 26.66 | 6 | C |
| ATOM | 1451 | NE1 | TRP A | 190 | 61.878 | 39.064 | 28.289 | 1.00 | 27.63 | 7 | N |
| ATOM | 1452 | CZ2 | TRP A | 190 | 59.347 | 38.977 | 28.443 | 1.00 | 26.20 | 6 | C |
| ATOM | 1453 | CZ3 | TRP A | 190 | 58.557 | 36.687 | 28.654 | 1.00 | 24.89 | 6 | C |
| ATOM | 1454 | CH2 | TRP A | 190 | 58.305 | 38.088 | 28.574 | 1.00 | 27.33 | 6 | C |
| ATOM | 1455 | C | TRP A | 190 | 64.964 | 34.324 | 28.037 | 1.00 | 30.14 | 6 | C |
| ATOM | 1456 | O | TRP A | 190 | 64.989 | 34.026 | 29.228 | 1.00 | 35.07 | 8 | O |
| ATOM | 1457 | N | ASN A | 191 | 65.413 | 33.530 | 27.127 | 1.00 | 32.14 | 7 | N |
| ATOM | 1458 | CA | ASN A | 191 | 65.949 | 32.197 | 27.148 | 1.00 | 35.02 | 6 | C |
| ATOM | 1459 | CB | ASN A | 191 | 67.447 | 32.173 | 26.994 | 1.00 | 40.16 | 6 | C |
| ATOM | 1460 | CG | ASN A | 191 | 68.181 | 30.939 | 27.415 | 1.00 | 39.38 | 6 | C |
| ATOM | 1461 | OD1 | ASN A | 191 | 68.376 | 29.912 | 26.770 | 1.00 | 39.89 | 8 | O |
| ATOM | 1462 | ND2 | ASN A | 191 | 68.617 | 31.096 | 28.668 | 1.00 | 42.80 | 7 | N |
| ATOM | 1463 | C | ASN A | 191 | 65.394 | 31.348 | 28.296 | 1.00 | 36.01 | 6 | C |
| ATOM | 1464 | O | ASN A | 191 | 66.044 | 30.562 | 29.017 | 1.00 | 37.38 | 8 | O |
| ATOM | 1465 | N | CYS A | 192 | 64.077 | 31.238 | 28.285 | 1.00 | 33.47 | 7 | N |
| ATOM | 1466 | CA | CYS A | 192 | 63.273 | 30.556 | 29.261 | 1.00 | 33.13 | 6 | C |
| ATOM | 1467 | CB | CYS A | 192 | 62.363 | 31.601 | 29.929 | 1.00 | 32.53 | 6 | C |
| ATOM | 1468 | SG | CYS A | 192 | 61.228 | 32.491 | 28.808 | 1.00 | 25.76 | 16 | S |
| ATOM | 1469 | C | CYS A | 192 | 62.384 | 29.475 | 28.667 | 1.00 | 33.65 | 6 | C |
| ATOM | 1470 | O | CYS A | 192 | 61.907 | 29.549 | 27.538 | 1.00 | 34.06 | 8 | O |
| ATOM | 1471 | N | PRO A | 193 | 62.029 | 28.528 | 29.523 | 1.00 | 31.85 | 7 | N |
| ATOM | 1472 | CD | PRO A | 193 | 62.530 | 28.367 | 30.923 | 1.00 | 30.41 | 6 | C |
| ATOM | 1473 | CA | PRO A | 193 | 61.089 | 27.478 | 29.133 | 1.00 | 28.79 | 6 | C |
| ATOM | 1474 | CB | PRO A | 193 | 60.922 | 26.700 | 30.436 | 1.00 | 30.28 | 6 | C |
| ATOM | 1475 | CG | PRO A | 193 | 62.243 | 26.878 | 31.120 | 1.00 | 32.19 | 6 | C |
| ATOM | 1476 | C | PRO A | 193 | 59.772 | 28.075 | 28.758 | 1.00 | 24.94 | 6 | C |
| ATOM | 1477 | O | PRO A | 193 | 59.584 | 29.190 | 29.295 | 1.00 | 26.27 | 8 | o |
| ATOM | 1478 | N | ASN A | 194 | 58.818 | 27.503 | 28.041 | 1.00 | 24.49 | 7 | N |
| ATOM | 1479 | CA | ASN A | 194 | 57.543 | 28.193 | 27.783 | 1.00 | 22.29 | 6 | C |
| ATOM | 1480 | CB | ASN A | 194 | 56.639 | 27.220 | 26.981 | 1.00 | 23.65 | 6 | C |
| ATOM | 1481 | CG | ASN A | 194 | 57.209 | 26.860 | 25.645 | 1.00 | 25.45 | 6 | C |
| ATOM | 1482 | OD1 | ASN A | 194 | 58.241 | 27.362 | 25.152 | 1.00 | 24.24 | 8 | O |
| ATOM | 1483 | ND2 | ASN A | 194 | 56.595 | 25.963 | 24.866 | 1.00 | 25.09 | 7 | N |
| ATOM | 1484 | C | ASN A | 194 | 56.761 | 28.713 | 28.962 | 1.00 | 20.12 | 6 | C |
| ATOM | 1485 | O | ASN A | 194 | 56.085 | 29.766 | 28.913 | 1.00 | 22.50 | 8 | O |
| ATOM | 1486 | N | GLY A | 195 | 56.753 | 28.011 | 30.073 | 1.00 | 26.62 | 7 | N |
| ATOM | 1487 | CA | GLY A | 195 | 56.115 | 28.289 | 31.322 | 1.00 | 26.43 | 6 | C |
| ATOM | 1488 | C | GLY A | 195 | 56.409 | 29.630 | 31.936 | 1.00 | 27.31 | 6 | C |
| ATOM | 1489 | O | GLY A | 195 | 55.527 | 30.281 | 32.463 | 1.00 | 26.30 | 8 | O |
| ATOM | 1490 | N | GLU A | 196 | 57.616 | 30.224 | 31.676 | 1.00 | 28.17 | 7 | N |
| ATOM | 1491 | CA | GLU A | 196 | 57.902 | 31.569 | 32.142 | 1.00 | 27.07 | 6 | C |
| ATOM | 1492 | CB | GLU A | 196 | 59.360 | 31.933 | 31.762 | 1.00 | 28.03 | 6 | C |
| ATOM | 1493 | CG | GLU A | 196 | 59.778 | 33.384 | 31.953 | 1.00 | 28.94 | 6 | C |
| ATOM | 1494 | CD | GLU A | 196 | 59.723 | 33.969 | 33.355 | 1.00 | 31.47 | 6 | C |
| ATOM | 1495 | OE1 | GLU A | 196 | 59.755 | 33.248 | 34.382 | 1.00 | 31.17 | 8 | O |
| ATOM | 1496 | OE2 | GLU A | 196 | 59.549 | 35.189 | 33.512 | 1.00 | 29.20 | 8 | O |
| ATOM | 1497 | C | GLU A | 196 | 56.956 | 32.613 | 31.682 | 1.00 | 24.33 | 6 | C |
| ATOM | 1498 | O | GLU A | 196 | 56.440 | 33.536 | 32.295 | 1.00 | 23.08 | 8 | O |
| ATOM | 1499 | N | LEU A | 197 | 56.452 | 32.441 | 30.398 | 1.00 | 26.15 | 7 | N |
| ATOM | 1500 | CA | LEU A | 197 | 55.581 | 33.376 | 29.747 | 1.00 | 27.39 | 6 | C |
| ATOM | 1501 | CB | LEU A | 197 | 55.341 | 33.039 | 28.289 | 1.00 | 26.19 | 6 | C |
| ATOM | 1502 | CG | LEU A | 197 | 56.482 | 33.342 | 27.303 | 1.00 | 25.11 | 6 | C |
| ATOM | 1503 | CD1 | LEU A | 197 | 57.837 | 32.764 | 27.722 | 1.00 | 21.88 | 6 | C |
| ATOM | 1504 | CD2 | LEU A | 197 | 56.103 | 32.760 | 25.920 | 1.00 | 23.70 | 6 | C |
| ATOM | 1505 | C | LEU A | 197 | 54.305 | 33.604 | 30.558 | 1.00 | 30.74 | 6 | C |
| ATOM | 1506 | O | LEU A | 197 | 54.011 | 34.819 | 30.477 | 1.00 | 33.28 | 8 | O |
| ATOM | 1507 | N | ARG A | 198 | 53.866 | 32.621 | 31.328 | 1.00 | 33.73 | 7 | N |
| ATOM | 1508 | CA | ARG A | 198 | 52.703 | 32.702 | 32.172 | 1.00 | 38.60 | 6 | C |
| ATOM | 1509 | CB | ARG A | 198 | 52.508 | 31.396 | 32.969 | 1.00 | 41.00 | 6 | C |
| ATOM | 1510 | CG | ARG A | 198 | 53.453 | 31.254 | 34.140 | 1.00 | 46.30 | 6 | C |
| ATOM | 1511 | CD | ARG A | 198 | 53.449 | 29.998 | 34.986 | 1.00 | 48.19 | 6 | C |
| ATOM | 1512 | NE | ARG A | 198 | 53.367 | 28.801 | 34.144 | 1.00 | 49.91 | 7 | N |
| ATOM | 1513 | CZ | ARG A | 198 | 52.239 | 28.126 | 33.953 | 1.00 | 52.06 | 6 | C |
| ATOM | 1514 | NH1 | ARG A | 198 | 51.165 | 28.574 | 34.606 | 1.00 | 52.69 | 7 | N |
| ATOM | 1515 | NH2 | ARG A | 198 | 52.154 | 27.056 | 33.193 | 1.00 | 51.48 | 7 | N |
| ATOM | 1516 | C | ARG A | 198 | 52.817 | 33.918 | 33.076 | 1.00 | 40.25 | 6 | C |
| ATOM | 1517 | O | ARG A | 198 | 51.839 | 34.645 | 33.277 | 1.00 | 38.34 | 8 | O |
| ATOM | 1518 | N | HIS A | 199 | 54.021 | 34.249 | 33.533 | 1.00 | 41.34 | 7 | N |
| ATOM | 1519 | CA | HIS A | 199 | 54.308 | 35.402 | 34.359 | 1.00 | 43.94 | 6 | C |
| ATOM | 1520 | CB | HIS A | 199 | 55.692 | 35.267 | 35.029 | 1.00 | 43.20 | 6 | C |
| ATOM | 1521 | CG | HIS A | 199 | 55.815 | 34.020 | 35.831 | 1.00 | 43.20 | 6 | C |
| ATOM | 1522 | CD2 | HIS A | 199 | 56.564 | 32.901 | 35.729 | 1.00 | 42.22 | 6 | C |
| ATOM | 1523 | ND1 | HIS A | 199 | 55.057 | 33.870 | 36.986 | 1.00 | 43.41 | 7 | N |
| ATOM | 1524 | CE1 | HIS A | 199 | 55.342 | 32.717 | 37.532 | 1.00 | 41.35 | 6 | C |
| ATOM | 1525 | NE2 | HIS A | 199 | 56.240 | 32.097 | 36.805 | 1.00 | 41.60 | 7 | N |
| ATOM | 1526 | C | HIS A | 199 | 54.292 | 36.727 | 33.611 | 1.00 | 46.20 | 6 | C |
| ATOM | 1527 | O | HIS A | 199 | 54.665 | 37.738 | 34.229 | 1.00 | 45.70 | 8 | O |
| ATOM | 1528 | N | HIS A | 200 | 53.885 | 36.816 | 32.353 | 1.00 | 45.71 | 7 | N |
| ATOM | 1529 | CA | HIS A | 200 | 53.824 | 38.027 | 31.563 | 1.00 | 46.94 | 6 | C |
| ATOM | 1530 | CB | HIS A | 200 | 55.011 | 38.096 | 30.590 | 1.00 | 43.85 | 6 | C |
| ATOM | 1531 | CG | HIS A | 200 | 56.349 | 38.102 | 31.246 | 1.00 | 41.89 | 6 | C |
| ATOM | 1532 | CD2 | HIS A | 200 | 57.101 | 39.133 | 31.714 | 1.00 | 39.90 | 6 | C |
| ATOM | 1533 | ND1 | HIS A | 200 | 57.051 | 36.937 | 31.486 | 1.00 | 39.39 | 7 | N |
| ATOM | 1534 | CE1 | HIS A | 200 | 58.192 | 37.261 | 32.053 | 1.00 | 38.66 | 6 | C |
| ATOM | 1535 | NE2 | HIS A | 200 | 58.250 | 38.565 | 32.187 | 1.00 | 38.30 | 7 | N |
| ATOM | 1536 | C | HIS A | 200 | 52.557 | 38.177 | 30.723 | 1.00 | 49.59 | 6 | C |
| ATOM | 1537 | O | HIS A | 200 | 52.552 | 38.374 | 29.495 | 1.00 | 49.25 | 8 | O |
| ATOM | 1538 | N | PRO A | 201 | 51.409 | 38.252 | 31.382 | 1.00 | 51.21 | 7 | N |
| ATOM | 1539 | CD | PRO A | 201 | 51.220 | 38.097 | 32.843 | 1.00 | 51.88 | 6 | C |
| ATOM | 1540 | CA | PRO A | 201 | 50.131 | 38.391 | 30.715 | 1.00 | 51.97 | 6 | C |
| ATOM | 1541 | CB | PRO A | 201 | 49.093 | 38.248 | 31.833 | 1.00 | 52.67 | 6 | C |
| ATOM | 1542 | CG | PRO A | 201 | 49.850 | 38.692 | 33.042 | 1.00 | 52.12 | 6 | C |
| ATOM | 1543 | C | PRO A | 201 | 49.932 | 39.702 | 29.989 | 1.00 | 51.85 | 6 | C |
| ATOM | 1544 | O | PRO A | 201 | 49.500 | 39.678 | 28.836 | 1.00 | 52.23 | 8 | O |
| ATOM | 1545 | N | GLN A | 202 | 50.223 | 40.839 | 30.618 | 1.00 | 52.04 | 7 | N |
| ATOM | 1546 | CA | GLN A | 202 | 49.956 | 42.117 | 29.973 | 1.00 | 51.98 | 6 | C |
| ATOM | 1547 | C | GLN A | 202 | 50.650 | 42.302 | 28.629 | 1.00 | 49.40 | 6 | C |
| ATOM | 1548 | O | GLN A | 202 | 50.015 | 42.725 | 27.657 | 1.00 | 48.97 | 8 | O |
| ATOM | 1549 | CB | GLN A | 202 | 50.327 | 43.269 | 30.909 | 1.00 | 54.78 | 6 | C |
| ATOM | 1550 | CG | GLN A | 202 | 49.300 | 43.490 | 32.015 | 1.00 | 57.14 | 6 | C |
| ATOM | 1551 | CD | GLN A | 202 | 49.431 | 44.907 | 32.561 | 1.00 | 58.39 | 6 | C |
| ATOM | 1552 | OE1 | GLN A | 202 | 50.506 | 45.509 | 32.561 | 1.00 | 59.46 | 8 | O |
| ATOM | 1553 | NE2 | GLN A | 202 | 48.310 | 45.452 | 33.008 | 1.00 | 58.40 | 7 | N |
| ATOM | 1554 | N | GLU A | 203 | 51.930 | 41.983 | 28.568 | 1.00 | 46.29 | 7 | N |
| ATOM | 1555 | CA | GLU A | 203 | 52.768 | 42.017 | 27.394 | 1.00 | 45.75 | 6 | C |
| ATOM | 1556 | CB | GLU A | 203 | 54.174 | 41.552 | 27.780 | 1.00 | 48.61 | 6 | C |
| ATOM | 1557 | CG | GLU A | 203 | 55.309 | 42.104 | 26.940 | 1.00 | 55.35 | 6 | C |
| ATOM | 1558 | CD | GLU A | 203 | 56.559 | 41.243 | 27.111 | 1.00 | 58.68 | 6 | C |
| ATOM | 1559 | OE1 | GLU A | 203 | 56.364 | 40.011 | 26.928 | 1.00 | 60.64 | 8 | O |
| ATOM | 1560 | OE2 | GLU A | 203 | 57.654 | 41.760 | 27.413 | 1.00 | 60.48 | 8 | O |
| ATOM | 1561 | C | GLU A | 203 | 52.298 | 41.077 | 26.267 | 1.00 | 42.01 | 6 | C |
| ATOM | 1562 | O | GLU A | 203 | 52.351 | 41.439 | 25.102 | 1.00 | 36.83 | 8 | O |
| ATOM | 1563 | N | ILE A | 204 | 51.887 | 39.875 | 26.653 | 1.00 | 37.26 | 7 | N |
| ATOM | 1564 | CA | ILE A | 204 | 51.375 | 38.902 | 25.672 | 1.00 | 34.86 | 6 | C |
| ATOM | 1565 | CB | ILE A | 204 | 51.125 | 37.516 | 26.295 | 1.00 | 33.38 | 6 | C |
| ATOM | 1566 | CG2 | ILE A | 204 | 50.235 | 36.647 | 25.414 | 1.00 | 32.50 | 6 | C |
| ATOM | 1567 | CG1 | ILE A | 204 | 52.491 | 36.837 | 26.506 | 1.00 | 32.44 | 6 | C |
| ATOM | 1568 | CD1 | ILE A | 204 | 52.526 | 35.510 | 27.199 | 1.00 | 33.69 | 6 | C |
| ATOM | 1569 | C | ILE A | 204 | 50.150 | 39.478 | 24.998 | 1.00 | 35.79 | 6 | C |
| ATOM | 1570 | O | ILE A | 204 | 49.942 | 39.318 | 23.783 | 1.00 | 33.96 | 8 | O |
| ATOM | 1571 | N | MET A | 205 | 49.292 | 40.164 | 25.762 | 1.00 | 36.04 | 7 | N |
| ATOM | 1572 | CA | MET A | 205 | 48.109 | 40.787 | 25.173 | 1.00 | 39.85 | 6 | C |
| ATOM | 1573 | CB | MET A | 205 | 47.299 | 41.516 | 26.258 | 1.00 | 42.17 | 6 | C |
| ATOM | 1574 | CG | MET A | 205 | 45.870 | 41.037 | 26.363 | 1.00 | 45.40 | 6 | C |
| ATOM | 1575 | SD | MET A | 205 | 44.877 | 41.345 | 24.930 | 1.00 | 49.73 | 16 | S |
| ATOM | 1576 | CE | MET A | 205 | 45.097 | 39.901 | 23.971 | 1.00 | 49.33 | 6 | C |
| ATOM | 1577 | C | MET A | 205 | 48.503 | 41.781 | 24.084 | 1.00 | 38.13 | 6 | C |
| ATOM | 1578 | O | MET A | 205 | 47.869 | 41.802 | 23.039 | 1.00 | 39.53 | 8 | O |
| ATOM | 1579 | N | GLN A | 206 | 49.485 | 42.626 | 24.339 | 1.00 | 39.17 | 7 | N |
| ATOM | 1580 | CA | GLN A | 206 | 49.938 | 43.628 | 23.385 | 1.00 | 38.73 | 6 | C |
| ATOM | 1581 | CB | GLN A | 206 | 51.053 | 44.495 | 23.943 | 1.00 | 43.09 | 6 | C |
| ATOM | 1582 | CG | GLN A | 206 | 50.545 | 45.751 | 24.654 | 1.00 | 47.58 | 6 | C |
| ATOM | 1583 | CD | GLN A | 206 | 51.648 | 46.364 | 25.504 | 1.00 | 50.94 | 6 | C |
| ATOM | 1584 | OE1 | GLN A | 206 | 52.392 | 47.242 | 25.045 | 1.00 | 52.20 | 8 | O |
| ATOM | 1585 | NE2 | GLN A | 206 | 51.705 | 45.838 | 26.727 | 1.00 | 51.79 | 7 | N |
| ATOM | 1586 | C | GLN A | 206 | 50.381 | 42.967 | 22.071 | 1.00 | 37.79 | 6 | C |
| ATOM | 1587 | O | GLN A | 206 | 50.020 | 43.460 | 20.994 | 1.00 | 34.91 | 8 | O |
| ATOM | 1588 | N | ILE A | 207 | 51.161 | 41.881 | 22.205 | 1.00 | 34.29 | 7 | N |
| ATOM | 1589 | CA | ILE A | 207 | 51.554 | 41.174 | 20.954 | 1.00 | 33.32 | 6 | C |
| ATOM | 1590 | CB | ILE A | 207 | 52.526 | 40.022 | 21.276 | 1.00 | 31.81 | 6 | C |
| ATOM | 1591 | CG2 | ILE A | 207 | 52.959 | 39.225 | 20.063 | 1.00 | 30.02 | 6 | C |
| ATOM | 1592 | CG1 | ILE A | 207 | 53.737 | 40.617 | 22.003 | 1.00 | 30.60 | 6 | C |
| ATOM | 1593 | CD1 | ILE A | 207 | 54.741 | 39.688 | 22.591 | 1.00 | 30.71 | 6 | C |
| ATOM | 1594 | C | ILE A | 207 | 50.325 | 40.619 | 20.281 | 1.00 | 32.72 | 6 | C |
| ATOM | 1595 | O | ILE A | 207 | 50.164 | 40.781 | 19.061 | 1.00 | 30.79 | 8 | O |
| ATOM | 1596 | N | CYS A | 208 | 49.415 | 39.982 | 21.055 | 1.00 | 30.93 | 7 | N |
| ATOM | 1597 | CA | CYS A | 208 | 48.215 | 39.426 | 20.459 | 1.00 | 32.17 | 6 | C |
| ATOM | 1598 | CB | CYS A | 208 | 47.373 | 38.602 | 21.471 | 1.00 | 30.26 | 6 | C |
| ATOM | 1599 | SG | CYS A | 208 | 48.207 | 37.141 | 22.108 | 1.00 | 28.13 | 16 | S |
| ATOM | 1600 | C | CYS A | 208 | 47.320 | 40.478 | 19.819 | 1.00 | 32.96 | 6 | C |
| ATOM | 1601 | O | CYS A | 208 | 46.643 | 40.223 | 18.835 | 1.00 | 32.33 | 8 | O |
| ATOM | 1602 | N | GLU A | 209 | 47.354 | 41.704 | 20.343 | 1.00 | 37.50 | 7 | N |
| ATOM | 1603 | CA | GLU A | 209 | 46.513 | 42.755 | 19.746 | 1.00 | 39.18 | 6 | C |
| ATOM | 1604 | CB | GLU A | 209 | 46.468 | 43.983 | 20.661 | 1.00 | 44.01 | 6 | C |
| ATOM | 1605 | CG | GLU A | 209 | 45.045 | 44.528 | 20.829 | 1.00 | 48.18 | 6 | C |
| ATOM | 1606 | CD | GLU A | 209 | 44.773 | 44.945 | 22.260 | 1.00 | 50.80 | 6 | C |
| ATOM | 1607 | OE1 | GLU A | 209 | 45.611 | 45.671 | 22.845 | 1.00 | 53.20 | 8 | O |
| ATOM | 1608 | OE2 | GLU A | 209 | 43.739 | 44.572 | 22.853 | 1.00 | 52.38 | 8 | O |
| ATOM | 1609 | C | GLU A | 209 | 47.042 | 43.114 | 18.364 | 1.00 | 37.08 | 6 | C |
| ATOM | 1610 | O | GLU A | 209 | 46.283 | 43.325 | 17.432 | 1.00 | 36.61 | 8 | O |
| ATOM | 1611 | N | GLU A | 210 | 48.364 | 43.119 | 18.206 | 1.00 | 35.73 | 7 | N |
| ATOM | 1612 | CA | GLU A | 210 | 49.001 | 43.407 | 16.932 | 1.00 | 33.99 | 6 | C |
| ATOM | 1613 | CB | GLU A | 210 | 50.500 | 43.623 | 17.066 | 1.00 | 36.31 | 6 | C |
| ATOM | 1614 | CG | GLU A | 210 | 50.947 | 44.920 | 17.709 | 1.00 | 37.10 | 6 | C |
| ATOM | 1615 | CD | GLU A | 210 | 52.408 | 45.056 | 17.955 | 1.00 | 39.26 | 6 | C |
| ATOM | 1616 | OE1 | GLU A | 210 | 53.241 | 44.246 | 17.452 | 1.00 | 38.74 | 8 | O |
| ATOM | 1617 | OE2 | GLU A | 210 | 52.864 | 46.010 | 18.660 | 1.00 | 39.33 | 8 | O |
| ATOM | 1618 | C | GLU A | 210 | 48.755 | 42.266 | 15.938 | 1.00 | 33.60 | 6 | C |
| ATOM | 1619 | O | GLU A | 210 | 48.447 | 42.526 | 14.771 | 1.00 | 31.24 | 8 | O |
| ATOM | 1620 | N | VAL A | 211 | 48.916 | 41.033 | 16.452 | 1.00 | 32.10 | 7 | N |
| ATOM | 1621 | CA | VAL A | 211 | 48.694 | 39.871 | 15.581 | 1.00 | 31.23 | 6 | C |
| ATOM | 1622 | CB | VAL A | 211 | 49.012 | 38.549 | 16.330 | 1.00 | 28.69 | 6 | C |
| ATOM | 1623 | CG1 | VAL A | 211 | 48.674 | 37.337 | 15.462 | 1.00 | 29.45 | 6 | C |
| ATOM | 1624 | CG2 | VAL A | 211 | 50.462 | 38.551 | 16.759 | 1.00 | 29.65 | 6 | C |
| ATOM | 1625 | C | VAL A | 211 | 47.284 | 39.828 | 15.074 | 1.00 | 31.39 | 6 | C |
| ATOM | 1626 | O | VAL A | 211 | 46.954 | 39.624 | 13.900 | 1.00 | 32.71 | 8 | O |
| ATOM | 1627 | N | ALA A | 212 | 46.335 | 40.103 | 15.991 | 1.00 | 30.93 | 7 | N |
| ATOM | 1628 | CA | ALA A | 212 | 44.924 | 40.091 | 15.689 | 1.00 | 32.89 | 6 | C |
| ATOM | 1629 | CB | ALA A | 212 | 44.116 | 40.300 | 16.976 | 1.00 | 32.30 | 6 | C |
| ATOM | 1630 | C | ALA A | 212 | 44.571 | 41.155 | 14.659 | 1.00 | 33.48 | 6 | C |
| ATOM | 1631 | O | ALA A | 212 | 43.774 | 40.939 | 13.749 | 1.00 | 36.23 | 8 | O |
| ATOM | 1632 | N | ALA A | 213 | 45.219 | 42.315 | 14.742 | 1.00 | 35.20 | 7 | N |
| ATOM | 1633 | CA | ALA A | 213 | 44.954 | 43.392 | 13.785 | 1.00 | 36.38 | 6 | C |
| ATOM | 1634 | CB | ALA A | 213 | 45.618 | 44.688 | 14.226 | 1.00 | 37.67 | 6 | C |
| ATOM | 1635 | C | ALA A | 213 | 45.391 | 43.011 | 12.375 | 1.00 | 37.84 | 6 | C |
| ATOM | 1636 | O | ALA A | 213 | 44.650 | 43.316 | 11.430 | 1.00 | 37.96 | 8 | O |
| ATOM | 1637 | N | VAL A | 214 | 46.542 | 42.358 | 12.206 | 1.00 | 36.16 | 7 | N |
| ATOM | 1638 | CA | VAL A | 214 | 46.937 | 41.939 | 10.865 | 1.00 | 34.52 | 6 | C |
| ATOM | 1639 | CB | VAL A | 214 | 48.417 | 41.471 | 10.889 | 1.00 | 34.87 | 6 | C |
| ATOM | 1640 | CG1 | VAL A | 214 | 48.877 | 40.966 | 9.532 | 1.00 | 33.28 | 6 | C |
| ATOM | 1641 | CG2 | VAL A | 214 | 49.261 | 42.632 | 11.389 | 1.00 | 32.43 | 6 | C |
| ATOM | 1642 | C | VAL A | 214 | 46.084 | 40.831 | 10.294 | 1.00 | 35.47 | 6 | C |
| ATOM | 1643 | O | VAL A | 214 | 45.731 | 40.804 | 9.104 | 1.00 | 35.36 | 8 | O |
| ATOM | 1644 | N | ILE A | 215 | 45.687 | 39.842 | 11.095 | 1.00 | 35.92 | 7 | N |
| ATOM | 1645 | CA | ILE A | 215 | 44.914 | 38.693 | 10.645 | 1.00 | 38.92 | 6 | C |
| ATOM | 1646 | CB | ILE A | 215 | 44.795 | 37.632 | 11.767 | 1.00 | 40.37 | 6 | C |
| ATOM | 1647 | CG2 | ILE A | 215 | 43.836 | 36.513 | 11.407 | 1.00 | 39.92 | 6 | C |
| ATOM | 1648 | CG1 | ILE A | 215 | 46.226 | 37.114 | 12.086 | 1.00 | 40.26 | 6 | C |
| ATOM | 1649 | CD1 | ILE A | 215 | 46.241 | 36.213 | 13.307 | 1.00 | 40.89 | 6 | C |
| ATOM | 1650 | C | ILE A | 215 | 43.513 | 39.061 | 10.191 | 1.00 | 41.16 | 6 | C |
| ATOM | 1651 | O | ILE A | 215 | 42.956 | 38.539 | 9.222 | 1.00 | 37.05 | 8 | O |
| ATOM | 1652 | N | GLU A | 216 | 42.912 | 39.991 | 10.932 | 1.00 | 45.02 | 7 | N |
| ATOM | 1653 | CA | GLU A | 216 | 41.560 | 40.454 | 10.614 | 1.00 | 50.87 | 6 | C |
| ATOM | 1654 | CB | GLU A | 216 | 41.066 | 41.428 | 11.678 | 1.00 | 54.57 | 6 | C |
| ATOM | 1655 | CG | GLU A | 216 | 39.570 | 41.689 | 11.610 | 1.00 | 58.44 | 6 | C |
| ATOM | 1656 | CD | GLU A | 216 | 38.733 | 40.449 | 11.858 | 1.00 | 61.16 | 6 | C |
| ATOM | 1657 | OE1 | GLU A | 216 | 38.851 | 39.862 | 12.957 | 1.00 | 62.37 | 8 | O |
| ATOM | 1658 | OE2 | GLU A | 216 | 37.953 | 40.055 | 10.964 | 1.00 | 62.50 | 8 | O |
| ATOM | 1659 | C | GLU A | 216 | 41.542 | 41.079 | 9.226 | 1.00 | 52.67 | 6 | C |
| ATOM | 1660 | O | GLU A | 216 | 40.689 | 40.791 | 8.382 | 1.00 | 51.01 | 8 | O |
| ATOM | 1661 | N | ARG A | 217 | 42.557 | 41.881 | 8.923 | 1.00 | 54.94 | 7 | N |
| ATOM | 1662 | CA | ARG A | 217 | 42.710 | 42.519 | 7.625 | 1.00 | 58.93 | 6 | C |
| ATOM | 1663 | CB | ARG A | 217 | 43.774 | 43.620 | 7.689 | 1.00 | 62.40 | 6 | C |
| ATOM | 1664 | CG | ARG A | 217 | 43.705 | 44.614 | 6.551 | 1.00 | 67.19 | 6 | C |
| ATOM | 1665 | CD | ARG A | 217 | 42.346 | 45.264 | 6.377 | 1.00 | 70.69 | 6 | C |
| ATOM | 1666 | NE | ARG A | 217 | 42.021 | 46.176 | 7.465 | 1.00 | 73.78 | 7 | N |
| ATOM | 1667 | CZ | ARG A | 217 | 42.651 | 47.308 | 7.746 | 1.00 | 75.90 | 6 | C |
| ATOM | 1668 | NH1 | ARG A | 217 | 43.690 | 47.736 | 7.031 | 1.00 | 76.39 | 7 | N |
| ATOM | 1669 | NH2 | ARG A | 217 | 42.234 | 48.047 | 8.774 | 1.00 | 77.03 | 7 | N |
| ATOM | 1670 | C | ARG A | 217 | 43.045 | 41.542 | 6.509 | 1.00 | 59.09 | 6 | C |
| ATOM | 1671 | O | ARG A | 217 | 42.801 | 41.773 | 5.325 | 1.00 | 56.80 | 8 | O |
| ATOM | 1672 | N | GLU A | 218 | 43.568 | 40.370 | 6.868 | 1.00 | 59.09 | 7 | N |
| ATOM | 1673 | CA | GLU A | 218 | 43.902 | 39.308 | 5.940 | 1.00 | 59.58 | 6 | C |
| ATOM | 1674 | CB | GLU A | 218 | 44.945 | 38.373 | 6.556 | 1.00 | 58.99 | 6 | C |
| ATOM | 1675 | CG | GLU A | 218 | 46.338 | 38.949 | 6.702 | 1.00 | 58.23 | 6 | C |
| ATOM | 1676 | CD | GLU A | 218 | 47.133 | 38.977 | 5.425 | 1.00 | 58.08 | 6 | C |
| ATOM | 1677 | OE1 | GLU A | 218 | 47.693 | 37.952 | 4.994 | 1.00 | 58.06 | 8 | O |
| ATOM | 1678 | OE2 | GLU A | 218 | 47.249 | 40.042 | 4.786 | 1.00 | 57.22 | 8 | O |
| ATOM | 1679 | C | GLU A | 218 | 42.676 | 38.498 | 5.527 | 1.00 | 60.35 | 6 | C |
| ATOM | 1680 | O | GLU A | 218 | 42.747 | 37.690 | 4.603 | 1.00 | 59.78 | 8 | O |
| ATOM | 1681 | N | GLY A | 219 | 41.548 | 38.679 | 6.200 | 1.00 | 60.81 | 7 | N |
| ATOM | 1682 | CA | GLY A | 219 | 40.319 | 38.004 | 5.870 | 1.00 | 62.25 | 6 | C |
| ATOM | 1683 | C | GLY A | 219 | 39.855 | 36.916 | 6.810 | 1.00 | 62.90 | 6 | C |
| ATOM | 1684 | O | GLY A | 219 | 38.933 | 36.173 | 6.465 | 1.00 | 63.53 | 8 | O |
| ATOM | 1685 | N | HIS A | 220 | 40.473 | 36.762 | 7.975 | 1.00 | 63.00 | 7 | N |
| ATOM | 1686 | CA | HIS A | 220 | 40.100 | 35.726 | 8.925 | 1.00 | 63.21 | 6 | C |
| ATOM | 1687 | CB | HIS A | 220 | 41.300 | 34.823 | 9.259 | 1.00 | 62.08 | 6 | C |
| ATOM | 1688 | CG | HIS A | 220 | 41.892 | 34.220 | 8.019 | 1.00 | 60.36 | 6 | C |
| ATOM | 1689 | CD2 | HIS A | 220 | 41.472 | 33.226 | 7.208 | 1.00 | 59.68 | 6 | C |
| ATOM | 1690 | ND1 | HIS A | 220 | 43.047 | 34.710 | 7.463 | 1.00 | 59.52 | 7 | N |
| ATOM | 1691 | CE1 | HIS A | 220 | 43.339 | 34.032 | 6.370 | 1.00 | 59.32 | 6 | C |
| ATOM | 1692 | NE2 | HIS A | 220 | 42.393 | 33.127 | 6.194 | 1.00 | 59.69 | 7 | N |
| ATOM | 1693 | C | HIS A | 220 | 39.514 | 36.288 | 10.215 | 1.00 | 63.87 | 6 | C |
| ATOM | 1694 | O | HIS A | 220 | 39.897 | 37.340 | 10.709 | 1.00 | 63.38 | 8 | O |
| ATOM | 1695 | N | ALA A | 221 | 38.564 | 35.535 | 10.761 | 1.00 | 65.10 | 7 | N |
| ATOM | 1696 | CA | ALA A | 221 | 37.863 | 35.905 | 11.973 | 1.00 | 65.68 | 6 | C |
| ATOM | 1697 | CB | ALA A | 221 | 36.442 | 35.347 | 11.963 | 1.00 | 65.64 | 6 | C |
| ATOM | 1698 | C | ALA A | 221 | 38.579 | 35.419 | 13.229 | 1.00 | 65.76 | 6 | C |
| ATOM | 1699 | O | ALA A | 221 | 38.544 | 34.231 | 13.546 | 1.00 | 66.16 | 8 | O |
| ATOM | 1700 | N | THR A | 222 | 39.159 | 36.370 | 13.948 | 1.00 | 65.60 | 7 | N |
| ATOM | 1701 | CA | THR A | 222 | 39.810 | 36.056 | 15.215 | 1.00 | 65.70 | 6 | C |
| ATOM | 1702 | CB | THR A | 222 | 41.271 | 35.616 | 15.087 | 1.00 | 66.10 | 6 | C |
| ATOM | 1703 | OG1 | THR A | 222 | 41.582 | 34.879 | 16.283 | 1.00 | 66.76 | 8 | O |
| ATOM | 1704 | CG2 | THR A | 222 | 42.226 | 36.786 | 14.959 | 1.00 | 66.34 | 6 | C |
| ATOM | 1705 | C | THR A | 222 | 39.707 | 37.277 | 16.127 | 1.00 | 64.58 | 6 | C |
| ATOM | 1706 | O | THR A | 222 | 39.414 | 38.381 | 15.662 | 1.00 | 65.05 | 8 | O |
| ATOM | 1707 | N | SER A | 223 | 39.953 | 37.082 | 17.405 | 1.00 | 62.62 | 7 | N |
| ATOM | 1708 | CA | SER A | 223 | 39.903 | 38.169 | 18.371 | 1.00 | 61.43 | 6 | C |
| ATOM | 1709 | CB | SER A | 223 | 38.750 | 37.955 | 19.352 | 1.00 | 61.09 | 6 | C |
| ATOM | 1710 | OG | SER A | 223 | 39.155 | 38.211 | 20.686 | 1.00 | 61.49 | 8 | O |
| ATOM | 1711 | C | SER A | 223 | 41.218 | 38.209 | 19.140 | 1.00 | 59.63 | 6 | C |
| ATOM | 1712 | O | SER A | 223 | 41.780 | 37.151 | 19.418 | 1.00 | 58.18 | 8 | O |
| ATOM | 1713 | N | ALA A | 224 | 41.634 | 39.397 | 19.566 | 1.00 | 58.25 | 7 | N |
| ATOM | 1714 | CA | ALA A | 224 | 42.870 | 39.497 | 20.337 | 1.00 | 57.60 | 6 | C |
| ATOM | 1715 | CB | ALA A | 224 | 43.258 | 40.929 | 20.651 | 1.00 | 57.58 | 6 | C |
| ATOM | 1716 | C | ALA A | 224 | 42.751 | 38.698 | 21.631 | 1.00 | 57.36 | 6 | C |
| ATOM | 1717 | O | ALA A | 224 | 43.704 | 38.040 | 22.054 | 1.00 | 55.22 | 8 | O |
| ATOM | 1718 | N | GLU A | 225 | 41.588 | 38.814 | 22.279 | 1.00 | 57.66 | 7 | N |
| ATOM | 1719 | CA | GLU A | 225 | 41.351 | 38.117 | 23.535 | 1.00 | 57.65 | 6 | C |
| ATOM | 1720 | CB | GLU A | 225 | 40.036 | 38.601 | 24.170 | 1.00 | 61.33 | 6 | C |
| ATOM | 1721 | CG | GLU A | 225 | 40.181 | 40.010 | 24.737 | 1.00 | 65.19 | 6 | C |
| ATOM | 1722 | CD | GLU A | 225 | 38.971 | 40.424 | 25.558 | 1.00 | 66.72 | 6 | C |
| ATOM | 1723 | OE1 | GLU A | 225 | 38.685 | 39.763 | 26.572 | 1.00 | 67.70 | 8 | O |
| ATOM | 1724 | OE2 | GLU A | 225 | 38.379 | 41.456 | 25.193 | 1.00 | 68.14 | 8 | O |
| ATOM | 1725 | C | GLU A | 225 | 41.334 | 36.610 | 23.361 | 1.00 | 54.22 | 6 | C |
| ATOM | 1726 | O | GLU A | 225 | 41.935 | 35.882 | 24.153 | 1.00 | 53.30 | 8 | O |
| ATOM | 1727 | N | ASP A | 226 | 40.657 | 36.144 | 22.315 | 1.00 | 50.88 | 7 | N |
| ATOM | 1728 | CA | ASP A | 226 | 40.547 | 34.718 | 22.030 | 1.00 | 46.63 | 6 | C |
| ATOM | 1729 | CB | ASP A | 226 | 39.654 | 34.428 | 20.835 | 1.00 | 48.51 | 6 | C |
| ATOM | 1730 | CG | ASP A | 226 | 38.182 | 34.727 | 21.070 | 1.00 | 50.00 | 6 | C |
| ATOM | 1731 | OD1 | ASP A | 226 | 37.416 | 34.660 | 20.086 | 1.00 | 50.52 | 8 | O |
| ATOM | 1732 | OD2 | ASP A | 226 | 37.820 | 35.044 | 22.210 | 1.00 | 50.96 | 8 | O |
| ATOM | 1733 | C | ASP A | 226 | 41.940 | 34.128 | 21.786 | 1.00 | 42.46 | 6 | C |
| ATOM | 1734 | O | ASP A | 226 | 42.332 | 33.087 | 22.324 | 1.00 | 39.02 | 8 | O |
| ATOM | 1735 | N | LEU A | 227 | 42.688 | 34.893 | 20.989 | 1.00 | 36.79 | 7 | N |
| ATOM | 1736 | CA | LEU A | 227 | 44.063 | 34.517 | 20.676 | 1.00 | 32.81 | 6 | C |
| ATOM | 1737 | CB | LEU A | 227 | 44.718 | 35.456 | 19.657 | 1.00 | 30.59 | 6 | C |
| ATOM | 1738 | CG | LEU A | 227 | 46.223 | 35.297 | 19.500 | 1.00 | 32.42 | 6 | C |
| ATOM | 1739 | CD1 | LEU A | 227 | 46.555 | 33.852 | 19.107 | 1.00 | 31.07 | 6 | C |
| ATOM | 1740 | CD2 | LEU A | 227 | 46.810 | 36.242 | 18.453 | 1.00 | 33.85 | 6 | C |
| ATOM | 1741 | C | LEU A | 227 | 44.910 | 34.486 | 21.937 | 1.00 | 29.60 | 6 | C |
| ATOM | 1742 | O | LEU A | 227 | 45.705 | 33.590 | 22.175 | 1.00 | 26.35 | 8 | O |
| ATOM | 1743 | N | ARG A | 228 | 44.694 | 35.466 | 22.846 | 1.00 | 30.78 | 7 | N |
| ATOM | 1744 | CA | ARG A | 228 | 45.458 | 35.523 | 24.084 | 1.00 | 31.06 | 6 | C |
| ATOM | 1745 | CB | ARG A | 228 | 45.124 | 36.799 | 24.862 | 1.00 | 33.22 | 6 | C |
| ATOM | 1746 | CG | ARG A | 228 | 45.941 | 37.033 | 26.124 | 1.00 | 34.67 | 6 | C |
| ATOM | 1747 | CD | ARG A | 228 | 45.273 | 36.387 | 27.333 | 1.00 | 38.78 | 6 | C |
| ATOM | 1748 | NE | ARG A | 228 | 43.824 | 36.507 | 27.313 | 1.00 | 40.70 | 7 | N |
| ATOM | 1749 | CZ | ARG A | 228 | 43.096 | 37.614 | 27.406 | 1.00 | 42.92 | 6 | C |
| ATOM | 1750 | NH1 | ARG A | 228 | 43.596 | 38.832 | 27.521 | 1.00 | 44.40 | 7 | N |
| ATOM | 1751 | NH2 | ARG A | 228 | 41.769 | 37.634 | 27.364 | 1.00 | 42.41 | 7 | N |
| ATOM | 1752 | C | ARG A | 228 | 45.157 | 34.315 | 24.971 | 1.00 | 30.66 | 6 | C |
| ATOM | 1753 | O | ARG A | 228 | 46.056 | 33.661 | 25.513 | 1.00 | 28.39 | 8 | O |
| ATOM | 1754 | N | ASP A | 229 | 43.881 | 34.006 | 25.096 | 1.00 | 31.66 | 7 | N |
| ATOM | 1755 | CA | ASP A | 229 | 43.449 | 32.873 | 25.930 | 1.00 | 31.43 | 6 | C |
| ATOM | 1756 | CB | ASP A | 229 | 41.932 | 32.799 | 25.970 | 1.00 | 35.87 | 6 | C |
| ATOM | 1757 | CG | ASP A | 229 | 41.328 | 33.930 | 26.796 | 1.00 | 38.05 | 6 | C |
| ATOM | 1758 | OD1 | ASP A | 229 | 42.051 | 34.551 | 27.586 | 1.00 | 38.95 | 8 | O |
| ATOM | 1759 | OD2 | ASP A | 229 | 40.118 | 34.107 | 26.571 | 1.00 | 40.48 | 8 | O |
| ATOM | 1760 | C | ASP A | 229 | 44.024 | 31.539 | 25.469 | 1.00 | 29.42 | 6 | C |
| ATOM | 1761 | O | ASP A | 229 | 44.449 | 30.702 | 26.273 | 1.00 | 26.73 | 8 | O |
| ATOM | 1762 | N | TYR A | 230 | 44.028 | 31.367 | 24.145 | 1.00 | 28.87 | 7 | N |
| ATOM | 1763 | CA | TYR A | 230 | 44.583 | 30.145 | 23.557 | 1.00 | 27.46 | 6 | C |
| ATOM | 1764 | CB | TYR A | 230 | 44.178 | 30.105 | 22.067 | 1.00 | 29.58 | 6 | C |
| ATOM | 1765 | CG | TYR A | 230 | 44.676 | 28.861 | 21.352 | 1.00 | 30.96 | 6 | C |
| ATOM | 1766 | CD1 | TYR A | 230 | 44.383 | 27.603 | 21.830 | 1.00 | 33.87 | 6 | C |
| ATOM | 1767 | CE1 | TYR A | 230 | 44.844 | 26.453 | 21.197 | 1.00 | 35.06 | 6 | C |
| ATOM | 1768 | CD2 | TYR A | 230 | 45.437 | 28.928 | 20.182 | 1.00 | 34.27 | 6 | C |
| ATOM | 1769 | CE2 | TYR A | 230 | 45.910 | 27.791 | 19.542 | 1.00 | 35.29 | 6 | C |
| ATOM | 1770 | CZ | TYR A | 230 | 45.598 | 26.551 | 20.054 | 1.00 | 36.17 | 6 | C |
| ATOM | 1771 | OH | TYR A | 230 | 46.018 | 25.375 | 19.458 | 1.00 | 36.87 | 8 | O |
| ATOM | 1772 | C | TYR A | 230 | 46.068 | 30.008 | 23.770 | 1.00 | 25.36 | 6 | C |
| ATOM | 1773 | O | TYR A | 230 | 46.579 | 28.972 | 24.171 | 1.00 | 24.45 | 8 | O |
| ATOM | 1774 | N | VAL A | 231 | 46.864 | 31.093 | 23.582 | 1.00 | 24.69 | 7 | N |
| ATOM | 1775 | CA | VAL A | 231 | 48.288 | 31.093 | 23.792 | 1.00 | 24.36 | 6 | C |
| ATOM | 1776 | CB | VAL A | 231 | 48.909 | 32.484 | 23.465 | 1.00 | 26.19 | 6 | C |
| ATOM | 1777 | CG1 | VAL A | 231 | 50.342 | 32.558 | 23.970 | 1.00 | 27.75 | 6 | C |
| ATOM | 1778 | CG2 | VAL A | 231 | 48.845 | 32.821 | 21.984 | 1.00 | 25.64 | 6 | C |
| ATOM | 1779 | C | VAL A | 231 | 48.617 | 30.727 | 25.227 | 1.00 | 23.15 | 6 | C |
| ATOM | 1780 | O | VAL A | 231 | 49.504 | 29.906 | 25.504 | 1.00 | 23.27 | 8 | O |
| ATOM | 1781 | N | MET A | 232 | 47.839 | 31.324 | 26.138 | 1.00 | 24.47 | 7 | N |
| ATOM | 1782 | CA | MET A | 232 | 48.097 | 30.982 | 27.566 | 1.00 | 28.80 | 6 | C |
| ATOM | 1783 | CB | MET A | 232 | 47.237 | 31.909 | 28.423 | 1.00 | 33.07 | 6 | C |
| ATOM | 1784 | CG | MET A | 232 | 47.804 | 33.350 | 28.394 | 1.00 | 36.34 | 6 | C |
| ATOM | 1785 | SD | MET A | 232 | 49.573 | 33.379 | 28.672 | 1.00 | 38.34 | 16 | S |
| ATOM | 1786 | CE | MET A | 232 | 49.643 | 33.480 | 30.463 | 1.00 | 38.38 | 6 | C |
| ATOM | 1787 | C | MET A | 232 | 47.830 | 29.504 | 27.869 | 1.00 | 26.80 | 6 | C |
| ATOM | 1788 | O | MET A | 232 | 48.488 | 28.805 | 28.646 | 1.00 | 26.62 | 8 | O |
| ATOM | 1789 | N | GLN A | 233 | 46.820 | 28.949 | 27.187 | 1.00 | 25.29 | 7 | N |
| ATOM | 1790 | CA | GLN A | 233 | 46.476 | 27.519 | 27.342 | 1.00 | 25.62 | 6 | C |
| ATOM | 1791 | CB | GLN A | 233 | 45.130 | 27.188 | 26.740 | 1.00 | 26.45 | 6 | C |
| ATOM | 1792 | CG | GLN A | 233 | 43.924 | 27.908 | 27.335 | 1.00 | 28.74 | 6 | C |
| ATOM | 1793 | CD | GLN A | 233 | 42.654 | 27.817 | 26.515 | 1.00 | 31.11 | 6 | C |
| ATOM | 1794 | OE1 | GLN A | 233 | 42.626 | 27.978 | 25.296 | 1.00 | 32.14 | 8 | O |
| ATOM | 1795 | NE2 | GLN A | 233 | 41.494 | 27.554 | 27.138 | 1.00 | 32.03 | 7 | N |
| ATOM | 1796 | C | GLN A | 233 | 47.594 | 26.641 | 26.764 | 1.00 | 24.07 | 6 | C |
| ATOM | 1797 | O | GLN A | 233 | 47.926 | 25.640 | 27.391 | 1.00 | 24.21 | 8 | O |
| ATOM | 1798 | N | VAL A | 234 | 48.177 | 27.079 | 25.639 | 1.00 | 22.55 | 7 | N |
| ATOM | 1799 | CA | VAL A | 234 | 49.307 | 26.352 | 25.079 | 1.00 | 22.93 | 6 | C |
| ATOM | 1800 | CB | VAL A | 234 | 49.674 | 26.935 | 23.688 | 1.00 | 22.37 | 6 | C |
| ATOM | 1801 | CG1 | VAL A | 234 | 50.994 | 26.335 | 23.240 | 1.00 | 24.31 | 6 | C |
| ATOM | 1802 | CG2 | VAL A | 234 | 48.533 | 26.584 | 22.738 | 1.00 | 26.04 | 6 | C |
| ATOM | 1803 | C | VAL A | 234 | 50.499 | 26.439 | 26.021 | 1.00 | 24.00 | 6 | C |
| ATOM | 1804 | O | VAL A | 234 | 51.164 | 25.418 | 26.279 | 1.00 | 26.18 | 8 | O |
| ATOM | 1805 | N | ILE A | 235 | 50.798 | 27.637 | 26.539 | 1.00 | 26.08 | 7 | N |
| ATOM | 1806 | CA | ILE A | 235 | 51.901 | 27.821 | 27.489 | 1.00 | 27.11 | 6 | C |
| ATOM | 1807 | CB | ILE A | 235 | 52.084 | 29.302 | 27.907 | 1.00 | 27.12 | 6 | C |
| ATOM | 1808 | CG2 | ILE A | 235 | 53.080 | 29.686 | 28.995 | 1.00 | 29.21 | 6 | C |
| ATOM | 1809 | CG1 | ILE A | 235 | 52.547 | 30.035 | 26.603 | 1.00 | 26.70 | 6 | C |
| ATOM | 1810 | CD1 | ILE A | 235 | 52.251 | 31.528 | 26.798 | 1.00 | 28.09 | 6 | C |
| ATOM | 1811 | C | ILE A | 235 | 51.883 | 26.998 | 28.772 | 1.00 | 29.93 | 6 | C |
| ATOM | 1812 | O | ILE A | 235 | 53.011 | 26.717 | 29.254 | 1.00 | 35.76 | 8 | O |
| ATOM | 1813 | N | ASP A | 236 | 50.676 | 26.703 | 29.262 | 1.00 | 31.31 | 7 | N |
| ATOM | 1814 | CA | ASP A | 236 | 50.357 | 25.923 | 30.434 | 1.00 | 32.18 | 6 | C |
| ATOM | 1815 | CB | ASP A | 236 | 48.920 | 26.067 | 30.960 | 1.00 | 32.45 | 6 | C |
| ATOM | 1816 | CG | ASP A | 236 | 48.651 | 27.287 | 31.818 | 1.00 | 33.88 | 6 | C |
| ATOM | 1817 | OD1 | ASP A | 236 | 49.637 | 27.867 | 32.359 | 1.00 | 33.21 | 8 | O |
| ATOM | 1818 | OD2 | ASP A | 236 | 47.474 | 27.706 | 31.890 | 1.00 | 35.80 | 8 | O |
| ATOM | 1819 | C | ASP A | 236 | 50.610 | 24.456 | 30.124 | 1.00 | 31.32 | 6 | C |
| ATOM | 1820 | O | ASP A | 236 | 51.182 | 23.715 | 30.921 | 1.00 | 32.51 | 8 | O |
| ATOM | 1821 | N | ALA A | 237 | 50.158 | 24.043 | 28.941 | 1.00 | 30.15 | 7 | N |
| ATOM | 1822 | CA | ALA A | 237 | 50.267 | 22.650 | 28.525 | 1.00 | 29.73 | 6 | C |
| ATOM | 1823 | CB | ALA A | 237 | 49.270 | 22.352 | 27.405 | 1.00 | 27.85 | 6 | C |
| ATOM | 1824 | C | ALA A | 237 | 51.655 | 22.249 | 28.117 | 1.00 | 29.97 | 6 | C |
| ATOM | 1825 | O | ALA A | 237 | 52.154 | 21.135 | 28.157 | 1.00 | 31.07 | 8 | O |
| ATOM | 1826 | N | THR A | 238 | 52.471 | 23.228 | 27.710 | 1.00 | 28.81 | 7 | N |
| ATOM | 1827 | CA | THR A | 238 | 53.863 | 22.954 | 27.315 | 1.00 | 29.70 | 6 | C |
| ATOM | 1828 | CB | THR A | 238 | 54.171 | 23.445 | 25.879 | 1.00 | 28.72 | 6 | C |
| ATOM | 1829 | OG1 | THR A | 238 | 53.944 | 24.856 | 25.824 | 1.00 | 28.32 | 8 | O |
| ATOM | 1830 | CG2 | THR A | 238 | 53.366 | 22.701 | 24.827 | 1.00 | 30.64 | 6 | C |
| ATOM | 1831 | C | THR A | 238 | 54.873 | 23.646 | 28.228 | 1.00 | 32.30 | 6 | C |
| ATOM | 1832 | O | THR A | 238 | 55.970 | 23.995 | 27.791 | 1.00 | 29.50 | 8 | O |
| ATOM | 1833 | N | ALA A | 239 | 54.499 | 23.837 | 29.479 | 1.00 | 32.51 | 7 | N |
| ATOM | 1834 | CA | ALA A | 239 | 55.241 | 24.622 | 30.447 | 1.00 | 36.82 | 6 | C |
| ATOM | 1835 | CB | ALA A | 239 | 54.603 | 24.442 | 31.824 | 1.00 | 35.83 | 6 | C |
| ATOM | 1836 | C | ALA A | 239 | 56.745 | 24.359 | 30.552 | 1.00 | 37.39 | 6 | C |
| ATOM | 1837 | O | ALA A | 239 | 57.486 | 25.340 | 30.666 | 1.00 | 37.58 | 8 | O |
| ATOM | 1838 | N | GLU A | 240 | 57.113 | 23.105 | 30.560 | 1.00 | 39.92 | 7 | N |
| ATOM | 1839 | CA | GLU A | 240 | 58.514 | 22.712 | 30.684 | 1.00 | 43.43 | 6 | C |
| ATOM | 1840 | CB | GLU A | 240 | 58.590 | 21.376 | 31.436 | 1.00 | 47.51 | 6 | C |
| ATOM | 1841 | CG | GLU A | 240 | 58.526 | 21.597 | 32.935 | 1.00 | 52.60 | 6 | C |
| ATOM | 1842 | CD | GLU A | 240 | 57.130 | 21.783 | 33.484 | 1.00 | 56.58 | 6 | C |
| ATOM | 1843 | OE1 | GLU A | 240 | 56.272 | 20.868 | 33.365 | 1.00 | 58.99 | 8 | O |
| ATOM | 1844 | OE2 | GLU A | 240 | 56.871 | 22.851 | 34.085 | 1.00 | 58.37 | 8 | O |
| ATOM | 1845 | C | GLU A | 240 | 59.271 | 22.673 | 29.375 | 1.00 | 41.19 | 6 | C |
| ATOM | 1846 | O | GLU A | 240 | 60.492 | 22.782 | 29.438 | 1.00 | 43.22 | 8 | O |
| ATOM | 1847 | N | ASN A | 241 | 58.626 | 22.679 | 28.220 | 1.00 | 39.11 | 7 | N |
| ATOM | 1848 | CA | ASN A | 241 | 59.334 | 22.632 | 26.952 | 1.00 | 37.18 | 6 | C |
| ATOM | 1849 | CB | ASN A | 241 | 58.291 | 22.479 | 25.833 | 1.00 | 39.30 | 6 | C |
| ATOM | 1850 | CG | ASN A | 241 | 57.501 | 21.179 | 25.964 | 1.00 | 41.56 | 6 | C |
| ATOM | 1851 | OD1 | ASN A | 241 | 56.664 | 21.010 | 25.067 | 1.00 | 41.45 | 8 | O |
| ATOM | 1852 | ND2 | ASN A | 241 | 57.777 | 20.355 | 26.956 | 1.00 | 40.47 | 7 | N |
| ATOM | 1853 | C | ASN A | 241 | 60.174 | 23.872 | 26.598 | 1.00 | 33.45 | 6 | C |
| ATOM | 1854 | O | ASN A | 241 | 59.885 | 24.949 | 27.062 | 1.00 | 31.38 | 8 | O |
| ATOM | 1855 | N | ILE A | 242 | 61.135 | 23.598 | 25.737 | 1.00 | 32.31 | 7 | N |
| ATOM | 1856 | CA | ILE A | 242 | 61.985 | 24.618 | 25.119 | 1.00 | 32.09 | 6 | C |
| ATOM | 1857 | CB | ILE A | 242 | 63.458 | 24.173 | 25.081 | 1.00 | 31.78 | 6 | C |
| ATOM | 1858 | CG2 | ILE A | 242 | 64.349 | 25.226 | 24.443 | 1.00 | 30.95 | 6 | C |
| ATOM | 1859 | CG1 | ILE A | 242 | 63.910 | 23.874 | 26.540 | 1.00 | 31.96 | 6 | C |
| ATOM | 1860 | CD1 | ILE A | 242 | 63.612 | 25.005 | 27.512 | 1.00 | 32.18 | 6 | C |
| ATOM | 1861 | C | ILE A | 242 | 61.427 | 24.796 | 23.717 | 1.00 | 30.11 | 6 | C |
| ATOM | 1862 | O | ILE A | 242 | 61.315 | 23.780 | 22.976 | 1.00 | 31.69 | 8 | O |
| ATOM | 1863 | N | SER A | 243 | 61.079 | 26.028 | 23.328 | 1.00 | 28.62 | 7 | N |
| ATOM | 1864 | CA | SER A | 243 | 60.516 | 26.217 | 21.990 | 1.00 | 29.16 | 6 | C |
| ATOM | 1865 | CB | SER A | 243 | 60.041 | 27.661 | 21.826 | 1.00 | 28.29 | 6 | C |
| ATOM | 1866 | OG | SER A | 243 | 61.160 | 28.560 | 21.760 | 1.00 | 26.85 | 8 | O |
| ATOM | 1867 | C | SER A | 243 | 61.448 | 25.858 | 20.854 | 1.00 | 28.52 | 6 | C |
| ATOM | 1868 | O | SER A | 243 | 62.680 | 25.795 | 20.958 | 1.00 | 28.35 | 8 | O |
| ATOM | 1869 | N | SER A | 244 | 60.873 | 25.762 | 19.629 | 1.00 | 26.17 | 7 | N |
| ATOM | 1870 | CA | SER A | 244 | 61.682 | 25.478 | 18.453 | 1.00 | 26.74 | 6 | C |
| ATOM | 1871 | CB | SER A | 244 | 60.878 | 25.251 | 17.163 | 1.00 | 27.44 | 6 | C |
| ATOM | 1872 | OG | SER A | 244 | 60.061 | 26.385 | 16.920 | 1.00 | 29.47 | 8 | O |
| ATOM | 1873 | C | SER A | 244 | 62.669 | 26.634 | 18.202 | 1.00 | 27.21 | 6 | C |
| ATOM | 1874 | O | SER A | 244 | 63.779 | 26.338 | 17.769 | 1.00 | 28.33 | 8 | O |
| ATOM | 1875 | N | MET A | 245 | 62.238 | 27.872 | 18.403 | 1.00 | 26.31 | 7 | N |
| ATOM | 1876 | CA | MET A | 245 | 63.106 | 29.042 | 18.197 | 1.00 | 22.50 | 6 | C |
| ATOM | 1877 | CB | MET A | 245 | 62.278 | 30.333 | 18.086 | 1.00 | 23.49 | 6 | C |
| ATOM | 1878 | CG | MET A | 245 | 63.175 | 31.469 | 17.467 | 1.00 | 20.17 | 6 | C |
| ATOM | 1879 | SD | MET A | 245 | 62.223 | 32.952 | 17.255 | 1.00 | 22.14 | 16 | S |
| ATOM | 1880 | CE | MET A | 245 | 61.471 | 32.506 | 15.645 | 1.00 | 20.01 | 6 | C |
| ATOM | 1881 | C | MET A | 245 | 64.208 | 29.146 | 19.245 | 1.00 | 29.11 | 6 | C |
| ATOM | 1882 | O | MET A | 245 | 65.384 | 29.350 | 18.868 | 1.00 | 29.77 | 8 | O |
| ATOM | 1883 | N | LEU A | 246 | 63.895 | 28.891 | 20.499 | 1.00 | 27.45 | 7 | N |
| ATOM | 1884 | CA | LEU A | 246 | 64.903 | 28.950 | 21.569 | 1.00 | 32.56 | 6 | C |
| ATOM | 1885 | CB | LEU A | 246 | 64.302 | 28.828 | 22.961 | 1.00 | 30.90 | 6 | C |
| ATOM | 1886 | CG | LEU A | 246 | 65.282 | 28.813 | 24.163 | 1.00 | 31.21 | 6 | C |
| ATOM | 1887 | CD1 | LEU A | 246 | 66.323 | 29.931 | 23.993 | 1.00 | 29.92 | 6 | C |
| ATOM | 1888 | CD2 | LEU A | 246 | 64.567 | 28.959 | 25.480 | 1.00 | 28.69 | 6 | C |
| ATOM | 1889 | C | LEU A | 246 | 65.919 | 27.846 | 21.313 | 1.00 | 34.75 | 6 | C |
| ATOM | 1890 | O | LEU A | 246 | 67.141 | 27.985 | 21.335 | 1.00 | 36.47 | 8 | O |
| ATOM | 1891 | N | GLN A | 247 | 65.420 | 26.696 | 20.863 | 1.00 | 35.19 | 7 | N |
| ATOM | 1892 | CA | GLN A | 247 | 66.256 | 25.568 | 20.503 | 1.00 | 39.33 | 6 | C |
| ATOM | 1893 | CB | GLN A | 247 | 65.370 | 24.447 | 19.957 | 1.00 | 43.06 | 6 | C |
| ATOM | 1894 | CG | GLN A | 247 | 65.911 | 23.064 | 20.267 | 1.00 | 47.74 | 6 | C |
| ATOM | 1895 | CD | GLN A | 247 | 64.868 | 22.020 | 19.898 | 1.00 | 49.30 | 6 | C |
| ATOM | 1896 | OE1 | GLN A | 247 | 63.699 | 22.209 | 20.237 | 1.00 | 51.62 | 8 | O |
| ATOM | 1897 | NE2 | GLN A | 247 | 65.271 | 20.975 | 19.205 | 1.00 | 50.76 | 7 | N |
| ATOM | 1898 | C | GLN A | 247 | 67.284 | 25.922 | 19.436 | 1.00 | 39.74 | 6 | C |
| ATOM | 1899 | O | GLN A | 247 | 68.450 | 25.488 | 19.457 | 1.00 | 40.51 | 8 | O |
| ATOM | 1900 | N | ASP A | 248 | 66.847 | 26.705 | 18.453 | 1.00 | 37.82 | 7 | N |
| ATOM | 1901 | CA | ASP A | 248 | 67.741 | 27.168 | 17.400 | 1.00 | 38.31 | 6 | C |
| ATOM | 1902 | CB | ASP A | 248 | 66.953 | 27.860 | 16.279 | 1.00 | 34.47 | 6 | C |
| ATOM | 1903 | CG | ASP A | 248 | 66.131 | 26.925 | 15.406 | 1.00 | 33.49 | 6 | C |
| ATOM | 1904 | OD1 | ASP A | 248 | 66.521 | 25.763 | 15.172 | 1.00 | 32.56 | 8 | O |
| ATOM | 1905 | OD2 | ASP A | 248 | 65.078 | 27.313 | 14.860 | 1.00 | 30.46 | 8 | O |
| ATOM | 1906 | C | ASP A | 248 | 68.770 | 28.112 | 18.041 | 1.00 | 39.62 | 6 | C |
| ATOM | 1907 | O | ASP A | 248 | 69.987 | 27.986 | 17.793 | 1.00 | 40.39 | 8 | O |
| ATOM | 1908 | N | ILE A | 249 | 68.282 | 29.059 | 18.836 | 1.00 | 39.67 | 7 | N |
| ATOM | 1909 | CA | ILE A | 249 | 69.146 | 30.030 | 19.502 | 1.00 | 42.53 | 6 | C |
| ATOM | 1910 | CB | ILE A | 249 | 68.350 | 30.925 | 20.466 | 1.00 | 42.48 | 6 | C |
| ATOM | 1911 | CG2 | ILE A | 249 | 69.244 | 31.662 | 21.462 | 1.00 | 42.45 | 6 | C |
| ATOM | 1912 | CG1 | ILE A | 249 | 67.435 | 31.896 | 19.718 | 1.00 | 42.13 | 6 | C |
| ATOM | 1913 | CD1 | ILE A | 249 | 68.067 | 33.084 | 19.041 | 1.00 | 39.99 | 6 | C |
| ATOM | 1914 | C | ILE A | 249 | 70.241 | 29.311 | 20.289 | 1.00 | 45.30 | 6 | C |
| ATOM | 1915 | O | ILE A | 249 | 71.435 | 29.434 | 20.012 | 1.00 | 45.09 | 8 | O |
| ATOM | 1916 | N | ARG A | 250 | 69.846 | 28.459 | 21.224 | 1.00 | 46.11 | 7 | N |
| ATOM | 1917 | CA | ARG A | 250 | 70.777 | 27.714 | 22.059 | 1.00 | 49.58 | 6 | C |
| ATOM | 1918 | CB | ARG A | 250 | 70.047 | 26.803 | 23.053 | 1.00 | 48.49 | 6 | C |
| ATOM | 1919 | CG | ARG A | 250 | 69.221 | 27.551 | 24.082 | 1.00 | 48.25 | 6 | C |
| ATOM | 1920 | CD | ARG A | 250 | 68.639 | 26.596 | 25.111 | 1.00 | 48.29 | 6 | C |
| ATOM | 1921 | NE | ARG A | 250 | 68.097 | 27.322 | 26.264 | 1.00 | 47.22 | 7 | N |
| ATOM | 1922 | CZ | ARG A | 250 | 67.460 | 26.725 | 27.258 | 1.00 | 46.33 | 6 | C |
| ATOM | 1923 | NH1 | ARG A | 250 | 66.984 | 27.465 | 28.256 | 1.00 | 46.74 | 7 | N |
| ATOM | 1924 | NH2 | ARG A | 250 | 67.260 | 25.432 | 27.274 | 1.00 | 46.59 | 7 | N |
| ATOM | 1925 | C | ARG A | 250 | 71.793 | 26.932 | 21.246 | 1.00 | 51.10 | 6 | C |
| ATOM | 1926 | O | ARG A | 250 | 72.931 | 26.733 | 21.687 | 1.00 | 52.50 | 8 | O |
| ATOM | 1927 | N | ALA A | 251 | 71.468 | 26.476 | 20.043 | 1.00 | 52.45 | 7 | N |
| ATOM | 1928 | CA | ALA A | 251 | 72.409 | 25.782 | 19.184 | 1.00 | 52.80 | 6 | C |
| ATOM | 1929 | CB | ALA A | 251 | 71.678 | 24.660 | 18.460 | 1.00 | 52.47 | 6 | C |
| ATOM | 1930 | C | ALA A | 251 | 73.051 | 26.710 | 18.161 | 1.00 | 52.93 | 6 | C |
| ATOM | 1931 | O | ALA A | 251 | 73.767 | 26.271 | 17.256 | 1.00 | 52.77 | 8 | O |
| ATOM | 1932 | N | LEU A | 252 | 72.734 | 27.996 | 18.195 | 1.00 | 53.59 | 7 | N |
| ATOM | 1933 | CA | LEU A | 252 | 73.215 | 28.974 | 17.242 | 1.00 | 54.61 | 6 | C |
| ATOM | 1934 | CB | LEU A | 252 | 74.708 | 29.198 | 17.439 | 1.00 | 56.65 | 6 | C |
| ATOM | 1935 | CG | LEU A | 252 | 75.284 | 29.550 | 18.776 | 1.00 | 57.62 | 6 | C |
| ATOM | 1936 | CD1 | LEU A | 252 | 76.693 | 29.114 | 18.901 | 1.00 | 58.39 | 6 | C |
| ATOM | 1937 | CD2 | LEU A | 252 | 74.966 | 30.934 | 19.276 | 1.00 | 58.87 | 6 | C |
| ATOM | 1938 | C | LEU A | 252 | 72.931 | 28.556 | 15.792 | 1.00 | 53.74 | 6 | C |
| ATOM | 1939 | O | LEU A | 252 | 73.830 | 28.702 | 14.957 | 1.00 | 53.37 | 8 | O |
| ATOM | 1940 | N | ARG A | 253 | 71.741 | 28.030 | 15.512 | 1.00 | 51.31 | 7 | N |
| ATOM | 1941 | CA | ARG A | 253 | 71.390 | 27.688 | 14.137 | 1.00 | 49.13 | 6 | C |
| ATOM | 1942 | CB | ARG A | 253 | 70.570 | 26.428 | 13.946 | 1.00 | 51.31 | 6 | C |
| ATOM | 1943 | CG | ARG A | 253 | 70.845 | 25.223 | 14.806 | 1.00 | 54.01 | 6 | C |
| ATOM | 1944 | CD | ARG A | 253 | 69.827 | 24.101 | 14.635 | 1.00 | 56.03 | 6 | C |
| ATOM | 1945 | NE | ARG A | 253 | 69.626 | 23.295 | 15.827 | 1.00 | 57.53 | 7 | N |
| ATOM | 1946 | CZ | ARG A | 253 | 68.614 | 23.330 | 16.679 | 1.00 | 58.76 | 6 | C |
| ATOM | 1947 | NH1 | ARG A | 253 | 67.556 | 24.113 | 16.564 | 1.00 | 59.59 | 7 | N |
| ATOM | 1948 | NH2 | ARG A | 253 | 68.643 | 22.508 | 17.729 | 1.00 | 60.24 | 7 | N |
| ATOM | 1949 | C | ARG A | 253 | 70.589 | 28.913 | 13.684 | 1.00 | 45.03 | 6 | C |
| ATOM | 1950 | O | ARG A | 253 | 70.057 | 29.608 | 14.561 | 1.00 | 42.83 | 8 | O |
| ATOM | 1951 | N | HIS A | 254 | 70.542 | 29.180 | 12.388 | 1.00 | 42.19 | 7 | N |
| ATOM | 1952 | CA | HIS A | 254 | 69.707 | 30.301 | 11.940 | 1.00 | 38.35 | 6 | C |
| ATOM | 1953 | CB | HIS A | 254 | 69.702 | 30.407 | 10.426 | 1.00 | 41.95 | 6 | C |
| ATOM | 1954 | CG | HIS A | 254 | 70.940 | 30.976 | 9.829 | 1.00 | 46.54 | 6 | C |
| ATOM | 1955 | CD2 | HIS A | 254 | 71.484 | 32.211 | 9.915 | 1.00 | 48.24 | 6 | C |
| ATOM | 1956 | ND1 | HIS A | 254 | 71.789 | 30.249 | 9.023 | 1.00 | 48.43 | 7 | N |
| ATOM | 1957 | CE1 | HIS A | 254 | 72.798 | 31.002 | 8.624 | 1.00 | 49.00 | 6 | C |
| ATOM | 1958 | NE2 | HIS A | 254 | 72.638 | 32.201 | 9.160 | 1.00 | 49.34 | 7 | N |
| ATOM | 1959 | C | HIS A | 254 | 68.256 | 30.056 | 12.401 | 1.00 | 34.21 | 6 | C |
| ATOM | 1960 | O | HIS A | 254 | 67.912 | 28.876 | 12.490 | 1.00 | 32.74 | 8 | O |
| ATOM | 1961 | N | THR A | 255 | 67.459 | 31.088 | 12.650 | 1.00 | 30.00 | 7 | N |
| ATOM | 1962 | CA | THR A | 255 | 66.108 | 30.900 | 13.101 | 1.00 | 27.94 | 6 | C |
| ATOM | 1963 | CB | THR A | 255 | 65.585 | 31.868 | 14.182 | 1.00 | 24.63 | 6 | C |
| ATOM | 1964 | OG1 | THR A | 255 | 65.252 | 33.118 | 13.631 | 1.00 | 25.36 | 8 | O |
| ATOM | 1965 | CG2 | THR A | 255 | 66.589 | 31.981 | 15.335 | 1.00 | 25.51 | 6 | C |
| ATOM | 1966 | C | THR A | 255 | 65.103 | 31.022 | 11.939 | 1.00 | 27.14 | 6 | C |
| ATOM | 1967 | O | THR A | 255 | 65.506 | 31.473 | 10.867 | 1.00 | 26.33 | 8 | O |
| ATOM | 1968 | N | GLU A | 256 | 63.836 | 30.684 | 12.178 | 1.00 | 25.22 | 7 | N |
| ATOM | 1969 | CA | GLU A | 256 | 62.797 | 30.829 | 11.173 | 1.00 | 22.10 | 6 | C |
| ATOM | 1970 | CB | GLU A | 256 | 61.778 | 29.665 | 11.126 | 1.00 | 25.40 | 6 | C |
| ATOM | 1971 | CG | GLU A | 256 | 62.408 | 28.371 | 10.595 | 1.00 | 26.74 | 6 | C |
| ATOM | 1972 | CD | GLU A | 256 | 61.481 | 27.152 | 10.560 | 1.00 | 31.16 | 6 | C |
| ATOM | 1973 | OE1 | GLU A | 256 | 61.114 | 26.755 | 9.408 | 1.00 | 28.77 | 8 | O |
| ATOM | 1974 | OE2 | GLU A | 256 | 61.147 | 26.609 | 11.630 | 1.00 | 28.79 | 8 | O |
| ATOM | 1975 | C | GLU A | 256 | 62.059 | 32.140 | 11.440 | 1.00 | 22.57 | 6 | C |
| ATOM | 1976 | O | GLU A | 256 | 60.989 | 32.372 | 10.887 | 1.00 | 21.91 | 8 | O |
| ATOM | 1977 | N | ILE A | 257 | 62.642 | 33.052 | 12.215 | 1.00 | 23.68 | 7 | N |
| ATOM | 1978 | CA | ILE A | 257 | 61.970 | 34.320 | 12.539 | 1.00 | 22.55 | 6 | C |
| ATOM | 1979 | CB | ILE A | 257 | 63.077 | 35.100 | 13.327 | 1.00 | 28.11 | 6 | C |
| ATOM | 1980 | CG2 | ILE A | 257 | 64.164 | 35.579 | 12.377 | 1.00 | 30.70 | 6 | C |
| ATOM | 1981 | CG1 | ILE A | 257 | 62.458 | 36.327 | 13.971 | 1.00 | 32.19 | 6 | C |
| ATOM | 1982 | CD1 | ILE A | 257 | 61.417 | 36.123 | 15.048 | 1.00 | 33.04 | 6 | C |
| ATOM | 1983 | C | ILE A | 257 | 61.581 | 35.145 | 11.335 | 1.00 | 19.40 | 6 | C |
| ATOM | 1984 | O | ILE A | 257 | 60.606 | 35.901 | 11.441 | 1.00 | 19.60 | 8 | O |
| ATOM | 1985 | N | ASP A | 258 | 62.279 | 35.076 | 10.179 | 1.00 | 20.73 | 7 | N |
| ATOM | 1986 | CA | ASP A | 258 | 61.912 | 35.873 | 9.032 | 1.00 | 20.84 | 6 | C |
| ATOM | 1987 | CB | ASP A | 258 | 63.044 | 35.747 | 7.984 | 1.00 | 24.46 | 6 | C |
| ATOM | 1988 | CG | ASP A | 258 | 64.263 | 36.542 | 8.446 | 1.00 | 26.53 | 6 | C |
| ATOM | 1989 | OD1 | ASP A | 258 | 64.120 | 37.758 | 8.496 | 1.00 | 25.11 | 8 | O |
| ATOM | 1990 | OD2 | ASP A | 258 | 65.256 | 35.868 | 8.711 | 1.00 | 30.20 | 8 | O |
| ATOM | 1991 | C | ASP A | 258 | 60.577 | 35.489 | 8.394 | 1.00 | 21.17 | 6 | C |
| ATOM | 1992 | O | ASP A | 258 | 59.935 | 36.190 | 7.623 | 1.00 | 22.70 | 8 | O |
| ATOM | 1993 | N | TYR A | 259 | 60.115 | 34.278 | 8.751 | 1.00 | 19.28 | 7 | N |
| ATOM | 1994 | CA | TYR A | 259 | 58.894 | 33.671 | 8.285 | 1.00 | 20.42 | 6 | C |
| ATOM | 1995 | CB | TYR A | 259 | 59.238 | 32.309 | 7.600 | 1.00 | 19.36 | 6 | C |
| ATOM | 1996 | CG | TYR A | 259 | 60.126 | 32.623 | 6.404 | 1.00 | 21.28 | 6 | C |
| ATOM | 1997 | CD1 | TYR A | 259 | 61.493 | 32.659 | 6.538 | 1.00 | 22.25 | 6 | C |
| ATOM | 1998 | CE1 | TYR A | 259 | 62.335 | 33.011 | 5.509 | 1.00 | 21.86 | 6 | C |
| ATOM | 1999 | CD2 | TYR A | 259 | 59.544 | 32.874 | 5.145 | 1.00 | 21.59 | 6 | C |
| ATOM | 2000 | CE2 | TYR A | 259 | 60.410 | 33.243 | 4.100 | 1.00 | 22.21 | 6 | C |
| ATOM | 2001 | CZ | TYR A | 259 | 61.756 | 33.315 | 4.286 | 1.00 | 24.74 | 6 | C |
| ATOM | 2002 | OH | TYR A | 259 | 62.563 | 33.695 | 3.246 | 1.00 | 23.85 | 8 | O |
| ATOM | 2003 | C | TYR A | 259 | 57.846 | 33.495 | 9.369 | 1.00 | 19.80 | 6 | C |
| ATOM | 2004 | O | TYR A | 259 | 56.850 | 32.765 | 9.251 | 1.00 | 19.43 | 8 | O |
| ATOM | 2005 | N | ILE A | 260 | 58.108 | 34.111 | 10.567 | 1.00 | 19.72 | 7 | N |
| ATOM | 2006 | CA | ILE A | 260 | 57.123 | 34.048 | 11.660 | 1.00 | 19.94 | 6 | C |
| ATOM | 2007 | CB | ILE A | 260 | 57.701 | 33.308 | 12.875 | 1.00 | 19.22 | 6 | C |
| ATOM | 2008 | CG2 | ILE A | 260 | 56.679 | 33.215 | 14.015 | 1.00 | 21.48 | 6 | C |
| ATOM | 2009 | CG1 | ILE A | 260 | 58.183 | 31.903 | 12.516 | 1.00 | 21.49 | 6 | C |
| ATOM | 2010 | CD1 | ILE A | 260 | 58.844 | 31.107 | 13.610 | 1.00 | 22.06 | 6 | C |
| ATOM | 2011 | C | ILE A | 260 | 56.828 | 35.507 | 11.976 | 1.00 | 20.82 | 6 | C |
| ATOM | 2012 | O | ILE A | 260 | 55.928 | 36.023 | 11.294 | 1.00 | 20.92 | 8 | O |
| ATOM | 2013 | N | ASN A | 261 | 57.581 | 36.199 | 12.839 | 1.00 | 18.77 | 7 | N |
| ATOM | 2014 | CA | ASN A | 261 | 57.164 | 37.602 | 13.069 | 1.00 | 20.51 | 6 | C |
| ATOM | 2015 | CB | ASN A | 261 | 57.688 | 38.211 | 14.363 | 1.00 | 21.31 | 6 | C |
| ATOM | 2016 | CG | ASN A | 261 | 57.425 | 37.271 | 15.558 | 1.00 | 20.99 | 6 | C |
| ATOM | 2017 | OD1 | ASN A | 261 | 58.010 | 36.188 | 15.684 | 1.00 | 19.55 | 8 | O |
| ATOM | 2018 | ND2 | ASN A | 261 | 56.696 | 37.802 | 16.521 | 1.00 | 22.13 | 7 | N |
| ATOM | 2019 | C | ASN A | 261 | 57.659 | 38.459 | 11.900 | 1.00 | 19.63 | 6 | C |
| ATOM | 2020 | O | ASN A | 261 | 56.856 | 39.374 | 11.591 | 1.00 | 21.43 | 8 | O |
| ATOM | 2021 | N | GLY A | 262 | 58.651 | 38.027 | 11.168 | 1.00 | 22.52 | 7 | N |
| ATOM | 2022 | CA | GLY A | 262 | 59.041 | 38.747 | 9.970 | 1.00 | 21.76 | 6 | C |
| ATOM | 2023 | C | GLY A | 262 | 57.932 | 38.760 | 8.939 | 1.00 | 25.21 | 6 | C |
| ATOM | 2024 | O | GLY A | 262 | 57.704 | 39.789 | 8.268 | 1.00 | 26.10 | 8 | O |
| ATOM | 2025 | N | PHE A | 263 | 57.174 | 37.674 | 8.758 | 1.00 | 24.05 | 7 | N |
| ATOM | 2026 | CA | PHE A | 263 | 56.047 | 37.664 | 7.837 | 1.00 | 21.20 | 6 | C |
| ATOM | 2027 | CB | PHE A | 263 | 55.440 | 36.273 | 7.679 | 1.00 | 22.11 | 6 | C |
| ATOM | 2028 | CG | PHE A | 263 | 54.274 | 36.162 | 6.736 | 1.00 | 23.66 | 6 | C |
| ATOM | 2029 | CD1 | PHE A | 263 | 54.480 | 36.263 | 5.363 | 1.00 | 25.61 | 6 | C |
| ATOM | 2030 | CD2 | PHE A | 263 | 53.003 | 35.896 | 7.189 | 1.00 | 23.05 | 6 | C |
| ATOM | 2031 | CE1 | PHE A | 263 | 53.407 | 36.105 | 4.494 | 1.00 | 24.77 | 6 | C |
| ATOM | 2032 | CE2 | PHE A | 263 | 51.912 | 35.790 | 6.360 | 1.00 | 24.86 | 6 | C |
| ATOM | 2033 | CZ | PHE A | 263 | 52.136 | 35.901 | 4.988 | 1.00 | 24.61 | 6 | C |
| ATOM | 2034 | C | PHE A | 263 | 54.966 | 38.592 | 8.356 | 1.00 | 23.52 | 6 | C |
| ATOM | 2035 | O | PHE A | 263 | 54.272 | 39.266 | 7.597 | 1.00 | 21.50 | 8 | O |
| ATOM | 2036 | N | LEU A | 264 | 54.677 | 38.582 | 9.690 | 1.00 | 22.18 | 7 | N |
| ATOM | 2037 | CA | LEU A | 264 | 53.630 | 39.465 | 10.213 | 1.00 | 23.10 | 6 | C |
| ATOM | 2038 | CB | LEU A | 264 | 53.478 | 39.301 | 11.723 | 1.00 | 24.44 | 6 | C |
| ATOM | 2039 | CG | LEU A | 264 | 52.263 | 39.978 | 12.390 | 1.00 | 23.36 | 6 | C |
| ATOM | 2040 | CD1 | LEU A | 264 | 50.992 | 39.178 | 12.198 | 1.00 | 24.30 | 6 | C |
| ATOM | 2041 | CD2 | LEU A | 264 | 52.609 | 40.193 | 13.860 | 1.00 | 27.78 | 6 | C |
| ATOM | 2042 | C | LEU A | 264 | 53.999 | 40.938 | 9.931 | 1.00 | 24.62 | 6 | C |
| ATOM | 2043 | O | LEU A | 264 | 53.072 | 41.705 | 9.604 | 1.00 | 23.80 | 8 | O |
| ATOM | 2044 | N | LEU A | 265 | 55.266 | 41.253 | 10.094 | 1.00 | 24.78 | 7 | N |
| ATOM | 2045 | CA | LEU A | 265 | 55.725 | 42.624 | 9.836 | 1.00 | 27.41 | 6 | C |
| ATOM | 2046 | CB | LEU A | 265 | 57.222 | 42.731 | 10.060 | 1.00 | 27.68 | 6 | C |
| ATOM | 2047 | CG | LEU A | 265 | 57.784 | 42.517 | 11.467 | 1.00 | 29.43 | 6 | C |
| ATOM | 2048 | CD1 | LEU A | 265 | 59.027 | 43.366 | 11.642 | 1.00 | 29.74 | 6 | C |
| ATOM | 2049 | CD2 | LEU A | 265 | 56.809 | 42.878 | 12.582 | 1.00 | 29.36 | 6 | C |
| ATOM | 2050 | C | LEU A | 265 | 55.504 | 42.986 | 8.353 | 1.00 | 29.14 | 6 | C |
| ATOM | 2051 | O | LEU A | 265 | 55.016 | 44.056 | 8.034 | 1.00 | 29.48 | 8 | O |
| ATOM | 2052 | N | ARG A | 266 | 55.899 | 42.061 | 7.470 | 1.00 | 26.87 | 7 | N |
| ATOM | 2053 | CA | ARG A | 266 | 55.698 | 42.290 | 6.025 | 1.00 | 29.62 | 6 | C |
| ATOM | 2054 | CB | ARG A | 266 | 56.284 | 41.102 | 5.219 | 1.00 | 30.88 | 6 | C |
| ATOM | 2055 | CG | ARG A | 266 | 57.815 | 41.163 | 5.241 | 1.00 | 31.87 | 6 | C |
| ATOM | 2056 | CD | ARG A | 266 | 58.439 | 40.296 | 4.164 | 1.00 | 32.86 | 6 | C |
| ATOM | 2057 | NE | ARG A | 266 | 58.243 | 38.865 | 4.346 | 1.00 | 33.01 | 7 | N |
| ATOM | 2058 | CZ | ARG A | 266 | 58.864 | 38.032 | 5.200 | 1.00 | 32.19 | 6 | C |
| ATOM | 2059 | NH1 | ARG A | 266 | 58.534 | 36.724 | 5.214 | 1.00 | 30.07 | 7 | N |
| ATOM | 2060 | NH2 | ARG A | 266 | 59.765 | 38.542 | 6.018 | 1.00 | 30.88 | 7 | N |
| ATOM | 2061 | C | ARG A | 266 | 54.239 | 42.483 | 5.666 | 1.00 | 31.12 | 6 | C |
| ATOM | 2062 | O | ARG A | 266 | 53.880 | 43.441 | 4.971 | 1.00 | 33.66 | 8 | O |
| ATOM | 2063 | N | ARG A | 267 | 53.303 | 41.697 | 6.201 | 1.00 | 30.85 | 7 | N |
| ATOM | 2064 | CA | ARG A | 267 | 51.890 | 41.814 | 5.921 | 1.00 | 30.08 | 6 | C |
| ATOM | 2065 | CB | ARG A | 267 | 51.026 | 40.604 | 6.326 | 1.00 | 29.69 | 6 | C |
| ATOM | 2066 | CG | ARG A | 267 | 51.394 | 39.338 | 5.552 | 1.00 | 29.63 | 6 | C |
| ATOM | 2067 | CD | ARG A | 267 | 51.301 | 39.578 | 4.042 | 1.00 | 32.69 | 6 | C |
| ATOM | 2068 | NE | ARG A | 267 | 49.980 | 39.976 | 3.615 | 1.00 | 34.59 | 7 | N |
| ATOM | 2069 | CZ | ARG A | 267 | 49.677 | 40.679 | 2.517 | 1.00 | 36.23 | 6 | C |
| ATOM | 2070 | NH1 | ARG A | 267 | 48.389 | 40.956 | 2.281 | 1.00 | 36.98 | 7 | N |
| ATOM | 2071 | NH2 | ARG A | 267 | 50.549 | 41.110 | 1.613 | 1.00 | 39.16 | 7 | N |
| ATOM | 2072 | C | ARG A | 267 | 51.293 | 43.051 | 6.609 | 1.00 | 32.47 | 6 | C |
| ATOM | 2073 | O | ARG A | 267 | 50.404 | 43.730 | 6.091 | 1.00 | 31.18 | 8 | O |
| ATOM | 2074 | N | ALA A | 268 | 51.851 | 43.354 | 7.784 | 1.00 | 33.41 | 7 | N |
| ATOM | 2075 | CA | ALA A | 268 | 51.397 | 44.529 | 8.539 | 1.00 | 37.78 | 6 | C |
| ATOM | 2076 | CB | ALA A | 268 | 52.144 | 44.592 | 9.863 | 1.00 | 37.31 | 6 | C |
| ATOM | 2077 | C | ALA A | 268 | 51.691 | 45.801 | 7.731 | 1.00 | 41.15 | 6 | C |
| ATOM | 2078 | O | ALA A | 268 | 50.908 | 46.750 | 7.637 | 1.00 | 40.60 | 8 | O |
| ATOM | 2079 | N | ARG A | 269 | 52.878 | 45.755 | 7.145 | 1.00 | 43.21 | 7 | N |
| ATOM | 2080 | CA | ARG A | 269 | 53.408 | 46.776 | 6.286 | 1.00 | 48.62 | 6 | C |
| ATOM | 2081 | CB | ARG A | 269 | 54.815 | 46.403 | 5.779 | 1.00 | 51.90 | 6 | C |
| ATOM | 2082 | CG | ARG A | 269 | 55.391 | 47.625 | 5.043 | 1.00 | 57.13 | 6 | C |
| ATOM | 2083 | CD | ARG A | 269 | 55.450 | 48.735 | 6.088 | 1.00 | 60.85 | 6 | C |
| ATOM | 2084 | NE | ARG A | 269 | 56.189 | 49.909 | 5.724 | 1.00 | 64.02 | 7 | N |
| ATOM | 2085 | CZ | ARG A | 269 | 57.400 | 49.943 | 5.189 | 1.00 | 65.87 | 6 | C |
| ATOM | 2086 | NH1 | ARG A | 269 | 58.123 | 48.869 | 4.900 | 1.00 | 66.08 | 7 | N |
| ATOM | 2087 | NH2 | ARG A | 269 | 57.936 | 51.128 | 4.916 | 1.00 | 66.42 | 7 | N |
| ATOM | 2088 | C | ARG A | 269 | 52.540 | 46.982 | 5.053 | 1.00 | 49.03 | 6 | C |
| ATOM | 2089 | O | ARG A | 269 | 52.229 | 48.124 | 4.731 | 1.00 | 49.58 | 8 | O |
| ATOM | 2090 | N | ALA A | 270 | 52.101 | 45.906 | 4.426 | 1.00 | 48.94 | 7 | N |
| ATOM | 2091 | CA | ALA A | 270 | 51.252 | 45.978 | 3.247 | 1.00 | 50.47 | 6 | C |
| ATOM | 2092 | CB | ALA A | 270 | 51.201 | 44.606 | 2.586 | 1.00 | 49.60 | 6 | C |
| ATOM | 2093 | C | ALA A | 270 | 49.858 | 46.506 | 3.555 | 1.00 | 51.98 | 6 | C |
| ATOM | 2094 | O | ALA A | 270 | 49.212 | 47.103 | 2.685 | 1.00 | 50.99 | 8 | O |
| ATOM | 2095 | N | HIS A | 271 | 49.364 | 46.309 | 4.767 | 1.00 | 52.89 | 7 | N |
| ATOM | 2096 | CA | HIS A | 271 | 48.057 | 46.770 | 5.182 | 1.00 | 54.55 | 6 | C |
| ATOM | 2097 | CB | HIS A | 271 | 47.368 | 45.729 | 6.076 | 1.00 | 55.17 | 6 | C |
| ATOM | 2098 | CG | HIS A | 271 | 46.967 | 44.484 | 5.347 | 1.00 | 56.38 | 6 | C |
| ATOM | 2099 | CD2 | HIS A | 271 | 47.371 | 43.198 | 5.448 | 1.00 | 55.91 | 6 | C |
| ATOM | 2100 | ND1 | HIS A | 271 | 45.990 | 44.508 | 4.373 | 1.00 | 56.70 | 7 | N |
| ATOM | 2101 | CE1 | HIS A | 271 | 45.814 | 43.293 | 3.892 | 1.00 | 57.04 | 6 | C |
| ATOM | 2102 | NE2 | HIS A | 271 | 46.642 | 42.485 | 4.530 | 1.00 | 57.14 | 7 | N |
| ATOM | 2103 | C | HIS A | 271 | 48.144 | 48.080 | 5.961 | 1.00 | 55.21 | 6 | C |
| ATOM | 2104 | O | HIS A | 271 | 47.131 | 48.726 | 6.231 | 1.00 | 54.83 | 8 | O |
| ATOM | 2105 | N | GLY A | 272 | 49.351 | 48.449 | 6.355 | 1.00 | 54.60 | 7 | N |
| ATOM | 2106 | CA | GLY A | 272 | 49.582 | 49.649 | 7.149 | 1.00 | 55.42 | 6 | C |
| ATOM | 2107 | C | GLY A | 272 | 49.021 | 49.512 | 8.557 | 1.00 | 54.48 | 6 | C |
| ATOM | 2108 | O | GLY A | 272 | 48.128 | 50.261 | 8.954 | 1.00 | 56.08 | 8 | O |
| ATOM | 2109 | N | ILE A | 273 | 49.537 | 48.555 | 9.323 | 1.00 | 52.89 | 7 | N |
| ATOM | 2110 | CA | ILE A | 273 | 49.108 | 48.292 | 10.686 | 1.00 | 50.36 | 6 | C |
| ATOM | 2111 | CB | ILE A | 273 | 48.547 | 46.863 | 10.852 | 1.00 | 50.50 | 6 | C |
| ATOM | 2112 | CG2 | ILE A | 273 | 48.292 | 46.533 | 12.315 | 1.00 | 49.35 | 6 | C |
| ATOM | 2113 | CG1 | ILE A | 273 | 47.305 | 46.647 | 9.991 | 1.00 | 49.58 | 6 | C |
| ATOM | 2114 | CD1 | ILE A | 273 | 46.175 | 47.615 | 10.248 | 1.00 | 49.55 | 6 | C |
| ATOM | 2115 | C | ILE A | 273 | 50.274 | 48.472 | 11.644 | 1.00 | 49.24 | 6 | C |
| ATOM | 2116 | O | ILE A | 273 | 51.372 | 47.968 | 11.372 | 1.00 | 49.40 | 8 | O |
| ATOM | 2117 | N | ALA A | 274 | 50.072 | 49.174 | 12.745 | 1.00 | 47.44 | 7 | N |
| ATOM | 2118 | CA | ALA A | 274 | 51.137 | 49.409 | 13.711 | 1.00 | 44.91 | 6 | C |
| ATOM | 2119 | CB | ALA A | 274 | 50.640 | 50.426 | 14.733 | 1.00 | 46.11 | 6 | C |
| ATOM | 2120 | C | ALA A | 274 | 51.558 | 48.127 | 14.409 | 1.00 | 44.18 | 6 | C |
| ATOM | 2121 | O | ALA A | 274 | 50.756 | 47.475 | 15.057 | 1.00 | 42.49 | 8 | O |
| ATOM | 2122 | N | VAL A | 275 | 52.845 | 47.773 | 14.328 | 1.00 | 42.23 | 7 | N |
| ATOM | 2123 | CA | VAL A | 275 | 53.330 | 46.575 | 15.019 | 1.00 | 41.70 | 6 | C |
| ATOM | 2124 | CB | VAL A | 275 | 53.374 | 45.397 | 14.027 | 1.00 | 41.14 | 6 | C |
| ATOM | 2125 | CG1 | VAL A | 275 | 52.021 | 44.784 | 13.716 | 1.00 | 39.29 | 6 | C |
| ATOM | 2126 | CG2 | VAL A | 275 | 54.052 | 45.839 | 12.731 | 1.00 | 41.40 | 6 | C |
| ATOM | 2127 | C | VAL A | 275 | 54.648 | 46.796 | 15.732 | 1.00 | 40.65 | 6 | C |
| ATOM | 2128 | O | VAL A | 275 | 55.694 | 46.177 | 15.518 | 1.00 | 39.82 | 8 | O |
| ATOM | 2129 | N | PRO A | 276 | 54.650 | 47.682 | 16.749 | 1.00 | 40.69 | 7 | N |
| ATOM | 2130 | CD | PRO A | 276 | 53.528 | 48.537 | 17.188 | 1.00 | 40.98 | 6 | C |
| ATOM | 2131 | CA | PRO A | 276 | 55.862 | 48.028 | 17.466 | 1.00 | 39.58 | 6 | C |
| ATOM | 2132 | CB | PRO A | 276 | 55.483 | 49.298 | 18.235 | 1.00 | 39.57 | 6 | C |
| ATOM | 2133 | CG | PRO A | 276 | 54.027 | 49.103 | 18.522 | 1.00 | 39.75 | 6 | C |
| ATOM | 2134 | C | PRO A | 276 | 56.427 | 46.930 | 18.335 | 1.00 | 38.04 | 6 | C |
| ATOM | 2135 | O | PRO A | 276 | 57.654 | 46.789 | 18.332 | 1.00 | 38.45 | 8 | O |
| ATOM | 2136 | N | GLU A | 277 | 55.584 | 46.177 | 19.033 | 1.00 | 35.64 | 7 | N |
| ATOM | 2137 | CA | GLU A | 277 | 56.088 | 45.083 | 19.873 | 1.00 | 34.74 | 6 | C |
| ATOM | 2138 | CB | GLU A | 277 | 54.980 | 44.499 | 20.733 | 1.00 | 35.92 | 6 | C |
| ATOM | 2139 | CG | GLU A | 277 | 54.156 | 45.470 | 21.552 | 1.00 | 40.50 | 6 | C |
| ATOM | 2140 | CD | GLU A | 277 | 55.030 | 46.419 | 22.349 | 1.00 | 42.93 | 6 | C |
| ATOM | 2141 | OE1 | GLU A | 277 | 56.117 | 46.052 | 22.843 | 1.00 | 43.29 | 8 | O |
| ATOM | 2142 | OE2 | GLU A | 277 | 54.597 | 47.586 | 22.488 | 1.00 | 45.14 | 8 | O |
| ATOM | 2143 | C | GLU A | 277 | 56.692 | 43.992 | 18.996 | 1.00 | 29.74 | 6 | C |
| ATOM | 2144 | O | GLU A | 277 | 57.754 | 43.483 | 19.329 | 1.00 | 29.09 | 8 | O |
| ATOM | 2145 | N | ASN A | 278 | 55.964 | 43.624 | 17.941 | 1.00 | 31.27 | 7 | N |
| ATOM | 2146 | CA | ASN A | 278 | 56.509 | 42.599 | 17.005 | 1.00 | 29.44 | 6 | C |
| ATOM | 2147 | CB | ASN A | 278 | 55.378 | 42.277 | 15.999 | 1.00 | 28.98 | 6 | C |
| ATOM | 2148 | CG | ASN A | 278 | 54.446 | 41.209 | 16.554 | 1.00 | 30.11 | 6 | C |
| ATOM | 2149 | OD1 | ASN A | 278 | 54.835 | 40.018 | 16.559 | 1.00 | 29.75 | 8 | O |
| ATOM | 2150 | ND2 | ASN A | 278 | 53.256 | 41.547 | 17.009 | 1.00 | 27.06 | 7 | N |
| ATOM | 2151 | C | ASN A | 278 | 57.784 | 43.018 | 16.304 | 1.00 | 29.12 | 6 | C |
| ATOM | 2152 | O | ASN A | 278 | 58.769 | 42.245 | 16.175 | 1.00 | 29.06 | 8 | O |
| ATOM | 2153 | N | THR A | 279 | 57.903 | 44.290 | 15.912 | 1.00 | 29.75 | 7 | N |
| ATOM | 2154 | CA | THR A | 279 | 59.150 | 44.803 | 15.340 | 1.00 | 32.13 | 6 | C |
| ATOM | 2155 | CB | THR A | 279 | 58.957 | 46.188 | 14.698 | 1.00 | 34.38 | 6 | C |
| ATOM | 2156 | OG1 | THR A | 279 | 58.462 | 47.117 | 15.677 | 1.00 | 38.52 | 8 | O |
| ATOM | 2157 | CG2 | THR A | 279 | 57.911 | 46.153 | 13.591 | 1.00 | 33.74 | 6 | C |
| ATOM | 2158 | C | THR A | 279 | 60.299 | 44.775 | 16.332 | 1.00 | 30.10 | 6 | C |
| ATOM | 2159 | O | THR A | 279 | 61.374 | 44.206 | 16.112 | 1.00 | 29.36 | 8 | O |
| ATOM | 2160 | N | ARG A | 280 | 60.079 | 45.255 | 17.577 | 1.00 | 31.39 | 7 | N |
| ATOM | 2161 | CA | ARG A | 280 | 61.139 | 45.156 | 18.581 | 1.00 | 32.07 | 6 | C |
| ATOM | 2162 | CB | ARG A | 280 | 60.647 | 45.698 | 19.939 | 1.00 | 34.98 | 6 | C |
| ATOM | 2163 | CG | ARG A | 280 | 61.710 | 45.630 | 21.024 | 1.00 | 39.35 | 6 | C |
| ATOM | 2164 | CD | ARG A | 280 | 61.318 | 46.478 | 22.238 | 1.00 | 43.50 | 6 | C |
| ATOM | 2165 | NE | ARG A | 280 | 60.534 | 45.725 | 23.187 | 1.00 | 47.87 | 7 | N |
| ATOM | 2166 | CZ | ARG A | 280 | 60.898 | 44.892 | 24.146 | 1.00 | 49.41 | 6 | C |
| ATOM | 2167 | NH1 | ARG A | 280 | 59.960 | 44.288 | 24.882 | 1.00 | 50.02 | 7 | N |
| ATOM | 2168 | NH2 | ARG A | 280 | 62.153 | 44.609 | 24.464 | 1.00 | 50.53 | 7 | N |
| ATOM | 2169 | C | ARG A | 280 | 61.646 | 43.736 | 18.758 | 1.00 | 28.75 | 6 | C |
| ATOM | 2170 | O | ARG A | 280 | 62.815 | 43.462 | 18.808 | 1.00 | 27.51 | 8 | O |
| ATOM | 2171 | N | LEU A | 281 | 60.733 | 42.757 | 18.957 | 1.00 | 29.52 | 7 | N |
| ATOM | 2172 | CA | LEU A | 281 | 61.115 | 41.378 | 19.148 | 1.00 | 29.08 | 6 | C |
| ATOM | 2173 | CB | LEU A | 281 | 59.878 | 40.482 | 19.401 | 1.00 | 30.92 | 6 | C |
| ATOM | 2174 | CG | LEU A | 281 | 59.000 | 40.892 | 20.605 | 1.00 | 33.95 | 6 | C |
| ATOM | 2175 | CD1 | LEU A | 281 | 57.733 | 40.042 | 20.666 | 1.00 | 32.63 | 6 | C |
| ATOM | 2176 | CD2 | LEU A | 281 | 59.817 | 40.766 | 21.876 | 1.00 | 33.55 | 6 | C |
| ATOM | 2177 | C | LEU A | 281 | 61.844 | 40.821 | 17.928 | 1.00 | 25.76 | 6 | C |
| ATOM | 2178 | O | LEU A | 281 | 62.818 | 40.076 | 18.070 | 1.00 | 21.64 | 8 | O |
| ATOM | 2179 | N | PHE A | 282 | 61.314 | 41.126 | 16.749 | 1.00 | 25.15 | 7 | N |
| ATOM | 2180 | CA | PHE A | 282 | 61.957 | 40.652 | 15.516 | 1.00 | 25.56 | 6 | C |
| ATOM | 2181 | CB | PHE A | 282 | 61.134 | 41.151 | 14.305 | 1.00 | 26.05 | 6 | C |
| ATOM | 2182 | CG | PHE A | 282 | 61.869 | 40.875 | 13.009 | 1.00 | 26.48 | 6 | C |
| ATOM | 2183 | CD1 | PHE A | 282 | 61.860 | 39.598 | 12.485 | 1.00 | 28.15 | 6 | C |
| ATOM | 2184 | CD2 | PHE A | 282 | 62.547 | 41.876 | 12.338 | 1.00 | 26.62 | 6 | C |
| ATOM | 2185 | CE1 | PHE A | 282 | 62.497 | 39.306 | 11.302 | 1.00 | 27.83 | 6 | C |
| ATOM | 2186 | CE2 | PHE A | 282 | 63.218 | 41.591 | 11.164 | 1.00 | 27.17 | 6 | C |
| ATOM | 2187 | CZ | PHE A | 282 | 63.201 | 40.302 | 10.662 | 1.00 | 29.01 | 6 | C |
| ATOM | 2188 | C | PHE A | 282 | 63.400 | 41.152 | 15.427 | 1.00 | 25.50 | 6 | C |
| ATOM | 2189 | O | PHE A | 282 | 64.317 | 40.363 | 15.278 | 1.00 | 24.99 | 8 | O |
| ATOM | 2190 | N | GLU A | 283 | 63.593 | 42.456 | 15.654 | 1.00 | 28.59 | 7 | N |
| ATOM | 2191 | CA | GLU A | 283 | 64.933 | 43.037 | 15.574 | 1.00 | 31.75 | 6 | C |
| ATOM | 2192 | CB | GLU A | 283 | 64.906 | 44.571 | 15.676 | 1.00 | 35.85 | 6 | C |
| ATOM | 2193 | CG | GLU A | 283 | 64.141 | 45.212 | 14.523 | 1.00 | 41.71 | 6 | C |
| ATOM | 2194 | CD | GLU A | 283 | 63.948 | 46.697 | 14.743 | 1.00 | 46.13 | 6 | C |
| ATOM | 2195 | OE1 | GLU A | 283 | 64.906 | 47.453 | 15.019 | 1.00 | 46.68 | 8 | O |
| ATOM | 2196 | OE2 | GLU A | 283 | 62.796 | 47.181 | 14.663 | 1.00 | 50.06 | 8 | O |
| ATOM | 2197 | C | GLU A | 283 | 65.865 | 42.483 | 16.641 | 1.00 | 29.73 | 6 | C |
| ATOM | 2198 | O | GLU A | 283 | 67.012 | 42.217 | 16.301 | 1.00 | 30.42 | 8 | O |
| ATOM | 2199 | N | MET A | 284 | 65.361 | 42.288 | 17.857 | 1.00 | 28.92 | 7 | N |
| ATOM | 2200 | CA | MET A | 284 | 66.143 | 41.676 | 18.907 | 1.00 | 28.74 | 6 | C |
| ATOM | 2201 | CB | MET A | 284 | 65.328 | 41.555 | 20.217 | 1.00 | 29.44 | 6 | C |
| ATOM | 2202 | CG | MET A | 284 | 64.839 | 42.819 | 20.892 | 1.00 | 31.83 | 6 | C |
| ATOM | 2203 | SD | MET A | 284 | 64.349 | 42.591 | 22.632 | 1.00 | 35.57 | 16 | S |
| ATOM | 2204 | CE | MET A | 284 | 65.824 | 41.621 | 23.060 | 1.00 | 26.72 | 6 | C |
| ATOM | 2205 | C | MET A | 284 | 66.665 | 40.306 | 18.567 | 1.00 | 25.37 | 6 | C |
| ATOM | 2206 | O | MET A | 284 | 67.827 | 39.970 | 18.774 | 1.00 | 27.80 | 8 | O |
| ATOM | 2207 | N | VAL A | 285 | 65.822 | 39.360 | 18.112 | 1.00 | 24.69 | 7 | N |
| ATOM | 2208 | CA | VAL A | 285 | 66.194 | 38.035 | 17.722 | 1.00 | 22.10 | 6 | C |
| ATOM | 2209 | CB | VAL A | 285 | 64.938 | 37.209 | 17.237 | 1.00 | 22.25 | 6 | C |
| ATOM | 2210 | CG1 | VAL A | 285 | 65.445 | 35.907 | 16.678 | 1.00 | 21.99 | 6 | C |
| ATOM | 2211 | CG2 | VAL A | 285 | 64.146 | 36.970 | 18.526 | 1.00 | 22.56 | 6 | C |
| ATOM | 2212 | C | VAL A | 285 | 67.239 | 38.084 | 16.574 | 1.00 | 22.61 | 6 | C |
| ATOM | 2213 | O | VAL A | 285 | 68.205 | 37.337 | 16.576 | 1.00 | 25.80 | 8 | O |
| ATOM | 2214 | N | LYS A | 286 | 67.045 | 38.916 | 15.598 | 1.00 | 26.81 | 7 | N |
| ATOM | 2215 | CA | LYS A | 286 | 67.995 | 39.104 | 14.490 | 1.00 | 30.63 | 6 | C |
| ATOM | 2216 | CB | LYS A | 286 | 67.449 | 40.148 | 13.520 | 1.00 | 32.69 | 6 | C |
| ATOM | 2217 | CG | LYS A | 286 | 66.202 | 39.697 | 12.760 | 1.00 | 33.80 | 6 | C |
| ATOM | 2218 | CD | LYS A | 286 | 66.463 | 38.370 | 12.067 | 1.00 | 35.04 | 6 | C |
| ATOM | 2219 | CE | LYS A | 286 | 67.293 | 38.445 | 10.807 | 1.00 | 34.54 | 6 | C |
| ATOM | 2220 | NZ | LYS A | 286 | 67.550 | 37.072 | 10.261 | 1.00 | 35.90 | 7 | N |
| ATOM | 2221 | C | LYS A | 286 | 69.376 | 39.526 | 15.006 | 1.00 | 30.82 | 6 | C |
| ATOM | 2222 | O | LYS A | 286 | 70.396 | 39.036 | 14.508 | 1.00 | 32.76 | 8 | O |
| ATOM | 2223 | N | ARG A | 287 | 69.431 | 40.400 | 16.013 | 1.00 | 31.10 | 7 | N |
| ATOM | 2224 | CA | ARG A | 287 | 70.714 | 40.816 | 16.604 | 1.00 | 30.96 | 6 | C |
| ATOM | 2225 | CB | ARG A | 287 | 70.583 | 42.005 | 17.529 | 1.00 | 30.38 | 6 | C |
| ATOM | 2226 | CG | ARG A | 287 | 70.116 | 43.375 | 17.152 | 1.00 | 32.68 | 6 | C |
| ATOM | 2227 | CD | ARG A | 287 | 69.484 | 44.170 | 18.348 | 1.00 | 33.81 | 6 | C |
| ATOM | 2228 | NE | ARG A | 287 | 68.533 | 45.133 | 17.764 | 1.00 | 36.54 | 7 | N |
| ATOM | 2229 | CZ | ARG A | 287 | 67.488 | 45.691 | 18.267 | 1.00 | 34.85 | 6 | C |
| ATOM | 2230 | NH1 | ARG A | 287 | 67.137 | 45.373 | 19.538 | 1.00 | 35.39 | 7 | N |
| ATOM | 2231 | NH2 | ARG A | 287 | 66.748 | 46.556 | 17.570 | 1.00 | 36.57 | 7 | N |
| ATOM | 2232 | C | ARG A | 287 | 71.356 | 39.638 | 17.308 | 1.00 | 32.30 | 6 | C |
| ATOM | 2233 | O | ARG A | 287 | 72.542 | 39.411 | 17.109 | 1.00 | 33.47 | 8 | O |
| ATOM | 2234 | N | LYS A | 288 | 70.600 | 38.785 | 18.034 | 1.00 | 32.62 | 7 | N |
| ATOM | 2235 | CA | LYS A | 288 | 71.145 | 37.597 | 18.640 | 1.00 | 33.94 | 6 | C |
| ATOM | 2236 | CB | LYS A | 288 | 70.111 | 36.854 | 19.507 | 1.00 | 35.23 | 6 | C |
| ATOM | 2237 | CG | LYS A | 288 | 69.608 | 37.710 | 20.659 | 1.00 | 35.37 | 6 | C |
| ATOM | 2238 | CD | LYS A | 288 | 68.867 | 36.870 | 21.687 | 1.00 | 37.31 | 6 | C |
| ATOM | 2239 | CE | LYS A | 288 | 68.151 | 37.797 | 22.658 | 1.00 | 37.75 | 6 | C |
| ATOM | 2240 | NZ | LYS A | 288 | 68.902 | 37.955 | 23.939 | 1.00 | 38.18 | 7 | N |
| ATOM | 2241 | C | LYS A | 288 | 71.713 | 36.605 | 17.641 | 1.00 | 37.09 | 6 | C |
| ATOM | 2242 | O | LYS A | 288 | 72.668 | 35.903 | 17.972 | 1.00 | 36.11 | 8 | O |
| ATOM | 2243 | N | GLU A | 289 | 71.075 | 36.440 | 16.481 | 1.00 | 41.18 | 7 | N |
| ATOM | 2244 | CA | GLU A | 289 | 71.519 | 35.514 | 15.452 | 1.00 | 44.56 | 6 | C |
| ATOM | 2245 | CB | GLU A | 289 | 70.568 | 35.544 | 14.260 | 1.00 | 46.83 | 6 | C |
| ATOM | 2246 | CG | GLU A | 289 | 69.201 | 34.909 | 14.469 | 1.00 | 49.19 | 6 | C |
| ATOM | 2247 | CD | GLU A | 289 | 68.529 | 34.899 | 13.100 | 1.00 | 50.64 | 6 | C |
| ATOM | 2248 | OE1 | GLU A | 289 | 68.442 | 36.038 | 12.597 | 1.00 | 52.06 | 8 | O |
| ATOM | 2249 | OE2 | GLU A | 289 | 68.146 | 33.861 | 12.537 | 1.00 | 51.29 | 8 | O |
| ATOM | 2250 | C | GLU A | 289 | 72.890 | 35.910 | 14.891 | 1.00 | 45.68 | 6 | C |
| ATOM | 2251 | O | GLU A | 289 | 73.818 | 35.137 | 14.677 | 1.00 | 44.19 | 8 | O |
| ATOM | 2252 | N | SER A | 290 | 73.013 | 37.221 | 14.674 | 1.00 | 48.65 | 7 | N |
| ATOM | 2253 | CA | SER A | 290 | 74.306 | 37.727 | 14.231 | 1.00 | 52.68 | 6 | C |
| ATOM | 2254 | CB | SER A | 290 | 74.278 | 39.186 | 13.806 | 1.00 | 52.54 | 6 | C |
| ATOM | 2255 | OG | SER A | 290 | 75.677 | 39.491 | 13.632 | 1.00 | 53.21 | 8 | O |
| ATOM | 2256 | C | SER A | 290 | 75.323 | 37.552 | 15.371 | 1.00 | 55.65 | 6 | C |
| ATOM | 2257 | O | SER A | 290 | 76.478 | 37.189 | 15.146 | 1.00 | 55.72 | 8 | O |
| ATOM | 2258 | N | GLU A | 291 | 74.872 | 37.757 | 16.592 | 1.00 | 58.14 | 7 | N |
| ATOM | 2259 | CA | GLU A | 291 | 75.584 | 37.641 | 17.833 | 1.00 | 61.11 | 6 | C |
| ATOM | 2260 | CB | GLU A | 291 | 76.917 | 36.906 | 17.785 | 1.00 | 62.15 | 6 | C |
| ATOM | 2261 | CG | GLU A | 291 | 76.937 | 35.556 | 18.465 | 1.00 | 64.10 | 6 | C |
| ATOM | 2262 | CD | GLU A | 291 | 76.620 | 34.394 | 17.555 | 1.00 | 65.44 | 6 | C |
| ATOM | 2263 | OE1 | GLU A | 291 | 76.734 | 33.228 | 17.991 | 1.00 | 66.46 | 8 | O |
| ATOM | 2264 | OE2 | GLU A | 291 | 76.263 | 34.563 | 16.379 | 1.00 | 66.51 | 8 | O |
| ATOM | 2265 | C | GLU A | 291 | 75.826 | 39.022 | 18.451 | 1.00 | 61.44 | 6 | C |
| ATOM | 2266 | O | GLU A | 291 | 75.122 | 39.336 | 19.425 | 1.00 | 62.15 | 8 | O |
| ATOM | 2267 | OT | GLU A | 291 | 76.698 | 39.760 | 17.949 | 1.00 | 62.85 | 8 | O |
| ATOM | 2268 | OW0 | WAT W | 1 | 50.217 | 13.385 | 8.209 | 1.00 | 18.85 | 8 | O |
| ATOM | 2269 | OW0 | WAT W | 2 | 51.099 | 13.263 | −8.741 | 1.00 | 23.12 | 8 | O |
| ATOM | 2270 | OW0 | WAT W | 3 | 59.588 | 23.064 | 3.410 | 1.00 | 20.25 | 8 | O |
| ATOM | 2271 | OW0 | WAT W | 4 | 44.433 | 27.839 | −0.089 | 1.00 | 21.90 | 8 | O |
| ATOM | 2272 | OW0 | WAT W | 5 | 62.584 | 30.723 | 1.451 | 1.00 | 21.49 | 8 | O |
| ATOM | 2273 | OW0 | WAT W | 6 | 53.660 | 4.312 | 5.088 | 1.00 | 23.56 | 8 | O |
| ATOM | 2274 | OW0 | WAT W | 7 | 42.461 | 16.502 | −7.442 | 1.00 | 19.28 | 8 | O |
| ATOM | 2275 | OW0 | WAT W | 8 | 50.471 | 4.408 | −0.984 | 1.00 | 28.60 | 8 | O |
| ATOM | 2276 | OW0 | WAT W | 9 | 49.743 | 18.658 | −13.374 | 1.00 | 32.32 | 8 | O |
| ATOM | 2277 | OW0 | WAT W | 10 | 44.689 | 29.884 | 1.451 | 1.00 | 28.29 | 8 | O |
| ATOM | 2278 | OW0 | WAT W | 11 | 49.899 | 34.479 | 2.565 | 1.00 | 38.46 | 8 | O |
| ATOM | 2279 | OW0 | WAT W | 12 | 41.559 | 11.016 | −5.279 | 1.00 | 27.37 | 8 | O |
| ATOM | 2280 | OW0 | WAT W | 13 | 46.109 | 24.308 | 29.035 | 1.00 | 30.91 | 8 | O |
| ATOM | 2281 | OW0 | WAT W | 14 | 53.259 | 27.112 | 7.872 | 1.00 | 28.13 | 8 | O |
| ATOM | 2282 | OW0 | WAT W | 15 | 65.303 | 30.595 | 0.296 | 1.00 | 26.04 | 8 | O |
| ATOM | 2283 | OW0 | WAT W | 16 | 58.733 | 26.677 | −12.881 | 1.00 | 28.60 | 8 | O |
| ATOM | 2284 | OW0 | WAT W | 17 | 61.058 | 27.725 | 25.573 | 1.00 | 28.38 | 8 | O |
| ATOM | 2285 | OW0 | WAT W | 18 | 56.024 | 5.831 | 13.865 | 1.00 | 26.82 | 8 | O |
| ATOM | 2286 | OW0 | WAT W | 19 | 57.892 | 36.531 | 2.311 | 1.00 | 32.51 | 8 | O |
| ATOM | 2287 | OW0 | WAT W | 20 | 64.032 | 17.458 | 6.356 | 1.00 | 25.67 | 8 | O |
| ATOM | 2288 | OW0 | WAT W | 21 | 53.531 | 26.503 | 2.690 | 1.00 | 24.96 | 8 | O |
| ATOM | 2289 | OW0 | WAT W | 22 | 48.109 | 10.443 | 12.991 | 1.00 | 33.12 | 8 | O |
| ATOM | 2290 | OW0 | WAT W | 23 | 63.052 | 29.353 | 14.789 | 1.00 | 27.25 | 8 | O |
| ATOM | 2291 | OW0 | WAT W | 24 | 58.658 | 19.577 | −18.215 | 1.00 | 31.14 | 8 | O |
| ATOM | 2292 | OW0 | WAT W | 25 | 69.574 | 45.629 | 15.048 | 1.00 | 23.96 | 8 | O |
| ATOM | 2293 | OW0 | WAT W | 26 | 64.033 | 32.895 | −3.472 | 1.00 | 30.38 | 8 | O |
| ATOM | 2294 | OW0 | WAT W | 27 | 45.265 | 26.382 | 30.985 | 1.00 | 37.20 | 8 | O |
| ATOM | 2296 | OW0 | WAT W | 29 | 64.316 | 33.139 | 9.120 | 1.00 | 28.20 | 8 | O |
| ATOM | 2297 | OW0 | WAT W | 30 | 69.017 | 43.023 | 14.050 | 1.00 | 36.84 | 8 | O |
| ATOM | 2298 | OW0 | WAT W | 31 | 56.424 | 6.042 | 9.451 | 1.00 | 45.62 | 8 | O |
| ATOM | 2299 | OW0 | WAT W | 32 | 65.473 | 25.882 | −12.349 | 1.00 | 31.77 | 8 | O |
| ATOM | 2300 | OW0 | WAT W | 33 | 54.047 | 28.809 | 12.233 | 1.00 | 32.98 | 8 | O |
| ATOM | 2301 | OW0 | WAT W | 34 | 71.715 | 11.099 | 21.805 | 1.00 | 28.72 | 8 | O |
| ATOM | 2302 | OW0 | WAT W | 35 | 44.866 | 23.772 | −10.264 | 1.00 | 31.01 | 8 | O |
| ATOM | 2303 | OW0 | WAT W | 36 | 42.891 | 8.225 | 0.183 | 1.00 | 45.65 | 8 | O |
| ATOM | 2304 | OW0 | WAT W | 37 | 70.091 | 9.899 | 9.438 | 1.00 | 42.64 | 8 | O |
| ATOM | 2305 | OW0 | WAT W | 38 | 55.173 | 20.751 | 30.386 | 1.00 | 47.17 | 8 | O |
| ATOM | 2306 | OW0 | WAT W | 39 | 64.556 | −1.132 | 7.808 | 1.00 | 41.30 | 8 | O |
| ATOM | 2307 | OW0 | WAT W | 40 | 43.741 | 44.189 | 17.880 | 1.00 | 46.34 | 8 | O |
| ATOM | 2308 | OW0 | WAT W | 41 | 65.303 | 17.923 | 15.681 | 1.00 | 36.89 | 8 | O |
| ATOM | 2309 | OW0 | WAT W | 42 | 65.926 | 5.299 | −3.431 | 1.00 | 35.15 | 8 | O |
| ATOM | 2310 | OW0 | WAT W | 43 | 48.814 | 25.326 | −10.907 | 1.00 | 36.32 | 8 | O |
| ATOM | 2311 | OW0 | WAT W | 44 | 71.256 | 20.768 | −5.622 | 1.00 | 45.70 | 8 | O |
| ATOM | 2312 | OW0 | WAT W | 45 | 43.487 | 7.489 | 10.572 | 1.00 | 41.45 | 8 | O |
| ATOM | 2313 | OW0 | WAT W | 46 | 53.848 | 5.218 | 8.719 | 1.00 | 47.18 | 8 | O |
| ATOM | 2314 | OW0 | WAT W | 47 | 65.635 | 33.112 | 2.964 | 1.00 | 34.64 | 8 | O |
| ATOM | 2315 | OW0 | WAT W | 48 | 40.726 | 28.714 | 23.437 | 1.00 | 38.77 | 8 | O |
| ATOM | 2316 | OW0 | WAT W | 49 | 43.440 | 32.012 | 14.188 | 1.00 | 44.70 | 8 | O |
| ATOM | 2317 | OW0 | WAT W | 50 | 76.019 | 8.562 | 0.201 | 1.00 | 32.02 | 8 | O |
| ATOM | 2318 | OW0 | WAT W | 51 | 61.127 | 48.537 | 25.049 | 1.00 | 56.13 | 8 | O |
| ATOM | 2319 | OW0 | WAT W | 52 | 65.781 | 33.797 | −0.396 | 1.00 | 45.54 | 8 | O |
| ATOM | 2320 | OW0 | WAT W | 53 | 42.177 | 13.006 | 3.059 | 1.00 | 41.61 | 8 | O |
| ATOM | 2321 | OW0 | WAT W | 54 | 66.903 | 22.848 | −12.476 | 1.00 | 29.57 | 8 | O |
| ATOM | 2322 | OW0 | WAT W | 55 | 42.048 | 30.487 | −1.394 | 1.00 | 56.64 | 8 | O |
| ATOM | 2323 | OW0 | WAT W | 56 | 60.361 | 22.604 | −15.611 | 1.00 | 32.16 | 8 | O |
| ATOM | 2324 | OW0 | WAT W | 57 | 52.219 | 28.431 | 10.189 | 1.00 | 27.74 | 8 | O |
| ATOM | 2325 | OW0 | WAT W | 58 | 49.598 | 30.240 | 30.888 | 1.00 | 37.18 | 8 | O |
| ATOM | 2326 | OW0 | WAT W | 59 | 64.471 | 34.651 | −7.853 | 1.00 | 40.08 | 8 | O |
| ATOM | 2327 | OW0 | WAT W | 60 | 37.370 | 33.351 | 9.660 | 1.00 | 41.05 | 8 | O |
| ATOM | 2328 | OW0 | WAT W | 61 | 59.810 | 24.408 | 9.179 | 1.00 | 24.99 | 8 | O |
| ATOM | 2329 | OW0 | WAT W | 62 | 54.904 | 22.048 | 9.493 | 1.00 | 26.52 | 8 | O |
| ATOM | 2330 | OW0 | WAT W | 63 | 59.801 | 22.030 | 7.519 | 1.00 | 31.69 | 8 | O |
| ATOM | 2331 | OW0 | WAT W | 64 | 61.589 | 20.737 | 9.336 | 1.00 | 37.29 | 8 | O |
| ATOM | 2332 | OW0 | WAT W | 65 | 54.273 | 30.364 | 2.612 | 1.00 | 31.63 | 8 | O |
| ATOM | 2333 | OW0 | WAT W | 66 | 51.721 | 9.464 | 18.710 | 1.00 | 41.21 | 8 | O |
| ATOM | 2334 | OW0 | WAT W | 67 | 39.698 | 15.341 | −4.655 | 1.00 | 41.50 | 8 | O |
| ATOM | 2335 | OW0 | WAT W | 68 | 65.849 | 18.347 | 10.526 | 1.00 | 36.90 | 8 | O |
| ATOM | 2336 | OW0 | WAT W | 69 | 63.148 | 4.922 | −12.137 | 1.00 | 44.78 | 8 | O |
| ATOM | 2337 | OW0 | WAT W | 70 | 59.514 | 48.884 | 18.189 | 1.00 | 41.71 | 8 | O |
| ATOM | 2338 | OW0 | WAT W | 71 | 67.904 | 3.430 | 15.950 | 1.00 | 29.67 | 8 | O |
| ATOM | 2339 | OW0 | WAT W | 72 | 60.457 | 28.514 | −12.39O | 1.00 | 41.62 | 8 | O |
| ATOM | 2340 | OW0 | WAT W | 73 | 64.593 | 19.854 | 7.512 | 1.00 | 38.96 | 8 | O |
| ATOM | 2341 | OW0 | WAT W | 74 | 62.953 | 18.712 | 9.638 | 1.00 | 31.50 | 8 | O |
| ATOM | 2342 | OW0 | WAT W | 75 | 58.999 | 18.160 | 14.139 | 1.00 | 36.51 | 8 | O |
| ATOM | 2343 | OW0 | WAT W | 76 | 60.140 | 11.877 | 22.642 | 1.00 | 50.94 | 8 | O |
| ATOM | 2344 | OW0 | WAT W | 77 | 45.591 | 40.024 | 2.139 | 1.00 | 79.57 | 8 | O |
| ATOM | 2345 | OW0 | WAT W | 78 | 69.205 | 23.538 | 21.104 | 1.00 | 37.43 | 8 | O |
| ATOM | 2346 | OW0 | WAT W | 79 | 41.543 | 31.614 | 11.031 | 1.00 | 59.04 | 8 | O |
| ATOM | 2347 | OW0 | WAT W | 80 | 68.389 | 27.416 | 1.198 | 1.00 | 62.23 | 8 | O |
| ATOM | 2348 | OW0 | WAT W | 81 | 44.130 | 10.194 | −5.136 | 1.00 | 36.84 | 8 | O |
| ATOM | 2349 | OW0 | WAT W | 82 | 66.868 | 21.240 | 9.977 | 1.00 | 37.21 | 8 | O |
| ATOM | 2350 | OW0 | WAT W | 83 | 58.659 | 17.118 | −18.714 | 1.00 | 35.94 | 8 | O |
| ATOM | 2351 | OW0 | WAT W | 84 | 48.207 | 28.942 | −15.536 | 1.00 | 80.94 | 8 | O |
| ATOM | 2352 | OW0 | WAT W | 85 | 59.985 | 35.614 | −7.715 | 1.00 | 63.41 | 8 | O |
| ATOM | 2353 | OW0 | WAT W | 86 | 68.848 | 7.807 | −12.897 | 1.00 | 54.20 | 8 | O |
| ATOM | 2354 | OW0 | WAT W | 87 | 50.732 | 8.107 | 10.734 | 1.00 | 29.14 | 8 | O |
| ATOM | 2355 | OW0 | WAT W | 88 | 57.598 | 31.502 | −15.596 | 1.00 | 67.97 | 8 | O |
| ATOM | 2356 | OW0 | WAT W | 89 | 45.523 | 35.059 | 4.411 | 1.00 | 43.42 | 8 | O |
| ATOM | 2357 | OW0 | WAT W | 90 | 43.953 | 31.511 | 16.602 | 1.00 | 58.48 | 8 | O |
| ATOM | 2358 | OW0 | WAT W | 91 | 62.804 | 19.278 | 16.014 | 1.00 | 48.73 | 8 | O |
| ATOM | 2359 | OW0 | WAT W | 92 | 55.227 | 19.291 | 15.486 | 1.00 | 41.12 | 8 | O |
| ATOM | 2360 | OW0 | WAT W | 93 | 74.125 | 5.232 | 2.711 | 1.00 | 37.31 | 8 | O |
| ATOM | 2361 | OW0 | WAT W | 94 | 47.921 | 47.666 | 16.091 | 1.00 | 46.05 | 8 | O |
| ATOM | 2362 | OW0 | WAT W | 95 | 44.648 | 30.547 | −2.533 | 1.00 | 56.72 | 8 | O |
| ATOM | 2363 | OW0 | WAT W | 96 | 43.159 | 13.302 | 12.753 | 1.00 | 31.02 | 8 | O |
| ATOM | 2364 | OW0 | WAT W | 97 | 76.289 | 13.068 | −6.287 | 1.00 | 60.16 | 8 | O |
| ATOM | 2365 | OW0 | WAT W | 98 | 40.454 | 31.194 | 22.897 | 1.00 | 47.18 | 8 | O |
| ATOM | 2366 | OW0 | WAT W | 99 | 56.619 | 24.955 | 22.006 | 1.00 | 34.02 | 8 | O |
| ATOM | 2367 | OW0 | WAT W | 100 | 57.214 | 26.810 | 17.742 | 1.00 | 42.53 | 8 | O |
| ATOM | 2368 | OW0 | WAT W | 101 | 72.485 | 31.810 | 15.402 | 1.00 | 48.53 | 8 | O |
| ATOM | 2369 | OW0 | WAT W | 102 | 58.554 | 25.994 | 33.289 | 1.00 | 42.04 | 8 | O |
| ATOM | 2370 | OW0 | WAT W | 103 | 58.901 | 33.000 | −8.347 | 1.00 | 35.50 | 8 | O |
| ATOM | 2371 | OW0 | WAT W | 104 | 48.684 | 42.585 | −0.562 | 1.00 | 53.83 | 8 | O |
| ATOM | 2372 | OW0 | WAT W | 105 | 72.840 | 7.443 | −6.312 | 1.00 | 34.41 | 8 | O |
| ATOM | 2373 | OW0 | WAT W | 106 | 60.649 | 41.443 | 6.936 | 1.00 | 46.14 | 8 | O |
| ATOM | 2374 | OW0 | WAT W | 107 | 55.930 | 20.902 | 12.923 | 1.00 | 42.34 | 8 | O |
| ATOM | 2375 | OW0 | WAT W | 108 | 56.276 | 10.614 | 22.445 | 1.00 | 83.32 | 8 | O |
| ATOM | 2376 | OW0 | WAT W | 109 | 59.177 | −2.036 | 11.105 | 1.00 | 87.00 | 8 | O |
| ATOM | 2377 | OW0 | WAT W | 110 | 68.330 | 19.216 | 11.389 | 1.00 | 70.09 | 8 | O |
| ATOM | 2378 | OW0 | WAT W | 111 | 62.535 | 3.830 | −3.900 | 1.00 | 39.80 | 8 | O |
| ATOM | 2379 | OW0 | WAT W | 112 | 41.144 | 15.596 | 2.174 | 1.00 | 40.32 | 8 | O |
| ATOM | 2380 | OW0 | WAT W | 113 | 69.370 | 17.211 | 4.420 | 1.00 | 48.23 | 8 | O |
| ATOM | 2381 | OW0 | WAT W | 114 | 68.319 | 23.527 | 25.851 | 1.00 | 57.14 | 8 | O |
| ATOM | 2382 | OW0 | WAT W | 115 | 64.404 | 45.735 | 19.468 | 1.00 | 41.92 | 8 | O |
| ATOM | 2383 | OW0 | WAT W | 116 | 45.942 | 2.698 | −1.562 | 1.00 | 76.01 | 8 | O |
| ATOM | 2384 | OW0 | WAT W | 117 | 57.931 | 44.072 | 22.182 | 1.00 | 40.11 | 8 | O |
| ATOM | 2386 | OW0 | WAT W | 119 | 56.524 | 7.343 | −12.363 | 1.00 | 48.25 | 8 | O |
| ATOM | 2387 | OW0 | WAT W | 120 | 40.832 | 27.869 | −4.695 | 1.00 | 68.68 | 8 | O |
| ATOM | 2388 | OW0 | WAT W | 121 | 50.854 | 48.038 | 19.585 | 1.00 | 48.13 | 8 | O |
| ATOM | 2389 | OW0 | WAT W | 122 | 51.900 | 26.821 | −17.165 | 1.00 | 87.36 | 8 | O |
| ATOM | 2390 | OW0 | WAT W | 123 | 59.155 | 22.302 | 22.180 | 1.00 | 39.59 | 8 | O |
| ATOM | 2391 | OW0 | WAT W | 124 | 61.429 | 20.643 | −17.207 | 1.00 | 56.75 | 8 | O |
| ATOM | 2392 | OW0 | WAT W | 125 | 60.712 | 37.193 | 1.919 | 1.00 | 55.44 | 8 | O |
| ATOM | 2393 | OW0 | WAT W | 126 | 73.565 | 18.085 | 2.425 | 1.00 | 68.15 | 8 | O |
| ATOM | 2394 | OW0 | WAT W | 127 | 59.651 | 30.426 | 35.039 | 1.00 | 33.15 | 8 | O |
| ATOM | 2395 | OW0 | WAT W | 128 | 62.379 | 37.582 | 4.161 | 1.00 | 61.18 | 8 | O |
| ATOM | 2396 | OW0 | WAT W | 129 | 70.181 | 31.887 | 16.650 | 1.00 | 36.26 | 8 | O |
| ATOM | 2397 | OW0 | WAT W | 130 | 37.334 | 19.934 | −1.505 | 1.00 | 58.69 | 8 | O |
| ATOM | 2398 | OW0 | WAT W | 131 | 61.399 | 20.771 | 25.090 | 1.00 | 50.02 | 8 | O |
| ATOM | 2399 | OW0 | WAT W | 132 | 68.076 | 35.145 | 25.475 | 1.00 | 37.05 | 8 | O |
| ATOM | 2400 | OW0 | WAT W | 133 | 60.505 | 7.047 | −14.800 | 1.00 | 37.95 | 8 | O |
| ATOM | 2401 | OW0 | WAT W | 134 | 54.115 | 16.074 | 7.368 | 1.00 | 38.68 | 8 | O |
| ATOM | 2402 | OW0 | WAT W | 135 | 70.773 | 38.203 | 11.598 | 1.00 | 65.07 | 8 | O |
| ATOM | 2403 | OW0 | WAT W | 136 | 70.458 | 19.171 | 2.818 | 1.00 | 47.13 | 8 | O |
| ATOM | 2404 | OW0 | WAT W | 137 | 50.264 | 27.459 | −12.167 | 1.00 | 94.45 | 8 | O |
| ATOM | 2405 | OW0 | WAT W | 138 | 72.688 | 2.033 | 4.919 | 1.00 | 55.96 | 8 | O |
| ATOM | 2406 | OW0 | WAT W | 139 | 66.483 | 0.919 | −1.206 | 1.00 | 68.63 | 8 | O |
| ATOM | 2407 | OW0 | WAT W | 140 | 44.865 | 5.901 | 6.299 | 1.00 | 49.31 | 8 | O |
| ATOM | 2408 | OW0 | WAT W | 141 | 52.797 | 4.031 | 18.728 | 1.00 | 51.39 | 8 | O |
| ATOM | 2410 | OW0 | WAT W | 143 | 45.567 | 39.187 | 29.528 | 1.00 | 55.02 | 8 | O |
| ATOM | 2411 | OW0 | WAT W | 144 | 75.438 | 16.139 | 2.640 | 1.00 | 50.00 | 8 | O |
| ATOM | 2412 | OW0 | WAT W | 145 | 69.844 | 33.346 | 6.328 | 1.00 | 59.27 | 8 | O |
| ATOM | 2413 | OW0 | WAT W | 146 | 39.466 | 30.238 | −0.842 | 1.00 | 56.38 | 8 | O |
| ATOM | 2414 | OW0 | WAT W | 147 | 44.062 | 5.699 | 8.806 | 1.00 | 52.80 | 8 | O |
| ATOM | 2415 | OW0 | WAT W | 148 | 59.516 | 1.238 | −5.799 | 1.00 | 41.14 | 8 | O |
| ATOM | 2416 | OW0 | WAT W | 149 | 47.532 | 2.806 | 3.371 | 1.00 | 51.28 | 8 | O |
| ATOM | 2418 | OW0 | WAT W | 151 | 43.200 | 31.185 | 29.288 | 1.00 | 44.17 | 8 | O |
| ATOM | 2419 | OW0 | WAT W | 152 | 53.785 | 43.131 | −0.198 | 1.00 | 81.11 | 8 | O |
| ATOM | 2420 | OW0 | WAT W | 153 | 64.038 | −2.169 | 5.299 | 1.00 | 46.34 | 8 | O |
| ATOM | 2421 | OW0 | WAT W | 154 | 38.979 | 21.681 | −8.628 | 1.00 | 36.79 | 8 | O |
| ATOM | 2422 | OW0 | WAT W | 155 | 58.390 | 19.129 | 16.402 | 1.00 | 55.19 | 8 | O |
| ATOM | 2423 | OW0 | WAT W | 156 | 68.504 | 22.779 | 8.616 | 1.00 | 40.79 | 8 | O |
| ATOM | 2424 | OW0 | WAT W | 157 | 55.387 | 40.436 | 34.674 | 1.00 | 49.21 | 8 | O |
| ATOM | 2425 | OW0 | WAT W | 158 | 53.019 | 39.301 | −1.129 | 1.00 | 96.43 | 8 | O |
| ATOM | 2426 | OW0 | WAT W | 159 | 41.477 | 15.278 | −1.137 | 1.00 | 32.11 | 8 | O |
| ATOM | 2427 | OW0 | WAT W | 160 | 38.535 | 9.180 | −2.033 | 1.00 | 46.62 | 8 | O |
| ATOM | 2428 | OW0 | WAT W | 161 | 60.419 | 23.551 | 11.903 | 1.00 | 57.65 | 8 | O |
| ATOM | 2429 | OW0 | WAT W | 162 | 47.468 | 44.822 | 27.591 | 1.00 | 55.55 | 8 | O |
| ATOM | 2430 | OW0 | WAT W | 163 | 56.215 | −1.570 | 0.715 | 1.00 | 75.61 | 8 | O |
| ATOM | 2431 | OW0 | WAT W | 164 | 62.001 | 48.322 | 17.294 | 1.00 | 60.03 | 8 | O |
| ATOM | 2432 | OW0 | WAT W | 165 | 44.207 | 22.903 | −12.975 | 1.00 | 33.12 | 8 | O |
| ATOM | 2433 | OW0 | WAT W | 166 | 65.534 | 18.704 | 13.254 | 1.00 | 58.88 | 8 | O |
| ATOM | 2434 | OW0 | WAT W | 167 | 36.524 | 38.366 | 27.592 | 1.00 | 96.00 | 8 | O |
| ATOM | 2435 | OW0 | WAT W | 168 | 52.063 | 6.514 | 19.512 | 1.00 | 58.91 | 8 | O |
| ATOM | 2436 | OW0 | WAT W | 169 | 72.416 | 10.815 | 4.633 | 1.00 | 37.38 | 8 | O |
| ATOM | 2437 | OW0 | WAT W | 170 | 36.786 | 39.018 | 22.128 | 1.00 | 47.31 | 8 | O |
| ATOM | 2438 | OW0 | WAT W | 171 | 55.889 | 4.109 | −8.888 | 1.00 | 45.08 | 8 | O |
| ATOM | 2439 | OW0 | WAT W | 172 | 70.959 | 4.313 | 6.468 | 1.00 | 39.12 | 8 | O |
| ATOM | 2440 | OW0 | WAT W | 173 | 60.991 | 15.486 | 21.392 | 1.00 | 46.15 | 8 | O |
| ATOM | 2441 | OW0 | WAT W | 174 | 65.401 | 7.600 | −12.565 | 1.00 | 43.95 | 8 | O |
| ATOM | 2442 | OW0 | WAT W | 175 | 53.786 | 48.388 | 9.725 | 1.00 | 41.68 | 8 | O |
| ATOM | 2443 | OW0 | WAT W | 176 | 53.110 | 5.798 | 13.932 | 1.00 | 33.46 | 8 | O |
| ATOM | 2444 | OW0 | WAT W | 177 | 58.883 | 4.598 | −12.625 | 1.00 | 44.97 | 8 | O |
| ATOM | 2445 | OW0 | WAT W | 178 | 62.227 | 39.315 | 7.259 | 1.00 | 42.67 | 8 | O |
| ATOM | 2446 | OW0 | WAT W | 179 | 73.940 | 41.878 | 19.168 | 1.00 | 51.61 | 8 | O |
| ATOM | 2447 | OW0 | WAT W | 180 | 68.342 | 19.961 | 1.138 | 1.00 | 50.35 | 8 | O |
| ATOM | 2448 | OW0 | WAT W | 181 | 55.358 | 44.850 | 2.862 | 1.00 | 49.87 | 8 | O |
| ATOM | 2449 | OW0 | WAT W | 182 | 79.585 | 32.525 | 13.716 | 1.00 | 66.67 | 8 | O |
| ATOM | 2451 | OW0 | WAT W | 184 | 64.518 | 23.932 | 15.951 | 1.00 | 44.99 | 8 | O |
| ATOM | 2453 | OW0 | WAT W | 186 | 51.154 | 31.181 | 36.820 | 1.00 | 61.70 | 8 | O |
| ATOM | 2454 | OW0 | WAT W | 187 | 51.013 | 15.442 | 17.418 | 1.00 | 45.79 | 8 | O |
| ATOM | 2455 | OW0 | WAT W | 188 | 39.771 | 41.527 | 18.524 | 1.00 | 49.74 | 8 | O |
| ATOM | 2456 | OW0 | WAT W | 189 | 72.438 | 9.840 | −7.353 | 1.00 | 40.07 | 8 | O |
| ATOM | 2457 | OW0 | WAT W | 190 | 46.902 | 14.835 | 15.177 | 1.00 | 46.39 | 8 | O |
| ATOM | 2458 | OW0 | WAT W | 191 | 57.675 | 20.757 | 10.803 | 1.00 | 50.78 | 8 | O |
| ATOM | 2459 | OW0 | WAT W | 192 | 69.479 | 12.450 | 0.686 | 1.00 | 31.78 | 8 | O |
| ATOM | 2460 | OW0 | WAT W | 193 | 61.498 | 42.666 | 4.391 | 1.00 | 49.75 | 8 | O |
| ATOM | 2461 | OW0 | WAT W | 194 | 40.573 | 23.622 | −4.508 | 1.00 | 49.50 | 8 | O |
| ATOM | 2462 | OW0 | WAT W | 195 | 63.239 | 22.036 | 17.365 | 1.00 | 60.76 | 8 | O |
| ATOM | 2463 | OW0 | WAT W | 196 | 50.230 | 38.893 | −1.160 | 1.00 | 78.76 | 8 | O |
| ATOM | 2464 | OW0 | WAT W | 197 | 71.530 | 40.221 | 22.322 | 1.00 | 55.72 | 8 | O |
| ATOM | 2465 | OW0 | WAT W | 198 | 75.489 | 8.467 | 5.178 | 1.00 | 65.56 | 8 | O |
| ATOM | 2466 | OW0 | WAT W | 199 | 45.696 | 45.482 | 25.583 | 1.00 | 49.39 | 8 | O |
| ATOM | 2467 | OW0 | WAT W | 200 | 37.019 | 43.489 | 24.116 | 1.00 | 57.82 | 8 | O |
| ATOM | 2468 | OW0 | WAT W | 201 | 40.688 | 15.783 | 8.184 | 1.00 | 48.78 | 8 | O |
| ATOM | 2470 | OW0 | WAT W | 203 | 62.458 | 2.230 | 1.187 | 1.00 | 40.73 | 8 | O |
| ATOM | 2471 | OW0 | WAT W | 204 | 52.440 | 32.379 | 2.873 | 1.00 | 34.91 | 8 | O |
| ATOM | 2472 | OW0 | WAT W | 205 | 57.846 | 25.412 | 19.786 | 1.00 | 38.28 | 8 | O |
| ATOM | 2473 | OW0 | WAT W | 206 | 67.361 | 33.501 | 10.026 | 1.00 | 49.47 | 8 | O |
| ATOM | 2474 | OW0 | WAT W | 207 | 67.350 | 13.501 | 1.221 | 1.00 | 45.88 | 8 | O |
| ATOM | 2475 | OW0 | WAT W | 208 | 53.533 | 40.792 | 1.526 | 1.00 | 46.36 | 8 | O |
| ATOM | 2476 | OW0 | WAT W | 209 | 41.314 | 31.067 | 18.590 | 1.00 | 50.24 | 8 | O |
| ATOM | 2477 | OW0 | WAT W | 210 | 54.131 | 15.501 | −14.694 | 1.00 | 42.77 | 8 | O |
| ATOM | 2478 | OW0 | WAT W | 211 | 60.554 | 17.337 | 17.933 | 1.00 | 53.34 | 8 | O |
| ATOM | 2479 | OW0 | WAT W | 212 | 67.523 | 14.077 | 19.660 | 1.00 | 50.05 | 8 | O |
| ATOM | 2480 | OW0 | WAT W | 213 | 40.813 | 42.013 | 26.973 | 1.00 | 54.37 | 8 | O |
| ATOM | 2481 | OW0 | WAT W | 214 | 66.196 | 27.079 | 11.041 | 1.00 | 46.02 | 8 | O |
| ATOM | 2482 | OW0 | WAT W | 215 | 55.181 | 36.388 | −13.734 | 1.00 | 53.91 | 8 | O |
| ATOM | 2483 | OW0 | WAT W | 216 | 48.057 | 35.571 | 4.408 | 1.00 | 42.80 | 8 | O |
| ATOM | 2484 | OW0 | WAT W | 217 | 52.470 | 1.706 | 5.097 | 1.00 | 45.08 | 8 | O |
| ATOM | 2485 | OW0 | WAT W | 218 | 38.558 | 17.450 | −0.591 | 1.00 | 52.74 | 8 | O |
| ATOM | 2486 | OW0 | WAT W | 219 | 48.977 | 22.868 | 15.771 | 1.00 | 53.86 | 8 | O |
| ATOM | 2487 | OW0 | WAT W | 220 | 59.800 | 7.098 | 18.579 | 1.00 | 45.62 | 8 | O |
| ATOM | 2488 | OW0 | WAT W | 221 | 58.023 | 35.899 | −6.089 | 1.00 | 48.40 | 8 | O |
| ATOM | 2489 | OW0 | WAT W | 222 | 61.021 | 27.781 | 14.513 | 1.00 | 46.58 | 8 | O |
| ATOM | 2490 | OW0 | WAT W | 223 | 47.444 | 22.887 | 12.782 | 1.00 | 56.07 | 8 | O |
| ATOM | 2491 | OW0 | WAT W | 224 | 50.705 | 37.582 | 1.322 | 1.00 | 48.64 | 8 | O |
| ATOM | 2492 | OW0 | WAT W | 225 | 66.183 | 26.023 | 30.771 | 1.00 | 48.27 | 8 | O |
| ATOM | 2493 | OW0 | WAT W | 226 | 48.692 | 34.988 | 33.176 | 1.00 | 52.17 | 8 | O |
| ATOM | 2494 | OW0 | WAT W | 227 | 42.523 | 6.385 | −1.745 | 1.00 | 51.24 | 8 | O |
| ATOM | 2495 | OW0 | WAT W | 228 | 68.475 | 30.124 | 0.782 | 1.00 | 55.52 | 8 | O |
| ATOM | 2496 | OW0 | WAT W | 229 | 46.706 | 18.388 | 15.459 | 1.00 | 57.98 | 8 | O |
| ATOM | 2497 | OW0 | WAT W | 230 | 35.641 | 36.427 | 9.282 | 1.00 | 55.60 | 8 | O |
| ATOM | 2498 | OW0 | WAT W | 231 | 72.587 | 4.072 | 9.251 | 1.00 | 54.93 | 8 | O |
| ATOM | 2499 | OW0 | WAT W | 232 | 41.618 | 10.474 | 1.751 | 1.00 | 54.38 | 8 | O |
| ATOM | 2500 | OW0 | WAT W | 233 | 57.543 | 50.282 | 15.470 | 1.00 | 58.67 | 8 | O |
| ATOM | 2501 | OW0 | WAT W | 234 | 44.867 | 42.335 | 0.710 | 1.00 | 57.80 | 8 | O |
| ATOM | 2502 | OW0 | WAT W | 235 | 57.128 | 25.399 | 15.459 | 1.00 | 58.90 | 8 | O |
| ATOM | 2503 | OW0 | WAT W | 236 | 68.901 | 21.881 | −0.580 | 1.00 | 53.19 | 8 | O |
| ATOM | 2504 | OW0 | WAT W | 237 | 53.588 | 2.879 | −6.515 | 1.00 | 59.80 | 8 | O |
| ATOM | 2505 | OW0 | WAT W | 238 | 44.473 | 7.462 | −3.404 | 1.00 | 53.96 | 8 | O |
| ATOM | 2506 | OW0 | WAT W | 239 | 74.820 | 10.403 | 6.976 | 1.00 | 54.01 | 8 | O |
| ATOM | 2507 | OW0 | WAT W | 240 | 56.842 | 9.771 | −13.835 | 1.00 | 55.94 | 8 | O |
| ATOM | 2508 | OW0 | WAT W | 241 | 38.570 | 22.438 | 2.343 | 1.00 | 57.94 | 8 | O |
| ATOM | 2509 | OW0 | WAT W | 242 | 65.608 | −0.573 | 10.932 | 1.00 | 56.24 | 8 | O |
| ATOM | 2510 | OW0 | WAT W | 243 | 67.275 | 33.989 | 6.398 | 1.00 | 59.57 | 8 | O |
| ATOM | 2511 | OW0 | WAT W | 244 | 38.494 | 27.454 | 26.177 | 1.00 | 56.65 | 8 | O |
| ATOM | 2512 | OW0 | WAT W | 245 | 79.415 | 38.544 | 19.049 | 1.00 | 55.94 | 8 | O |
| ATOM | 2513 | OW0 | WAT W | 246 | 41.428 | 43.667 | 23.851 | 1.00 | 48.67 | 8 | O |
| ATOM | 2514 | OW0 | WAT W | 247 | 45.669 | 8.862 | −7.019 | 1.00 | 54.07 | 8 | O |
| ATOM | 2515 | OW0 | WAT W | 248 | 57.584 | 17.844 | 11.964 | 1.00 | 57.11 | 8 | O |
| ATOM | 2516 | OW0 | WAT W | 249 | 63.334 | 28.290 | −11.159 | 1.00 | 52.99 | 8 | O |
| ATOM | 2517 | OW0 | WAT W | 250 | 50.101 | 5.187 | 11.030 | 1.00 | 58.96 | 8 | O |
| ATOM | 2518 | OW0 | WAT W | 251 | 55.531 | 46.308 | 9.306 | 1.00 | 49.04 | 8 | O |
| ATOM | 2519 | OW0 | WAT W | 252 | 73.201 | 1.607 | −5.272 | 1.00 | 58.26 | 8 | O |
| ATOM | 2520 | OW0 | WAT W | 253 | 71.038 | 8.861 | −13.814 | 1.00 | 56.92 | 8 | O |
| ATOM | 2521 | OW0 | WAT W | 254 | 65.195 | 4.663 | −5.993 | 1.00 | 57.84 | 8 | O |
| ATOM | 2522 | OW0 | WAT W | 255 | 69.290 | 21.864 | 3.630 | 1.00 | 60.04 | 8 | O |
| ATOM | 2523 | OW0 | WAT W | 256 | 60.282 | 28.198 | 33.064 | 1.00 | 52.37 | 8 | O |
| ATOM | 2524 | OW0 | WAT W | 257 | 54.999 | 26.806 | 34.737 | 1.00 | 59.86 | 8 | O |
| ATOM | 2525 | OW0 | WAT W | 258 | 69.644 | 21.889 | −3.484 | 1.00 | 55.95 | 8 | O |
| ATOM | 2526 | OW0 | WAT W | 259 | 41.512 | 12.680 | 8.317 | 1.00 | 57.27 | 8 | O |
| ATOM | 2527 | OW0 | WAT W | 260 | 38.620 | 20.840 | −3.595 | 1.00 | 60.58 | 8 | O |
| ATOM | 2528 | OW0 | WAT W | 261 | 42.796 | 29.152 | −3.495 | 1.00 | 57.83 | 8 | O |
| ATOM | 2529 | OW0 | WAT W | 262 | 43.194 | 45.599 | 12.173 | 1.00 | 60.81 | 8 | O |
| ATOM | 2530 | OW0 | WAT W | 263 | 43.689 | 32.979 | −2.364 | 1.00 | 61.53 | 8 | O |
| ATOM | 2531 | OW0 | WAT W | 264 | 55.096 | 12.692 | −14.255 | 1.00 | 56.51 | 8 | O |
| ATOM | 2532 | OW0 | WAT W | 265 | 66.719 | 21.323 | 27.411 | 1.00 | 60.18 | 8 | O |
| ATOM | 2533 | OW0 | WAT W | 266 | 48.925 | 24.154 | −13.326 | 1.00 | 59.99 | 8 | O |
| ATOM | 2534 | OW0 | WAT W | 267 | 50.424 | 34.508 | −0.963 | 1.00 | 58.47 | 8 | O |
| ATOM | 2535 | OW0 | WAT W | 268 | 69.496 | 0.033 | 1.507 | 1.00 | 60.26 | 8 | O |
| ATOM | 2536 | OW0 | WAT W | 269 | 57.574 | 46.046 | 3.695 | 1.00 | 59.77 | 8 | O |
| ATOM | 2537 | OW0 | WAT W | 270 | 57.679 | 42.153 | 24.392 | 1.00 | 59.49 | 8 | O |
| ATOM | 2538 | OW0 | WAT W | 271 | 46.096 | 35.339 | 31.158 | 1.00 | 60.76 | 8 | O |
| ATOM | 2539 | OW0 | WAT W | 272 | 38.455 | 35.588 | 24.574 | 1.00 | 60.34 | 8 | O |

We claim:

1. A crystal of ketopantoate reductase having a tetragonal space group P4₁2₁2 with unit cell dimensions of a=103.7±0.2, b=103.7±0.2, c=55.7±0.2 Å.

2. A crystal of ketopantoate reductase having a resolution better than 2 Å.

3. A crystal of ketopantoate reductase having the structure defined by the co-ordinates of Table 1.

4. A method for crystallizing a selenium atom KPR derivative which comprises producing KPR by recombinant production in a bacterial host in the presence of selenomethionine, recovering a selenium atom KPR derivative from the host and growing crystals from the recovered selenium atom KPR derivative.

5. A computer-based method of rational drug design which comprises:
providing the structure of the KPR as defined by the coordinates of Table 1;
providing the structure of a candidate modulator molecule; and
fitting the structure of candidate to the structure of the KPR of Table 1.

6. A computer-based method of rational drug design which comprises:
providing the coordinates of at least two atoms of Table 1 of the KPR (“selected coordinates”;
providing the structure of a candidate modulator molecule; and
fitting the structure of candidate to the selected coordinates of the KPR.

7. A computer-based method of rational drug design which comprises:
providing the coordinates of at least a sub-domain of the KPR;
providing the structure of a candidate modulator molecule; and
fitting the structure of the candidate to the coordinates of the KPR sub-domain provided.

8. The method of claim 5, 6 or 7 which further comprises the steps of:
obtaining or synthesising the candidate modulator; and
contacting the candidate modulator with KPR to determine the ability of the candidate modulator to interact with KPR.

9. The method of claim 5, 6 or 7 which further comprises the steps of:
obtaining or synthesising said candidate modulator;
forming a complex of KPR and said potential modulator; and
analysing said complex by X-ray crystallography to determine the ability of said candidate modulator to interact with KPR.

10. A compound having a chemical structure selected using the method of claim 5, 6 or 7, said compound being an inhibitor of KPR.

11. A computer system, intended to generate structures and/or perform rational drug design for KPR or complexes of KPR with a potential modulator, the systems containing either (a) atomic coordinate data according to Table 1, said data defining the three-dimensional structure of KPR or at least one sub-domain thereof, or (b) structure factor data for KPR, said structure factor data being derivable from the atomic coordinate data of Table 1.

12. A computer readable media with either (a) atomic coordinate data according to Table 1 recorded thereon, said data defining the three-dimensional structure of KPR, at least one atom or at least one sub-domain thereof, or (b) structure factor data for KPR recorded thereon, the structure factor data being derivable from the atomic coordinate data of Table 1.
